(19) United States
(12) Patent Application Publication
(10) Pub. No.: US 2006/0223837 A1
(43) Pub. Date: Oct. 5, 2006

(54) Biaryl derived amide modulators of vanilloid VR1 receptor

(76) Inventors: Ellen Codd, Blue Bell, PA (US); Scott L. Dax, Landenberg, PA (US); Christopher Flores, Lansdale, PA (US); Michelle Jetter, Norristown, PA (US); Mark Youngman, Warminster, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/387,894

(22) Filed: Mar. 23, 2006

Related U.S. Application Data
(60) Provisional application No. 60/665,028, filed on Mar. 24, 2005.

(51) Int. Cl. A61K 31/506; A61K 31/4709; A61K 31/444; C07D 401/02

(52) U.S. Cl. 514/275; 514/310; 514/332; 544/331; 546/146; 546/255; 546/256

(57) ABSTRACT

The invention is directed to novel vanilloid receptor type 1 (VR1) ligands. More specifically, the invention relates to novel biaryl-derived amides that are potent antagonists or agonists of VR1. Pharmaceutical and veterinary compositions and methods of treating mild to severe pain and various diseases using compounds of the invention are also described.

### CROSS-REFERENCE TO RELATED APPLICATIONS

This Application claims priority to U.S. Provisional Patent Application No. 60/665,028, filed Mar. 24, 2005, which is hereby incorporated by reference in its entirety.

### STATEMENT REGARDING FEDERALLY SPONSORED RESEARCH OR DEVELOPMENT

The research and development of the invention described below was not federally sponsored.

### FIELD OF THE INVENTION

This invention is directed to novel vanilloid receptor type 1 (VR1) ligands. More particularly, this invention relates to novel biaryl-derived amides that are potent antagonists or agonists of VR1 and exhibit activity in animal models of hyperalgesia and colitis, and are useful for the treatment and prevention of human pain conditions including arthritis, and for the treatment of irritable-bowel syndrome and associated conditions.

### BACKGROUND OF THE INVENTION

Noxious chemical, thermal and mechanical stimuli excite peripheral nerve endings of small diameter sensory neurons (nociceptors) deriving from sensory ganglia (e.g., dorsal root, nodose and trigeminal ganglia) and initiate signals that are perceived as pain. Such nociceptors neurons are crucial for the detection of harmful or potentially harmful stimuli (e.g., noxious heat, acidosis, and/or stretch) that arise from changes in the extracellular environment during inflammatory, ischemic or otherwise traumatic conditions and that cause or have the potential to cause tissue damage (Wall, P. D., and Melzack, R., Textbook of Pain, 1994, New York: Churchill Livingstone). Nociceptors transduce noxious stimuli into membrane depolarization that leads to an action potential, its subsequent conduction to the CNS, and ultimately to the perception of pain, discomfort, etc. as well as to certain responses thereto. At the molecular level, nociception is carried out by ion channels and/or receptors. Plant-derived vanilloid compounds (e.g., capsaicin and resiniferatoxin) are known to selectively depolarize nociceptors and elicit sensations of burning pain—the sensation that is typically obtained by capsaicin-containing hot chili peppers. Therefore, capsaicin mimics the action of physiological/endogenous stimuli that activate the “nociceptive pathway”. Recent advances in pain biology have identified a receptor, called VR1 (a.k.a. capsaicin receptor or TRPV1) for vanilloids, protons and noxious heat. Because nociceptors are involved with unwanted pain and inflammatory conditions in human beings and animals, modulation of their function is a validated strategy palliative and other therapies.

Compounds that are modulators (competitive and non-competitive agonists or antagonists [with respect to capsaicin and/or its recognition site] and allosteric modulators) at the vanilloid type 1 receptor (VR1) have broad therapeutic potential, as demonstrated by the clinical usefulness of marketed, VR1 -targeted pharmaceutical agents or the efficacy of VR1 modulators in animal models of disease. Furthermore, it is recognized that agonist modulators of VR1 may possess clinical utility deriving from their agonist properties, per se, and/or from their ability to produce an agonist-mediated desensitization, which would indirectly manifest as a functional antagonism. Similarly, antagonist modulators could exhibit direct antagonist (competitive or non-competitive) properties and/or indirect antagonist properties via the aforementioned desensitization mechanism. It is further recognized that postitive and negative allosteric modulators may produce any or all of the aforementioned functional consequences and, as such, may also have clinical utility. Accordingly, this invention is directed to each of these types of modulators.

The effective use of VR1 agonists has been demonstrated in inflammatory, neuropathic, and visceral pain states. In an experimental human pain model, dermal capsaicin pretreatment reduced the pain caused by intradermal injection of an acidic solution (Bianco, E. D.; Geppetti, P.; Zippi, P.; Isolani, D.; Magini, B.; Cappugi, P. Brit J of Clin Pharmacol 1996, 41, 1-6), suggesting the benefit of VR1 agonists in the treatment of inflammatory pain. A particular role for VR1 agonists has been shown in inflammation and inflammatory pain: for example, resiniferatoxin prevented inflammatory hypersensitivity and edema induction by carrageenan (Kissin, I.; Bright, C. A.; Bradley, E. L., Jr. Anesth Analg 2002, 94,1253-1258). Additionally, capsaicin-containing creams (for example, Axcain and Lidocare) are marketed for dermal relief of pain related to diabetic neuropathy and postherpetic neuralgia, indicative of the usefulness of VR1 agonists in the treatment of neuropathic pain states. Furthermore, such creams have been shown to reduce postsurgical neuropathic pain (Ellison, N., Loprinzi, C. L., Kugler, J., Hatfield, A. K., Miser, A., Sloan, J. A., Wender, D. B., Rowland, K. M., Molina, R., Cascino, T. L., Vukov, A. M., Dhaliwal, H. S. and Ghosh, C. J. Clin. Oncol. 15:2974-2980,1997). And in cancer patients, capsaicin contained in a taffy vehicle, was shown to substantially reduce oral mucositis pain caused by chemotherapy and radiation therapy (Berger, A., Henderson, M., Naadoolman, W., Duffy, V., Cooper, D., Saberski, L. and Bartoshuk, L. J. Pain Sympt Mgmt 10:243-248, 1995.

VR1 also plays a role in the physiology of bladder emptying. VR1 is expressed by bladder sensory neurons, where they modulate bladder responsivity to liquid filling. The VR1 agonist resiniferatoxin desensitized bladder afferents in a dose-dependent manner (Avelino, A.; Cruz, F.; Coimbra, A. Eur. J Pharmacol. 1999, 378, 17-22), supporting its usefulness for the treatment of overactive bladder (Chancellor, M. B.; De Groat, W. C. J. Urol. (Baltimore) 1999, 162, 3-11). Indeed, intravesical administration of capsaicin or resiniferatoxin inhibited bladder contraction in both normal and spinal cord injured rats (Komiyama, I.; Igawa, Y.; Ishizuka, O.; Nishizawa, O.; Andersson, K.-E. J. Urol. (Baltimore) 1999, 161, 314-319), indicative of the usefulness of VR1 agonists in nerve-injured incontinent patients. The effectiveness of capsaicin or resiniferatoxin treatment of incontinence in spinal cord injured patients was confirmed in a clinical study (de Seze, M.; Wiart, L.; de Seze, M.-P.; Soyeur, L.; Dosque, J.-P.; Blajezewski, S.; Moore, N.; Brochet, B.; Mazaux, J.-M.; Barat, M.; Joseph, P.-A. Journal of Urology (Hagerstown, Md., United States) 2003, 171, 251-255).

VR1 agonists also modulate body temperature and fever. In ferret, rat and mouse, administration of resiniferatoxin induced marked hypothermia (Woods, A. J.; Stock, M. J.; Gupta, A. N.; Wong, T. T. L.; Andrews, P. L. R. Eur. J. Pharmacol. 1994, 264,125-133). Additionally, phase I of LPS (lipopolysaccharide)-induced fever did not occur in animals desensitized with low intraperitoneal doses of capsaicin (Romanovsky, A. A. Frontiers in Bioscience 2004, 9, 494-504).

The effectiveness of VR1 agonists in the reduction of elevated blood pressure is suggested by capsaicin reduction in blood pressure in SHR and WKY rats (Li, J.; Kaminski, N. E.; Wang, D. H. Hypertension 2003, 41, 757-762.). Capsaicin was also gastroprotective with respect to gastric antral ulcers (Yamamoto, H.; Horie, S.; Uchida, M.; Tsuchiya, S.; Murayama, T.; Watanabe, K. Eur. J Pharmacol. 2001, 432, 203-210).

VR1 antagonists also may be useful in the treatment of inflammatory, neuropathic and visceral pain. For example, the therapeutic utility of VR1 antagonists has been demonstrated in visceral inflammatory conditions. VR1 is elevated in colonic nerve fibers in patients with inflammatory bowel disease, and VR1 antagonists relieved pain and dysmotility (Yiangou, Y.; Facer, P.; Dyer, N. H.; Chan, C. L.; Knowles, C.; Williams, N. S.; Anand, P. Lancet 2001, 357, 1338-1339). Intestinal inflammation induced by toxin A or dextran sulfate sodium in rodents was attenuated by VR1 antagonists (McVey, D. C.; Schmid, P. C.; Schmid, H. H. O.; Vigna, S. R. J. Pharmacol. Exp. Ther. 2003, 304, 713-722). In addition, a synthetic VR1 antagonist reduced colitis disease scores at several important endpoints, including macroscopic damage, microscopic epithelial damage, myeloperoxidase levels, and diarrhea scores, strongly supporting the therapeutic use of VR1 antagonists in inflammatory bowel diseases (Kimball, E. S.; Wallace, N. H.; Schneider, C. R.; D'Andrea, M. R.; Hornby, P. J. Neurogasteroenterology 2004, 16, 811-818). The VR1 antagonists capsazepine and BCTC reversed mechanical hyperalgesia in models of inflammatory and neuropathic pain in guinea pigs (Walker, K. M.; Urban, L.; Medhurst, S. J.; Patel, S.; Panesar, M.; Fox, A. J.; Mcintyre, P. J. Pharmacol. Exp. Ther. 2003, 304, 56-62) and rats (Pomonis, J. D.; Harrison, J. E.; Mark, L.; Bristol, D. R.; Valenzano, K. J.; Walker, K. J. Pharmacol. Exp. Ther. 2003, 306, 387-393).

LPS-induced fever was attenuated in VR1 knock out mice (Lida, T.; Shimizu, I.; Nealen, M. L.; Campbell, A.; Caterina, M. Neurosci. Lett. 2005, 378, 28-33). VR1 agonist-induced rises in core body temperature were suppressed by capsazepine, indicative of the usefulness of VR1 antagonists in the treatment of pyresis (Ohnluki, K.; Haramizu, S.; Watanabe, T.; Yazawa, S.; Fushiki, T. J. Nutr. Sci. Vitaminol. (Tokyo) 2001, 47, 295-298). The therapeutic potential of VR1 antagonists in inflammatory bronchial conditions is demonstrated by the finding that they antagonize capsaicin- and acid-induced bronchoconstriction (Nault, M. A.; Vincent, S. G.; Fisher, J. T. J. Physiol. 1999, 515, 567-578). Related findings demonstrate that the VR1 antagonist capsazepine attenuates anandamide-induced cough in guinea pigs (Jia, Y.; McLeod, R. L.; Wang, X.; Parra, L. E.; Egan, R. W.; Hey, J. A. Brit. J. Pharmacol. 2002, 137, 831-836).

The VR1 antagonist capsazepine was demonstrated to significantly reduce anxiety-like behaviors in rats using the elevated plus maze (Kasckow, J. W.; Mulchahey, J. J.; Geracioti, T. D. Jr. Progress in Neuro-Psychopharmacol. and Biological Psychiatry 2004, 28, 291-295). Thus, VR1 antagonists may have utility in the treatment of anxiety, panic disorders, phobias or other non-adaptive stress responses.

United States Patent application US 2003/0135055 discloses aminosulfonylbiphenyl derivatives as inhibitors of factors Xa and VIIa. This application, however, does not disclose or suggest the compounds, compositions or methods of the present invention.

United States Patent application US 2003/0065176 discloses aryl-amidine derivatives as inhibitors of factor Xa. This application, however, does not disclose or suggest the compounds, compositions, or methods of the present invention.

PCT application W02004/056774 discloses substituted biphenyl-4-carboxylic acid arylamide analogues as capsaicin receptor modulators. This application, however, does not disclose or suggest the compounds, compositions, or methods of the present invention.

United States Patent application US 2004/0087798 discloses novel amide compounds as antagonists of 5-hydroxytryptamine (5-HT). This application, however, does not disclose or suggest the compounds, compositions, or methods of the present invention.

Thus, there is a need for potent modulators of VR, and in particular, for novel biaryl-derived amides that exhibit potent binding affinity for the human and rat VR1 ion channel. There is also a need for novel biaryl-derived amides that act as potent functional antagonists and/or agonists of the human and rat VR1 ion channel. Finally, there is a need for novel biaryl-derived amides that bind with high affinity to VR1 and also act as potent functional antagonists of the human and rat VR1 ion channel.

### SUMMARY OF THE INVENTION

The present invention is directed to a compound of Formula (I): 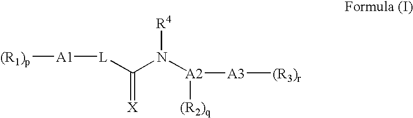
wherein:
A1 is phenyl, naphthalenyl, pyridinyl, or thienyl;
R₁ is independently hydroxy; halogen; C₁₋₈alkanyl optionally substituted with one or more substituents independently selected from the group consisting of halogen, trifluoromethylsulfonyl, trifluoromethylsulfinyl, fluorinated alkanyl, and C₁₋₈alkanyloxy; C₁₋₈alkanyloxy optionally substituted with one or more substituents independently selected from the group consisting of halogen, fluorinated alkanyl, and C₁₋₈alkanyloxy; fluorinated alkanyloxy; fluorinated alkanyl; C₁₋₈alkanylthio optionally substituted with one or more substituents independently selected from the group consisting of halogen, fluorinated alkanyl and C₁₋₈alkanyloxy; C₁₋₈alkanylsulfinyl optionally substituted with one or more substituents independently selected from the group consisting of halogen and C₁₋₈alkanyloxy; C₁₋₈alkanylsulfonyl optionally substituted with one or more substituents independently selected from the group consisting of halogen and C₁₋₈alkanyloxy; C₃₋₈cycloalkanyl; C₃₋₈cycloalkanyloxy; nitro; amino; C₁₋₈alkanylamino; C₁₋₈dialkanylamino; C₃₋₈cycloalkanylamino; N—C₁₋₈alkanyl-N—C₃₋₈cycloalkanylamino, cyano; carboxy; C₁₋₇alkanyloxycarbonyl; C₁₋₇alkanylcarbonyloxy; C₁₋₇alkanylaminocarbonyl; C₁₋₇alkanylcarbonylamino; diC₁₋₇alkanylaminocarbonyl; formyl; aminosulfonyl; C₁₋₈alkanylaminosulfonyl; di(C₁₋₈)alkanylaminosulfonyl; or cyano;
p is 0, 1 or 2;
L is C₂₋₈alkandiyl, C₂₋₈alkendiyl, or C₂₋₈alkyndiyl and L is optionally substituted with a substituent selected from the group consisting of C₁₋₈alkanyl, C₃₋₈cycloalkanyl and phenyl optionally substituted with one to three substituents independently selected from the group consisting of C₁₋₈alkanyl, C₁₋₈alkanyloxy, halogen, hydroxy, fluorinated alkanyl, fluorinated alkanyloxy, amino, di(C₁₋₃)alkanylamino, and C₁₋₃alkanylamino;
X is O or S;
A2 is selected from the group consisting of phenyl, thien-2-yl, naphthalenyl, pyridinyl, pyrimidinyl, pyrazinyl, quinolinyl, and isoquinolinyl;
R₂ is independently selected from the group consisting of C₁₋₈alkanyl, C₁₋₈alkanyloxy, hydroxy(C₁₋₈)alkanyl, fluorinated C₁₋₈alkanyl, hydroxyl, halogen, carboxy, C₁₋₈alkanyloxycarbonyl, and aminocarbonyl;
q is 0, 1, or 2;
A3 is selected from the group consisting of phenyl, thienyl, naphthalenyl, pyridinyl, pyrimidinyl, pyrazinyl, imidazolyl, oxazolyl, 4,5-dihydro-oxazolyl, pyrazolyl, dihydro-pyrazolyl, thiazolyl, thiadiazolyl, triazolyl, tetrazolyl, quinolinyl, isoquinolinyl, oxathiadiazolyl, benzimidazolyl, tetrahydrobenzimidazolyl, tetrahydroindazolyl, oxathiadiazolyl 2-oxide, oxadiazolyl, tetrahydroisoquinolinyl, tetrahydroquinolinyl, and indolyl; such that when A3 is tetrahydrobenzimidazolyl, tetrahydroindazolyl, oxathiadiazolyl 2-oxide, tetrahydroisoquinolinyl, or tetrahydroquinolinyl, then r is O;
R₃ is independently selected from the group consisting of hydroxy; halogen; C₁₋₈alkanyl; hydroxy(C₁₋₈)alkanyl; C₁₋₈alkanyloxy optionally substituted with amino, C₁₋₈alkanylamino, or C₁₋₈dialkanylamino; fluorinated alkanyloxy; fluorinated alkanyl; C₁₋₈alkanylthio; nitro; amino; C₁₋₈alkanylamino; C₁₋₈dialkanylamino; C₃₋₈cycloalkanylamino; cyano; aminosulfonyl; carboxy; C₁₋₈alkanylsuIfonylamino; aminocarbonyl; C₁₋₈alkanyloxycarbonyl; C₁₋₄alkanyloxycarbonylamino; C₁₋₈alkanylaminocarbonyl; C₁₋₈alkanylcarbonylamino; diC₁₋₈alkanylaminocarbonyl; oxo when A3 is dihydro-pyrazolyl; and formyl; and wherein the C₁₋₈alkanyl group of any C₁₋₈alkanylamino and C₁₋₈dialkanylamino containing substituent of R₃ is optionally substituted with hydroxy;
r is 0, 1, or 2;
R₄ is hydrogen or C₁₋₈alkyl;
provided that a compound of Formula 1 is other than
a compound wherein p is 1, R₁ is 4-trifluoromethylsulfonyl, A1 is phenyl, L is trans —CH═CH—, X is O, A2 is pyridin-3-yl, q is 1, R₂ is 5-methyl, A3 is imidazol-1-yl, r is 2, R₃ is 4,5-dichloro, and R₄ is hydrogen;
a compound wherein p is 1, R₁ is 4-trifluoromethyl, A1 is phenyl, L is —(CH₂)₂—, X is O, A2 is phenyl, q is 1, R₂ is 3-trifluoromethyl, A3 is pyrazol-1-yl, r is 2, R₃ is 3,5-dimethyl, and R₄ is hydrogen;
a compound wherein p is 1, R₁ is 4-trifluoromethyl, A1 is phenyl, L is —(CH₂)₂—, X is O, A2 is phenyl, q is 1, R₂ is 3-trifluoromethyl, A3 is imidazol-1-yl, r is 1, R₃ is 4-carboxy, and R₄ is hydrogen; and
enantiomers, diastereomers, tautomers, solvates, and pharmaceutically acceptable salts thereof.

Finally, the present invention is directed to pharmaceutical compositions containing compounds of Formula (I), as well as to methods of treatment of diseases and conditions by administration of these compositions, and also to pharmaceutical kits containing them.

### DETAILED DESCRIPTION OF THE INVENTION

As used herein, the following underlined terms are intended to have the following meanings:

“C_{a-b}” (where a and b are integers) refers to a radical containing from a to b carbon atoms inclusive. For example, C₁₋₃ denotes a radical containing 1, 2 or 3 carbon atoms.

“Fluorinated alkyl” refers to a saturated branched or straight chain hydrocarbon radical derived by removal of 1 hydrogen atom from the parent alkane; the parent alkane contains from 1 to 6 carbon atoms with 1 or more hydrogen atoms substituted with fluorine atoms up to and including substitution of all hydrogen atoms with fluorine. Preferred fluorinated alkyls include trifluoromethyl substituted alkyls and perfluorinated alkyls; more preferred fluorinated alkyls include trifluoromethyl, perfluoroethyl, 2,2,2-trifluoroethyl, perfluoropropyl, 3,3,3-trifluoroprop-1-yl, 3,3,3-trifluoroprop-2-yl, 1,1,1,3,3,3-hexafluoroprop-2-yl; a particularly preferred fluorinated alkyl, is trifluoromethyl.

“Fluorinated alkanyloxy” refers to a radical derived from a fluorinated alkyl, radical attached to an oxygen atom with the oxygen atom having one open valence for attachment to a parent structure.

“Alkyl:” refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Where specific levels of saturation are intended, the nomenclature “alkanyl”, “alkenyl” and/or “alkynyl” is used, as defined below. In preferred embodiments, the alkyl groups are (C₁₋₈) alkyl, with (C₁₋₃) being particularly preferred.]

“Alkanyl:” refers to a saturated branched, straight-chain or cyclic monovalent hydrocarbon radical derived by the removal of one hydrogen atom from a single carbon atom of a parent alkane. Typical alkanyl groups include, but are not limited to, methanyl; ethanyl; propanyls such as propan-1-yl, propan-2-yl, cyclopropan-1-yl, etc.; butyanyls such as butan-1-yl, butan-2-yl, 2-methyl-propan-1-yl, 2-methyl-propan-2-yl, cyclobutan-1-yl, etc.; and the like. In preferred embodiments, the alkanyl groups are (C₁₋₈) alkanyl, with (C₁₋₃) being particularly preferred.

“Alkenyl:” refers to an unsaturated branched, straight-chain or cyclic monovalent hydrocarbon radical having at least one carbon-carbon double bond derived by the removal of one hydrogen atom from a single carbon atom of a parent alkene. The radical may be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. In preferred embodiments, the alkenyl group is (C₂₋₈) alkenyl, with (C₂₋₃) being particularly preferred.

“Alkynyl:” refers to an unsaturated branched, straight-chain or cyclic monovalent hydrocarbon radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. In preferred embodiments, the alkynyl group is (C₂₋₈) alkynyl, with (C₂₋₃) being particularly preferred.

“Alkyldiyl:” refers to a saturated or unsaturated, branched, straight-chain or cyclic divalent hydrocarbon radical derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers can form bonds with the same or different atoms. Typical alkyldiyls include, but are not limited to methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methylprop-2-en-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkandiyl, alkendiyl and/or alkyndiyl is used. In preferred embodiments, the alkyldiyl group is (C₁₋₈) alkyldiyl, with (C₁₋₈) being particularly preferred. Also preferred are saturated acyclic alkandiyl radicals in which the radical centers are at the terminal carbons, e.g., methandiyl; ethan-1,2-diyl; propan-1,3-diyl; butan-1,4-diyl; and the like (also referred to as alkylenos, as defined infra).

“Vic Alkyldiyl:” refers to a saturated or unsaturated, branched, straight-chain or cyclic hydrocarbon radical having two adjacent monovalent radical centers derived by the removal of one hydrogen atom from each of two adjacent carbon atoms of a parent alkane, alkene or alkyne. The two monovalent radical centers can form bonds with the same or different atom(s). Typical vic alkyldiyls include, but are not limited to vic ethyldiyls such as ethan-1,2-diyl, ethen-1,2-diyl; vic propyldiyls such as propan-1,2-diyl, cyclopropan-1,2-diyl, prop-1-en-1,2-diyl, prop-2-en-1,2-diyl, cycloprop-1-en-1,2-diyl, etc.; vic butyldiyls such as butan-1,2-diyl, 2-methyl-propan-1,2-diyl, cyclobutan-1,2-diyl, but-1-en-1,2-diyl, cyclobut-1-en-1,2-diyl, buta-1,3-dien-1,2-diyl, cyclobuta-1,3-dien-1,2-diyl, but-3-yn-1,2-diyl, etc.; and the like. Where specific levels of saturation are intended, the nomenclature vic alkandiyl, vic alkendiyl and/or vic alkyndiyl is used. In preferred embodiments, the vic alkyldiyl group is (C₂₋₈) vic alkyldiyl, with (C₂₋₃) being particularly preferred.

“Alkylidene:” refers to a saturated or unsaturated, branched, straight-chain or cyclic divalent hydrocarbon radical derived by removal of two hydrogen atoms from the same carbon atom of a parent alkane, alkene or alkyne. The divalent radical center forms a double bond with a single atom. Typical alkylidene radicals include, but are not limited to, methanylidene, ethylidenes such as ethanylidene, ethenylidene; propylidenes such as propan-1-ylidene, propan-2-ylidene, cyclopropan-1-ylidene, prop-1-en-1-ylidene, prop-2-en-1-ylidene, cycloprop-2-en-1-ylidene, etc.; butylidenes such as butan-1-ylidene, butan-2-ylidene, 2-methyl-propan-1-ylidene, cyclobutan-1-ylidene, but-1-en-1-ylidene, but-2-en-1-ylidene, but-3-en-1-ylidene, buta-1,3-dien-1-ylidene; cyclobut-2-en-1-ylidene, etc.; and the like. Where specific levels of saturation are intended, the nomenclature alkanylidene, alkenylidene and/or alkynylidene is used. In preferred embodiments, the alkylidene group is (C₁₋₈) alkylidene, with (C₁₋₃) being particularly preferred. Also preferred are acyclic saturated alkanylidene radicals in which the divalent radical is at a terminal carbon, e.g., methanylidene, ethan-1-ylidene, propan-1-ylidene, butan-1-ylidene, 2-methyl-propan-1-ylidene, and the like.

“Alkylidyne:” refers to a saturated or unsaturated, branched or straight-chain trivalent hydrocarbon radical derived by removal of three hydrogen atoms from the same carbon atom of a parent alkane, alkene or alkyne. The trivalent radical center forms a triple bond with a single atom. Typical alkylidyne radicals include, but are not limited to, methanylidyne; ethanylidyne; propylidynes such as propan-1-ylidyne, prop-2-en-1-ylidyne, prop-2-yn-1-ylidyne; butylidynes such as butan-1-ylidyne, 2-methyl-propan-1-ylidyne, but-2-en-1-ylidyne, but-3-en-1-ylidyne, buta-2,3-dien-1-ylidyne, but-2-yn-1-ylidyne, but-3-yn-1-ylidyne, etc.; and the like. Where specific levels of saturation are intended, the nomenclature alkanylidyne, alkenylidyne and/or alkynylidyne is used. In preferred embodiments, the alkylidyne group is (C₁₋₈) alkylidyne, with (C₁₋₃) being particularly preferred. Also preferred are saturated alkanylidyne radicals, e.g., methanylidyne, ethanylidyne, propan-1-ylidyne, butan-1-ylidyne, 2-methyl-propan-1-ylidyne, and the like.

“Heteroalkyl, Heteroalkanyl, Heteroalkenyl, Heteroalkynyl, Heteroalkylidene, Heteroalkylidyne, Heteroalkyldiyl, Vic Heteralkyldiyl, Gem Heteroalkyldiyl, Heteroalkyleno and Heteroalkyldiylidene:” refer to alkyl, alkanyl, alkenyl, alkynyl, alkylidene, alkylidyne, alkyldiyl, vic alkyldiyl, gem alkyldiyl, alkyleno and alkyldiylidene radicals, respectively, in which one or more carbon atoms (and any necessary associated hydrogen atoms) are independently replaced with the same or different heteroatoms (including any necessary hydrogen or other atoms). Typical heteroatoms to replace the carbon atom(s) include, but are not limited to, N, P, O, S, Si, etc. Preferred heteroatoms are O, N and S. Thus, heteroalkyl, heteroalkanyl, heteroalkenyl, heteroalkynyl, heteroalkylidene, heteroalkylidyne, heteroalkyldiyl, vic heteroalkyldiyl, gem heteroalkyldiyl, heteroalkyleno and heteroalkyldiylidene radicals can contain one or more of the same or different heteroatomic groups, including, by way of example and not limitation, epoxy (—O—), epidioxy (—O—O—), thioether (—S—), epidithio (—SS—), epoxythio (—O—S—), epoxyimino (—O—NR′—), imino (—NR′—), biimmino (—NR′—NR′—), azino (═N—N═), azo (—N═N—), azoxy (—N—O—N—), azimino (—NR′—N═N—), phosphano (—PH—), λ⁴-sulfano (—SH₂—), sulfonyl (—-S(O)₂—), and the like, where each R′ is independently hydrogen or (C₁-C₆) alkyl.

“Parent-Aromatic Ring System:” refers to an unsaturated cyclic or polycyclic ring system having a conjugated π electron system. Specifically included within the definition of “parent aromatic ring system” are fused ring systems in which one or more rings are aromatic and one or more rings are saturated or unsaturated, such as, for example, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Aryl:” refers to a monovalent aromatic hydrocarbon. radical derived by the removal of one hydrogen atom from a single carbon atom of a parent aromatic ring system. Typical aryl groups include, but are not limited to, radicals derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In preferred embodiments, the aryl group is (C₅₋₂₀) aryl, with (C₅₋₁₀) being particularly preferred. Particularly preferred aryl groups are phenyl and naphthyl groups.

“Arylalkyl:” refers to an acyclic alkyl group in which one of the hydrogen atoms bonded to a carbon atom, typically a terminal carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended,-the nomenclature arylalkanyl, arylakenyl and/or arylalkynyl is used. [In preferred embodiments, the arylalkyl group is (C₆₋₂₆) arylalkyl, e.g., the alkanyl, alkenyl or alkynyl moiety of the arylalkyl group is (C₁₋₆) and the aryl moiety is (C₅₋₂₀). In particularly preferred embodiments the arylalkyl group is (C₆₋₁₃), e.g., the alkanyl, alkenyl or alkynyl moiety of the arylalkyl group is (C₁₋₃) and the aryl moiety is (C₅₋₁₀). Even more preferred arylalkyl groups are phenylalkanyls.

“Alkanyloxy:” refers to a saturated branched, straight-chain or cyclic monovalent hydrocarbon alcohol radical derived by the removal of the hydrogen atom from the hydroxide oxygen of the alcohol. Typical alkanyloxy groups include, but are not limited to, methanyl; ethanyloxy; propanyloxy groups such as propan-1-yloxy (CH₃CH₂CH₂O—), propan-2-yloxy ((CH₃)₂CHO—), cyclopropan-1-yloxy, etc.; butyanyloxy groups such as butan-1-yloxy, butan-2-yloxy, 2-methyl-propan-1-yloxy, 2-methyl-propan-2-yloxy, cyclobutan-1-yloxy, etc.; and the like. In preferred embodiments, the alkanyloxy groups are (C₁₋₈) alkanyloxy groups, with (C₁₋₃) being particularly preferred.

“Parent Heteroaromatic Ring System:” refers to a parent aromatic ring system in which one or more carbon atoms are each independently replaced with a heteroatom. Typical heteratoms to replace the carbon atoms include, but are not limited to, N, P, O, S, Si etc. Specifically included within the definition of “parent heteroaromatic ring systems” are fused ring systems in which one or more rings are aromatic and one or more rings are saturated or unsaturated, such as, for example, arsindole, chromane, chromene, indole, indoline, xanthene, etc. Typical parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl:” refers to a monovalent heteroaromatic radical derived by the removal of one hydrogen atom from a single atom of a parent heteroaromatic ring system. Typical heteroaryl groups include, but are not limited to, radicals derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In preferred embodiments, the heteroaryl group is a 5-20 membered heteroaryl, with 5-10 membered heteroaryl being particularly preferred. Specific preferred heteroaryls for the present invention are quinoline, isoquinoline, pyridine, pyrimidine, furan, thiophene and imidazole.

“Substituted:” refers to a radical in which one or more hydrogen atoms are each independently replaced with the same or different substituent(s). Typical substituents include, but are not limited to, —X, —R, —O—, ═O, —OR, —O—OR, —SR, —S—, ═S, —NRR, ═NR, —CX₃, —CN, —OCN, —SCN, —NCO, —NCS, —NO, —NO₂, ═N₂, —N₃, —NHOH, —S(O)₂O—, —S(O)₂OH, —S(O)₂R, —P(O)(O⁻)₂, —P(O)(OH)₂, —C(O)R, —C(O)X, —C(S)R, —C(S)X, —C(O)OR, —C(O)O⁻, —C(S)OR, —C(O)SR, —C(S)SR, —C(O)NRR, —C(S)NRR and —C(NR)NRR, where each X is independently a halogen (preferably —F, —Cl or —Br) and each R is independently —H, alkyl, alkanyl, alkenyl, alkynyl, alkylidene, alkylidyne, aryl, arylalkyl, arylheteroalkyl, heteroaryl, heteroarylalkyl or heteroaryl-heteroalkyl, as defined herein. Preferred substituents include hydroxy, halogen, C₁₋₈alkyl, C₁₋₈alkanyloxy, fluorinated alkanyloxy, fluorinated alkyl, C₁₋₈alkylthio, C₃₋₈cycloalkyl, C₃₋₈cycloalkanyloxy, nitro, amino, C₁₋₈alkylamino, C₁₋₈dialkylamino, C₃₋₈cycloalkylamino, cyano, carboxy, C₁₋₇alkanyloxycarbonyl, C₁₋₇alkylcarbonyloxy, formyl, carbamoyl, phenyl, aroyl, carbamoyl, amidino, (C₁₋₈alkylamino)carbonyl, (arylamino)carbonyl and aryl(C₁₋₈alkyl)carbonyl

“Aroyl” refers to arylacyl substituents.

“Acyl” refers to alkylcarbonyl substituents.

For the purposes of the present invention, the term “antagonist” is used to refer to a compound capable of producing a functional antagonism of the VR1 ion channel, including but not limited to competitive antagonists, non-competitive antagonists, desensitizing agonists, and partial agonists.

With reference to substituents, the term “independently” means that when more than one of such substituent is possible, such substituents may be the same or different from each other.

Throughout this disclosure, the terminal portion of the designated side chain is described first, followed by the adjacent functionality toward the point of attachment. Thus, for example, a “phenylC₁₋₆alkanylaminocarbonylC₁₋₆alkyl” substituent refers to a group of the formula

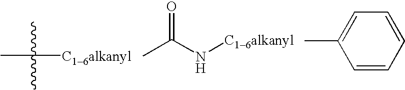

The present invention is directed to a compound of Formula (l): 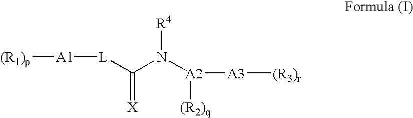
wherein:
A1 is phenyl, naphthalenyl, pyridinyl, or thienyl;
R₁ is independently hydroxy; halogen; C₁₋₈alkanyl optionally substituted with one or more substituents independently selected from the group consisting of halogen, trifluoromethylsulfonyl, trifluoromethylsulfinyl, fluorinated alkanyl, and C₁₋₈alkanyloxy; C₁₋₈alkanyloxy optionally substituted with one or more substituents independently selected from the group consisting of halogen, fluorinated alkanyl, and C₁₋₈alkanyloxy; fluorinated alkanyloxy; fluorinated alkanyl; C₁₋₈alkanylthio optionally substituted with one or more substituents independently selected from the group consisting of halogen, fluorinated alkanyl and C₁₋₈alkanyloxy; C₁₋₈alkanylsulfinyl optionally substituted with one or more substituents independently selected from the group consisting of halogen and C₁₋₈alkanyloxy; C₁₋₈alkanylsulfonyl optionally substituted with one or more substituents independently selected from the group consisting of halogen and C₁₋₈alkanyloxy; C₃₋₈cycloalkanyl; C₃₋₈cycloalkanyloxy; nitro; amino; C₁₋₈alkanylamino; C₁₋₈dialkanylamino; C₃₋₈cycloalkanylamino; N—C₁₋₈alkanyl-N—C₃₋₈cycloalkanylamino, cyano; carboxy; C₁₋₇alkanyloxycarbonyl; C₁₋₇alkanylcarbonyloxy; C₁₋₇alkanylaminocarbonyl; C₁₋₇alkanylcarbonylamino; diC₁₋₇alkanylaminocarbonyl; formyl; aminosulfonyl; C₁₋₈alkanylaminosulfonyl; di(C₁₋₈)alkanylaminosulfonyl; or cyano;
p is 0, 1 or 2;
L is C₂₋₈alkandiyl, C₂₋₈alkendiyl, or C₂₋₈alkyndiyl and L is optionally substituted with a substituent selected from the group consisting of C₁₋₈alkanyl, C₃₋₈cycloalkanyl and phenyl optionally substituted with one to three substituents independently selected from the group consisting of C₁₋₈alkanyl, C₁₋₈alkanyloxy, halogen, hydroxy, fluorinated alkanyl, fluorinated alkanyloxy, amino, di(C₁₋₃)alkanylamino, and C₁₋₃alkanylamino;
X is O or S;
A2 is selected from the group consisting of phenyl, thien-2-yl, naphthalenyl, pyridinyl, pyrimidinyl, pyrazinyl, quinolinyl, and isoquinolinyl;
R₂ is independently selected from the group consisting of C₁₋₈alkanyl, C₁₋₈alkanyloxy, hydroxy(C₁₋₈)alkanyl, fluorinated C₁₋₈alkanyl, hydroxyl, halogen, carboxy, C₁₋₈alkanyloxycarbonyl, and aminocarbonyl;
q is 0, 1, or 2;
A3 is selected from the group consisting of phenyl, thienyl, naphthalenyl, pyridinyl, pyrimidinyl, pyrazinyl, imidazolyl, oxazolyl, 4,5-dihydro-oxazolyl, pyrazolyl, dihydro-pyrazolyl, thiazolyl, thiadiazolyl, triazolyl, tetrazolyl, quinolinyl, isoquinolinyl, oxathiadiazolyl, benzimidazolyl, tetrahydrobenzimidazolyl, tetrahydroindazolyl, oxathiadiazolyl 2-oxide, oxadiazolyl, tetrahydroisoquinolinyl, tetrahydroquinolinyl, and indolyl;
such that when A3 is tetrahydrobenzimidazolyl, tetrahydroindazolyl, oxathiadiazolyl 2-oxide, tetrahydroisoquinolinyl, or tetrahydroquinolinyl, then r is 0;
R₃ is independently selected from the group consisting of hydroxy; halogen; C₁₋₈alkanyl; hydroxy(C₁₋₈)alkanyl; C₁₋₈alkanyloxy optionally substituted with amino, C₁₋₈alkanylamino, or C₁₋₈dialkanylamino; fluorinated alkanyloxy; fluorinated alkanyl; C₁₋₈alkanylthio; nitro; amino; C₁₋₈alkanylamino; C₁₋₈dialkanylamino; C₃₋₈cycloalkanylamino; cyano; aminosulfonyl; carboxy; C₁₋₈alkanylsulfonylamino; aminocarbonyl; C₁₋₈alkanyloxycarbonyl; C₁₋₄alkanyloxycarbonylamino; C₁₋₈alkanylaminocarbonyl; C₁₋₈alkanylcarbonylamino; diC₁₋₈alkanylaminocarbonyl; oxo when A3 is dihydro-pyrazolyl; and formyl; and wherein the C₁₋₈alkanyl group of any C₁₋₈alkanylamino and C₁₋₈dialkanylamino containing substituent of R₃ is optionally substituted with hydroxy; r is 0, 1, or 2;
R₄ is hydrogen or C₁₋₈alkyl;
provided that a compound of Formula 1 is other than
a compound wherein p is 1, R₁ is 4-trifluoromethylsulfonyl, A1 is phenyl, L is trans —CH═CH—, X is O, A2 is pyridin-3-yl, q is 1, R₂ is 5-methyl, A3 is imidazol-1-yl, r is 2, R₃ is 4,5-dichloro, and R₄ is hydrogen;
a compound wherein p is 1, R₁ is 4-trifluoromethyl, A1 is phenyl, L is —(CH₂)₂—, X is O, A2 is phenyl, q is 1, R₂ is 3-trifluoromethyl, A3 is pyrazol-1-yl, r is 2, R₃ is 3,5-dimethyl, and R₄ is hydrogen;
a compound wherein p is 1, R, is 4-trifluoromethyl, A1 is phenyl, L is —(CH₂)₂—, X is O, A2 is phenyl, q is 1, R₂ is 3-trifluoromethyl, A3 is imidazol-1-yl, r is 1, R₃ is 4-carboxy, and R₄ is hydrogen; and
enantiomers, diastereomers, tautomers, solvates, and pharmaceutically acceptable salts thereof.

The present invention is further directed to a compound of Formula (I) 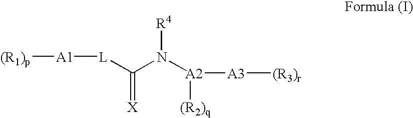
wherein:
A1 is phenyl, naphthalenyl, or thienyl;
R₁ is independently hydroxy; halogen; C₁₋₈alkanyl optionally substituted with one or more substituents independently selected from the group consisting of halogen, fluorinated alkanyl and C₁₋₈alkanyloxy; C₁₋₈alkanyloxy optionally substituted with one or more substituents independently selected from the group consisting of halogen, fluorinated alkanyl and C₁₋₈alkanyloxy; fluorinated alkanyloxy; fluorinated alkanyl; C₁₋₈alkanylthio optionally substituted with one or more substituents independently selected from the group consisting of halogen, fluorinated alkanyl and C₁₋₈alkanyloxy, C₃₋₈cycloalkanyl; C₃₋₈cycloalkanyloxy; nitro; amino; C₁₋₈alkanylamino; C₁₋₈dialkanylamino; C₃₋₈cycloalkanylamino; cyano; carboxy; C₁₋₇alkanyloxycarbonyl; C₁₋₇alkanylcarbonyloxy; C₁₋₇alkanylaminocarbonyl; C₁₋₇alkanylcarbonylamino; diC₁₋₇alkanylaminocarbonyl; formyl; C₁₋₈alkanylamino; aminosulfonyl; C₁₋₈alkanylaminosulfonyl; di(C_{1 8})alkanylaminosulfonyl; or cyano;
p is 0, 1 or 2;
L is C₂₋₈alkandiyl, C₂₋₈alkendiyl, or C₂₋₈alkyndiyl and L is optionally substituted with a substituent selected from the group consisting of C₁₋₈alkanyl, C₃₋₈cycloalkanyl and phenyl optionally substituted with one to three substituents independently selected from the group consisting of C₁₋₈alkanyl, C₁₋₈alkanyloxy, halogen, hydroxy, fluorinated alkanyl, fluorinated alkanyloxy, amino, di(C₁₋₃)alkanylamino, and C₁₋₃alkanylamino;
X is O or S;
A2 is selected from the group consisting of phenyl, thien-2-yl, naphthalenyl, pyridinyl, pyrimidinyl, pyrazinyl, quinolinyl, and isoquinolinyl;
R₂ is independently selected from the group consisting of C₁₋₈alkanyl, C₁₋₈alkanyloxy, hydroxy(C₁₋₈)alkanyl, fluorinated C₁₋₈alkanyl, hydroxyl, halogen, carboxy, C₁₋₈alkanyloxycarbonyl, and aminocarbonyl;
q is 0, 1, or 2;
A3 is selected from the group consisting of phenyl, thienyl, naphthalenyl, pyridinyl, pyrimidinyl, pyrazinyl, imidazolyl, oxazolyl, 4,5-dihydro-oxazolyl, pyrazolyl, dihydro-pyrazolyl, thiazolyl, thiadiazolyl, triazolyl, tetrazolyl, quinolinyl, isoquinolinyl, and indolyl;
R₃ is independently selected from the group consisting of hydroxy; halogen; C₁₋₈alkanyl; hydroxy(C₁₋₈)alkanyl; C₁₋₈alkanyloxy optionally substituted with amino, C₁₋₈alkanylamino, or C₁₋₈dialkanylamino; fluorinated alkanyloxy; fluorinated alkanyl; C₁₋₈alkanylthio; nitro; amino; C₁₋₈alkanylamino; C₁₋₈dialkanylamino; C₃₋₈cycloalkanylamino; cyano; aminosulfonyl; carboxy; C₁₋₈alkanylsulfonylamino; aminocarbonyl; C₁₋₈alkanyloxycarbonyl; C₁₋₄alkanyloxycarbonylamino; C₁₋₈alkanylaminocarbonyl; C₁₋₈alkanylcarbonylamino; diC₁₋₈alkanylaminocarbonyl; oxo when A3 is dihydro-pyrazolyl; and formyl; and wherein the C₁₋₈alkanyl group of any C₁₋₈alkanylamino and C₁₋₈dialkanylamino containing substituent of R₃ is optionally substituted with hydroxy;
r is 0, 1, or 2;
R₄ is hydrogen or C₁₋₈alkyl; and
enantiomers, diastereomers, tautomers, solvates, and pharmaceutically acceptable salts thereof.

Embodiments of the present invention include compounds of Formula (I) wherein:
a) A1 is phenyl, pyridinyl, or thienyl;
b) A1 is phenyl, pyridinyl, or thienyl, and p is 1 or 2;
c) A1 is phenyl or thienyl;
d) A1 is phenyl or thienyl, and p is 1 or 2;
e) p is 1 and Al is phenyl substituted at the 4-position with R₁, or thien-2-yl substituted at the 5-position with R₁;
f) R₁ is independently is C₁₋₆alkanyl; fluorinated C₁₋₆alkanyl; C₁₋₈alkanylsulfonyl substituted with one to three fluoro substituents; C₁₋₈alkanylthio substituted with one to three fluoro substituents; C₁₋₈alkanylsulfinyl substituted with one to three fluoro substituents; chloro; fluoro; or N—C₁₋₄alkanyl-N-cyclohexylamino,
g) R₁ is independently is C₁₋₄alkanyl; fluorinated C₁₋₄alkanyl; trifluoromethylsulfonyl; trifluoromethylthio; trifluoromethylsulfinyl; chloro; or N-methyl-N-cyclohexylamino;
h) R₁ is independently is t-butyl, trifluoromethyl, trifluoromethylsulfonyl, or trifluoromethylthio;
i) R₁ independently is C₁₋₆alkanyl or fluorinated C₁₋₆alkanyl;
j) R₁ independently is C₁₋₄alkanyl or fluorinated C₁₋₄alkanyl;
k) R₁ independently is t-butyl or trifluoromethyl;
l) p is 1 or 2;
m) p is 1;
n) L is C₂₋₈alkandiyl, C₂₋₈alkendiyl, or C₂₋₈alkyndiyl and L is optionally substituted with a substituent selected from the group consisting of C₁₋₄alkanyl, C₃₋₈cycloalkanyl and phenyl;
o) L is C₂₋₄alkandiyl or C₂₋₄alkendiyl, and L is optionally substituted with a substituent selected from the group consisting of C₁₋₄alkanyl, C₃₋₈cycloalkanyl and phenyl;
p) L is —CH₂CH₂— or —CH═CH—;
q) X is O or S;
r) X is O;
s) q is 0, 1, or 2 and A2 is selected from the group consisting of phenyl, thien-2-yl, pyridinyl, pyrimidinyl, pyrazinyl, quinolinyl, and isoquinolinyl;
t) q is 0, 1, or 2 and A2 is selected from the group consisting of phenyl, pyridinyl, and isoquinolinyl;
u) q is 0, 1, or 2 and A2 is selected from the group consisting of phenyl, pyridin-3-yl, and isoquinolin-5-yl;
v) R₂ is independently selected from the group consisting of C₁₋₄alkanyl, hydroxy(C₁₋₄)alkanyl, fluorinated C₁₋₄alkanyl, and fluoro;
w) R₂ is independently selected from the group consisting of methyl, hydroxymethyl, trifluoromethyl, and fluoro;
x) R₂ is independently selected from the group consisting of methyl, hydroxymethyl, and fluoro;
y) q is 1 or 2;
z) r is 1 or 2 and A3 is selected from the group consisting of phenyl, thienyl, pyridinyl, pyrimidinyl, and imidazolyl;
aa) r is 1 or 2 and when A2 is phenyl, A3 is 4-phenyl, 3-phenyl, 2-pyridin-4-yl, 2-pyridin-2-yl, 3-pyridin-4-yl, 2-pyridin-3-yl, 4-pyridin-4-yl, 4-pyridin-3-yl, 4-pyridin-2-yl, 4-pyrimidin-5-yl, 3-pyrimidin-2-yl, or 4-imidazol-1-yl; and when A2 is isoquinolin-5-yl, A3 is 8-pyridin-4-yl or 8-phenyl; and when A2 is pyridin-3-yl, A3 is 6-phenyl, 6-pyridin-3-yl, 6-pyridin-2-yl, 6-pyridin-4-yl, or 6-imidazol-1-yl;
bb) r is 1 or 2 and when A2 is phenyl, A3 is selected from the group consisting of 4-phenyl, 3-phenyl, 2-pyridin-2-yl, 4-pyridin-4-yl, 4-pyridin-3-yl, 4-pyridin-2-yl, 4-pyrimidin-5-yl, 3-pyrimidin-2-yl, and 4-imidazol-1-yl; and when A2 is isoquinolin-5-yl, A3 is 8-pyridin-4-yl or 8-phenyl; and when A2 is pyridin-3-yl, A3 is 6-phenyl, 6-pyridin-3-yl, 6-pyridin-2-yl, 6-pyridin-4-yl, or 6-imidazol-1-yl;
cc) r is 1 or 2 and A3 is selected from the group consisting of phenyl, thienyl, pyridinyl, and pyrimidinyl;
dd) r is 1 or 2 and when A2 is phenyl, A3 is 4-phenyl, 3-phenyl, 2-pyridin-4-yl, 2-pyridin-2-yl, 3-pyridin-4-yl, 2-pyridin-3-yl, 4-pyridin-4-yl, 4-pyridin-3-yl, 4-pyridin-2-yl, 4-pyrimidin-5-yl, or 3-pyrimidin-2-yl; and when A2 is isoquinolin-5-yl, A3 is 8-pyridin-4-yl or 8-phenyl; and when A2 is pyridin-3-yl, A3 is 6-phenyl, 6-pyridin-3-yl, 6-pyridin-2-yl, or 6-pyridin-4-yl;
ee) r is 1 or 2 and when A2 is phenyl, A3 is 4-phenyl, 3-phenyl, 2-pyridin-2-yl, 4-pyridin-4-yl, 4-pyridin-3-yl, 4-pyridin-2-yl, 4-pyrimidin-5-yl, or 3-pyrimidin-2-yl; and when A2 is isoquinolin-5-yl, A3 is 8-pyridin-4-yl or 8-phenyl; and when A2 is pyridin-3-yl, A3 is 6-phenyl, 6-pyridin-3-yl, 6-pyridin-2-yl, or 6-pyridin-4-yl;
ff) R₃ is independently selected from the group consisting of hydroxy; fluoro; chloro; C₁₋₄alkanyl; hydroxy(C₁₋₄)alkanyl; C₁₋₄alkanyloxy optionally substituted with amino, C₁₋₄alkanylamino, or C₁₋₄dialkanylamino; fluorinated alkanyloxy; fluorinated alkanyl; amino; C₁₋₄alkanylamino; C₁₋₄dialkanylamino; C₃-₈cycloalkanylamino; cyano; aminosulfonyl; C₁₋₄alkanylsulfonylamino; C₁₋₄alkanyloxycarbonyl; C₁₋₄alkanyloxycarbonylamino; aminocarbonyl; C₁₋₄alkanylaminocarbonyl; C₁₋₄alkanylcarbonylamino; diC₁₋₄alkanylaminocarbonyl; and formyl; and wherein the C₁₋₄alkanyl group of any C₁₋₄alkanylamino and C₁₋₄dialkanylamino containing substituent of R₃ is optionally substituted with hydroxy;
gg) R₃ is independently selected from the group consisting of hydroxy; fluoro; chloro; methyl; hydroxymethyl; C₁₋₃alkanyloxy optionally substituted with amino, methylamino, or dimethylamino; trifluoromethoxy; trifluoromethyl; methylsulfonylamino; t-butoxycarbonylamino; aminocarbonyl; and methylcarbonylamino;
hh) R₃ is independently selected from the group consisting of hydroxy, fluoro, chloro, methyl, hydroxymethyl, 2-aminoethoxy, methylsulfonylamino, aminocarbonyl, and methylcarbonylamino;
ii) R₃ is independently selected from the group consisting of hydrogen; hydroxy; fluoro; methyl; hydroxymethyl; C₁₋₃alkanyloxy optionally substituted with amino, methylamino, or dimethylamino; trifluoromethoxy; trifluoromethyl; methylsulfonylamino; t-butoxycarbonylamino; aminocarbonyl; and methylcarbonylamino;
jj) R₃ is independently selected from the group consisting of hydroxy, fluoro, methyl, hydroxymethyl, 2-aminoethoxy, methylsulfonylamino, aminocarbonyl, and methylcarbonylamino;
kk) r is 1 or 2;
ll) r is 1;
mm) R₄ is hydrogen or C₁₋₄alkyl;
nn) R₄ is hydrogen;
and combinations of a) through nn) above.

The present invention is further directed to a compound of Formula 1 wherein:
A1 is phenyl, pyridinyl, or thienyl;
R₁ is independently is C₁₋₆alkanyl; fluorinated C₁₋₆alkanyl; C₁₋₈alkanylsulfonyl substituted with one to three fluoro substituents; C₁₋₈alkanylthio substituted with one to three fluoro substituents; C₁₋₈alkanylsulfinyl substituted with one to three fluoro substituents; chloro; fluoro; or N—C₁₋₄alkanyl-N-cyclohexylamino;
p is 1 or 2;
L is C₂₋₈alkandiyl, C₂₋₈alkendiyl, or C₂₋₈alkyndiyl and L is optionally substituted with a substituent selected from the group consisting of C₁₋₄alkanyl, C₃₋₈cycloalkanyl and phenyl;
X is O or S;
A2 is selected from the group consisting of phenyl, thienyl, pyridinyl, pyrimidinyl, pyrazinyl, quinolinyl, and isoquinolinyl;
R₂ is independently selected from the group consisting of C₁₋₄alkanyl, hydroxy(C₁₋₄)alkanyl, fluorinated C₁₋₄alkanyl, and fluoro;
q is 1 or 2;
A3 is selected from the group consisting of phenyl, thienyl, pyridinyl, pyrimidinyl, and imidazolyl;
R₃ is independently selected from the group consisting of hydroxy; fluoro; chloro; C₁₋₄alkanyl; hydroxy(C₁₋₄)alkanyl; C₁₋₄alkanyloxy optionally substituted with amino, C₁₋₄alkanylamino, or C₁₋₄dialkanylamino; fluorinated alkanyloxy; fluorinated alkanyl; amino; C₁₋₄alkanylamino; C₁₋₄dialkanylamino; C₃₋₈cycloalkanylamino; cyano; aminosulfonyl; C₁₋₄alkanylsulfonylamino; C₁₋₄alkanyloxycarbonyl; C₁₋₄alkanyloxycarbonylamino; aminocarbonyl; C₁₋₄alkanylaminocarbonyl; C₁₋₄alkanylcarbonylamino; diC₁₋₄alkanylaminocarbonyl; and formyl; and wherein C₁₋₄alkanyl in any of the foregoing C₁₋₄alkanylamino and C₁₋₄dialkanylamino containing substituent of R₃ is optionally substituted with hydroxy;
R₄ is hydrogen or C₁₋₄alkyl;
provided that a compound of Formula 1 is other than
a compound wherein p is 1, R₁ is 4-trifluoromethylsulfonyl, A1 is phenyl, L is trans —CH═CH—, X is O, A2 is pyridin-3-yl, q is 1, R₂ is 5-methyl, A3 is imidazol-1-yl, r is 2, R₃ is 4,5-dichloro, and R₄ is hydrogen; and
enantiomers, diastereomers, tautomers, solvates, and pharmaceutically acceptable salts thereof.

Another embodiment of the present invention is a compound of Formula 1 wherein:
A1 is phenyl, pyridinyl, or thienyl;
R₁ is independently is C₁₋₄alkanyl; fluorinated C₁₋₄alkanyl; trifluoromethylsulfonyl; trifluoromethylthio; trifluoromethylsulfinyl; chloro; or N-methyl-N-cyclohexylamino;
p is 1;
L is C₂₋₄alkandiyl or C₂₋₄alkendiyl, and L is optionally substituted with a substituent selected from the group consisting of C₁₋₄alkanyl, C₃₋₈cycloalkanyl and phenyl;
X is O;
A2 is selected from the group consisting of phenyl, thienyl, pyridinyl, and isoquinolinyl;
R₂ is independently selected from the group consisting of methyl, hydroxymethyl, trifluoromethyl, and fluoro;
q is 0, 1, or 2;
A3 is 4-phenyl, 3-phenyl, 2-pyridin-4-yl, 2-pyridin-2-yl, 3-pyridin-4-yl, 2-pyridin-3-yl, 4-pyridin-4-yl, 4-pyridin-3-yl, 4-pyridin-2-yl, 4-pyrimidin-5-yl, 3-pyrimidin-2-yl or 4-imidazol-1-yl when A2 is phenyl; or when A2 is isoquinolin-5-yl, A3 is 8-pyridin-4-yl or 8-phenyl; or when A2 is pyridin-3-yl, A3 is 6-phenyl, 6-pyridin-3-yl, 6-pyridin-2-yl, 6-pyridin-4-yl or 6-imidazol-1-yl;
R₃ is independently selected from the group consisting of hydroxy; fluoro; chloro; methyl; hydroxymethyl; C₁₋₃alkanyloxy optionally substituted with amino, methylamino, or dimethylamino; trifluoromethoxy; trifluoromethyl; methylsulfonylamino; t-butoxycarbonylamino; aminocarbonyl; and methylcarbonylamino;
r is 1;
R₄ is hydrogen;
provided that a compound of Formula 1 is other than a compound wherein p is 1, R₁ is 4-trifluoromethylsulfonyl, A1 is phenyl, L is trans —CH═CH—, X is O, A2 is pyridin-3-yl, q is 1, R₂ is 5-methyl, A3 is imidazol-1-yl, r is 2, R₃ is 4,5-dichloro, and R₄ is hydrogen. and
enantiomers, diastereomers, tautomers, solvates, and pharmaceutically acceptable salts thereof.

Another embodiment of the present invention is directed to compositions comprising a compound of Formula 1a: 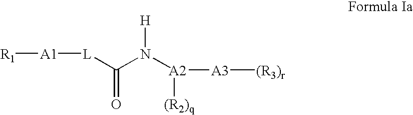
wherein:
A1 is phenyl substituted at the 4-position with R₁, or thiophen-2-yl substituted at the 5-position with R₁;
R₁ is independently is t-butyl, trifluoromethyl, trifluoromethylsulfonyl, or trifluoromethylthio;
L is —CH₂CH₂— or —CH═CH—;
A2 is selected from the group consisting of phenyl, pyridin-3-yl, and isoquinolin-5-yl optionally substituted with R₂;
R₂ is independently selected from the group consisting of methyl, hydroxymethyl, trifluoromethyl, and fluoro;
q is 0, 1, or 2;
A3 is 4-phenyl, 3-phenyl, 2-pyridin-2-yl, 4-pyridin-4-yl, 4-pyridin-3-yl, 4-pyridin-2-yl, 4-pyrimidin-5-yl, 3-pyrimidin-2-yl, or 4-imidazol-1-yl when A2 is phenyl; and when A2 is isoquinolin-5-yl, A3 is 8-pyridin-4-yl or 8-phenyl; and when A2 is pyridin-3-yl, A3 is 6-phenyl, 6-pyridin-3-yl, 6-pyridin-2-yl, 6-pyridin-4-yl, or 6-imidazol-1-yl;
R₃ is independently selected from the group consisting of hydroxy, fluoro, chloro, methyl, hydroxymethyl, 2-aminoethoxy, methylsulfonylamino, aminocarbonyl, and methylcarbonylamino;
provided that a compound of Formula 1 is other than a compound wherein p is 1, R, is 4-trifluoromethylsulfonyl, A1 is phenyl, L is trans —CH═CH—, X is O, A2 is pyridin-3-yl, q is 1, R₂ is 5-methyl, A3 is imidazol-1-yl, r is 2, R₃ is 4,5-dichloro, and R₄ is hydrogen. and
enantiomers, diastereomers, tautomers, solvates, and pharmaceutically acceptable salts thereof.

The present invention is further directed to a compound of Formula 1 wherein:
A1 is phenyl or thienyl;
R₁ independently is C₁₋₆alkanyl or fluorinated C₁₋₆alkanyl;
p is 1 or 2;
L is C₂₋₈alkandiyl, C₂₋₈alkendiyl, or C₂₋₈alkyndiyl and L is optionally substituted with a substituent selected from the group consisting of C₁₋₄alkanyl, C₃₋₈cycloalkanyl and phenyl;
X is O or S;
A2 is selected from the group consisting of phenyl, thienyl, pyridinyl, pyrimidinyl, pyrazinyl, quinolinyl, and isoquinolinyl;
R₂ is independently selected from the group consisting of C₁₋₄alkanyl, hydroxy(C₁₋₄)alkanyl, fluorinated C₁₋₄alkanyl, and fluoro;
q is 1 or 2;
A3 is selected from the group consisting of phenyl, thienyl, pyridinyl, and pyrimidinyl;
R₃ is independently selected from the group consisting of hydroxy; fluoro; chloro; C₁₋₄alkanyl; hydroxy(C₁₋₄)alkanyl; C₁₋₄alkanyloxy optionally substituted with amino, C₁₋₄alkanylamino, or C₁₋₄dialkanylamino; fluorinated alkanyloxy; fluorinated alkanyl; amino; C₁₋₄alkanylamino; C₁₋₄dialkanylamino; C₃₋₈cycloalkanylamino; cyano; aminosulfonyl; C₁₋₄alkanylsulfonylamino; C₁₋₄alkanyloxycarbonyl; C₁₋₄alkanyloxycarbonylamino; aminocarbonyl; C₁₋₄alkanylaminocarbonyl; C₁₋₄alkanylcarbonylamino; diC₁₋₄alkanylaminocarbonyl; and formyl; and wherein C₁₋₄alkanyl in any of the foregoing C₁₋₄alkanylamino and C₁₋₄dialkanylamino containing substituent of R₃ is optionally substituted with hydroxy;
R₄ is hydrogen or C₁₋₄alkyl; and
enantiomers, diastereomers, tautomers, solvates, and pharmaceutically acceptable salts thereof.

Another embodiment of the present invention is a compound of Formula 1 wherein:
A1 is phenyl or thienyl;
R₁ is independently C₁₋₄alkanyl or fluorinated C₁₋₄alkanyl;
p is 1;
L is C₂₋₄alkandiyl or C₂₋₄alkendiyl, and L is optionally substituted with a substituent selected from the group consisting of C₁₋₄alkanyl, C₃₋₈cycloalkanyl and phenyl;
X is O;
A2 is selected from the group consisting of phenyl, thienyl, pyridinyl, and isoquinolinyl;
R₂ is independently selected from the group consisting of methyl, hydroxymethyl, and fluoro;
q is 0, 1, or 2;
A3 is 4-phenyl, 3-phenyl, 2-pyridin-4-yl, 2-pyridin-2-yl, 3-pyridin-4-yl, 2-pyridin-3-yl, 4-pyridin-4-yl, 4-pyridin-3-yl, 4-pyridin-2-yl, 4-pyrimidin-5-yl or 3-pyrimidin-2-yl when A2 is phenyl; and when A2 is isoquinolin-5-yl, A3 is 8-pyridin-4-yl or 8-phenyl; and when A2 is pyridin-3-yl, A3 is 6-phenyl, 6-pyridin-3-yl, 6-pyridin-2-yl, or 6-pyridin-4-yl;
R₃ is independently selected from the group consisting of hydroxy; fluoro; methyl; hydroxymethyl; C, ₃alkanyloxy optionally substituted with amino, methylamino, or dimethylamino; trifluoromethoxy; trifluoromethyl; methylsulfonylamino; t-butoxycarbonylamino; aminocarbonyl; and methylcarbonylamino;
r is 1;
R₄ is hydrogen; and
enantiomers, diastereomers, tautomers, solvates, and pharmaceutically acceptable salts thereof.

Another embodiment of the present invention is directed to compositions comprising a compound of Formula 1a: 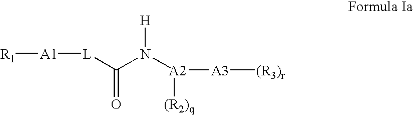
wherein:
A1 is phenyl substituted at the 4-position with R₁, or thiophen-2-yl substituted at the 5-position with R₁;
R₁ is independently t-butyl or trifluoromethyl;
L is —CH₂CH₂— or —CH═CH—;
A2 is selected from the group consisting of phenyl, pyridin-3-yl, and isoquinolin-5-yl optionally substituted with R₂;
R₂ is independently selected from the group consisting of hydrogen, methyl, hydroxymethyl, and fluoro;
q is 0, 1, or 2;
A3 is 4-phenyl, 3-phenyl, 2-pyridin-2-yl, 4-pyridin-4-yl, 4-pyridin-3-yl, 4-pyridin-2-yl, 4-pyrimidin-5-yl, or 3-pyrimidin-2-yl when A2 is phenyl; and when A2 is isoquinolin-5-yl, A3 is 8-pyridin-4-yl or 8-phenyl; and when A2 is pyridin-3-yl, A3 is 6-phenyl, 6-pyridin-3-yl, 6-pyridin-2-yl, or 6-pyridin-4-yl;
R₃ is independently selected from the group consisting of hydroxy, fluoro, methyl, hydroxymethyl, 2-aminoethoxy, methylsulfonylamino, aminocarbonyl, and methylcarbonylamino, and
enantiomers, diastereomers, tautomers, solvates, and pharmaceutically acceptable salts thereof.

Another embodiment of the present invention is directed to compositions comprising a compound of Formula (Ia) 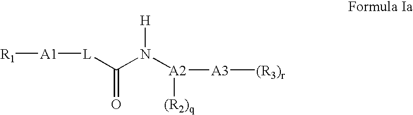
selected from the group consisting of:
a compound of Formula (Ia) wherein R₁ is 4-t-butyl, A1 is phenyl, L is —(CH₂)₂—, A2 is phenyl, q is 0; R₂ is absent; A3 is 2-phenyl, r is 1, and R3is 4-hydroxy;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 0, R₂ is absent, A3 is 2-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 0, R₂ is absent, A3 is 2-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-nitro;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-trifluoromethyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-methylcarbonyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-chloro;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-fluoro;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-amino;
a compound of Formula (Ia) wherein R₁ is 4-t-butyl, A1 is phenyl, L is —(CH₂)₂—, A2 is phenyl, q is 0, R₂ is absent, A3 is 2-phenyl, r is 1, and R₃ is 3-hydroxymethyl;
a compound of Formula (Ia) wherein R₁ is 4-t-butyl, A1 is phenyl, L is —(CH₂)₂—, A2 is phenyl, q is 0, R₂ is absent, A3 is 2-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 3-hydroxymethyl;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-hydroxymethyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-cyano;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-trifluoromethoxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-methoxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-methyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-methoxycarbonyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-carboxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-aminocarbonyl;
a compound of Formula (Ia) wherein R₁ is 4-t-butyl, A1 is phenyl, L is trans —CH═CH—, A2 is phenyl, q is 0, R₂ is absent, A3 is 3-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 3-dimethylaminocarbonyl;
a compound of Formula (Ia) wherein R₁ is 4-t-butyl, A1 is phenyl, L is trans —CH═CH—, A2 is phenyl, q is 0, R₂ is absent, A3 is 4-phenyl, r is 1, and R₃ is 3-aminocarbonyl;
a compound of Formula (Ia) wherein R₁ is 4-t-butyl, A1 is phenyl, L is trans —CH═CH—, A2 is phenyl, q is 0, R₂ is absent, A3 is 4-phenyl, r is 1, and R₃ is 3-dimethylaminocarbonyl;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 3-dimethylaminocarbonyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-dimethylaminocarbonyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-methylsulfonylamino;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-t-butoxycarbonylamino;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-t-butoxycarbonylaminomethyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-aminomethyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-ureido;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-(2-amino-ethoxy);
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-(2-hydroxy-ethylamino);
a compound of Formula (Ia) wherein R₁ is 4-trifluoromethyl, A1 is phenyl, L is trans —CH═CH—, A2 is phenyl, q is 0, R₂ is absent, A3 is 4-phenyl, r is 1, and R₃ is 3-aminocarbonyl;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 2-methyl, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 2-fluoro, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 3-fluoro, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 2-methyl, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 2-fluoro, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 3-fluoro, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 2-methyl, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 2-fluoro, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 3-fluoro, A3 is 4-phenyl, r is 1, and R₃ is 3-hydroxy;
a compound of Formula (Ia) wherein R₁ is 4-t-butyl, A1 is phenyl,. L,is —(CH₂)₂—, A2 is phenyl, q is 0, R₂ is absent, A3 is 4-phenyl, r is 1, and R₃ is 3-hydroxy;
a compound of Formula (Ia) wherein R₁ is 5-t-butyl, A1 is thiophen-2-yl, L is —(CH₂)₂—, A2 is phenyl, q is 0, R₂ is absent, A3 is 4-phenyl, r is 1, and R₃ is 3-aminocarbonyl;
a compound of Formula (Ia) wherein R₁ is 5-t-butyl, A1 is thiophen-2-yl, L is —(CH₂)₂—, A2 is phenyl, q is 0, R₂ is absent, A3 is 4-phenyl, r is 1, and R₃ is 3-methylcarbonylamino;
a compound of Formula (Ia) wherein R₁ is 5-t-butyl, A1 is thiophen-2-yl, L is —(CH₂)₂—, A2 is phenyl, q is 0, R₂ is absent, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 2-carboxy, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 2-carboxy, A3 is 4-phenyl, r is 1, and R₃ is 3-methylaminocarbonyl;
a compound of Formula (Ia) wherein R₁ is 4-t-butyl, A1 is phenyl, L is —(CH₂)₂—, A2 is phenyl, q is 1, R₂ is 2-carboxy, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 2-hydroxymethyl, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 2-hydroxymethyl, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 2-hydroxymethyl, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 3-carboxy, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 3-carboxy, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 3-carboxy, A3 is 4-phenyl, r is 1, and R₃ is 3-hydroxy;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 3-methylcarbonylamino;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 2-hydroxy;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 3-hydroxy;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 4-hydroxy;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 3-hydroxymethyl;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is naphthalen-1-yl, q is 0, R₂ is absent, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is naphthalen-1-yl, q is 0, R₂ is absent, A3 is 4-phenyl, r is 1, and R₃ is 3-hydroxy;
a compound of Formula (Ia) wherein R₁ is 4-trifluoromethyl, A1 is phenyl, L is —(CH₂)₂—, A2 is naphthalen-1-yl, q is 0, R₂ is absent, A3 is 4-phenyl, r is 1, and R₃ is 3-aminocarbonyl;
a compound of Formula (Ia) wherein R₁ is 4-trifluoromethyl, A1 is phenyl, L is —(CH₂)₂—, A2 is naphthalen-1-yl, q is 0, R₂ is absent, A3 is 4-phenyl, r is 1, and R₃ is 3-methylcarbonylamino;
a compound of Formula (Ia) wherein R₁ is 4-trifluoromethyl, A1 is phenyl, L is —(CH₂)₂—, A2 is naphthalen-1-yl, q is 0, R₂ is absent, A3 is 4-phenyl, r is 1, and R₃is 3-hydroxy;
a compound of Formula (Ia) wherein R₁ is 4-t-butyl, A1 is phenyl, L is —(CH₂)₂—, A2 is phenyl, q is 2, R₂ is 3,5-dimethyl, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 2, R₂ is 3,5-dimethyl, A3 is 4-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 2, R₂ is 3,5-dimethyl, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 2, R₂ is 3,5-difluoro, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 2, R₂ is 3,5-difluoro, A3 is 4-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 2, R₂ is 3,5-difluoro, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-phenyl, r is 1, and R₃ is 3-hydroxymethyl;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 1, R₂ is 3-fluoro, A3 is 4-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 1, R₂ is 3-fluoro, A3 is 4-phenyl, r is 1, and R₃ is 4-hydroxy
a compound of Formula (Ia) wherein R₁ is 4-t-butyl, A1 is phenyl, L is —(CH₂)₂—, A2 is phenyl, q is 1, R₂ is 3-fluoro, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 0, R₂ is absent, A3 is 2-pyridin-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 3-pyridin-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 2-pyridin-3-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 3-pyridin-3-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-pyridin-3-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 2-pyridin-2-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 3-pyridin-2-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-pyridin-2-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-pyrimidin-2-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-pyrimidin-5-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 3-pyridin-2-yl, r is 1, and R₃ is 6-hydroxy;
a compound of Formula (Ia) wherein R₁ is 4-t-butyl, A1 is phenyl, L is —(CH₂)₂—, A2 is phenyl, q is 0, R₂ is absent, A3 is 4-pyridin-2-yl, r is 1, and R₃ is 3-hydroxy;
a compound of Formula (Ia) wherein R₁ is 4-t-butyl, A1 is phenyl, L is —(CH₂)₂—, A2 is phenyl, q is 0, R₂ is absent, A3 is 3-pyrimidin-2-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 3-pyrimidin-5-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 3-pyridin-2-yl, r is 1, and R₃ is 6-methoxy;
a compound of Formula (Ia) wherein R₁ is 4-t-butyl, A1 is phenyl, L is —(CH₂)₂—, A2 is phenyl, q is 0, R₂ is absent, A3 is 3-pyridin-3-yl, r is 1, and R₃ is 6-methoxy;
a compound of Formula (Ia) wherein R₁ is 4-t-butyl, A1 is phenyl, L is —(CH₂)₂—, A2 is phenyl, q is 0, R₂ is absent, A3 is 4-pyridin-2-yl, r is 1, and R₃ is 6-methoxy;
a compound of Formula (Ia) wherein R₁ is 4-t-butyl, A1 is phenyl, L is —(CH₂)₂—, A2 is phenyl, q is 0, R₂ is absent, A3 is 4-pyridin-3-yl, r is 1, and R₃ is 6-methoxy;
a compound of Formula (Ia) wherein R₁ is 4-t-butyl, A1 is phenyl, L is —(CH₂)₂—, A2 is phenyl, q is 0, R₂ is absent, A3 is 4-1 H-indol-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-1 H-indol-6-yl, r is 0, and R₃ is absent;
a compound of Formula (Ia) wherein R₁ is 4-t-butyl, A1 is phenyl, L is —(CH₂)₂—, A2 is pyridin-2-yl, q is 0, R₂ is absent, A3 is 5-phenyl, r is 1, and R₃ is 3-aminocarbonyl;
a compound of Formula (Ia) wherein R₁ is 4-t-butyl, A1 is phenyl, L is —(CH₂)₂—, A2 is pyridin-2-yl, q is 0, R₂ is absent, A3 is 5-phenyl, r is 1, and R₃ is 3-methylcarbonylamino;
a compound of Formula (Ia) wherein R₁ is 4-t-butyl, A1 is phenyl, L is —(CH₂)₂—, A2 is pyridin-2-yl, q is 0, R₂ is absent, A3 is 6-phenyl, r is 1, and R₃ is 3-hydroxy;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 3-aminocarbonyl;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 3-methylcarbonylamino;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 3-hydroxy;
a compound of Formula (Ia) wherein R₁ is 4-t-butyl, A1 is phenyl, L is-(CH₂)₂—, A2 is pyrimidin-2-yl, q is 0, R₂ is absent, A3 is 5-phenyl, r is 1, and R₃ is 3-aminocarbonyl;
a compound of Formula (Ia) wherein R₁ is 4-t-butyl, A1 is phenyl, L is —(CH₂)₂—, A2 is pyrazin-2-yl, q is 0, R₂ is absent, A3 is 5-phenyl, r is 1, and R₃ is 3-aminocarbonyl;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 2-hydroxy;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 4-hydroxy;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 3-hydroxymethyl;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 4-hydroxymethyl;
a compound of Formula (Ia) wherein R₁ is 4-t-butyl, A1 is phenyl, L is —(CH₂)₂—, A2 is pyridin-3-yl, q is 0, R₂ is absent, A3 is 6-pyridin-3-yl, r is 0, and R₃ is absent;
a compound of Formula (Ia) wherein R₁ is 4-t-butyl, A1 is phenyl, L is —(CH₂)₂—, A2 is pyridin-3-yl, q is 0, R₂ is absent, A3 is 6-pyridin-4-yl, r is 0, and R₃ is absent;
a compound of Formula (Ia) wherein R₁ is 4-t-butyl, A1 is thiophen-2-yl, L is —(CH₂)₂—, A2 is phenyl, q is 0, R₂ is absent, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 2-carboxy, A3 is 4-pyridin-4-yl, r is 0, and R₃ is absent;

a compound of Formula (Ia) wherein R₁ is 4-t-butyl, A1 is phenyl, L is —(CH₂)₂—, A2 is phenyl, q is 1, R₂ is 2-hydroxymethyl, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-carboxy, A3 is 4-pyridin-4-yl, r is 0, and R₃ is absent;
a compound of Formula (Ia) wherein R₁ is 4-trifluoromethyl , A1 is phenyl, L is —(CH₂)₂—, A2 is phenyl, q is 0, R₂ is absent, A3 is 4-pyridin-3-yl, r is 0, and R₃ is absent;
a compound of Formula (Ia) wherein R, is 4-trifluoromethyl , A1 is phenyl, L is —(CH₂)₂—, A2 is phenyl, q is 0, R₂ is absent, A3 is 4-pyridin-4-yl, r is 0, and R₃ is absent;
a compound of Formula (Ia) wherein R₁ is 4-trifluoromethyl , A1 is phenyl, L is —(CH₂)₂—, A2 is pyridin-3-yl, q is 0, R₂ is absent, A3 is 6-phenyl, r is 1, and R_{3 l is} 3-aminocarbonyl;
a compound of Formula (Ia) wherein R₁ is 4-trifluoromethyl , A1 is phenyl, L is —(CH₂)₂—, A2 is pyridin-3-yl, q is 0, R₂ is absent, A3 is 6-phenyl, r is 1, and R_{3 l is} 3-methylcarbonylamino;
a compound of Formula (Ia) wherein R₁ is 4-trifluoromethyl , A1 is phenyl, L is —(CH₂)₂—, A2 is pyridin-3-yl, q is 0, R₂ is absent, A3 is 6-phenyl, r is 1, and R₃ is 2-hydroxy;
a compound of Formula (Ia) wherein R₁ is 4-trifluoromethyl , A1 is phenyl, L is —(CH₂)₂—, A2 is pyridin-3-yl, q is 0, R₂ is absent, A3 is 6-phenyl, r is 1, and R₃ is 3-hydroxy;
a compound of Formula (Ia) wherein R₁ is 4-trifluoromethyl , A1 is phenyl, L is —(CH₂)₂—, A2 is pyridin-3-yl, q is 0, R₂ is absent, A3 is 6-phenyl, r is 1, and 4-hydroxy;
a compound of Formula (Ia) wherein R₁ is 4-trifluoromethyl , A1 is phenyl, L is —(CH₂)₂—, A2 is pyridin-3-yl, q is 0, R₂ is absent, A3 is,6-phenyl, r is 1, and R₃ is 3-hydroxymethyl;
a compound of Formula (Ia) wherein R₁ is 4-trifluoromethyl , A1 is phenyl, L is —(CH₂)₂—, A2 is pyridin-3-yl, q is 0, R₂ is absent, A3 is 6-phenyl, r is 1, and R₃ is 4-hydroxymethyl;
a compound of Formula (Ia) wherein R₁ is 4-trifluoromethyl , A1 is phenyl, L is —(CH₂)₂—, A2 is pyridin-3-yl, q is 0, R₂ is absent, A3 is 6-pyridin-3-yl, r is 0, and R₃ is absent;
a compound of Formula (Ia) wherein R₁ is 4-trifluoromethyl , A1 is phenyl, L is —(CH₂)₂—, A2 is pyridin-3-yl, q is 0, R₂ is absent, A3 is 6-pyridin-4-yl, r is 0, and R₃ is absent;
a compound of Formula (Ia) wherein R₁ is 4-t-butyl , A1 is phenyl, L is —(CH₂)₂—, A2 is naphthalen-1-yl, q is 0, R₂ is absent, A3 is 4-pyridin-4-yl, r is 0, and R₃ is absent;
a compound of Formula (Ia) wherein R₁ is 4-t-butyl , A1 is phenyl, L is —(CH₂)₂—, A2 is isoquinolin-5-yl, q is 0, R₂ is absent, A3 is 8-phenyl, r is 1, and R₃ is 3-methylcarbonylamino;
a compound of Formula (Ia) wherein R₁ is 4-t-butyl, A1 is phenyl, L is —(CH₂)₂—, A2 is isoquinolin-5-yl, q is 0, R₂ is absent, A3 is 8-phenyl, r is 1, and R₃ is 3-hydroxy;
a compound of Formula (Ia) wherein R₁ is 4-t-butyl, A1 is phenyl, L is —(CH₂)₂—, A2 is isoquinolin-5-yl, q is 0, R₂ is absent, A3 is 8-pyridin-4-yl, r is 0, and R₃ is absent;
a compound of Formula (Ia) wherein R₁ is 4-trifluoromethyl , A1 is phenyl, L is —(CH₂)₂—, A2 is naphthalen-1-yl, q is 0, R₂ is absent, A3 is 4-pyridin-4-yl, r is 0, and R₃ is absent;
a compound of Formula (Ia) wherein R₁ is 4-trifluoromethyl , A1 is phenyl, L is —(CH₂)₂—, A2 is isoquinolin-5-yl, q is 0, R₂ is absent, A3 is 8-phenyl, r is 1, and R₃ is 3-hydroxy;
a compound of Formula (Ia) wherein R₁ is 4-trifluoromethyl , A1 is phenyl, L is —(CH₂)₂—, A2 is isoquinolin-5-yl, q is 0, R₂ is absent, A3 is 8-pyridin-4-yl, r is 0, and R₃ is absent;
a compound of Formula (Ia) wherein R₁ is 4-t-butyl , A1 is phenyl, L is —(CH₂)₂—, A2 is pyridin-3-yl, q is 1, R₂ is 5-methyl, A3 is 6-phenyl, r is 1, and R₃ is 2-hydroxy;
a compound of Formula (Ia) wherein R₁ is 4-t-butyl , A1 is phenyl, L is —(CH₂)₂—, A2 is pyridin-3-yl, q is 1, R₂ is 5-methyl, A3 is 6-phenyl, r is 1, and R₃ is 3-hydroxy;
a compound of Formula (Ia) wherein R₁ is 4-t-butyl , A1 is phenyl, L is —(CH₂)₂—, A2 is pyridin-3-yl, q is 1, R₂ is 5-methyl, A3 is 6-phenyl, r is 1, and R₃ is 4-hydroxy;
a compound of Formula (Ia) wherein R₁ is 4-t-butyl , A1 is phenyl, L is —(CH₂)₂—, A2 is pyridin-3-yl, q is 1, R₂ is 5-methyl, A3 is 6-phenyl, r is 1, and R₃ is 4-hydroxymethyl;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-pyridin-3-yl, r is 0, and R₃ is absent;
a compound of Formula (Ia) wherein R₁ is 4-t-butyl , A1 is phenyl, L is —(CH₂)₂—, A2 is pyridin-3-yl, q is 1, R₂ is 5-methyl, A3 is 6-pyridin-4-yl, r is 0, and R₃ is absent;
a compound of Formula (Ia) wherein R₁ is 4-t-butyl , A1 is phenyl, L is —(CH₂)₂—, A2 is phenyl, q is 2, R₂ is 3,5-difluoro, A3 is 4-pyridin-3-yl, r is 0, and R₃ is absent;
a compound of Formula (Ia) wherein R₁ is 4-t-butyl, A1 is phenyl, L is —(CH₂)₂—, A2 is phenyl, q is 2, R₂ is 3,5-dimethyl, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 2, R₂ is 3,5-difluoro, A3 is 4-pyridin-3-yl, r is 0, and R₃ is absent;
a compound of Formula (Ia) wherein R₁ is 4-t-butyl , A1 is phenyl, L is —(CH₂)₂—, A2 is phenyl, q is 2, R₂ is 3,5-difluoro, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-pyridin-3-yl, r is 0, and R₃ is absent;
a compound of Formula (Ia) wherein R, is 4-t-butyl, A1 is phenyl, L is —(CH₂)₂—, A2 is phenyl, q is 1, R₂ is 3-hydroxymethyl, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-methyl, A3 is 4-pyridin-2-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-fluoro, A3 is 4-pyridin-2-yl, r is 0, and R₃ is absent;
a compound of Formula (Ia) wherein R₁ is 4-t-butyl , A1 is phenyl, L is —(CH₂)₂—, A2 is phenyl, q is 2, R₂ is 3,5-dimethyl, A3 is 4-pyridin-2-yl, r is 0, and R₃ is absent;
a compound of Formula (Ia) wherein R₁ is 4-t-butyl , A1 is phenyl, L is —(CH₂)₂—, A2 is phenyl, q is 2, R₂ is 3,5-difluoro, A3 is 4-pyridin-2-yl, r is 0, and R₃ is absent;
a compound of Formula (Ia) wherein R₁ is 4-t-butyl, A1 is phenyl, L is —(CH₂)₂—, A2 is pyridin-3-yl, q is 0, R₂ is absent, A3 is 6-pyridin-2-yl, r is 0, and R₃ is absent;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-pyridin-2-yl, r is 0, and R₃ is absent;
a compound of Formula (Ia) wherein R₁ is 4-t-butyl, A1 is phenyl, L is —(CH₂)₂—, A2 is pyrazin-2-yl, q is 0, R₂ is absent, A3 is 5-pyridin-2-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-methyl, A3 is 4-pyridin-3-yl, r is 0, and R₃ is absent;
a compound of Formula (Ia) wherein R₁ is 4-t-butyl , A1 is phenyl, L is —(CH₂)₂—, A2 is phenyl, q is 1, R₂ is 3-methyl, A3 is 4-pyridin-4-yl, r is 0, and R₃ is absent;
a compound of Formula (Ia) wherein R₁ is 4-t-butyl , A1 is phenyl, L is —(CH₂)₂—, A2 is phenyl, q is 1, R₂ is 3-fluoro, A3 is 4-pyridin-3-yl, r is 0, and R₃ is absent;
a compound of Formula (Ia) wherein R₁ is 4-t-butyl , A1 is phenyl, L is —(CH₂)₂—, A2 is phenyl, q is 1, R₂ is 3-fluoro, A3 is 4-pyridin-4-yl, r is 0, and R₃ is absent;
a compound of Formula (Ia) wherein R₁ is 4-t-butyl , A1 is phenyl, L is —(CH₂)₂—, A2 is pyrazin-2-yl, q is 0, R₂ is absent, A3 is 5-phenyl, r is 1, and R₃ is 2-hydroxy;
a compound of Formula (Ia) wherein R₁ is 4-t-butyl , A1 is phenyl, L is —(CH₂)₂—, A2 is pyrazin-2-yl, q is 0, R₂ is absent, A3 is 5-phenyl, r is 1, and R₃ is 3-hydroxy;
a compound of Formula (Ia) wherein R₁ is 4-t-butyl , A1 is phenyl, L is —(CH₂)₂—, A2 is pyrazin-2-yl, q is 0, R₂ is absent, A3 is 5-phenyl, r is 1, and R₃ is 4-hydroxy;
a compound of Formula (Ia) wherein R₁ is 4-t-butyl , A1 is phenyl, L is —(CH₂)₂—, A2 is pyrazin-2-yl, q is 0, R₂ is absent, A3 is 5-phenyl, r is 1, and R₃ is 4-hydroxymethyl;
a compound of Formula (Ia) wherein R₁ is 4-t-butyl, A1 is phenyl, L is —(CH₂)₂—, A2 is pyrazin-2-yl, q is 0, R₂ is absent, A3 is 5-pyridin-3-yl, r is 0, and R₃ is absent;
a compound of Formula (Ia) wherein R₁ is 4-t-butyl, A1 is phenyl, L is —(CH₂)₂—, A2 is pyrazin-2-yl, q is 0, R₂ is absent, A3 is 5-pyridin-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-imidazol-2-yl, r is 1, and R₃ is 1-methyl;
a compound of Formula (Ia) wherein R₁ is 4-t-butyl, A1 is phenyl, L is —(CH₂)₂—, A2 is phenyl, q is 0, R₂ is absent, A3 is 4-imidazol-4-yl, r is 1, and R₃ is 1-methyl;
a compound of Formula (Ia) wherein R₁ is 4-t-butyl, A1 is phenyl, L is —(CH₂)₂—, A2 is phenyl, q is 0, R₂ is absent, A3 is 4-imidazol-5-yl, r is 1, and R₃ is 1-methyl;
a compound of Formula (Ia) wherein R, is 4-t-butyl, A1 is phenyl, L is —(CH₂)₂—, A2 is phenyl, q is 0, R₂ is absent, A3 is 4-imidazol-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-oxazol-5-yl, r is 0, and R₃ is absent;
a compound of Formula (Ia) wherein R, is 4-t-butyl, A1 is phenyl, L is —(CH₂)₂—, A2 is phenyl, q is 0, R₂ is absent, A3 is 4-(4,5-dihydro-oxazol-5-yl), r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-thiazol-4-yl, r is 1, and R₃ is 2-methyl;
a compound of Formula (Ia) wherein R₁ is 4-t-butyl, A1 is phenyl, L is —(CH₂)₂—, A2 is phenyl, q is 0, R₂ is absent, A3 is 4-pyrazol-1-yl, r is 2, and R₃ is 3,5-dimethyl
a compound of Formula (Ia) wherein R₁ is 4-t-butyl, A1 is phenyl, L is —(CH₂)₂—, A2 is phenyl, q is 0, R₂ is absent, A3 is 4-thiadiazol-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-(1 ,2,4-triazol-1-yl), r is 0, and R₃ is absent;
a compound of Formula (Ia) wherein R, is 4-t-butyl , A1 is phenyl, L is —(CH₂)₂—, A2 is phenyl, q is 0, R₂ is absent, A3 is 4-1 H-tetrazol-5-yl, r is 0, and R₃ is absent;
a compound of Formula (Ia) wherein R₁ is 4-t-butyl , A1 is phenyl, L is —(CH₂)₂—, A2 is phenyl, q is 0, R₂ is absent, A3 is 4-imidazol-3-yl, r is 2, and R₃ is 4,5-dichloro; and
a compound of Formula (Ia) wherein R₁ is 4-t-butyl , A1 is phenyl, L is —(CH₂)₂—, A2 is phenyl, q is 0, R₂ is absent, A3 is 4-(2,4-dihydro-pyrazol-2-yl), r is 2, and R₃ is 3-oxo, 5-methyl.

The compounds of the present invention may also be present in the form of pharmaceutically acceptable salts. For use in medicine, the salts of the compounds of this invention refer to non-toxic “pharmaceutically acceptable. salts” (Ref. International J. Pharm., 1986, 33, 201-217, J. Pharm. Sci., 1997 (January), 66, 1, 1). Other salts well known to those in the art, however, may be useful in the preparation of compounds according to this invention or of their pharmaceutically acceptable salts. Representative organic or inorganic acids include, but are not limited to, hydrochloric, hydrobromic, hydroiodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, ptoluenesulfonic, cyclohexanesulfamic, salicylic, saccharinic and trifluoroacetic acid. Representative organic or inorganic bases include, but are not limited to, basic or cationic salts such as benzathine, chloroprocaine, choline, diethanolamine, ethylenediamine, meglumine, procaine, aluminum, calcium, lithium, magnesium, potassium, sodium and zinc.

The present invention includes within its scope prodrugs of the compounds of this invention. In general, such prodrugs will be functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. bundgaard, Elsevier, 1985.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Where the processes for the preparation of the compounds according to the present invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds, for example, may be resolved into their component enantiomers by standard techniques, such as the formation of diastereomeric pairs by salt formation with an optically active acid, such as (−)-di-p-toluoyl-d-tartaric acid and/or (+)-di-p-toluoyl-1-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973, and T.W. Greene & P.G.M. Wuts, Protective Groups in Organic Synthesis, John Wiley & Sons, 1991. The protecting groups may be removed at a convenient subsequent stage using methods known from the art.

Even though the compounds of the present invention (including their pharmaceutically, acceptable salts and pharmaceutically acceptable solvates) can be administered alone, they will generally be administered in admixture with a pharmaceutical carrier, excipient, or diluent selected with regard to the intended route of administration and standard pharmaceutical or veterinary practice. Thus, the present invention is directed to pharmaceutical and veterinary compositions comprising compounds of Formula (I) and one or more pharmaceutically acceptable carriers, excipients or diluents.

By way of example, in the pharmaceutical and veterinary compositions of the present invention, the compounds of the present invention may be admixed with any suitable binder(s), lubricant(s), suspending agent(s), coating agent(s), and/or solubilising agent(s).

Tablets or capsules of the compounds may be administered singly or two or more at a time, as appropriate. It is also possible to administer the compounds in sustained release formulations.

Alternatively, the compounds of the general Formulae (I) and (la) can be administered by inhalation (intratracheal or intranasal) or in the form of a suppository or pessary, or they may be applied topically in the form of a lotion, solution, cream, ointment or dusting powder. An alternative means of transdermal administration is by use of a skin patch. For example, the compounds can be incorporated into a cream consisting of an aqueous emulsion of polyethylene glycols or liquid paraffin. Alternatively, they can also be incorporated, at a concentration of between 1 and 10% by weight, into an ointment consisting of a white wax or white soft paraffin base together with such stabilisers and preservatives as may be required.

For some applications, preferably the compositions are administered orally in the form of tablets containing excipients such as starch or lactose, or in capsules or ovules either alone or in admixture with excipients, or in the form of elixirs, solutions or suspensions containing flavouring or coloring agents.

The compositions, as well as the compounds alone, can also be injected parenterally, for example intracavernosally, intravenously, intramuscularly or subcutaneously, intradermally or intrathecally. In this case, the compositions will comprise a suitable carrier or diluent.

For parenteral administration, the compositions are best used in the form of a sterile aqueous solution which may contain other substances, for example enough salts or monosaccharides to make the solution isotonic with respect to blood.

For buccal or sublingual administration the compositions may be administered in the form of tablets or lozenges which can be formulated in a conventional manner.

By way of further example, pharmaceutical and veterinary compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral, etc.). Thus for liquid oral preparations such as suspensions, elixirs and solutions, suitable carriers and additives include water, glycols, oils, alcohols, flavoring agents, preservatives, stabilizers, coloring agents and the like, for solid oral preparations, such as powders, capsules and tablets, suitable carriers and additives include starches, sugars, diluents, granulating agents, lubricants, binders, disintegrating agents and the like. Solid oral preparations also may be coated with substances such as sugars or be entericly-coated so as to modulate the major site of absorption. For parenteral administration, the carrier will usually consist of sterile water and other ingredients may be added to increase solubility or preservation. Injectable suspensions or solutions may also be prepared utilizing aqueous carriers along with appropriate additives.

Advantageously, compounds of the present invention may be administered in a single daily dose, or the total daily dosage may be administered in divided doses of two, three or four times daily. Furthermore, compounds of the present invention can be administered in intranasal form via topical use of suitable intranasal vehicles or via transdermal skin patches well known to those skilled in that art.

A therapeutically effective amount of the instant compounds or a pharmaceutical composition thereof comprises a dose range of from about 0.001 mg to about 1,000 mg, in particular from about 0.1 mg to about 500 mg or, more particularly from about 1 mg to about 250 mg of active ingredient per day for an average (70 kg) human.

For oral administration, a pharmaceutical composition is preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the subject to be treated.

It is also apparent to one skilled in the art that the therapeutically effective dose for active compounds of the invention or a pharmaceutical composition thereof will vary according to the desired effect. Therefore, optimal dosages to be administered may be readily determined and will vary with the particular compound used, the mode of administration, the strength of the preparation, and the disease condition and/or the stage thereof. In addition, factors associated with the particular subject being treated, including subject age, weight and diet, will result in the need to adjust the dose to achieve an appropriate therapeutic level. The above dosages are thus exemplary of the average case. Of course, there can be individual instances wherein higher or lower dosage ranges are merited, and such are within the scope of this invention.

Compounds of this invention may be administered in any of the foregoing compositions and dosage regimens or by means of those compositions and dosage regimens established in the art whenever use of the compounds of the invention as vanilloid receptor modulators is required for a subject in need thereof.

The invention also provides a pharmaceutical or veterinary pack or kit comprising one or more containers filled with one or more of the ingredients of the pharmaceutical and veterinary compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

As modulators of the VR1 ion channel, the compounds of Formulae (I) and (Ia) are useful in methods for treating or preventing a disease or condition in a mammal which disease or condition is affected by the modulation of one or more vanilloid receptors. Such methods comprise administering to a mammal in need of such treatment or prevention a therapeutically effective amount of a compound, salt or solvate of Formulae (I) and (Ia). In particular, the compounds of Formulae (I) and (Ia) are useful for preventing or treating chronic or acute pain causing diseases or conditions and pulmonary dysfunction, and more particularly, in treating diseases or conditions that cause inflammatory pain, burning pain, itch or urinary incontinence, and chronic obstructive pulmonary disease.

By way of example only, the compounds of Formulae (I) and (Ia) are useful for treating diseases and conditions selected from the group consisting of osteoarthritis, rheumatoid arthritis, fibromyalgia, migraine, headache, odontalgia, fever, burn, sunburn, snake bite (in particular, venomous snake bite), spider bite, insect sting, neurogenic/overactive bladder, urinary incontinence, benign prostatic hypertrophy, interstitial cystitis, urinary tract infection, cough, asthma, chronic obstructive pulmonary disease, rhinitis, contact dermatitis/hypersensitivity, itch, eczema, anxiety, panic disorders, pharyngitis, mucositis, enteritis, cellulits, peripheral neuropathy, bilateral peripheral neuropathy, diabetic neuropathy, central pain, neuropathies associated with spinal cord injury, stroke, ALS, Parkinson's disease, or multiple sclerosis, postherpetic neuralgia, trigeminal neuralgia, causalgia, sciatic neuritis, mandibular joint neuralgia, peripheral neuritis, polyneuritis, stump pain, phantom limb pain, bony fractures, post-operative ileus, irritable bowel syndrome, inflammatory bowel diseases such as Crohn's Disease and ulcerative colitis, cholecystitis, pancreatitis, postmastectomy pain syndrome, menstrual pain, endometriosis, dysmenorrhea, oral neuropathic pain, Charcot's pain, complex regional pain syndrome I and II (CRPS I/II), radiculopathy, Guillain-barre syndrome, meralgia paresthetica, burning-mouth syndrome, optic neuritis, postfebrile neuritis, migrating neuritis, segmental neuritis, Gombault's neuritis, neuronitis, cervicobrachial neuralgia, cranial neuralgia, geniculate neuralgia, glossopharyngial neuralgia, migrainous neuralgia, idiopathic neuralgia, intercostals neuralgia, mammary neuralgia, Morton's neuralgia, nasociliary neuralgia, occipital neuralgia, red neuralgia, Sluder's neuralgia, splenopalatine neuralgia, supraorbital neuralgia, vidian neuralgia, sinus headache, tension headache, pain associated with labor and childbirth, hot flash, cancer pain, especially that associated with bone cancer, and trauma.

While the present invention comprises compositions comprising one or more of the compounds of Formulae (I) and (Ia) the present invention also comprises compositions comprising intermediates used in the manufacture of compounds of Formulae (I) and (Ia).

Representative IUPAC names for the compounds of the present invention were derived using the ACD/LABS SOFTWARE™ Index Name Pro Version 4.5 nomenclature software program provided by Advanced Chemistry Development, Inc., Toronto, Ontario, Canada.

Abbreviations used in the instant specification, particularly the Schemes and Examples, are as follows:
Boc=tert-butoxycarbonyl
BOP—Cl=bis(2-oxo-3oxazolidinyl)phosphonic chloride
BuLi=n-butyllithium
Cmpd or Cpd=compound
d=day/days
DCM=dichloromethane
DIPEA=diisopropylethylamine
DMAP=4-dimethylaminopyridine
DMC=2-chloro-1,3-dimethyl-4,5-dihydro-1H-imidazolium chloride
DMSO=dimethylsulfoxide
dppf=diphenylphosphinoferrocene
EDCl=1-(3-Dimethylaminopropyl)-3-ethylcarbodiimide hydrochloride
EtOAc=ethyl acetate
EtOH=ethanol
h=hour/hours
HBTU=O-benzotriazol-1-yl-N,N,N′,N′-tetramethyluronium hexafluorophosphate
HOBt=hydroxybenzotriazole
LDA=lithium diisopropyamide
LPS=lipopolysaccharide
M=molar
MeCN=acetonitrile
MeOH=methanol
min=mindtes
rt/RT=room temperature
THF=tetrahydrofuran
TFA=trifluoroacetic acid
TFAA=trifluoroacetic anhydride

### General Synthetic Methods

Representative compounds of the present invention can be synthesized in accordance with the general synthetic methods described below and are illustrated in the schemes that follows. Since the schemes are an illustration, the invention should not be construed as being limited by the chemical reactions and conditions expressed. The preparation of the various starting materials used in the schemes is well within the skill of persons versed in the art.

Compounds of the present invention can be synthesized using the methods described in the schemes that follow. Scheme AA illustrates the general synthesis for amides of formula AA3.

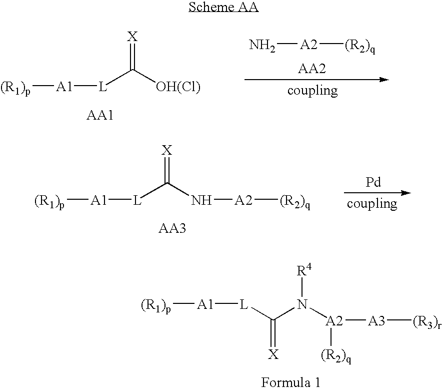

A carboxylic acid or acid chloride of formula AA1 may be reacted with an amine of formula AA2 in the presence of an appropriate coupling agent, base, and solvent to provide amide intermediates of the present invention. One example of an appropriate set of coupling reagents is DMC with diisopropylethylamine as a base in dichloromethane solvent. Upon amide formation, compounds of formula AA3 wherein ring A2 is appropriately activated may undergo a palladium cross coupling reaction in the presence of a base such as cesium carbonate or the like to install ring A3. Examples of palladium cross coupling procedures include Suzuki and Stille couplings, both of which are well known to those versed in the art.

Alternatively, rings A2 and A3 may be coupled first, followed by the formation of the amide linkage of Formula 1. As shown in Scheme BB, ring A2 of formula BB1 is substituted with a nitro group and an iodo group, and may be reacted under standard Suzuki coupling conditions with a boronic acid of ring A3 to afford compounds of formula BB3.

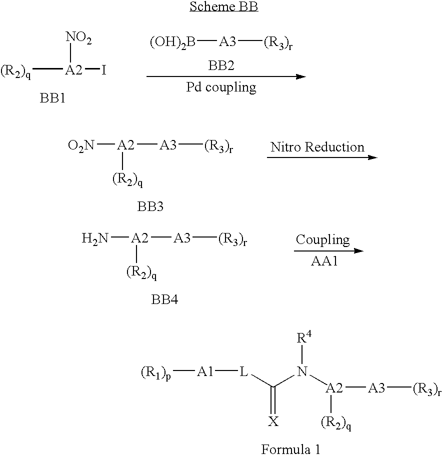

The nitro group may then be reduced to the corresponding amine of formula BB4 by hydrogenation in the presence of a suitable catalyst, or in the presence of other suitable reducing agents. Other methods commonly used for the reduction of nitro groups include treatment with zinc catalyst in the presence of acid, treatment with tin (II) chloride, or treatment with palladium on carbon in the presence of ammonium acetate. Compounds of formula BB4 may then be coupled with an intermediate of formula AA1 using methods described herein to afford compounds of the present invention.

The chemistry described in Scheme BB may be varied to prepare compounds of the present invention. For instance, the intermediates of formula BB4 can be prepared by modifying the precursors such that ring A2 bears a boronic acid, and ring A3 is substituted with a halogen or triflate. In addition, one skilled in the art will recognize that the amino portion of ring A2 may be derived from a protected amino group rather than a nitro group.

Manipulations of R₂ and R₃ or precursors of R₂ and R₃ may be performed to prepare certain compounds of the present invention. For example, to prepare compounds in which R₃ is an aminocarbonyl or the like, compounds of formula BB1 can be coupled with compounds of formula BB2 wherein A3 is substituted with a methyl group. The methyl substituent may be oxidized using conventional oxidation chemistry, such as treatment with Jones reagent, to afford the corresponding carboxylic acid. The carboxylic acid may be elaborated to an amide functionality via an acid chloride in reaction with appropriate amines, or via a standard coupling of the carboxylic acid with appropriate amines in the presence of a coupling agent such as EDCl, DCM, or the like. Alternatively, compounds in which R₂ and/or R₃ are a hydroxyalkyl substituent as defined herein may be derived from a carboxylic acid when treated with a hydride source such as lithium aluminum hydride or borane.

When R₃ is a hydroxy group, it can be elaborated through reaction with a bis-alkylating agent, such that one leaving group is displaced by the hydroxy group, and the other leaving group is displaced by an amino group or an amino group synthon, such as potassium phthalimide. At a later stage of the synthesis, the phthalimide group can be removed using a reagent such as hydrazine to afford amino-alkoxy substituents of R₃ of the present invention. When R₃ is an amino group, it can be alkylated with a hydroxy-substituted alkylating agent to arrive at hydroxylated alkylamino substituents of R₃ as defined herein.

Compounds of the present invention wherein ring A1 is a thiophene can be prepared according to Scheme CC.

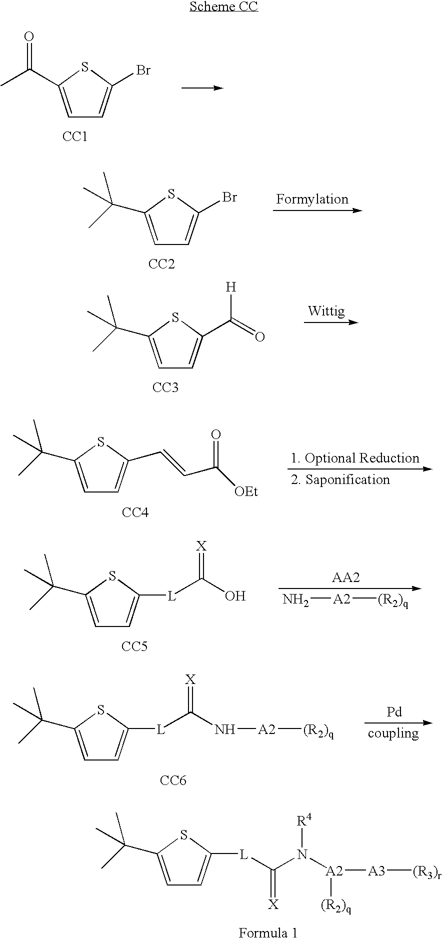

A 2-bromo, 5-acetyl-substituted thiophene can be reacted with dimethylzinc and titanium tetrachloride to effect the conversion of the 5-acetyl to a 5-tert-butyl substituent as illustrated in intermediate CC2. Treatment of intermediate CC2 with an organometallic base such as n-butyllithium in the presence of DMF installs a formyl substituent in the 2-position. The formyl group can then undergo a Wittig reaction with an aldehyde such as (carbethoxymethylene)-triphenylphosphorane or ketone to install the L group as an alkenyldiyl linker. The L group can optionally be reduced to an alkanyidiyl linker by hydrogention in the presence of palladium catalyst, or using other suitable reducing agents. The carbethoxy group can then be saponified to its corresponding carboxylic acid CC5. The carboxylic acid can be coupled with an aniline of formula AA2 using methods described herein to form compounds of formula CC6. Ring A3 may be installed as described herein to afford compounds of formula 1.

Scheme DD describes the preparation of certain intermediates of the present invention wherein A2 is phenyl or pyridinyl, and A3 is imidazolyl, pyrazolyl, indolyl, benzimidazolyl, triazolyl, dihydropyrazolyl, tetrahydrobenzimidazolyl, tetrahydroindazolyl, tetrahydroquinolinyl, or-quinolinyl.

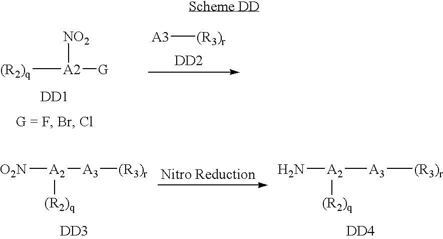

Compounds of formula DD1 which are substituted with a nitro group and a para-substituted halogen (G) such as fluorine, bromine or chlorine may be reacted with compounds of formula DD2 (where A3 is imidazolyl, pyrazolyl, indolyl, triazolyl or dihydropyrazolyl) in the presence of an appropriate base such as potassium hydroxide in a solvent such as DMSO to provide compounds of formula DD3 wherein ring A2 is phenyl or pyridyl and in which ring A3 is linked through a nitrogen atom to ring A2. The nitro group may then be reduced to the corresponding amine of formula DD4 by hydrogenation in the presence of a suitable catalyst, or in the presence of other suitable reducing agents. Other methods commonly used for the reduction of nitro groups include treatment with zinc catalyst in the presence of acid, treatment with tin (II) chloride, or treatment with palladium on carbon in the presence of ammonium acetate. Compounds of formula DD4 may then be coupled with an intermediate of formula AA1 using methods described herein to afford compounds of Formula 1.

Scheme EE describes the preparation of certain intermediates of the present invention wherein A3 is imidazolyl, pyrazolyl, indolyl, benzimidazolyl, triazolyl, dihydropyrazolyl, tetrahydrobenzimidazolyl, tetrahydroindazolyl, tetrahydroquinolinyl, or tetrahydroisoquinolinyl.

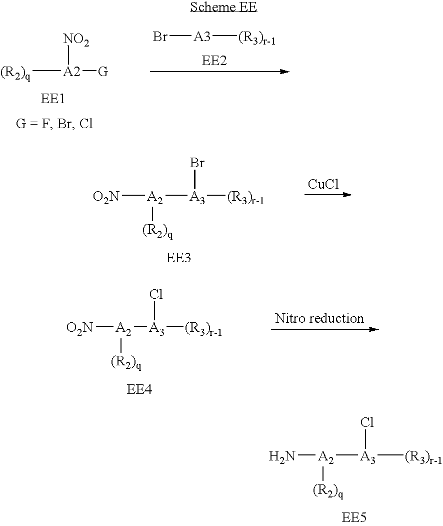

A compound of formula EE3 may be prepared by the reaction of a compound of formula EE1 with a compound of formula EE2 (wherein one of R₃ is bromol in the presence of an appropriate base such as potassium carbonate, in a solvent such as acetonitrile. The compound of formula EE3 may be reacted with copper (I) chloride in a solvent such as DMSO and heated to a temperature between 150-200 C to give the corresponding chlorinated compound of formula EE4. The compound of formula EE4 may then be reduced to the compound of formula EE5 by reaction with a suitable reducing agent such as zinc catalyst in the presence of acid, treatment with tin (II) chloride, or treatment with palladium on carbon in the presence of ammonium acetate. The compound of formula EE5 may then be utilized as described in Scheme DD to afford compounds of Formula 1.

Scheme FF describes the preparation of certain carboxylic acid and acid chloride intermediates of the present invention wherein ring A1 is R₁-substituted with an appropriate amine, herein defined as —NR₁ₐR_{1b}. 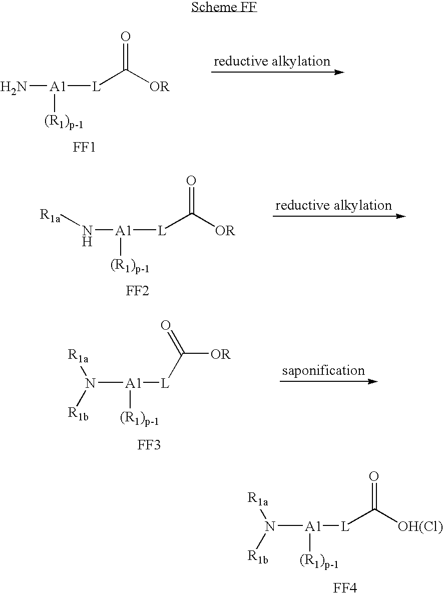
wherein R₁ₐ is H or C₁₋₈R_{1b}-substituted aldehyde in the presence of a reducing agent such as tetramethylammonium triacetoxyborohydride, sodium triacetoxyborohydride, sodium cyanoborohydride and the like, in a suitable solvent such as dichloroethane, dichloromethane, chloroform, methanol, tetrahydrofuran and the like, at a temperature in the range of ambient temperature to a temperature of about 70-100 C to yield the corresponding amine of formula FF3. The compound of formula FF3 may be saponified by reaction with a suitable base such as sodium hydroxide, potassium hydroxide, lithium hydroxide, sodium carbonate, potassium carbonate and the like, in a solvent such as ethanol, methanol, aqueous tetrahydrofuran and the like, at a temperature from ambient temperature to a temperature of about 70-100 C to yield the corresponding compound of formula FF4. The compound of formula FF4 may then be coupled with an intermediate of formula AA2 using methods described herein to afford compounds of Formula 1.

Scheme GG describes the preparation of certain carboxylic acid and acid chloride intermediates of the present invention wherein ring A1 is phenyl or naphthalenyl, and A1 is substituted with R₁, wherein R₁ is trifluoromethylsulfinyl, trifluoromethylthio, or trifluoromethylsulfonyl. In Scheme HH, L is as defined herein, such that L does not exceed 8 carbons in chain length.

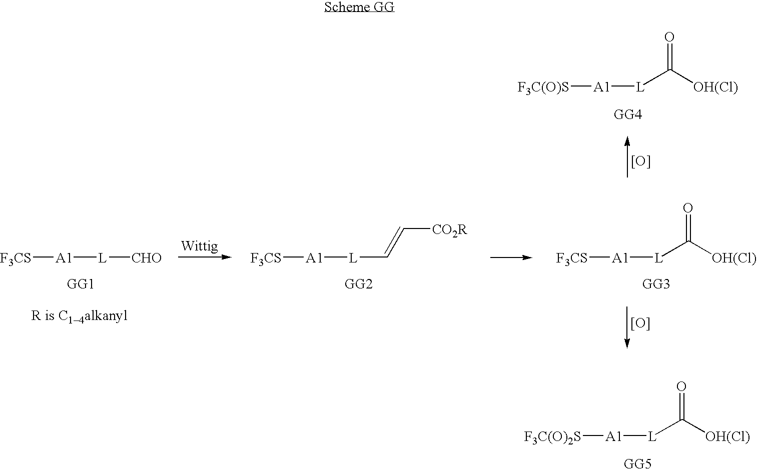

Specifically, an aldehyde of formula GG1 can be treated with a Wittig reagent such as ethyl(triphenylphosphoranylidene)acetate (purchased from Aldrich Chemicals) in a suitable solvent such a benzene or toluene at an elevated temperature, preferably at a temperature in a range of 80-100 C to yield a compound of formula GG2. This compound may then be saponified by reaction with suitable base such as sodium hydroxide, potassium hydroxide, lithium hydroxide, sodium carbonate, potassium carbonate and the like, in a solvent such as ethanol, methanol or aqueous tetrahydrofuran, at a temperature from ambient temperature to a temperature of about 70-100 C to yield the corresponding carboxylic acids of formula GG3. The carboxylic acids of formula GG3 can be oxidized with a suitable oxidizing agent such as m-chloroperbenzoic acid in a solvent such as trifluoroacetic acid to yield the compound(s) of formula GG4 and GG5. Compounds of Scheme HH in which L is an alkenyldiyl may be reduced to its corresponding alkanyl form using convention reduction chemistry. For example, compound(s) of formulae GG3, GG4 and GG5 can be reduced by treatment with hydrogen gas at an elevated pressure in the range of about 40-50 psi in a suitable solvent such as ethanol or methanol, in the presence of a catalyst such as 10% palladium on carbon at ambient temperature. Alternative methods of reduction include reaction with cyclohexene, cyclohexadiene or ammonium formate in a suitable solvent such as ethanol using a catalyst such as 10% palladium on carbon. The carboxylic acids of formula GG3-GG5 can be reacted with a chlorinating agent such as oxalyl chloride or thionyl chloride in the presence of an acylation catalyst such as DMF in a suitable solvent such as methylene chloride, chloroform or dichloroethane at a temperature of about 0 C to yield the corresponding acid chlorides. The compound(s) of formula GG3-G5 may then be coupled with an intermediate of formula AA2 using methods described herein to afford compounds of Formula 1.

Scheme HH describes the preparation of certain intermediates of the present invention, wherein ring A1 is pyridinyl.

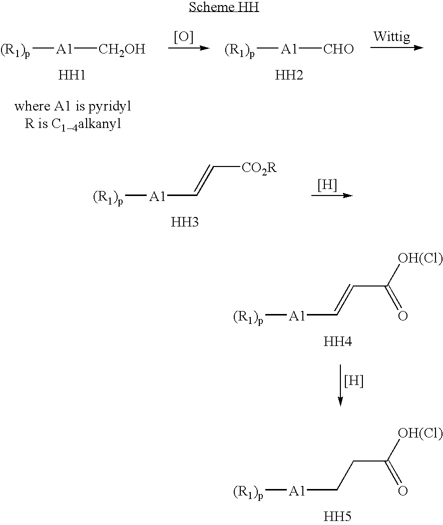

An alcohol of formula HH1 may be oxidized by a suitable oxidizing agent, such as manganese dioxide, in a solvent such as methylene chloride to yield a compound of formula HH 2. Compounds of forrmula HH2 may then be reacted with a Wittig reagent such as ethyl(triphenylphosphoranylidene)acetate (purchased from Aldrich Chemicals) in a suitable solvent such a benzene or toluene at an elevated temperature, preferably at a temperature in a range of 80-100 C to yield the compound of formula HH3. The compound of formula HH3 may then be saponified by reaction with suitable base such as sodium hydroxide, potassium hydroxide, lithium hydroxide, sodium carbonate, potassium carbonate and the like in a solvent such as ethanol, methanol or aqueous tetrahydrofuran, at a temperature from ambient temperature to a temperature of about 70-100 C to yield the corresponding carboxylic acids of formula HH4. The compound of formula HH4 may be reduced by treating with hydrogen gas at an elevated pressure in the range of about 40-50 psi in a suitable solvent such as ethanol or methanol, in the presence of a catalyst such as 10% palladium on carbon at ambient temperature to yield the corresponding carboxylic acid of formula HH5. Alternative methods of reduction include reaction with cyclohexene, cyclohexadiene, or ammonium formate in a suitable solvent such as ethanol using a catalyst such as 10% palladium on carbon. The carboxylic acid(s) of formula HH4 and HH5 may be reacted with a chlorinating agent such as oxalyl chloride or thionyl chloride in the presence of an acylation catalyst such as DMF in a suitable solvent such as methylene chloride, chloroform or dichloroethane at ambient temperature or below to yield the corresponding acid chloride of formula HH4 and HH5. The compound(s) of formula HH4 and HH5 may then be coupled with an intermediate of formula AA2 using methods described herein to afford compounds of Formula 1.

Scheme JJ describes the preparation of certain intermediates of the present invention, wherein ring A3 is tetrahydrobenzimidazolyl, and r is 0.

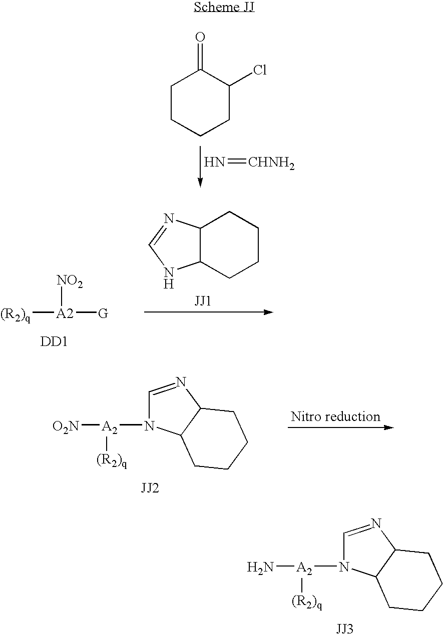

A compound of formula JJ1 may be synthesized by reaction of 2-chloro-cyclohexanone with formamidine hydrochloride in the presence of a base such as potassium carbonate in a solvent such as toluene. The compound of formula JJ1 can be reacted with a compound of formula DD1 in the presence of an appropriate base such as potassium hydroxide in a solvent such as DMSO to provide compounds of formula JJ2 wherein ring A2 is phenyl or pyridyl and a tetrahydrobenzimidazole of formula A3 is N-linked to ring A2. The nitro group may then be reduced to the corresponding amine of formula JJ3 by hydrogenation in the presence of a suitable catalyst, or in the presence of other suitable reducing agents. Other methods commonly used for the reduction of nitro groups include treatment with zinc catalyst in the presence of acid, treatment with tin (II) chloride, or treatment with palladium on carbon in the presence of ammonium acetate. Compounds of formula JJ3 may then be coupled with an intermediate of formula DD1 using methods described herein to afford compounds of Formula 1.

Other compounds of the present invention wherein ring A3 is a N-linked tetrahydroindazoyl, tetrahydroisoquinolyl or tetrahydroquinolinyl (and r is 0), may be prepared according to Scheme JJ, substituting the appropriately commercially available A3 for the compound of formula JJ1.

Protecting group manipulations may be needed at various stages of the syntheses depending upon substituents and functional groups that are present on the reactants. Microwave accelerated reactions were performed using a Personal Chemistry Smith Synthesizer microwave instrument.

It is generally preferred that the respective product of each process step be separated from other components of the reaction mixture and subjected to purification before its use as a starting material in a subsequent step. Separation techniques typically include evaporation, extraction, precipitation and filtration. Purification techniques typically include column chromatography (Still, W. C. et. al., J. Org. Chem. 1978, 43, 2921), thin-layer chromatography, crystallization and distillation. The structures of the final products, intermediates and starting materials are confirmed by spectroscopic, spectrometric and analytical methods including nuclear magnetic resonance (NMR), mass spectrometry (MS) and liquid chromatography (HPLC). In the descriptions for the preparation of compounds of this invention, ethyl ether, tetrahydrofuran and dioxane are common examples of an ethereal solvent, benzene, toluene, hexanes and cyclohexane are typical hydrocarbon solvents and dichloromethane and dichloroethane are representative halogenhydrocarbon solvents. In those cases wherein the product is isolated as the acid addition salt the free base may be obtained by techniques known to those skilled in the art. In those cases in which the product is isolated as an acid addition salt, the salt may contain one or more equivalents of the acid.

Representative compounds of the present invention can be synthesized in accordance with the general synthetic methods described above and are illustrated more particularly in the schemes that follow. Since the schemes are illustrations, the invention should not be construed as being limited by the chemical reactions and conditions expressed. The preparation of the various starting materials used in the schemes is well within the skill of persons versed in the art.

### EXAMPLE 1

### 3-(4-tert-butyl-phenyl)-N-(3′-hydroxy-biphenyl-4-yl)-propionamide (Cpd 6)

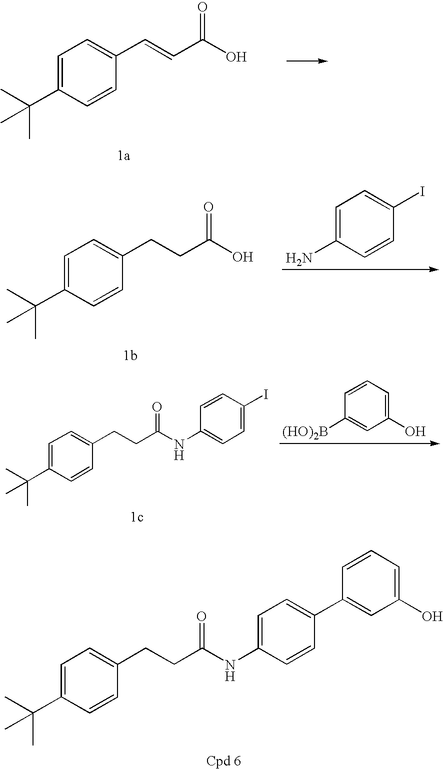

A. A sample of 3-(4-tert-butyl-phenyl)-acrylic acid (12.28 g, 60.1 mmol) was slurried in a mixture of ethanol (100 mL) and 10% palladium on carbon (0.60 g). The mixture was hydrogenated at 50 psi hydrogen for 1.5 h, then filtered over a pad of Celite. The solvents were removed in vacuo to yield 3-(4-tert-butyl-phenyl)-propionic acid as a white crystalline powder (12.28 g, 59.5 mmol). ¹H NMR (d₆-DMSO): δ 12.08 (s, 1H), 7.28 (d, 2H), 7.13 (d, 2H), 2.77 (t, 2H), 2.50 (t, 2H), 1.24 (s, 9H).

b. To a suspension of Cpd 1b (6.21 g, 30.1 mmol) in methylene chloride (200 mL) was subsequently added diisopropylethylamine (12.0 mL, 68.9 mmol), para-iodoaniline (6.62 g, 30.2 mmol) and DMC coupling reagent (5.62 g, 33.2 mmol). The reaction was stirred at ambient temperature for 16 h, then washed twice with 1 N HCl (200 mL) and twice with saturated NaHCO₃ solution (200 mL). The organics were dried with Na₂SO₄, filtered and evaporated in vacuo. The residue obtained was triturated with 1:1 hexanes/ethyl acetate (25 mL). The resultant solid was collected by filtration and rinsed once with 1:1 hexanes/ethyl acetate (10 mL) and dried. Compound 1c (8.31 g, 20.4 mmol) was obtained as a white crystalline powder. MS: M+H⁺=407.9, ¹H NMR (CDCl₃): δ 7.59 (d, 2H), 7.33 (d, 2H), 7.18 (t, 4H), 6.88 (br s, 1H), 3.02 (t, 2H), 2.66 (t, 2H), 1.31 (s, 9H).

C. To a solution of 3-(4-tert-butyl-phenyl)-N-(4-iodo-phenyl)-propionamide (0.408 g, 1.00 mmol) in 25 mL dioxane was added 3-hydroxy phenylboronic acid (0.154 g, 1.12 mmol), cesium fluoride (0.52 g, 3.42 mmol) and PdCl₂(dppf) (0.039 g, 0.05 mmol). The reaction was heated at reflux under a nitrogen atmosphere for 21 h. An additional portion of 3-hydroxy phenyl boronic acid (0.154 g, 1.12 mmol), cesium fluoride (0.53 g, 3.49 mmol) and PdCl₂(dppf) (0.040 g, 0.05 mmol) were added and the reaction was heated at reflux for another 1.5 h. A 5 mL volume of ethanol was introduced into the reaction and the reaction was heated for an additional 2.5 h. The solvents were removed in vacuo and the residue partitioned between 50 mL ethyl acetate and 25 mL water. The solvent mixture was filtered over a pad of Celite and then the layers were separated. The organic phase was isolated and washed sequentially with 25 mL saturated NaHCO₃ solution and 25 mL brine, then dried over Na₂SO₄, and evaporated. The resultant residue was chromatographed over silica gel (30-50% EtOAc/Hexanes) to give the title compound (0.082 g, 0.22 mmol) as a tan powder. MS: M+H⁺=374.1, ¹H NMR (d₆-DMSO): δ 9.98 (s, 1H), 9.48 (s, 1H), 7.68 (d, 2H), 7.56 (d, 2H), 7.32 (d, 2H), 7.27-7.16 (m, 3H), 7.06 (d, 1H), 6.99 (s, 1H), 6.73 (dd, 1H), 2.89 (t, 2H), 2.63 (t, 2H), 1.27 (s, 9H).

### EXAMPLE 2

### 3-(4-tert-butyl-phenyl)-N-(3-pyridin-2-yl-phenyl)-propionamide hydrochloride (Cpd 109)

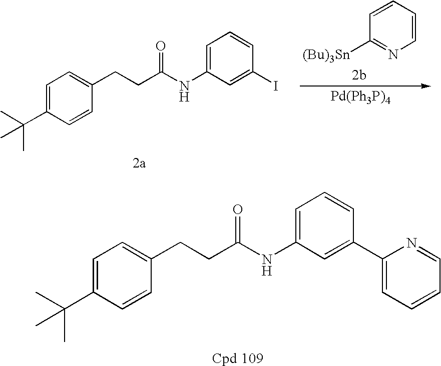

A. To a solution of 3-(4-tert-butyl-phenyl)-N-(3-iodo-phenyl)-propionamide, Cpd 2a, (0.204 g, 0.50 mmol) in 5 mL dioxane in a small pressure tube was added 2-tributylstannanyl-pyridine (0.165 mL, 0.52 mmol) and Pd(Ph₃P)₄ (0.088 g, 0.08 mmol). The vessel was capped and heated to 100 C for 24 h. The reaction mixture was evaporated in vacuo and the resultant residue chromatographed over silica gel (0-40% EtOAc/Hexanes). The resultant product was then converted to its hydrochloride salt using ethereal HCl to afford the title compound (0.044 g, 0.11 mmol). MS: M+H⁺=359.3, ¹H NMR (CDCl₃): δ 8.84 (br s, 1H), 8.63 (d, 1H), 8.20 (s, 1H), 7.94 (t, 1H), 7.79 (d, 2H), 7.64-7.52 (m, 2H), 7.38-7.21 (m, 5H), 3.08 (t, 2H), 2.89 (t, 2H), 1.26 (s, 9H).

### EXAMPLE 3

### 3-(4-tert-butyl-phenyl)-N-(4-pyridin-4-yl-phenyl)-propionamide (Cpd 111)

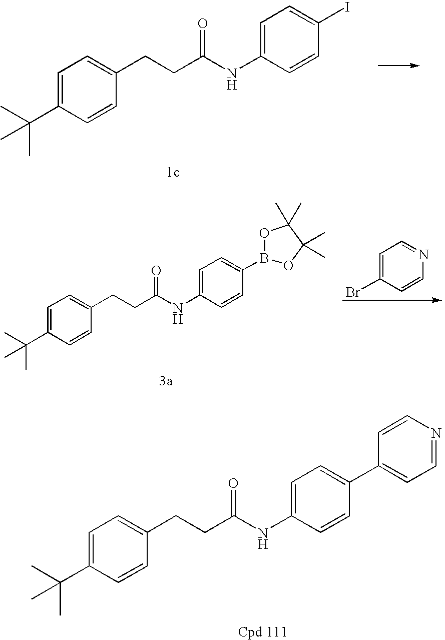

A. To a solution of Cpd 1c (1.222 g, 3.00 mmol) in 18 mL DMSO in a pressure tube was added KOAc (0.887 g, 9.04 mmol), 4,4,5,5,4′,4′,5′,5′-octamethyl-[2,2′]bi[[1,3,2]dioxaborolanyl] (0.843 g, 3.32 mmol), and PdCl₂(dppf) (0.066 g, 0.09 mmol). The vessel was capped and heated on an 80 C oil bath for 16 h. The reaction mixture was added to 100 mL benzene and washed three times with 100 mL water. The aqueous and organic phases were separated, and the organic phase was dried over Na₂SO₄. Evaporation of the organic phase yielded a residue that was purified via silica gel chromatography (0-75% EtOAc/Hexanes) to give compound 3a (0.807 g, 1.98 mmol) as an off-white powder. MS: M+H⁺=407.8, ¹H NMR (CDCl₃): δ 7.77 (d, 2H), 7.43 (d, 2H), 7.34 (d, 2H), 7.20 (d, 2H), 6.97 (br s, 1H), 3.06 (t, 2H), 2.68 (t, 2H), 1.38-1.29 (m, 21H).

B. To a solution of Cpd 3a (0.196 g, 0.48 mmol) in 4 mL toluene and 1 mL ethanol was added 2 M aqueous Na₂CO₃ (0.75 mL, 1.50 mmol), 4-bromopyridine hydrochloride (0.106 g, 0.55 mmol), and Pd(Ph₃P)₄ (0.028 g, 0.02 mmol). The vessel was capped and heated on a 100 C oil bath for 21 h. The reaction mixture was added to 25 mL EtOAc, washed twice with 25 mL of 10% Na₂CO₃ solution, and concentrated in vacuo to a residue. The residue was purified via silica gel chromatography (0-5% 2 M ammonia in MeOH/dichloromethane) to give the title compound (0.170 g, 0.47 mmol). MS: M+H⁺=359.2, ¹H NMR (CDCl₃): δ 8.65 (d, 2H), 7.62-7.52 (m, 4H), 7.48 (d, 2H), 7.37 (d, 2H), 7.20 (d, 2H), 7.16 (br, s, 1H), 3.07 (t, 2H), 2.71 (t, 2H), 1.32 (s, 9H).

### EXAMPLE 4

### 4′-[3-(4-tert-butyl-phenyl)-propionylamino]-biphenyl-3-carboxylic acid methyl ester (Cpd 29)

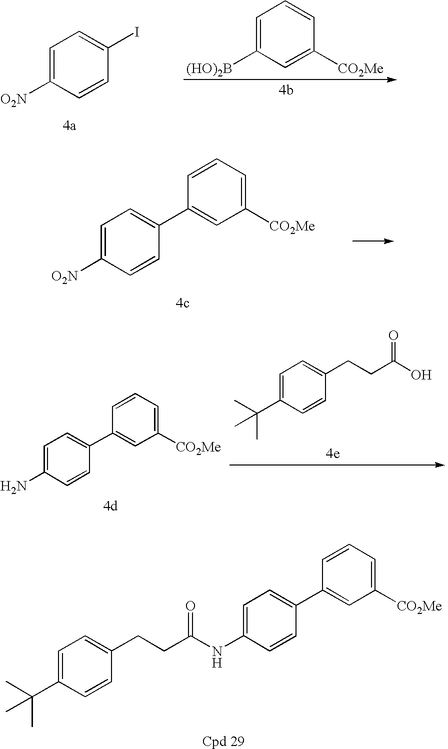

A. To a solution of 1-iodo-4-nitro-benzene (1.249 g, 5.02 mmol) in 30 mL dioxane in a pressure tube was added 3-carbomethoxyphenylboronic acid (0.993 g, 5.52 mmol), PdCl₂(dppf) (0.185 g, 0.25 mmol), and cesium fluoride (2.61 g, 17.2 mmol). The vessel was capped and heated in a microwave oven at 100 C for 20 min, then heated without irradiation at 100 C for 14 h, and then lastly heated again in a microwave at 100 C for 20 min. The reaction mixture was added to 200 mL EtOAc and sequentially washed, twice with 100 mL of 10% Na₂CO₃ solution, once with 100 mL brine, and then dried over Na₂SO₄. Evaporation of the organic phase in vacuo yielded a residue that was chromatographed over silica gel (100% CHCl₃) to provide a crude product. The product was titurated with 30 mL of boiling 5:1 hexanes/EtOAc. Upon cooling, the solid was isolated by filtration and dried to give Compound 4c (0.941 g, 3.66 mmol) as an orange crystalline powder. ¹H NMR (CDCl₃): δ 8.38-8.31 (m, 3H), 8.14 (d, 1H), 7.87-7.76 (m, 3H), 7.59 (t, 1H), 3.99 (s, 3H).

b. Compound 4c (0.938 g, 3.65 mmol) and 10% palladium on carbon (0.095 g) in 25 mL methanol were hydrogenated (50 psi hydrogen gas) for 1 h. The reaction mixture was filtered over a pad of Celite and the filtrate was concentrated in vacuo to yield Compound 4d as an orange oil (0.87 g). MS: M+H⁺=227.9, ¹H NMR (CDCl₃): δ 8.25 (d, 1H), 7.98 (dd, 1H), 7.77 (t, 1H), 7.57 (d, 1H), 7.52-7.42 (m, 2H), 7.09 (d, 1H), 6.79 (d, 1H), 3.96 (s, 3H).

C. Compound 4d (3.65 mmol) and Compound 4e (0.754 g, 3.66 mmol) were dissolved in 10 mL DMF. A portion of i-Pr₂NEt (0.70 mL, 4.02 mmol) and HbtU (1.416 g, 3.73 mmol) were added and the reaction was stirred for 19 h. The reaction mixture was diluted into 100 mL EtOAc and sequentially washed, twice with 100 mL saturated NaHCO₃ and once with 100 mL brine. The aqueous and organic phases were separated, and the organic phase was dried over Na₂SO₄, filtered, and evaporated in vacuo to give a residue. The resultant residue was purified via silica gel chromatography (0-5% MeOH/CHCl₃) to give a crude product. The product was further purified by trituration using 4 mL of 1:1 EtOAc/Hexanes to afford Compound 29 (0.629 g, 1.51 mmol) as a tan-orange powder. MS: M+H⁺=416.2, ¹H NMR (CDCl₃): δ 8.27 (s,1 H), 8.02 (d,1 H), 7.77 (d, 1H), 7.62-7.47 (m, 5H), 7.36 (d, 2H), 7.20 (d, 2H), 7.03 (br s, 1H), 3.96 (s, 3H), 3.07 (t, 2H), 2.69 (t, 2H), 1.32 (s, 9H).

### EXAMPLE 5

### 4′-[3-(4-tert-butyl-phenyl)-propionylamino]-biphenyl-3-carboxylic acid (Cpd 30)

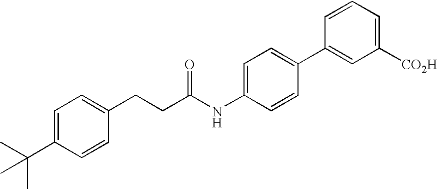

To a solution of Compound 29 (0.208 g, 0.50 mmol) in 30 mL of 5:1 tHF/H₂O was added LiOH.H₂O (0.042 g, 1.00 mmol). The reaction was stirred for 41 h and the volume of the reaction was reduced to near dryness by evaporating the solvent in vacuo. To the resultant wet residue was added 25 mL water and 10 mL 1 N HCl. The precipitate was collected via filtration and dried under vacuum to give Compound 30 (0.190 g, 0.47 mmol) as a cream-colored powder. MS: M−H⁺=400.0, ¹H NMR (d₆-DMSO): δ 13.09 (br s, 1H), 10.08 (s, 1H), 8.17 (s, 1H), 7.90 (d, 2H), 7.75-7.63 (m, 4H), 7.58 (t, 1H), 7.32 (d, 2H), 7.19 (d, 2H), 2.91 (t, 2H), 2.65 (t, 2H), 1.26 (s, 9H).

### EXAMPLE 6

### 4′-[3-(4-tert-butyl-phenyl)-propionylamino]-biphenyl-3-carboxylic acid amide (Cpd 31)

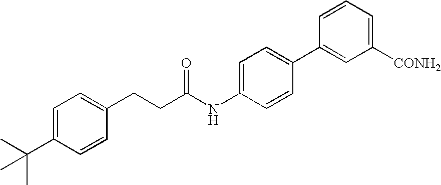

As per the literature procedure (Syn. Com. (1982), 989-93), to a suspension of Compound 29 (0.208 g, 0.50 mmol) in 5 mL benzene in a pressure tube was added CH₃Al(Cl)NH₂ (2.3 mL, 0.67 M in benzene/toluene, 1.5 mmol). The vessel was capped and heated at 50 C for 23 h. The reaction mixture was diluted-with 25 mL 1 N HCl/25 mL EtOAc. The insoluble salts were removed by filtration, the phases were separated, and the organic phase was evaporated in vacuo to a residue. The residue was purified by reverse-phase chromatography to give Compound 31 (0.081 g, 0.20 mmol) as a pale yellow powder. MS: M+H⁺=401.1, ¹H NMR (d₆-DMSO): δ 10.08 (s, 1H), 8.14 (s, 1H), 8.09 (brs, 1H), 7.83 (t, 2H), 7.72 (s, 4H), 7.53 (t, 1H), 7.43 (br s, 1H), 7.32 (d, 2H), 7.19 (d, 2H), 2.91 (t, 2H), 2.63 (t, 2H), 1.28 (s, 9H).

### EXAMPLE 7

### 4′-[3-(4-tert-butyl-phenyl)-acryloylamino]-biphenyl-3-carboxylic acid amide (Cpd 35)

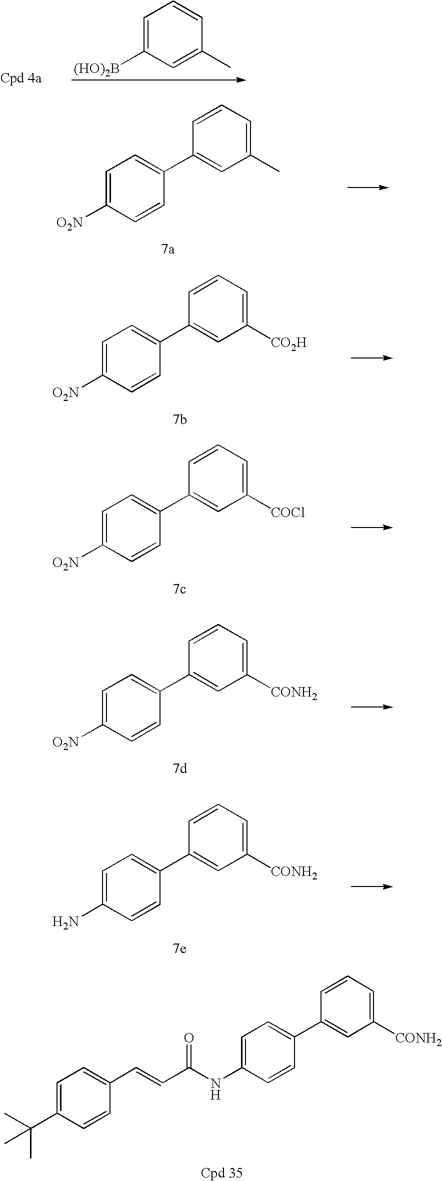

A. To a solution of Compound 4a (9.14 g, 36.7 mmol) and 3-methyl phenylboronic acid (5.08 g, 37.4 mmol) in a mixture of 90 mL acetone and 100 mL water was added K₂CO₃ (12.67 g, 91.7 mmol) and palladium(II) acetate (0.255 g, 1.1 mmol). Under an atmosphere of nitrogen, the reaction mixture was heated at reflux for 17 h. Additional palladium(II) acetate (0.503 g, 2.2 mmol) was added and the reaction was heated at reflux for an additional 4 h. After cooling, the reaction mixture was extracted twice with 200 mL diethyl ether. The combined organic extracts were washed once with 250 mL brine, dried over MgSO₄, treated with charcoal, filtered, and evaporated to give a residue. This residue was purified via silica gel chromatography (0-5% EtOAc/Hexanes) to give Compound 7a (5.99 g, 28.1 mmol) as a yellow oil. ¹H NMR (CDCl₃): δ 8.31 (d, 2H), 7.73 (d, 2H), 7.48-7.36 (m, 3H), 7.28 (d, 1 H), 2.47 (t, 3H).

B. A solution of Compound 7a (5.99 g, 28.1 mmol) in 30 mL pyridine and 60 mL water was heated at reflux. A portion of KMnO₄ (22.17 g, 140 mmol) was carefully introduced into the refluxing solution via the reflux condenser. The reaction mixture was heated at reflux for 1 h, and then filtered while hot over a pad of Celite. The filter cake was washed once with 100 mL of hot water. The combined filtrates were washed three times with 50 mL diethyl ether then diluted to a volume of 1 L with additional water. A 35 mL volume of concentrated HCl was added, and the resultant solid was isolated by filtration, rinsed with water and dried under vacuum to give Compound 7b (3.825 g, 15.7 mmol) as a white powder. MS: M−H⁺=242.0, ¹H NMR (d₆-DMSO): δ13.20 (s, 1H), 8.39-8.25 (m, 3H), 8.09-7.97 (m, 4H), 7.68 (t, 1 H).

C. To Compound 7b (3.65 g, 15.0 mmol) was added excess thionyl chloride. The reaction mixture was heated at reflux for 1.5 h, then evaporated in vacuo to give Compound 7c. A portion of Compound 7c (7.5 mmol) in 25 mL chloroform was added dropwise into a mixture of 30% aqueous ammonia (5 mL) and 3 N NaOH (25 mL). After 1 h, a precipitate was collected by vacuum filtration. The organic portion of the filtrate was isolated, washed once with 25 mL 1 N NaOH, dried over Na₂SO₄, filtered, and combined with the previously isolated precipitate. Together they were concentrated in vacuo to give Compound 7d. Compound 7d was taken up in 50 mL methanol containing 10% palladium on carbon (0.211 g) and hydrogenated at 50 psi hydrogen gas for 30 min. The reaction mixture was filtered over a pad of Celite and the filtrate was concentrated in vacuo to yield Compound 7e (1.58 g, 7.44 mmol) as an off-white powder. MS: M+H⁺=213.1, ¹H NMR (d₆-DMSO): δ 8.04 (s, 2H), 7.71 (t, 2H), 7.50-7.48 (m, 3H), 7.33 (s, 1H), 6.66 (d, 2H), 5.28 (s, 2H).

D. Compound 7e (0.212 g, 1.00 mmol) was dissolved in 5 mL DMF. To this was added sequentially i-Pr₂NEt (0.20 mL, 1.15 mmol), 3-(4-tert-butyl-phenyl)-acrylic acid (0.205 g, 1.00 mmol), and HBTU (0.387 g, 1.02 mmol). After 92 h the reaction mixture was diluted into 50 mL EtOAc and was washed twice with 50 mL 1 N HCl. The resultant solid and the organic phase were together concentrated in vacuo and purified by reverse phase chromatography to yield Compound 35 (0.239 g, 0.60 mmol). MS: M+H⁺=398.8, ¹H NMR (d₆-DMSO): δ 10.32 (s, 1H), 8.16 (s, 1H), 8.09 (br s, 1H), 7.88-7.78 (m, 4H), 7.73 (d, 2H), 7.63-7.38 (m, 7H), 6.82 (d, 1H), 1.28 (s, 9H).

### EXAMPLE 8

### 3-(4-tert-butyl-phenyl)-N-[6-(2-hydroxy-phenyl)-5-methyl-pyridin-3-yl]-propionamide (Cpd 160)

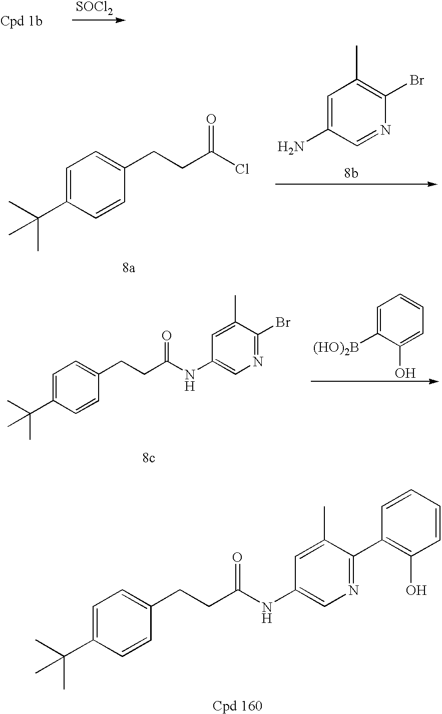

A. To Cpd 1b (3.42 g, 16.6 mmol) was added 15 mL thionyl chloride (15 mL, 206 mmol). The solution was heated at reflux for 2 h. The reaction was evaporated in vacuo, dissolved in benzene and evaporated, and the process was repeated. Compound 8a (3.67 g, 16.3 mmol) was obtained as a light yellow liquid and used for subsequent reactions without further purification.

B. To a solution of Compound 8b (0.936 g, 5.00 mmol) in 15 mL acetonitrile was added iPr₂NEt (0.96 mL, 5.51 mmol) and 3-(4-tert-butyl-phenyl)-propionyl chloride (1.07 mL, 5.00 mmol). The reaction was stirred at ambient temperature for 18 hours then evaporated in vacuo. The resulting solid was triturated with 5 mL acetonitrile, filtered off and rinsed with a few mL additional acetonitrile. The solid was dried under a stream of air to yield Compound 8c (1.412 g, 3.76 mmol) as an off-white crystalline powder. MS: M+H⁺=375.1, ¹H NMR (d₆-DMSO): 8.39 (s, 1H), 8.00 (s, 1H), 7.30 (d, 2H), 7.17 (d, 2H), 2.88 (t, 2H), 2.64 (t, 2H), 2.30 (s, 3H), 1.25 (s, 9H).

C. Compound 8c (0.094 g, 0.25 mmol) was dissolved in 5 mL 5:1 dioxane/ethanol solution in a microwave pressure vessel. To this was added 2-hydroxy phenyl boronic acid (0.040 g, 0.29 mmol), cesium carbonate (0.178 g, 0.55 mmol), and PdCl₂(dppf) (0.012 g, 0.016 mmol). The vessel was flushed with argon, capped, and reacted in a microwave apparatus at 100 C for 15 min then at 120 C for 15 min. Additional 2-hydroxy phenyl boronic acid (0.040 g, 0.29 mmol) and PdCl₂(dppf) (0.012 g, 0.016 mmol) was added to the reaction vessel. The vessel was flushed with argon, capped, and microwaved at 120 C for 15 min. The reaction mixture was diluted into 50 mL EtOAc and washed with 50 mL water with 5 mL brine. The organic phases were dried over Na₂SO₄, filtered and evaporated to a residue. This residue was purified by reverse-phase chromatography (25-95% acetonitrile/water+0.1% TFA). The title compound was isolated as a tan powder (0.058 g, 0.15 mmol). MS: M+H⁺=389.2, ¹H NMR (d₆-DMSO): δ 10.60 (s, 1H), 8.88 (s, 1H), 8.19 (s, 1H), 7.37 (t, 1H), 7.34-7.29 (m, 3H), 7.20 (d, 2H), 7.02 (d, 1H), 6.98 (t, 1 H), 2.92 (t, 2H), 2.72 (t, 2H), 2.27 (s, 3H), 1.27 (s, 9H).

### EXAMPLE9

### 3-(4-tert-butyl-phenyl)-N-[6-(3-hydroxy-phenyl)-5-methyl-pyridin-3-yl]-propionamide (Cpd 161)

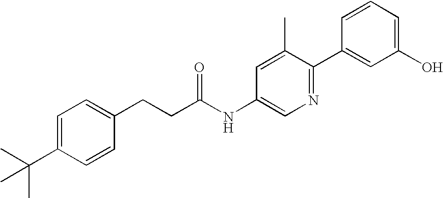

The title compound was obtained using the methods described in Example 8, substituting 3-hydroxy phenyl boronic acid for 2-hydroxy phenyl boronic acid. MS: M+H⁺=389.2, ¹H NMR (d₆-DMSO): δ 10.42 (s, 1 H), 9.69 (br s,1 H), 8.77 (s, 1H), 8.11 (s, 1H), 7.34-7.28 (m, 3H), 7.18 (d, 2H), 6.99-6.92 (m, 2H), 6.88 (d, 1H), 2.92 (t, 2H), 2.70 (t, 2H), 2.33 (s, 3H), 1.26 (s, 9H).

### EXAMPLE 10

### 3-(4-tert-butyl-phenyl)-N-(3-methyl-[2,4′]bipyridinyl-5-yl)-propionamide (Cpd 165)

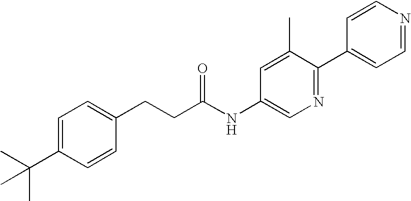

The title compound was obtained using the methods described in Example 8, substituting 4-pyridyl boronic acid for 2-hydroxy phenyl boronic acid. MS: M+H⁺=374.2, ¹H NMR (d₆-DMSO): δ 10.35 (s, 1H), 8.88 (d, 2H), 8.75 (s, 1H), 8.11 (s, 1H), 7.98 (d, 2H), 7.32 (d, 2H), 7.19 (d, 2H), 2.91 (t, 2H), 2.70 (t, 2H), 2.43 (s, 3H),1.26 (s, 9H).

### EXAMPLE 11

### 3-(4-tert-butyl-phenyl)-N-[6-(4-hydroxymethyl-phenyl)-5-methyl-pyridin-3-yl]-propionamide (Cpd 163)

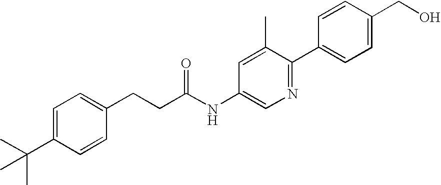

The title compound was obtained using the methods described in Example 8, substituting 4-hydroxymethylphenylboronic acid for 2-hydroxy phenyl boronic acid. MS: M+H⁺=403.2, ¹H NMR (d₆-DMSO): δ 10.45 (s, 1H), 8.81 (s, 1H), 8.14 (s, 1H), 7.53 (d, 2H), 7.47 (d, 2H), 7.31 (d, 2H), 7.18 (d, 2H), 4.59 (s, 2H), 2.92 (t, 2H), 2.70 (t, 2H), 2.34 (s, 3H), 1.26 (s, 9H).

### EXAMPLE 12

### 3-(4-tert-butyl-phenyl)-N-[6-(4-hydroxy-phenyl)-5-methyl-pyridin-3-yl]-propionamide (Cpd 162)

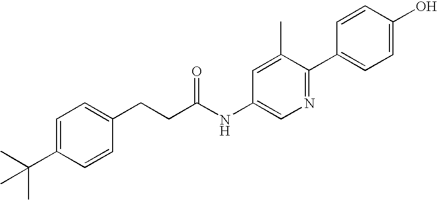

The title compound was obtained using the methods described in Example 8, substituting 4-hydroxyphenylboronic acid for 2-hydroxyphenylboronic acid. MS: M+H⁺=389.2, ¹H NMR (d₆-DMSO): δ 10.48 (s,1 H), 9.88 (br s,1 H), 8.79 (s, 1H), 8.12 (s, 1H), 7.43 (d, 2H), 7.31 (d, 2H), 7.18 (d, 2H), 6.91 (d, 2H), 2.91 (t, 2H), 2.69 (t, 2H), 2.37 (s, 3H), 1.24 (s, 9H).

### EXAMPLE 13

### 3-(4-tert-butyl-phenyl)-N-(3-methyl-[2,3′]bipyridinyl-5-yl)-propionamide (Cpd 164)

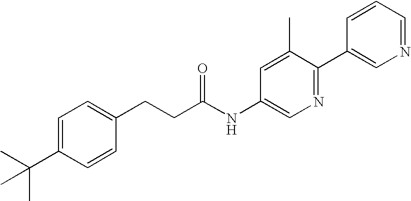

The title compound was obtained using the methods described in Example 8, substituting 4-pyridylboronic acid for 2-hydroxyphenylboronic acid. MS: M+H⁺=374.2, ¹H NMR (d₆-DMSO): δ 10.31 (s, 1H), 8.93 (s, 1H), 8.79 (d, 1H), 8.74 (s, 1H), 8.35 (d, 1H), 8.09 (s, 1H), 7.80 (dd, 1 H), 7.32 (d, 2H), 7.19 (d, 2H), 2.92 (t, 2H), 2.69 (t, 2H), 2.40 (s, 3H), 1.27 (s, 9H).

### EXAMPLE 14

### 3-(4-tert-butyl-phenyl)-N-(3′-hydroxy-2,6-dimethyl-biphenyl-4-yl)-propionamide (Cpd 86)

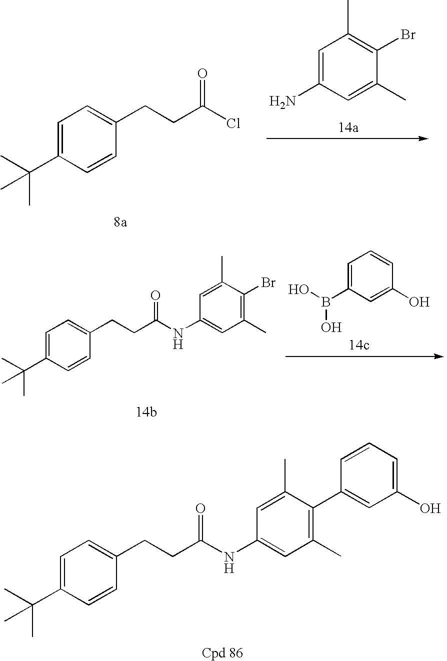

A. To a solution of Cpd 14a (1.001 g, 5.00 mmol) in 15 mL acetonitrile was added iPr₂NEt (0.96 mL, 5.51 mmol) and Cpd 8a (1.07 mL, 5.00 mmol). The reaction was stirred at ambient temperature for 18 h then evaporated in vacuo. The resulting residue was dissolved in 100 mL EtOAc and washed twice with 50 mL saturated NaHCO₃ and once with 50 mL brine. The organic phases were dried with Na₂SO₄, filtered and evaporated in vacuo. The resulting solid was triturated with 5 mL 10% EtOAc/Hexanes, collected by filtration, and rinsed with additional 10% EtOAc/Hexanes. The solid was dried under a stream of air to yield Cpd 14b (1.765 g, 4.54 mmol) as an off-white powder. MS: M+H⁺=388.1, ¹H NMR (d₆-DMSO): δ 7.42 (s, 2H), 7.30 (d, 2H), 7.17 (d, 2H), 2.87 (t, 2H), 2.59 (t, 2H), 2.31 (s, 6H), 1.26 (s, 9H).

B. Cpd 14b (0.097 g, 0.25 mmol) was dissolved in 5 mL 5:1 dioxane/ethanol solution in a microwave pressure vessel. To this was added 3-hydroxyphenylboronic acid (0.041 g, 0.30 mmol), cesium carbonate (0.170 g, 0.52 mmol), and PdCl₂(dppf) (0.010 g, 0.014 mmol). The vessel was flushed with argon, capped, and reacted in a microwave apparatus at 100 C for 15 min. The reaction mixture was diluted into 50 mL EtOAc and washed with 50 mL water with 5 mL brine. The organic phases were dried over Na₂SO₄, filtered, and evaporated to a residue. This residue was purified by reverse-phase chromatography (25-95% acetonitrile/water+0.1% TFA). Compound 86 (0.069 g, 0.17 mmol) was obtained as a tan-brown powder. MS: M+H⁺=402.3, ¹H NMR (d₆-DMSO): δ 9.78 (s, 1H), 9.39 (brs, 1H), 7.37-7.29 (m, 4H), 7.27-7.16 (m, 3H), 6.74 (d, 1H), 6.54-6.48 (m, 2H), 2.88 (t, 2H), 2.61 (t, 2H), 1.94 (s, 6H), 1.27 (s, 9H).

### EXAMPLE 15

### 3-(4-tert-butyl-phenyl)-N-(3′-hydroxy-2-methyl-biphenyl-4-yl)-propionamide (Cpd 59)

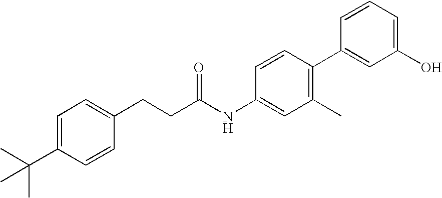

A. Using the method described in Step A of Example 14 and substituting 4-bromo-3-methyl-phenylamine for Compound 14a to obtain N-(4-bromo-3-methyl-phenyl)-3-(4-tert-butyl-phenyl)-propionamide.

B. Using the method described in Step B of Example 14 and substituting N-(4-bromo-3-methyl-phenyl)-3-(4-tert-butyl-phenyl)-propionamide for Compound 14b, the title compound was obtained. MS: M+H⁺=387.9, ¹H NMR (d₆-DMSO): δ 9.90 (s, 1H), 9.43 (br s, 1H), 7.53-7.44 (m, 2H), 7.31 (d, 2H), 7.23-7.15 (m, 3H), 7.09 (d, 1H), 6.77-6.65 (m, 3H), 2.89 (t, 2H), 2.62 (t, 2H), 2.20 (s, 3H), 1.27 (s, 9H).

### EXAMPLE 16

### 3-(4-tert-butyl-phenyl)-N-(3,5-dimethyl-4-pyridin-4-yl-phenyl)-propionamide (Cpd 167)

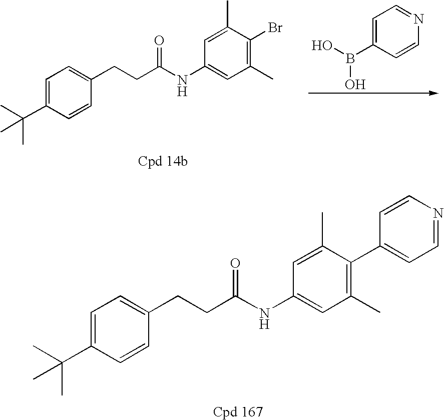

Compound 14b (0.097 g, 0.25 mmol) was dissolved in 5 mL 5:1 dioxane/ethanol solution in a microwave pressure vessel. To this was added 4-pyridylboronic acid (0.041 g, 0.33 mmol), cesium carbonate (0.180 g, 0.55 mmol), and PdCl₂(dppf) (0.011 g, 0.015 mmol). The vessel was flushed with argon, capped, and reacted in a microwave apparatus at 100 C for 15 min. The reaction mixture was diluted into 50 mL EtOAc and washed with 50 mL water with 5 mL brine. The organic phases were dried over Na₂SO₄, filtered and evaporated to a residue. This residue was purified by reverse-phase chromatography (25-95% acetonitrile/water+0.1% TFA). Compound 167 was obtained as a TFA salt (0.091 g, 0.18 mmol) as a cream-colored powder. MS: M+H⁺=387.3, ¹H NMR (d₆-DMSO): δ 9.91 (s, 1H), 8.84 (d, 2H), 7.62 (d, 2H), 7.41 (s, 2H), 7.32 (d, 2H), 7.18 (d, 2H), 2.88 (t, 2H), 2.62 (t, 2H), 1.98 (s, 6H), 1.27 (s, 9H).

### EXAMPLE 17

### 3-(5-tert-butyl-thiophen-2-yl)-N-(3′-hydroxy-biphenyl-4-yl)-propionamide (Cpd 65)

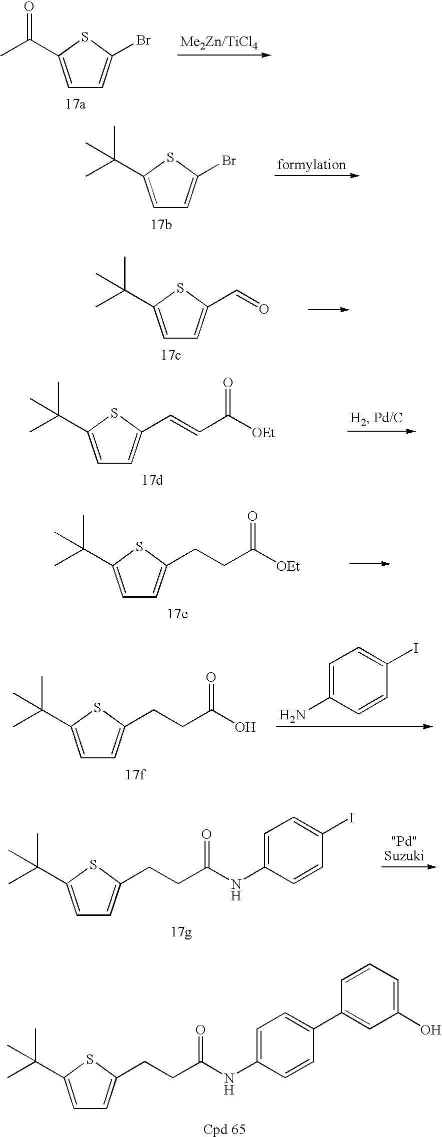

A. To a nitrogen-flushed flask was added 158 mL anhydrous DCM and titanium tetrachloride solution (42 mL, 1 M in DCM, 42 mmol). The solution was cooled on a dry ice/acetonitrile bath to 50 C. To the cooled solution was added dimethyl zinc solution (42 mL, 1 M in heptane, 42 mmol). The orange-brown slurry was stirred for 15 min, and then a solution of 1-(5-bromo-thiophen-2-yl)-ethanone (4.107 g, 20.0 mmol) in 40 mL DCM was added dropwise over 30 min. The ice was allowed to melt and the reaction warmed up. After 4.5 h the reaction mixture was quenched with 500 mL ice/water. The layers were separated and the aqueous phase was extracted twice more with 100 mL DCM. The combined organic phases were washed twice with 100 mL 1 N HCl and once with 100 mL brine, dried over MgSO₄, treated with charcoal, filtered, and evaporated. The residue was purified via silica gel chromatography (100% heptane) to give Compound 17b (3.14 g, 14.3 mmol) as a colorless oil. ¹H NMR (CDCl₃): δ 6.84 (d, 1H), 6.58 (d, 1H), 1.34 (s, 9H).

B. To compound 17b (3.14 g, 14.3 mmol) under a nitrogen atmosphere was added 100 mL tHF. The solution was cooled on a dry ice/acetone bath then n-BuLi (6.3 mL, 2.5 M in hexanes, 15.8 mmol) was added over 3 min. The ice was allowed to melt and the reaction to warm up overnight. The solution was cooled back down with a dry ice/acetone bath and a solution of 2.5 mL DMF in 10 mL tHF was added. The ice was allowed to melt and the reaction to warm up. After about 7 h the reaction mixture was poured into 500 mL ethyl ether and washed twice with 100 mL saturated NH₄Cl, once with 100 mL water, and 100 mL brine. The organic phase was dried over Na₂SO₄, filtered, evaporated to a residue and purified via silica gel chromatography (0-10% EtOAc/heptane) to give Compound 17c (0.504 g, 3.00 mmol) as a yellow-orange oil. MS: M+H⁺=168.9, ¹H NMR (CDCl₃): δ 9.83 (s, 1H), 7.62 (d, 1H), 6.98 (d, 1H), 1.42 (s, 9H).

C. To a solution of compound 17c (0.497 g, 2.95 mmol) in 50 mL benzene was added (carbethoxymethylene)-triphenylphosphorane (1.046 g, 3.00 mmol). The solution was heated at reflux for about 16 h then the solvents were evaporated in vacuo. This residue was purified via silica gel chromatography (0-10% EtOAc/heptane) to give Compound 17d (0.654 g, 2.74 mmol) as an orange oil. MS: M+H⁺=238.9, ¹H NMR (CDCl₃): δ 7.72 (d, 1 H), 7.07 (d, 1 H), 6.78 (d, 1H), 6.13 (d, 1H), 4.23 (q, 2H), 1.39 (s, 9H), 1.32 (t, 3H).

D. A solution of compound 17d (0.643 g, 2.70 mmol) in 25 mL ethanol with 10% palladium on carbon (0.199 g) was hydrogenated at about 50 psi for 19 h. The mixture was filtered and the filtrate was then evaporated to yield Compound 17e (0.616 g, 2.56 mmol) as a yellow oil. MS: M+H⁺=240.9, ¹H NMR (CDCl₃): δ 6.60 (t, 2H), 4.17 (q, 2H), 3.10 (t, 2H), 2.67 (t, 2H), 1.36 (s, 9H), 1.26 (t, 3H).

E. Compound 17e (0.613 g, 2.55 mmol) was dissolved in 25 mL of a 5:1 mixture of THF/H₂O. A portion of LiOH-H₂O (0.214 g, 5.10 mmol) was added to the reaction. After about 15 h the reaction was evaporated in vacuo. The residue was dissolved in 25 mL water and treated with 10 mL 1 N HCl. The precipitate that formed was collected by filtration, rinsed with water, and dried under vacuum at 50 C to yield compound 17f (0.459 g, 2.16 mmol) as an off-white powder. MS: M+H⁺=213.0, ¹H NMR (d₆-DMSO): δ 12.19 (s, 1H), 6.67-6.32 (m, 2H), 2.95 (t, 2H), 2.55 (t, 2H), 1.30 (s, 9H).

F. To a solution of compound 17f (0.318 g, 1.50 mmol) in 4 mL DMF was added 4-iodo-phenylamine (0.332 g, 1.52 mmol) and i-Pr₂NEt (0.29 mL, 1.66 mmol). A portion of HBTU (0.576 g, 1.52 mmol) was added and the reaction stirred for 17 h. The reaction mixture was poured into 100 mL water. The precipitate that formed was collected by filtration, rinsed with water, and dried under vacuum at 50 C to yield compound 17g (0.573 g, 1.39 mmol) as a tan powder. MS: M+H⁺=413.7, ¹H NMR (d₆-DMSO): δ 10.06 (s, 1H), 7.63 (d, 2H), 7.43 (d, 2H), 6.64 (s, 2H), 3.02 (t, 2H), 2.63 (t, 2H), 1.29 (s, 9H).

G. Compound 17g (0.103 g, 0.25 mmol) was dissolved in 5 mL 5:1 dioxane/ethanol solution in a microwave pressure vessel. To this was added 3-hydroxyphenylboronic acid (0.040 g, 0.29 mmol), cesium carbonate (0.172 g, 0.53 mmol), and PdCl₂(dppf) (0.019 g, 0.026 mmol). The vessel was flushed with argon, capped, and purified by reverse-phase chromatography at 100 C for 15 min. The reaction mixture was diluted into 50 mL EtOAc and washed with 50 mL water, then with 5 mL brine. The organic phase was dried over Na₂SO₄, filtered and evaporated to a residue. This residue was chromatographed reverse-phase (25-95% acetonitrile/water+0.1% TFA) to give the title Compound 65 (0.061 g, 0.16 mmol) as a pale yellow powder. MS: M+H⁺=379.8, ¹H NMR (d₆-DMSO): δ 10.02 (s, 1H), 9.56 (br s, 1H), 7.68 (d, 2H), 7.56 (d, 2H), 7.23 (t,1H), 7.03 (d, 1H), 6.99 (s, 1H), 6.73 (d, 1H), 6.67 (s, 2H), 3.07 (t, 2H), 2.68 (t, 2H), 1.30 (s, 9H).

### EXAMPLE 18

### trans-N-[4-(4,5-Dichloro-imidazol-1-yl)-3-trifluoromethyl-phenyl]-3-(5-trifluoromethyl-pyridin-2-yl)-acrylamide (Cpd 235)

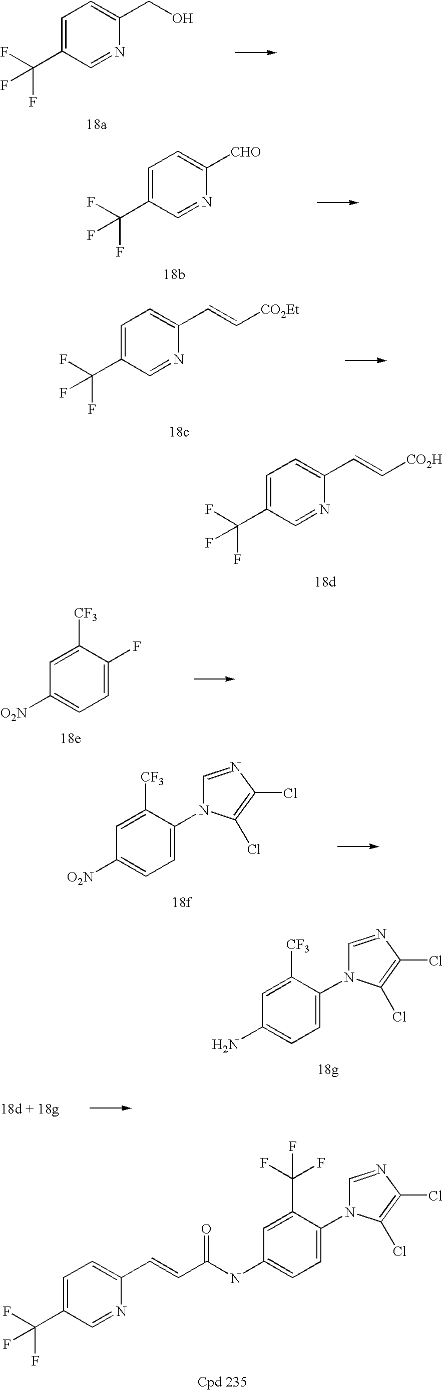

A. To a solution of compound 18a (3.84 g, 21.7 mmol), in 25 mL CHCl₃ was added manganese dioxide (MnO₂) (8.04 g). After stirring at ambient temperature for 15 hours the suspension was heated at reflux under a nitrogen atmosphere for an additional 23 hours. The reaction mixture was filtered over a pad of celite and the filter cake washed with additional CHCl₃. The filtrate was evaporated in vacuo to yield the product, compound 18b as an orange oil. MS: M+H⁺=175.8, ¹H NMR (CDCl₃): δ 10.15 (s, 1H), 9.06 (s, 1H), 8.17-8.06 (m, 2H).

B. To a solution of compound 18b (3.36 g, 19.2 mmol) in 100 mL benzene was added (triphenyl-λ⁵-phosphanylidene)-acetic acid ethyl ester (6.71 g, 19.3 mmol). The reaction mixture was refluxed under a nitrogen atmosphere for 5 hours. The solvents were removed in vacuo and the resulting material was triturated with 50 mL diethyl ether and filtered. The filtrate was evaporated in vacuo and purified via silica gel chromatography (5-15% EtOAc/heptane). The desired pure trans isomer was obtained as a yellow solid (compound 18c). MS: M+H⁺=246.1, ¹H NMR (d₆-DMSO): δ 9.02 (s, 1H), 8.31 (dd, 1H), 8.01 (d, 1H), 7.75 (d, 1H), 7.02 (d, 1H), 4.23 (q, 2H), 1.28 (t, 3H).

C. To a solution of compound 18c (1.808 g, 7.37 mmol), in a mixture of THF/H₂O (50 mL, 5:1) was added LiOH—H₂O (0.315 g, 7.51 mmol). The reaction mixture was stirred for 46 hours then evaporated in vacuo until a water solution remained. The solution was diluted with an additional 25 mL of water and acidified with 1N HCl (7.5 mL, 7.5 mmol). The precipitate that formed was collected by filtration, rinsed with additional water, air dried and then dried under vacuum to give the product, compound 18d as an off-white powder. MS: M+H⁺=217.8, ¹H NMR (d₆-DMSO): δ12.81 (s, 1H), 9.02 (s, 1H), 8.30 (dd, 1H), 7.97 (d, 1H), 7.69 (d, 1H), 6.96 (d, 1H).

D. To a solution of compound 18e (0.70 mL, 5.10 mmol) in 30 mL CH₃CN was added 4,5-dichloro-1H-imidazole (1.048 g, 7.65 mmol) and K₂CO₃ (1.06 g, 7.67 mmol). The mixture was heated at reflux for 5 hours, cooled, filtered over a pad of Celite, and rinsed with CH₃CN. The filtrate was evaporated in vacuo and the resulting residue was purified via silica gel chromatography (20-40% EtOAc/heptane) to give the product compound 18f as a yellow oil. MS: M+H⁺=325.9, ¹H NMR (d₆-DMSO): 8.78 (d, 1H), 8.71 (s, 1H), 8.22-8.14 (m, 2H).

E. To a solution of compound 18e (1.66 g, 5.09 mmol) in 15 mL 4:1 EtOH/H₂O was added NH₄Cl (0.415 g, 7.76 mmol) and Zn powder (2.68 g, 41.0 mmol, <10 micron). The reaction was stirred under a nitrogen atmosphere for 22 hours then partitioned between 75 mL EtOAc and 75 mL H₂O. This mixture was filtered over a pad of Celite, the organics isolated and washed once with 25 mL brine. The organics were dried over Na₂SO₄, filtered, and evaporated in vacuo to give the product compound 18g as a tan powder. MS: M+H⁺=295.9, ¹H NMR (d₆-DMSO): δ7.92 (s, 1H), 7.27 (d, 1H), 7.02 (s, 1H), 6.88 (d, 1H), 6.16 (s, 2).

F. To compound 18d (0.043 g, 0.20 mmol) was added a solution of DMAP in DCM (5 mL, 0.08 M, 0.40 mmol) and BOP—Cl (0.078 g, 0.31 mmol). After stirring for 25 minutes compound 18g (0.061 g, 0.21 mmol) was added. After stirring another 30 hours more BOP—Cl (0.079 g, 0.31 mmol) was added. After an additional 16 hours more DMAP in DCM solution was added (5 mL, 0.08 M, 0.40 mmol). After 6 more hours an additional amount of BOP—Cl (0.081 g, 0.021 mmol) and DMAP in DCM solution was added (5 mL, 0.08 M, 0.40 mmol). After 2 more hours additional compound 18d (0.012 g, 0.06 mmol) was added. After another 17 hours more compound 18d (0.011 g, 0.05 mmol) and BOP—Cl (0.039 g, 0.15 mmol) was added. After 2 hours the reaction-was washed with 5 mL saturated NaHCO₃ solution, dried over Na₂SO₄, filtered, evaporated to a residue. This residue was purified by reverse-phase chromatography (25-95% acetonitrile/water+0.1% TFA). The proper fractions were lyophilized to give the title compound 235 as a tan-orange powder. MS: M+H⁺=494.9, ¹H NMR (d₆-DMSO): δ11.13 (s, 1H), 9.06 (s, 1H), 8.41 (d, 1H), 8.33 (dd, 1H), 8.13-8.07 (m, 2H), 7.93 (d, 1H), 7.81 (d, 1H), 7.76 (d, 1H), 7.46 (d, 1H).

### EXAMPLE 19

### N-[4-(4-Methyl-imidazol-1-yl)-3-trifluoromethyl-phenyl]-3-(5-trifluoromethyl-pyridin-2-yl)-propionamide (Cpd 233)

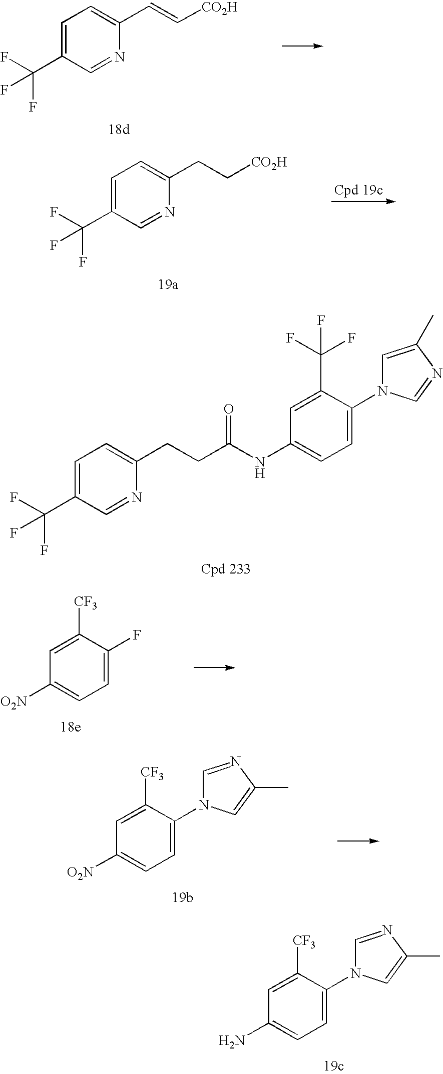

A. Compound 18d (0.434 g, 2.00 mmol) and 5% palladium on carbon (0.051 g) in 50 mL ethanol were hydrogenated at 50 psi hydrogen gas for 2 hours. The reaction mixture was filtered over a pad of Celite, a nylon disk, and the solvents were removed in vacuo to yield compound 19a as a light yellow waxy solid. MS: M+H⁺=220.0, ¹H NMR (d₆-DMSO): δ12.15 (s, 1H), 8.86 (s, 1H), 8.11 (dd, 1H), 7.54 (d, 1H), 3.08 (t, 2H), 2.72 (t, 2H).

B. To a solution of compound 18e (0.70 mL, 5.10 mmol) in 30 mL CH₃CN was added 4-methyl-1H-imidazole (0.630 g, 7.67 mmol) and K₂CO₃ (1.06 g, 7.89 mmol). The mixture was heated at reflux for 18 hours, cooled, and filtered over a pad of Celite. The filtrate was evaporated in vacuo and the resulting residue was purified via silica gel chromatography (60-100% EtOAc/heptane) to give the product compound 19b as a green oil. MS: M+H⁺=272.1, ¹H NMR (d₆-DMSO): δ8.66-8.61 (m, 2H), 7.88 (d, 1H), 7.80 (s, 1H), 7.20 (s,1 H), 2.18 (s, 3H).

C. Compound 19b (1.003 g, 3.70 mmol) and 5% palladium on carbon (0.108 g) in 50 mL ethanol were hydrogenated at 50psi hydrogen gas for 30 hours. The reaction mixture was filtered over a pad of Celite, a nylon disk, and the solvents were removed in vacuo to yield compound 19c as a tan powder. MS: M+H⁺=242.1, ¹H NMR (d₆-DMSO): δ 7.48 (s, 1H), 7.10 (d, 1H), 6.97 (d, 1H), 6.90 (s, 1H), 6.82 (dd, 1H), 5.91 (s, 2H), 2.12 (s, 3H).

D. To compound 19a (0.044 g, 0.20 mmol) was added a solution of DMAP in DCM (5 mL, 0.08 M, 0.40 mmol) and BOP—Cl (0.079 g, 0.31 mmol). After stirring for 25 minutes compound 19c (0.050 g, 0.21 mmol) was added. After stirring another 30 hours more BOP—Cl (0.081 g, 0.32 mmol) was added. After an additional 16 hours more DMAP in DCM solution was added (5 mL, 0.08 M, 0.40 mmol). After 6 more hours an additional amount of BOP—Cl (0.079 g, 0.031 mmol) and DMAP in DCM solution was added (5 mL, 0.08 M, 0.40 mmol). After 2 more hours additional compound 19a (0.023 g, 0.10 mmol) was added. After another 17 hours more of compound 19a (0.013 g, 0.06 mmol) and BOP—Cl (0.041 g, 0.16 mmol) was added. After 2 hours the reaction was washed with 5 mL saturated NaHCO₃ solution, dried over Na₂SO₄, filtered, evaporated to a residue. This residue was purified by reverse-phase chromatography (25-95% acetonitrile/water+0.1% TFA). The proper fractions were lyophilized to give the title compound 233 as an orange solid. MS: M+H⁺=443.0, ¹H NMR (d₆-DMSO): δ10.72 (s, 1H), 9.32 (s, 1H), 8.88 (s, 1H), 8.28 (d, 1H), 8.18 (dd, 1H), 7.98 (dd, 1H), 7.75 (d, 1H), 7.71 (s, 1H), 7.59 (d, 1H), 3.22 (t, 2H), 2.94 (t, 2H), 2.35 (s, 3H).

### EXAMPLE 20

### 3-(4-Trifluoromethyl-phenyl)-N-[3-trifluoromethyl-4-(5-trifluoromethyl-[1,2,4]oxadiazol-3-yl)-phenyl]-propionamide (Cpd 265)

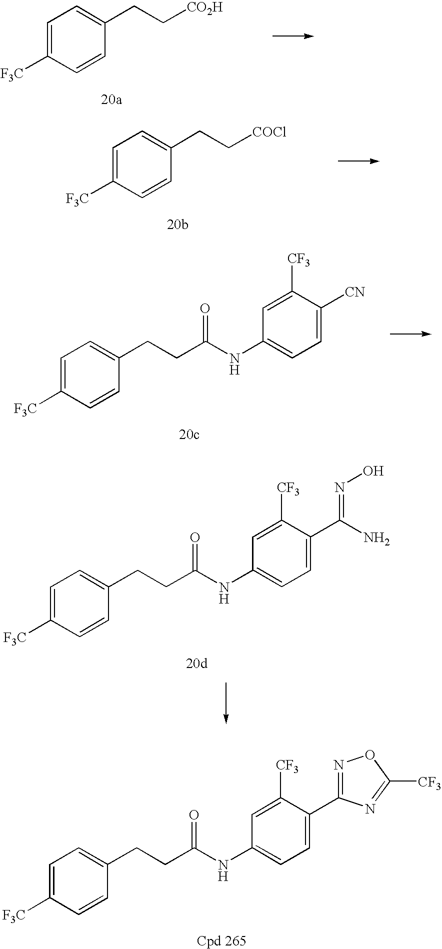

A. A solution of compound 20a (6.55 g, 30.0 mmol) in 30 mL SOCl₂ was heated at reflux for 3 hours. The reaction mixture was evaporated in vacuo, treated with benzene, and evaporated again to yield compound 20b as a yellow oil.

B. To a solution of 4-amino-2-trifluoromethyl-benzonitrile (3.724 g, 20.0 mmol) in 50 mL CH₃CN was added iPr₂NEt (3.8 mL, 21.8 mmol) and compound 20b (4.94 g, 20.9 mmol). After stirring for 24 hours an additional amount of compound 20b (0.65 g, 2.7 mmol) was added. After an additional hour of stirring, the reaction was evaporated in vacuo to a residue. The residue was purified via silica gel chromatography (eluent 20-50% EtOAc/heptane). The product fractions were collected, evaporated in vacuo, and the material was triturated with 30 mL 5:1 heptane/EtOAc. The solid was collected by filtration and air dried to give compound 20c as a cream-colored powder. MS: M+H⁺=386.9, ¹H NMR (d₆-DMSO): δ10.75 (s, 1H), 8.25 (s, 1H), 8.10 (d, 1H), 7.98 (d, 1H), 7.68 (d, 2H), 7.50 (d, 2H), 3.03 (t, 2H), 2.79 (t, 2H).

C. To compound 20c (4.564 g, 11.8 mmol) in 50 mL EtOH was added K₂CO₃ (8.20 g, 59.3 mmol) and H₂NOH—HCl (4.13 g, 59.4 mmol). The reaction mixture was heated at reflux for 19 hours, cooled, filtered, and the filter cake was rinsed with EtOH. The combined filtrates were evaporated in vacuo and the resulting material was triturated with 25 mL EtOAc, filtered and rinsed with EtOAc. The combined filtrates were evaporated in vacuo and the resulting material was triturated with 25 mL Et₂O, filtered and rinsed with Et₂O. The combined filtrates were evaporated in vacuo and the resulting residue was purified via silica gel chromatography (eluent 60-100% EtOAc/heptane) to give compound 20d, as a pale green powder. MS: M+H⁺=420.0, ¹H NMR (d₆-DMSO): δ10.32 (s, 1H), 9.48 (s, 1H), 8.04 (d, 1H), 7.77 (dd, 1H), 7.66 (d, 2H), 7.52-7.42 (m, 3H), 5.78 (s, 2H), 3.02 (t, 2H), 2.72 (t, 2H).

E. To a suspension of compound 20d (0.042 g, 0.10 mmol) in 5mL DCM was added excess trifluoroacetic anhydride (TFAA, 0.10 mL, 0.7 mmol). After 3 hours an additional amount of TFAA (0.40 mL, 2.9 mmol) was added. After 90 additional hours the reaction was evaporated in vacuo to a residue. This residue was purified by reverse-phase chromatography (25-95% acetonitrile/water+0.1% TFA). The proper fractions were lyophilized to give the title compound 265 as an off-white powder. MS: M+H⁺=497.7, ¹H NMR (d₆-DMSO): δ10.62 (s, 1H), 8.30 (s, 1H), 8.03 (d, 1H), 7.93 (d, 1H), 7.66 (d, 2H), 7.50 (d, 2H), 3.05 (t, 2H), 2.78 (t, 2H).

### EXAMPLE 21

### N-[4-(5-Methyl-[1,2,4]oxadiazol-3-yl)-3-trifluoromethyl-phenyl]-3-(4-trifluoromethyl-phenyl)-propionamide (Cpd 266)

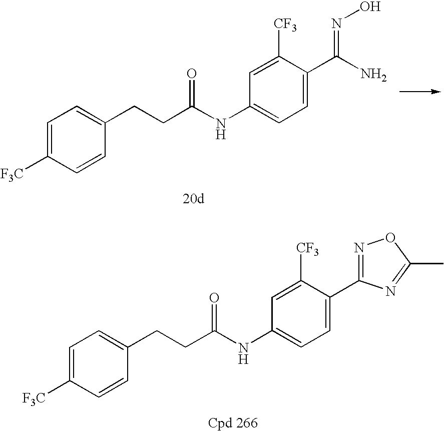

A. To a suspension of compound 20d (0.042 g, 0.10 mmol) in 5 mL DCM was added 0.5 mL Ac₂O. After 70 hours the reaction was evaporated in vacuo and re-dissolved in 5 mL CH₃CN. The mixture was then heated at reflux. After 4 hours of heating a pellet of NaOH was added and the heating was continued for 2 more hours. The reaction was evaporated in vacuo to a residue. This residue was purified by reverse-phase chromatography (25-95% acetonitrile/water+0.1% TFA). The proper fractions were lyophilized to give the title compound 266 as an off-white powder. MS: M+H⁺=443.9, ¹H NMR (d₆-DMSO): δ 10.53 (s,1H), 8.27 (s,1H), 7.99 (d, 1H), 7.82 (d, 1H), 7.69 (d, 2H), 7.51 (d, 2H), 3.04 (t, 2H), 2.77 (t, 2H), 2.69 (s, 3H).

### EXAMPLE 22

### N-[4-(2-Oxo-2,3-dihydro-2λ⁴-[1,2,3,5]oxathiadiazol-4-yl)-3-trifluoromethyl-phenyl]-3-(4-trifluoromethyl-phenyl)-propionamide (Cpd 245)

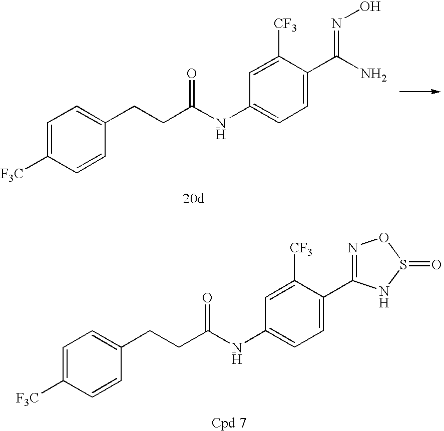

A. To a suspension of compound 20d (0.042 g, 0.10 mmol) in 5 mL DCM was added pyridine (0.040 mL, 0.49 mmol). The mixture was cooled on an ice bath and thionyl chloride (0.018 mL, 0.25 mmol) was added. After 10 minutes the reaction was quenched with 0.1 mL MeOH and the ice bath was removed. After stirring overnight, the reaction was diluted with additional MeOH and evaporated in vacuo to a residue. This residue was purified by reverse-phase chromatography (25-95% acetonitrile/water+0.1% TFA). The proper fractions were Iyophilized to give the title compound 245 as an off-white powder. MS: M+H⁺=466.0, ¹H NMR (d₆-DMSO): δ12.08 (br s, 1H), 10.54 (s, 1H), 8.22 (s, 1H), 7.98 (d, 1H), 7.72-7.59 (m, 3H), 7.51 (d, 2H), 3.06 (t, 2H), 2.78 (t, 2H).

### EXAMPLE 23

### N-[6-(4,5-Dichloro-imidazol-1-yl)-5-methyl-pyridin-3-yl]-3-(4-trifluoromethyl-phenyl)-propionamide (Cpd 203)

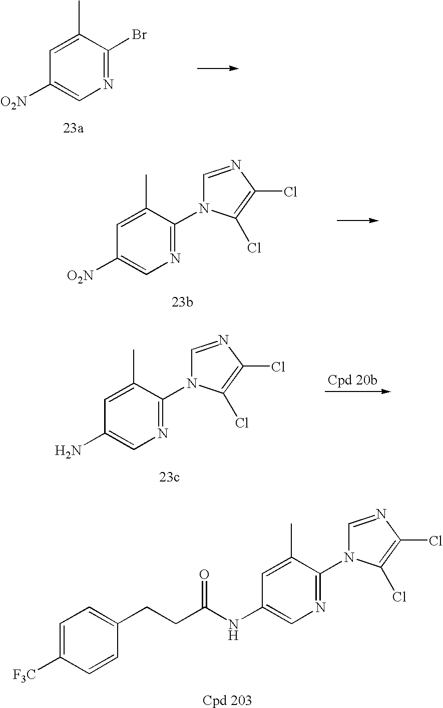

A. To a solution of compound 23a (1.09 g, 5.02 mmol) in 30 mL CH₃CN was added 4,5-dichloro-1H-imidazole (1.04 g, 7.59 mmol) and K₂CO₃ (1.08 g, 7.81 mmol). The mixture was heated at reflux for 19 hours, cooled, and filtered over a pad of Celite. The filtrate was evaporated in vacuo and the resulting residue was purified via silica gel chromatography (20-40% EtOAc/heptane) to give compound 23b as an orange oil. MS: M+H⁺=272.9, ¹H NMR (d₆-DMSO): δ 9.30 (s, 1H), 8.92 (s, 1H), 8.25 (s, 1H), 2.34 (s, 3H).

B. To a solution of compound 23b (1.313 g, 4.81 mmol) in 15 mL 4:1 EtOH/H₂O was added NH₄Cl (0.391 g, 7.31 mmol) and Zn powder (2.53 g, 38.7 mmol, <10 micron). The reaction was stirred under a nitrogen atmosphere for 22 hours then partitioned between 75 mL EtOAc and 75 mL H₂O. This mixture was filtered over a pad of Celite, the organics isolated and washed once with 50 mL saturated NH₄Cl then 50 mL brine. The organics were dried with Na₂SO₄, filtered, and evaporated in vacuo to give compound 23c as a tan powder. MS: M+H⁺=243.0, ¹H NMR (d₆-DMSO): δ 7.95 (s, 1H), 7.72 (d, 1H), 6.95 (d, 1H), 5.80 (s, 2H), 1.94 (s, 3H).

C. To compound 23c (0.061 g, 0.25 mmol) in 1 mL CH₃CN was added iPr₂NEt (0.05 mL, 0.29 mmol) and compound 20b (0.05 mL, 0.27 mmol). After 21 hours the reaction was diluted with MeOH containing a small amount of TFA. The reaction was evaporated in vacuo to a residue. This residue was purified by reverse-phase chromatography (25-95% acetonitrile/water+0.1% TFA). The proper fractions were lyophilized to give the title compound 203 as a light yellow powder. MS: M+H⁺=443.1, ¹H NMR (d₆-DMSO): δ 10.46 (s, 1H), 8.58 (s, 1H), 8.21 (s, 1H), 8.10 (s, 1H), 7.69 (d, 2H), 7.52 (d, 2H), 3.05 (t, 2H), 2.78 (t, 2H), 2.12 (s, 3H).

### EXAMPLE 24

### 3-(4-tert-Butyl-phenyl)-N-[4-(4,5-dichloro-imidazol-1-yl)-3-trifluoromethyl-phenyl]-propionamide (Cpd 212)

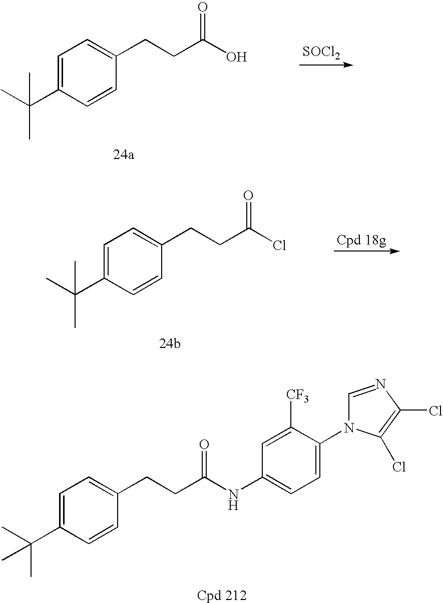

A. A solution of 3-(4-tert-butyl-phenyl)-propionic acid (Cpd 24a, 0.518 g, 2.51 mmol) in 10 mL SOCl₂ was heated at reflux for 2.5 hours. The reaction mixture was evaporated in vacuo, treated with benzene, and evaporated again to yield compound 24b as a yellow-orange oil.

B. To a solution of compound 18g (0.074 g, 0.025 mmol) in 1 mL CH₃CN was added iPr₂NEt (0.05 mL, 0.29 mmol) and compound 24b (0.06 mL, 0.28 mmol). After 21 hours additional compound 24b (0.01 mL, 0.05 mmol) was added. After 2 more hours the reaction was diluted with MeOH containing a small amount of TFA. The reaction was evaporated in vacuo to a residue. This residue was purified by reverse-phase chromatography (25-95% acetonitrile/water+0.1% TFA). The proper fractions were lyophilized to give the title compound 212 as a cream-colored powder. MS: M+H⁺=484.1, ¹H NMR (d₆-DMSO): δ10.57 (s, 1H), 8.29 (s, 1H), 8.05 (s, 1H), 8.00 (d, 1H), 7.69 (d, 1H), 7.32 (d, 2H), 7.18 (d, 2H), 2.91 (t, 2H), 2.69 (t, 2H), 1.25 (s, 9H).

### EXAMPLE 25

### N-[4-(4-Methyl-imidazol-1-yl)-3-trifluoromethyl-phenyl]-3-(4-trifluoromethyl-phenyl)-propionamide (Cpd 204)

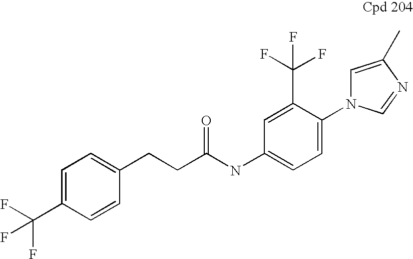

A. To a solution of compound 20b (0.328 g, 1.50 mmol) in 30 mL DCM was added DMAP (0.366 g, 3.00 mmol) and BOP—Cl (0.578 g, 2.27 mmol). After 10 minutes compound 19c (0.365 g, 1.51 mmol) was added. After 18 hours the reaction was washed with 30 mL saturated NaHCO₃, dried over Na₂SO₄, filtered, and evaporated in vacuo to a residue. This residue was purified by reverse-phase chromatography (25-95% acetonitrile/water+0.1% TFA). The proper fractions were mixed with poly(vinylpyridine), filtered and lyophilized to give the title compound 204 as an off-white powder. MS: M+H⁺=442.2, ¹H NMR (d₆-DMSO): δ 10.61 (s, 1H), 8.94 (s, 1H), 8.24 (d, 1H), 7.96 (dd, 1H), 7.71-7.64 (m, 3H), 7.56 (s, 1H), 7.50 (d, 2H), 3.04 (t, 2H), 2.77 (t, 2H), 2.30 (s, 3H).

### EXAMPLE 26

### 3-(4-tert-Butyl-phenyl)-N-(6-imidazol-1-yl-5-trifluoromethyl-pyridin-3-yl)-propionamide (Cpd 202)

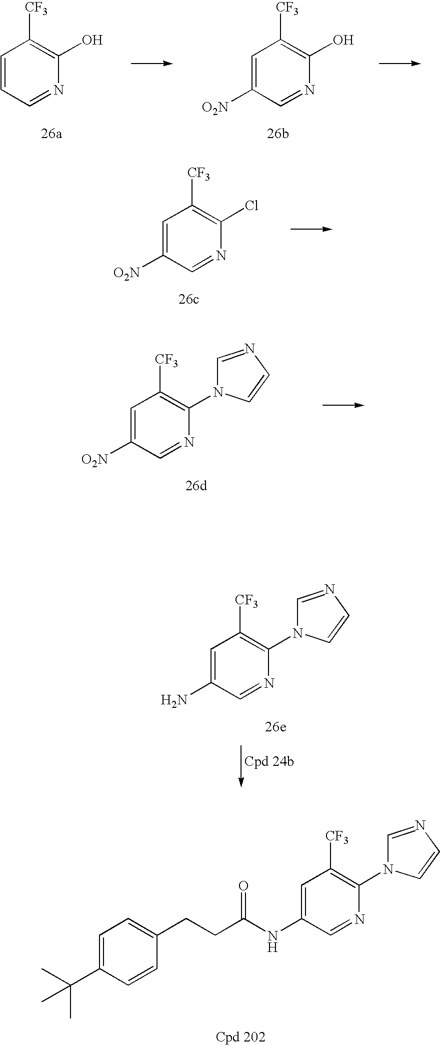

A. To an ice-cooled solution of compound 26a (5.93 g, 36.4 mmol) in 35 mL concentrated H₂SO₄ was added concentrated HNO₃ (2.6 mL, 40.8 mmol) dropwise. After 30 minutes the ice bath was removed and the reaction was stirred at ambient temperature for 15 hours. The reaction mixture was then warmed to 60 C for 5 hours, cooled, and added to 150 g of ice. The resulting precipitate was collected by filtration, rinsed with additional water, and air-dried to afford the first batch of product. Another crop of product was obtained after evaporating the mother liquor to less than 100 mL volume, cooling on an ice bath, and adding NaOH (25.34 g). This solid was filtered off, rinsed with water, and air-dried to provide another batch of product of compound 26b. ¹H NMR (d₆-DMSO): δ 13.49 (br s, 1H), 8.96 (s, 1H), 8.47 (s, 1H).

B. To a mixture of compound 26b (3.84 g, 18.5 mmol) in 20 mL SOCl₂ was added 0.5 mL DMF. The mixture was heated to reflux for 4 hours then concentrated in vacuo. The residue was dissolved in benzene and concentrated again in vacuo. This residue was taken up in 50 mL EtOAc and washed with 50 mL saturated NaHCO₃, and 50 mL brine, then dried over Na₂SO₄, and filtered and concentrated in vacuo. The resulting residue was chromatographed over silica gel (0-10% EtOAc/heptane) to give compound 26c as a pale yellow oil. ¹H NMR (d₆-DMSO): δ 9.52 (s, 1H), 8.92 (s, 1H).

C. To a solution of compound 26c (1.136 g, 5.01 mmol) in 50 mL CH₃CN was added imidazole (0.514 g, 7.55 mmol) and iPr₂NEt (1.75 mL, 10.0 mmol). The reaction was heated at reflux for 1 hour then concentrated in vacuo to a residue. The residue was purified via silica gel chromatography (40-80% EtOAc/heptane) to give compound 26d as a waxy yellow solid. MS: M+H⁺=258, ¹H NMR (d₆-DMSO): δ 9.66 (s, 1H), 9.13 (s, 1H), 8.13 (s, 1H), 7.65 (s, 1H), 7.21 (s, 1H).

D. To a solution of compound 26d (0.857 g, 3.32 mmol) in 15 mL 4:1 EtOH/H₂O was added NH₄Cl (0.269 g, 5.03 mmol) and Zn powder (1.76 g, 26.9 mmol, <10 micron). The reaction was stirred under a nitrogen atmosphere for 18 hours then partitioned between 75 mL EtOAc and 75 mL H₂O. This mixture was filtered over a pad of Celite, and the organic phase was partitioned. The aqueous phase was extracted twice more with 50 mL EtOAc. The combined organic phases were washed once with 25 mL brine then evaporated in vacuo. The residue was taken up in MeOH, filtered, evaporated in vacuo, re-dissolved in EtOH, filtered again, and evaporated in vacuo to give the crude product compound 26e as a rust-orange powder. MS: M+H⁺=229.1.

E. To a solution of Cpd 26e (0.057 g, 0.025 mmol) in 1 mL CH₃CN was added iPr₂NEt (0.05 mL, 0.29 mmol) and compound 24b (0.06 mL, 0.28 mmol). After 21 hours additional compound 24b (0.01 mL, 0.05 mmol) was added. After 2 more hours the reaction was diluted with MeOH a little TFA was added. The reaction was evaporated in vacuo to a residue. This residue was purified by reverse-phase chromatography (25-95% acetonitrile/water+0.1% TFA). The proper fractions were lyophilized to give the title compound 202 as a peach-colored powder. MS: M+H⁺=417.2, ¹H NMR (d6-DMSO): δ 10.80 (s, 1H), 8.92 (s, 1H), 8.77-8.69 (m, 2H), 7.80 (s, 1H), 7.50 (s, 1H), 7.31 (d, 2H), 7.18 (d, 2H), 2.92 (t, 2H), 2.73.(t, 2H), 1.26 (s, 9H).

### EXAMPLE 27

### N-[6-(3-Hydroxy-phenyl)-5-trifluoromethyl-pyridin-3-yl]-3-(4-trifluoromethyl-phenyl)-propionamide (Cpd 210)

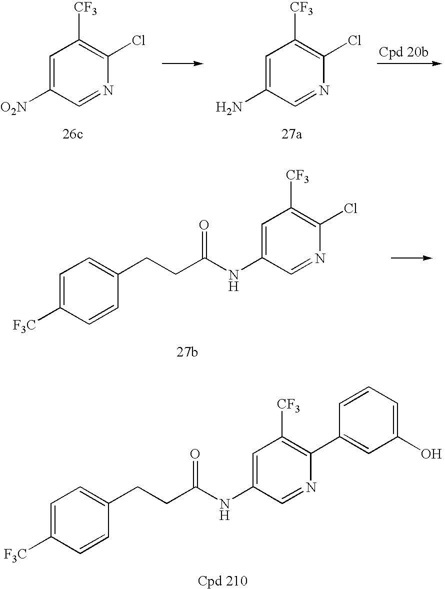

A. To a solution of compound 26c (1.478 g, 6.52 mmol) in 20 mL 4:1 EtOH/H₂O was added NH₄Cl (0.524 g, 9.80 mmol) and Zn powder (3.44 g, 52.6 mmol, <10 micron). The reaction was stirred under a nitrogen atmosphere for 18 hours then partitioned between 75 mL EtOAc and 75 mL H₂O. This solvent mixture was filtered over a pad of Celite, 50 mL brine was added to help with the emulsion. The mixture was filtered again over Celite. The organic phase was isolated and dried over Na₂SO₄, filtered, and evaporated in vacuo to a residue. The residue was purified via silica gel chromatography (30-60% EtOAc/heptane) to give compound 27a as a yellow-orange powder. MS: M+H⁺=197.0, ¹H NMR (d6-DMSO): δ 7.93 (d, 1H), 7.39 (d, 1H), 6.02 (s, 2H).

B. To compound 27a (0.241 g, 1.23 mmol) in 4 mL CH₃CN was added iPr2NEt (0.24 mL, 1.38 mmol) and compound 20b (0.24 mL, 1.32 mmol). After 18 hours the reaction was evaporated in vacuo, taken up in 25 mL EtOAc and washed successively with 25 mL saturated NaHCO₃ then 25 mL brine. The organic phase was dried over Na₂SO₄, filtered, and evaporated in vacuo to a residue. The residue was purified via silica gel chromatography (30-60% EtOAc/heptane) to give compound 27b as a yellow-tan powder. MS: M+H⁺=397.0, ¹H NMR (d₆-DMSO): δ 10.67 (s, 1H), 8.80 (s, 1H), 8.59 (s, 1H), 7.69 (d, 2H), 7.51 (d, 2H), 3.02 (t, 2H), 2.78 (t, 2H).

C. To compound 27b (0.040 g, 0.10 mmol) in a 2 mL solution of 5:1 dioxane/EtOH was added 3-hydroxyphenylboronic acid (0.017 g, 0.12 mmol), Cs₂CO₃ (0.078 g, 0.24 mmol), and PdCl₂(dppf) (0.006 g, 0.008 mmol). The reaction mixture was heated under microwave irradiation to 100 C for 15 minutes, then 100 C for 15 minutes again. More PdCl₂(dppf) (0.010 g, 0.014 mmol) was added and the reaction was heated again at 100 C for 15 minutes. Additional 3-hydroxyphenylboronic acid (0.010 g, 0.07 mmol) was added and the reaction was heated again at 120 C for 15 minutes. Addtional PdCl₂(dppf) (0.009 g, 0.012 mmol) was added and the reaction was heated once more at 120 C for 15 minutes. The reaction mixture was partitioned between 20 mL EtOAc and 22 mL brine/water. The organic phase was evaporated in vacuo to a residue and purified by reverse-phase chromatography (25-95% acetonitrile/water+0.1% TFA). The proper fractions were lyophilized to give the title compound 210 as a tan powder. MS: M+H⁺=455.1, ¹H NMR (d₆-DMSO): δ 10.58 (s,1H), 9.58 (br s, 1H), 8.91 (s, 1H), 8.52 (s, 1H), 7.66 (d, 2H), 7.51 (d, 2H), 7.23 (t, 1H), 6.89-6.81 (m, 3H), 3.03 (t, 2H), 2.78 (t, 2H).

### EXAMPLE 28

### trans-3-[4-(Cyclohexyl-methyl-amino)-phenyl]-N-[6-(4,5-dichloro-imidazol-1-yl)-5-methyl-pyridin-3-yl]-acrylamide (Cpd 241)

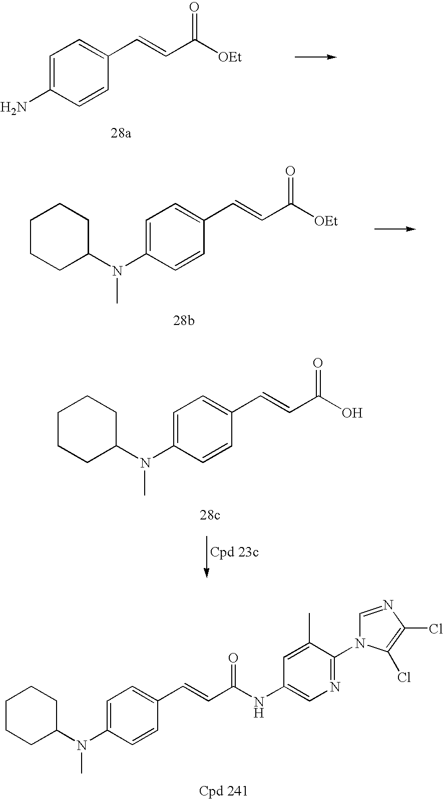

A. To a solution of compound 28a (1.914 g, 10.01 mmol) in 30 mL DCE was added cyclohexanone (1.04 mL, 10.03 mmol) and Me₄NeBH(OAc)₃ (5.325 g, 20.2 mmol). The reaction mixture was heated at reflux for 19 hours, cooled, and paraformaldehyde (1.505 g, 50.1 mmol) was added along with additional Me₄N-BH(OAc)₃ (5.276 g, 20.1 mmol). The reaction was again heated at reflux for 20 hours, cooled, and diluted with 50 mL DCM. The organics were washed twice with 50 mL water, once with 50 mL saturated NaHCO₃, once with 50 mL brine, dried over Na₂SO₄, filtered, and evaporated in vacuo to a residue. The residue was purified via silica gel chromatography (0-7.5% EtOAc/hexanes) to give compound 28b as a yellow oil. ¹H NMR (CDCl₃): δ 7.62 (d, 1H), 7.40 (d, 2H), 6.70 (d, 2H), 6.21 (d, 1H), 4.23 (q, 2H), 3.63 (m, 1H), 2.83 (s, 3H), 1.92-7.66 (m, 5H), 1.58-1.23 (m, 7H), 1.16 (m, 1H).

B. To a solution of compound 28b (1.672 g, 5.82 mmol) in 60 mL 5:1 THF/H₂O was added LiOH-H₂O (0.252 g, 6.01 mmol). After stirring at ambient temperature for 4 days, the reaction was heated at reflux for 26 hours, more LiOH—H₂O (0.084 g, 2.00 mmol) was added, and the mixture was refluxed for another 18 hours. The reaction mixture was evaporated in vacuo to give an aqueous residue which was diluted with 50 mL water and acidified with 8.0 mL 1N HCl. The precipitated solid was collected by filtration, rinsed with water and hexanes and dried under vacuum at 50 C to yield compound 28c as a yellow powder. MS: M+H⁺=260.1, ¹H NMR (d₆-DMSO): δ 11.92 (s, 1H), 7.48 (m, 3H), 6.78 (d, 2H), 6.22 (d, 1H), 3.70 (m, 1H), 2.80 (s, 3H), 1.78 (br d, 2H), 1.65 (br s, 3H), 1.58-1.29 (m, 4H), 1.13 (m, 1H).

C. To a solution of compound 28c (0.065 g, 0.25 mmol) in 5 mL DCM was added DMAP (0.062 g, 0.51 mmol) and BOP—Cl (0.099 g, 0.39 mmol). After less than 5 minutes compound 23c (0.062 g, 0.26 mmol) was added. After 20 hours the reaction mixture was diluted with 5 mL DCM, washed with 5 mL saturated NaHCO₃, dried over Na₂SO₄, and evaporated to a residue. The residue was purified by reverse-phase chromatography (25-95% acetonitrile/water+0.1% TFA). The proper fractions were lyophilized to give the title compound 241 as a yellow powder. MS: M+H⁺=484.3, ¹H NMR (d₆-DMSO): 10.51 (s, 1H), 8.69 (d, 1H), 8.30 (d, 1H), 8.10 (s, 1H), 7.55 (d, 1H), 7.48 (d, 2H), 6.85 (brd, 2H), 6.56 (d, 1H), 3.75-3.66 (m, 1H), 2.82 (s, 3H), 2.14 (s, 3H), 1.79 (br d, 2H), 1.71-1.60 (m, 3H), 1.56-1.33 (m, 4H), 1.20-1.07 (m, 1H).

### EXAMPLE 29

### N-[4-(4,5,6,7-Tetrahydro-benzoimidazol-1-yl)-3-trifluoromethyl-phenyl]-3-(4-trifluoromethyl-phenyl)-propionamide (Cpd 270)

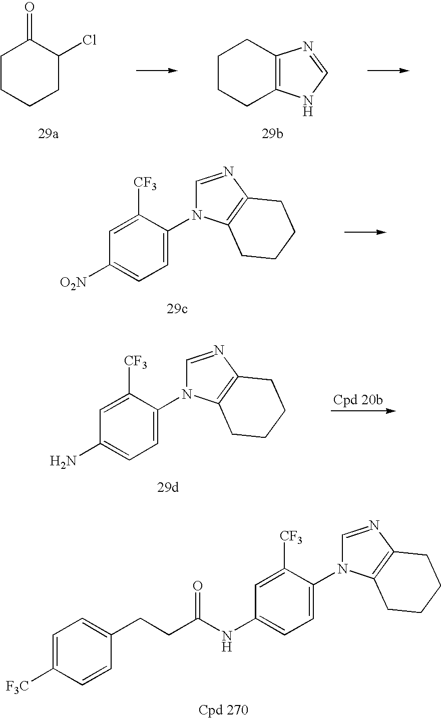

A. To a solution of compound 29a (3.37 g, 25.4 mmol) in 50 mL toluene was added formamidine hydrochloride (10.44 g, 130 mmol) and K₂CO₃ (17.59 g, 127 mmol). The reaction was heated at reflux for 4 hours, cooled and the solids were collected by filtration. The filter cake was successively washed with toluene then with DCM. The DCM wash was evaporated to a solid. This solid was dissolved in 10 mL hot benzene along with a small amount of Et₂O and DCM. The clear upper layer was decanted and evaporated in vacuo to yield compound 29b as a waxy tan solid. MS: M+H⁺=123.2.

B. To a solution of compound 18e (0.70 mL, 5.10 mmol) in 30 mL CH₃CN was added compound 29b (0.79 g, 6.5 mmol) and K₂CO₃ (1.06 g, 7.89 mmol). The mixture was heated to reflux for 15 hours, cooled, and filtered over a pad of Celite and rinsed with CH₃CN. The filtrate was evaporated in vacuo and the resulting residue was purified via silica gel chromatography (60-100% EtOAc/heptane) to give compound 29c (1.177 g, 3.78 mmol). MS: M+H⁺=312.1, ¹H NMR (CDCl₃): δ 8.72 (d, 1H), 8.54 (dd, 1H), 7.54 (d, 1H), 7.45 (s, 1H), 2.72-2.65 (m, 2H), 2.31-2.22 (m, 2H), 1.90-1.76 (m, 4H).

C. Compound 29c (0.504 g, 1.62 mmol) and 5% palladium on carbon (0.052 g) in 25 mL ethanol were hydrogenated at 50 psi of hydrogen gas for 19 hours. The reaction mixture was filtered over a pad of Celite, a nylon disk, and the solvents were removed in vacuo. Hexanes were added and the solvent was removed in vacuo to yield compound 29d as a tan powder. MS: M+H⁺=282.1, ¹H NMR (CDCl₃): δ 7.36 (s, 1H), 7.04 (d, 1H), 7.01 (d, 1H), 6.83 (dd, 1H), 4.07 (s, 2H), 2.69-2.60 (m, 2H), 2.27-2.18 (m, 2H), 1.86-1.71 (m, 4H).

D. To a solution of compound 20b (0.055 g, 0.25 mmol) in 5 mL DCM was added DMAP (0.063 g, 0.52 mmol) and BOP—Cl (0.101 g, 0.40 mmol). After less than 5 minutes compound 29d (0.070 g, 0.25 mmol) was added. After 15 hours the reaction mixture was diluted with 5 mL DCM, washed with 5 mL saturated NaHCO₃, dried over Na₂SO₄, and evaporated to a residue. The residue was purified by reverse-phase chromatography (25-95% acetonitrile/water +0.1% TFA). The proper fractions were lyophilized to give the title compound 270 as a white powder. MS: M+H⁺=482.2, ¹H NMR (d₆-DMSO): δ 10.67 (s, 1H), 9.23 (s, 1H), 8.29 (d, 1H), 8.01 (dd, 1H), 7.74 (d, 1H), 7.67 (d, 2H), 7.50 (d, 2H), 3.04 (t, 2H), 2.78 (t, 2H), 2.72-2.64 (m, 2H), 2.38-2.27 (m, 1H), 2.19-2.9 (m, 1H), 1.84-1.69 (m, 4H).

### EXAMPLE 30

### 3-(4-Trifluoromethyl-phenyl)-N-[3-trifluoromethyl-4-(4-trifluoromethyl-imidazol-1-yl)-phenyl]-propionamide (Cpd 238)

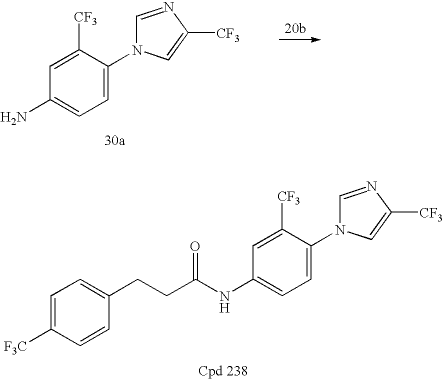

A. 4-Trifluoromethyl-1H-imidazole was prepared according to the methods described in the scientific literature (Moazzamm M. and Parrick, J. Indian Journal of Chemistry, Section B (1988) 1051-1053). Using the methods described in Example 19, Steps A, B, and C, and substituting 4-trifluoromethyl-1H-imidazole for 4-methyl-1H-imidazole in Step B, compound 30a was prepared.

B. Using the procedure described in Example 25, substituting compound 30a for compound 19c, the title compound 238 was prepared. MS: M+H⁺=496.2, ¹H NMR (d₆-DMSO): 10.55 (s, 1H), 8.22 (d, 1H), 8.12 (s, 1H), 8.06 (s, 1H), 7.93 (dd, 1H), 7.69-7.63 (m, 3H), 7.50 (d, 2H), 3.04 (t, 2H), 2.76 (t, 2H).

### EXAMPLE 31

### N-[4-(4-Chloro-imidazol-1-yl)-3-trifluoromethyl-phenyl]-3-(4-trifluoromethyl-phenyl)-propionamide (Cpd 227)

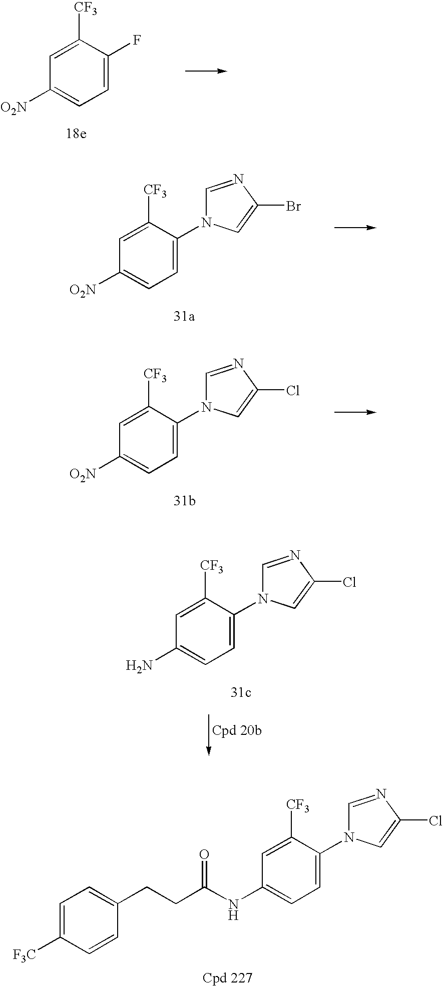

A. To a solution of 4-bromo-1H-imidazole (1.78 g, 12.1 mmol) in 60 mL CH₃CN was added compound 18e (1.50 mL, 10.9 mmol) and K₂CO₃ (1.669 g, 12.1 mmol). The mixture was heated at reflux for 2 hours, cooled, filtered, and concentrated in vacuo. The residue was triturated with 50 mL water, the solid isolated by filtration and rinsed with additional water. The material was dried under vacuum to give compound 31a as a grey-tan powder. MS: M+H⁺=336.0, ¹H NMR (d₆-DMSO): δ 8.70-8.63 (m, 2H), 8.02-7.96 (m, 2H), 7.75 (s, 1H).

B. To a solution of compound 31a (3.36 g, 10.0 mmol) in 100 mL DMSO was added CuCl (9.92 g, 100.2 mmol). The reaction was heated at 110 C for 17 hours. The reaction mixture was cooled and then diluted into 1600 mL of a 1:1 mixture of water/EtOAc. The mixture was filtered over Celite and the organic phase was isolated and washed once with 250 mL brine. The organic phase was evaporated in vacuo to a residue. The residue was purified via silica gel chromatography (20-50% EtOAc/heptane) to give compound 31 b as a yellow oil. MS: M+H⁺=292.0, ¹H NMR (d₆-DMSO): δ 8.72-8.62 (m, 2H), 8.04-7.95 (m, 2H), 7.72 (s, 1H).

C. To a solution of compound 32b (0.31 g, 1.06 mmol) in 25 mL EtOH was added 2 mL concentrated HCl and Zn granules (0.70 g, 10.7 mmol, 20 mesh). After 1.5 hours the reaction was filtered and evaporated in vacuo to a residue. The residue was taken up in 100 mL EtOAc, washed once with 50 mL saturated NaHCO₃, dried over Na₂SO₄, filtered, and evaporated in vacuo to yield compound 31c. MS: M+H⁺=262.0, ¹H NMR (d₆-DMSO): δ 7.68 (s, 1H), 7.42 (s, 1H), 7.20 (d, 1H), 6.99 (d, 1H), 6.83 (dd, 1H), 6.02 (s, 2H).

D. To a solution of compound 20b (0.218 g, 1.00 mmol) in 20 mL DCM was added DMAP (0.252 g, 2.06 mmol) and BOP—Cl (0.391 g, 1.54 mmol). After less than 10 minutes compound 31c (0.280 g, 1.00 mmol) was added. After 19 hours the reaction mixture was washed with 20 mL saturated NaHCO₃, dried over Na₂SO₄, and evaporated to a residue. The residue was purified by reverse-phase chromatography (25-95% acetonitrile/water+0.1% TFA). The proper fractions were mixed with poly(vinylpyridine), filtered and lyophilized to give the title compound227 as a pale yellow powder. MS: M+H⁺=462.1, ¹H NMR (d₆-DMSO): δ 10.52 (s,1H), 8.20 (d, 1H), 7.91 (dd, 1. H), 7.81 (s,1H), 7.66 (d, 2H), 7.59 (d, 1H), 7.55 (s, 1H), 7.50 (d, 2H), 3.04 (t, 2H), 2.75 (t, 2H).

### EXAMPLE 32

### trans-N-[4-(4-Methyl-imidazol-1-yl)-3-trifluoromethyl-phenyl]-3-(4-trifluoromethanesulfonyl-phenyl)-acrylamide (Cpd 205)

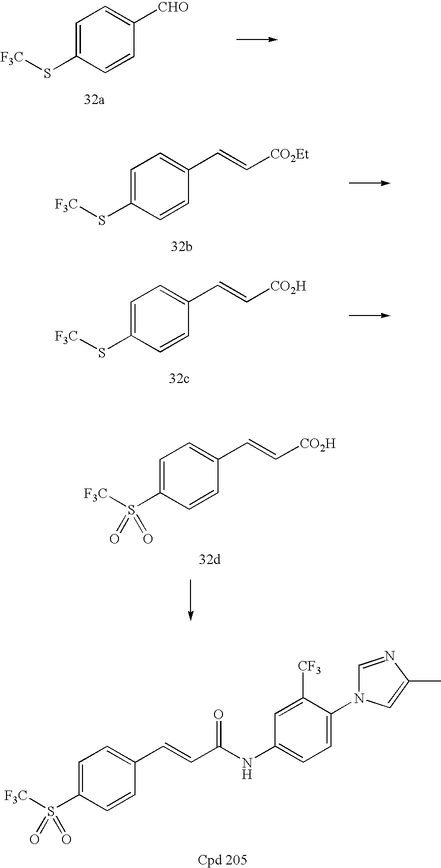

A. To a solution of compound 32a (15.46 g, 75.0 mmol) in 350 mL benzene was added (triphenyl-λ⁵-phosphanylidene)-acetic acid ethyl ester (26.14 g, 75.0 mmol). The reaction mixture was refluxed under a nitrogen atmosphere for 6 hours. The solvents were removed in vacuo and the resulting material was triturated with 350 mL diethyl ether and filtered. The filtrate was concentrated in vacuo and triturated once more with 50 mL diethyl ether and filtered. The filtrate was evaporated in vacuo and purified via silica gel chromatography (0-10% EtOAc/heptanes). A mixture of cis-trans products were obtained, as well as the desired pure trans isomer compound 32b as a white solid. MS: M+H⁺=277.0, ¹H NMR (d₆-DMSO): δ 7.89 (d, 2H), 7.75 (d, 2H), 7.70 (d, 1H), 6.77 (d, 1H), 4.21 (q, 2H), 1.27 (t, 3H).

B. To a solution of compound 32b (10.31 g, 37.3 mmol) in 300 mL ethanol was added 3 N aqueous NaOH solution (13.0 mL, 39.0 mmol). The reaction mixture was stirred for 21 hours then evaporated in vacuo. The residue was dissolved in 250 mL water and 1 N aqueous HCl (45 mL, 45 mmol) was added. The resulting precipitate was collected by filtration, rinsed with water and dried under a stream of air to yield compound 32c as a white powder. ¹H NMR (d₆-DMSO): δ 12.58 (s, 1H), 7.85 (d, 2H), 7.74 (d, 2H), 7.63 (d, 1H), 6.66 (d, 1H).

C. To a suspension of compound 32c (2.483 g, 10.01 mmol) in 50 mL TFA was added 30% H₂O₂ solution (8 mL, 83 mmol). The reaction was stirred for 21 hours, and then poured into 250 mL of ice water. The resulting precipitate was collected by filtration, rinsed with water, and dried under reduced pressure at 50 C to yield compound 32d as a white powder. MS: M-H⁺=278.9, ¹H NMR (d₆-DMSO): δ 12.80 (s, 1H), 8.15 (s, 4H), 7.73 (d, 1H), 6.83 (d, 1H).

D. To compound 32d (0.056 g, 0.20 mmol) was added a solution of DMAP in DCM (5 mL, 0.080 M, 0.40 mmol) and BOP—Cl (0.080 g, 0.31 mmol). After 20 minutes compound 19c (0.052 g, 0.22 mmol) was added. After 3 days the reaction mixture was washed with 5 mL saturated NaHCO₃, dried over Na₂SO₄, and evaporated to a residue. The residue was purified by reverse-phase chromatography (25-95% acetonitrile/water+0.1% TFA). The proper fractions were lyophilized to give the title compound, Compound 205, as a light tan powder. MS: M+H⁺=504.0, ¹H NMR (d₆-DMSO): δ 11.10 (s,1H), 9.19 (s, 1H), 8.40 (d, 1H), 8.23 (d, 2H), 8.15-8.07 (m, 3H), 7.86-7.78 (m, 2H), 7.69 (s, 1H), 7.09 (d, 1H), 2.34 (s, 3H).

### EXAMPLE 33

### trans-N-[4-(4-Methyl-imidazol-1-yl)-3-trifluoromethyl-phenyl]-3-(4-trifluoromethylsulfinyl-phenyl)-acrylamide (Cpd 208)

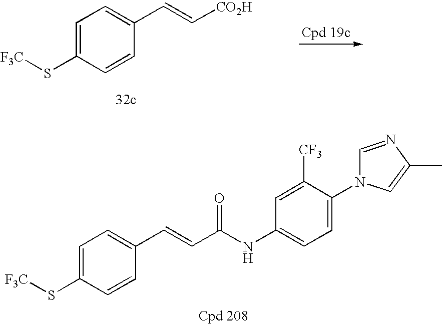

A. To compound 32c (0.050 g, 0.20 mmol) was added a solution of DMAP in DCM (5mL, 0.080 M, 0.40 mmol) and BOP—Cl (0.081 g, 0.32 mmol). After 20 minutes compound 19c (0.049 g, 0.20 mmol) was added. After 3 days the reaction mixture was washed with 5 mL saturated NaHCO₃, dried over Na₂SO₄, and evaporated to a residue. The residue was purified by reverse-phase chromatography (25-95% acetonitrile/water+0.1% TFA). The proper fractions were lyophilized to give compound 205 as a white powder. MS: M+H⁺=472.0, ¹H NMR (d₆-DMSO): δ 11.00 (s, 1H), 9.17 (s, 1H), 8.40 (d, 1H), 8.12 (dd, 1H), 7.82 (s, 4H), 7.79 (d, 1H), 7.74 (d, 1H), 7.68 (s, 1H), 6.94 (d, 1H), 2.34 (s, 3H).

### EXAMPLE 34

### N-[6-(4,5-Dichloro-imidazol-1-yl)-5-methyl-pyridin-3-yl]-3-(4-trifluoromethylsulfinyl-phenyl)-propionamide (Cpd 272)

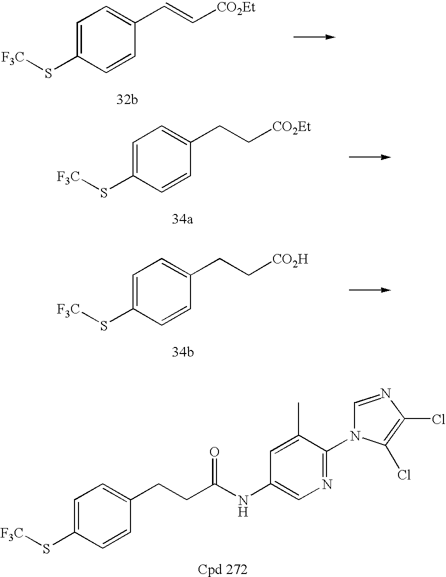

A. Compound 32b (2.798 g, 10.1 mmol) and 5% palladium on carbon (0.351 g) in 100 mL ethanol were hydrogenated at 50 psi of hydrogen gas for 22 hours. An additional amount of 10% palladium on carbon (0.524 g) was added and the hydrogenation was run for 18 more hours. The reaction mixture was filtered over a pad of Celite, followed by filtration through a nylon disk, and the solvents were removed in vacuo to yield compound 34a as a nearly colorless oil. ¹H NMR (d₆-DMSO): δ 7.63 (d, 2H), 7.40 (d, 2H), 4.03 (q, 2H), 2.91 (t, 2H), 2.66 (t, 2H), 1.13 (t, 3H).

B. To a solution of compound 34a (2.618 g, 9.4 mmol) in 50 mL 5:1 THF/H₂O was added LiOH—H₂O (0.790 g, 18.8 mmol). The reaction mixture was stirred for 18 hours then evaporated in vacuo until a water solution remained. It was diluted with an additional 100 mL of water and filtered over a nylon disk. The filtrate was acidified with 25 mL 1 N HCl. The resultant precipitate was collected by filtration, rinsed with additional water, air dried and then dried by vacuum at 50 C to give compound 34b as a white fine crystalline powder. MS: M−H⁺=249.0, ¹H NMR (d₆-DMSO): δ 12.21 (s, 1H), 7.63 (d, 2H), 7.41 (d, 2H), 2.88 (t, 2H), 2.58 (t, 2H).

C. To compound 34b (0.050 g, 0.20 mmol) was added a solution of DMAP in DCM (5mL, 0.080 M, 0.40 mmol), BOP—Cl (0.078 g, 0.31 mmol), and compound 23c (0.051 g, 0.21 mmol). After 3 days the reaction mixture was washed with 5 mL saturated NaHCO₃, dried over Na₂SO₄, and evaporated to a residue. The residue was purified by reverse-phase chromatography (25-95% acetonitrile/water+0.1% TFA). The proper fractions were lyophilized to give the title compound272 as a cream-colored powder. MS: M+H⁺=474.8, ¹H NMR (d₆-DMSO): δ 10.45 (s, 1H), 8.56 (d, 1H), 8.19 (d, 1H), 8.08 (s, 1H), 7.65 (d, 2H), 7.44 (d, 2H), 3.01 (t, 2H), 2.75 (t, 2H), 2.11 (s, 3H).

Compounds 1 through 280 of Formula (Ia) (wherein p is 1, R₄ is H and X is O) in the table below were synthesized using the procedures described above.

| | |
|---|---|
| | Formula Ia |
| 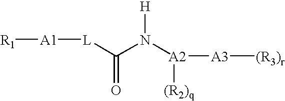 | |

| Cpd | R₁ | A1 | L | A2 | q | R₂ | A3 | r | R₃ |
|---|---|---|---|---|---|---|---|---|---|
| 1 | 4-t-butyl | phenyl | —(CH₂)₂— | phenyl | 0 | absent | 2-phenyl | 1 | 4-hydroxy |
| 2 | 4-t-butyl | phenyl | —(CH₂)₂— | phenyl | 0 | absent | 3-phenyl | 1 | 4-hydroxy |
| 3 | 4-t-butyl | phenyl | —(CH₂)₂— | phenyl | 0 | absent | 4-phenyl | 1 | 4-hydroxy |
| 4 | 4-t-butyl | phenyl | —(CH₂)₂— | phenyl | 0 | absent | 2-phenyl | 1 | 3-hydroxy |
| 5 | 4-t-butyl | phenyl | —(CH₂)₂— | phenyl | 0 | absent | 3-phenyl | 1 | 3-hydroxy |
| 6 | 4-t-butyl | phenyl | —(CH₂)₂— | phenyl | 0 | absent | 4-phenyl | 1 | 3-hydroxy |
| 8 | 4-t-butyl | phenyl | —(CH₂)₂— | phenyl | 0 | absent | 2-phenyl | 1 | 2-hydroxy |
| 9 | 4-t-butyl | phenyl | —(CH₂)₂— | phenyl | 0 | absent | 3-phenyl | 1 | 2-hydroxy |
| 10 | 4-t-butyl | phenyl | —(CH₂)₂— | phenyl | 0 | absent | 4-phenyl | 1 | 2-hydroxy |
| 11 | 4-t-butyl | phenyl | —(CH₂)₂— | phenyl | 0 | absent | 4-phenyl | 1 | 3-nitro |
| 12 | 4-t-butyl | phenyl | —(CH₂)₂— | phenyl | 0 | absent | 4-phenyl | 1 | 3-trifluoromethyl |
| 13 | 4-t-butyl | phenyl | —(CH₂)₂— | phenyl | 0 | absent | 4-phenyl | 1 | 3-methylcarbonyl |
| 14 | 4-t-butyl | phenyl | —(CH₂)₂— | phenyl | 0 | absent | 4-phenyl | 1 | 3-chloro |
| 15 | 4-t-butyl | phenyl | —(CH₂)₂— | phenyl | 0 | absent | 4-phenyl | 1 | 3-fluoro |
| 16 | 4-t-butyl | phenyl | —(CH₂)₂— | phenyl | 0 | absent | 4-phenyl | 1 | 3-methylcarbonyl- |
| | | | | | | | | | amino |
| 17 | 4-t-butyl | phenyl | —(CH₂)₂— | phenyl | 0 | absent | 4-phenyl | 1 | 3-amino |
| 19 | 4-t-butyl | phenyl | —(CH₂)₂— | phenyl | 0 | absent | 2-phenyl | 1 | 3-hydroxymethyl |
| 20 | 4-t-butyl | phenyl | —(CH₂)₂— | phenyl | 0 | absent | 2-phenyl | 1 | 4-hydroxymethyl |
| 21 | 4-t-butyl | phenyl | —(CH₂)₂— | phenyl | 0 | absent | 3-phenyl | 1 | 3-hydroxymethyl |
| 22 | 4-t-butyl | phenyl | —(CH₂)₂— | phenyl | 0 | absent | 3-phenyl | 1 | 4-hydroxymethyl |
| 23 | 4-t-butyl | phenyl | —(CH₂)₂— | phenyl | 0 | absent | 4-phenyl | 1 | 3-hydroxymethyl |
| 24 | 4-t-butyl | phenyl | —(CH₂)₂— | phenyl | 0 | absent | 4-phenyl | 1 | 4-hydroxymethyl |
| 25 | 4-t-butyl | phenyl | —(CH₂)₂— | phenyl | 0 | absent | 4-phenyl | 1 | 3-cyano |
| 26 | 4-t-butyl | phenyl | —(CH₂)₂— | phenyl | 0 | absent | 4-phenyl | 1 | 3-trifluoromethoxy |
| 27 | 4-t-butyl | phenyl | —(CH₂)₂— | phenyl | 0 | absent | 4-phenyl | 1 | 3-methoxy |
| 28 | 4-t-butyl | phenyl | —(CH₂)₂— | phenyl | 0 | absent | 4-phenyl | 1 | 3-methyl |
| 29 | 4-t-butyl | phenyl | —(CH₂)₂— | phenyl | 0 | absent | 4-phenyl | 1 | 3-methoxycarbonyl |
| 30 | 4-t-butyl | phenyl | —(CH₂)₂— | phenyl | 0 | absent | 4-phenyl | 1 | 3-carboxy |
| 31 | 4-t-butyl | phenyl | —(CH₂)₂— | phenyl | 0 | absent | 4-phenyl | 1 | 3-aminocarbonyl |
| 31 | 4-t-butyl | phenyl | —(CH₂)₂— | phenyl | 0 | absent | 4-phenyl | 1 | 3-aminocarbonyl |
| 33 | 4-t-butyl | phenyl | trans-CH═CH— | phenyl | 0 | absent | 3-phenyl | 1 | 3-aminocarbonyl |
| 34 | 4-t-butyl | phenyl | —(CH₂)₂— | phenyl | 0 | absent | 3-phenyl | 1 | 3-dimethylamino- |
| | | | | | | | | | carbonyl |
| 35 | 4-t-butyl | phenyl | trans-CH═CH— | phenyl | 0 | absent | 4-phenyl | 1 | 3-aminocarbonyl |
| 36 | 4-t-butyl | phenyl | trans-CH═CH— | phenyl | 0 | absent | 4-phenyl | 1 | 3-dimethylamino- |
| | | | | | | | | | carbonyl |
| 37 | 4-t-butyl | phenyl | —(CH₂)₂— | phenyl | 0 | absent | 3-phenyl | 1 | 3-aminocarbonyl |
| 38 | 4-t-butyl | phenyl | —(CH₂)₂— | phenyl | 0 | absent | 3-phenyl | 1 | 3-dimethylamino- |
| | | | | | | | | | carbonyl |
| 39 | 4-t-butyl | phenyl | —(CH₂)₂— | phenyl | 0 | absent | 4-phenyl | 1 | 3-dimethylamino- |
| | | | | | | | | | carbonyl |
| 40 | 4-t-butyl | phenyl | —(CH₂)₂— | phenyl | 0 | absent | 4-phenyl | 1 | 3-methylsulfonylamino |
| 41 | 4-t-butyl | phenyl | —(CH₂)₂— | phenyl | 0 | absent | 4-phenyl | 1 | 3-t-butoxycarbonyl- |
| | | | | | | | | | amino |
| 42 | 4-t-butyl | phenyl | —(CH₂)₂— | phenyl | 0 | absent | 4-phenyl | 1 | 3-t-butoxycarbonyl- |
| | | | | | | | | | aminomethyl |
| 43 | 4-t-butyl | phenyl | —(CH₂)₂— | phenyl | 0 | absent | 4-phenyl | 1 | 3-aminomethyl |
| 44 | 4-t-butyl | phenyl | —(CH₂)₂— | phenyl | 0 | absent | 4-phenyl | 1 | 3-ureido |
| 45 | 4-t-butyl | phenyl | —(CH₂)₂— | phenyl | 0 | absent | 4-phenyl | 1 | 3-(2-amino-ethoxy) |
| 46 | 4-t-butyl | phenyl | —(CH₂)₂— | phenyl | 0 | absent | 4-phenyl | 1 | 3-(2-hydroxy- |
| | | | | | | | | | ethylamino) |
| 47 | 4-trifluoromethyl | phenyl | trans-CH═CH— | phenyl | 0 | absent | 4-phenyl | 1 | 3-aminocarbonyl |
| 48 | 4-trifluoromethyl | phenyl | —(CH₂)₂— | phenyl | 0 | absent | 4-phenyl | 1 | 3-aminocarbonyl |
| 50 | 4-t-butyl | phenyl | —(CH₂)₂— | phenyl | 1 | 2-methyl | 4-phenyl | 1 | 3-aminocarbonyl |
| 51 | 4-t-butyl | phenyl | —(CH₂)₂— | phenyl | 1 | 3-methyl | 4-phenyl | 1 | 3-aminocarbonyl |
| 52 | 4-t-butyl | phenyl | —(CH₂)₂— | phenyl | 1 | 2-fluoro | 4-phenyl | 1 | 3-aminocarbonyl |
| 53 | 4-t-butyl | phenyl | —(CH₂)₂— | phenyl | 1 | 3-fluoro | 4-phenyl | 1 | 3-aminocarbonyl |
| 54 | 4-t-butyl | phenyl | —(CH₂)₂— | phenyl | 11 | 2-methyl | 4-phenyl | 1 | 3-methylcarbonyl- |
| | | | | | | | | | amino |
| 55 | 4-t-butyl | phenyl | —(CH₂)₂— | phenyl | 1 | 3-methyl | 4-phenyl | 1 | 3-methylcarbonyl- |
| | | | | | | | | | amino |
| 56 | 4-t-butyl | phenyl | —(CH₂)₂— | phenyl | 1 | 2-fluoro | 4-phenyl | 1 | 3-methylcarbonyl- |
| | | | | | | | | | amino |
| 57 | 4-t-butyl | phenyl | —(CH₂)₂— | phenyl | 1 | 3-fluoro | 4-phenyl | 1 | 3-methylcarbonyl- |
| | | | | | | | | | amino |
| 58 | 4-t-butyl | phenyl | —(CH₂)₂— | phenyl | 1 | 2-methyl | 4-phenyl | 1 | 3-hydroxy |
| 59 | 4-t-butyl | phenyl | —(CH₂)₂— | phenyl | 1 | 3-methyl | 4-phenyl | 1 | 3-hydroxy |
| 60 | 4-t-butyl | phenyl | —(CH₂)₂— | phenyl | 1 | 2-fluoro | 4-phenyl | 1 | 3-hydroxy |
| 61 | 4-t-butyl | phenyl | —(CH₂)₂— | phenyl | 1 | 3-fluoro | 4-phenyl | 1 | 3-hydroxy |
| 62 | 4-t-butyl | phenyl | —(CH₂)₂— | phenyl | 0 | absent | 4-phenyl | 1 | 3-hydroxy |
| 63 | 5-t-butyl | thio- | —(CH₂)₂— | phenyl | 0 | absent | 4-phenyl | 1 | 3-aminocarbonyl |
| | | phen-2-yl | | | | | | | |
| 64 | 5-t-butyl | thio- | —(CH₂)₂— | phenyl | 0 | absent | 4-phenyl | 1 | 3-methylcarbonyl- |
| | | phen-2-yl | | | | | | | amino |
| 65 | 5-t-butyl | thio- | —(CH₂)₂— | phenyl | 0 | absent | 4-phenyl | 1 | 3-hydroxy |
| | | phen-2-yl | | | | | | | |
| 66 | 4-t-butyl | phenyl | —(CH₂)₂— | phenyl | 1 | 2-carboxy | 4-phenyl | 1 | 3-aminocarbonyl |
| 67 | 4-t-butyl | phenyl | —(CH₂)₂— | phenyl | 1 | 2-carboxy | 4-phenyl | 1 | 3-methylamino- |
| | | | | | | | | | carbonyl |
| 68 | 4-t-butyl | phenyl | —(CH₂)₂— | phenyl | 1 | 2-carboxy | 4-phenyl | 1 | 3-hydroxy |
| 69 | 4-t-butyl | phenyl | —(CH₂)₂— | phenyl | 1 | 2-hydroxy- | 4-phenyl | 1 | 3-aminocarbonyl |
| | | | | | | methyl | | | |
| 70 | 4-t-butyl | phenyl | —(CH₂)₂— | phenyl | 1 | 2-hydroxy- | 4-phenyl | 1 | 3-methylcarbonyl- |
| | | | | | | methyl | | | amino |
| 71 | 4-t-butyl | phenyl | —(CH₂)₂— | phenyl | 1 | 2-hydroxy- | 4-phenyl | 1 | 3-hydroxy |
| | | | | | | methyl | | | |
| 72 | 4-t-butyl | phenyl | —(CH₂)₂— | phenyl | 1 | 3-carboxy | 4-phenyl | 1 | 3-aminocarbonyl |
| 73 | 4-t-butyl | phenyl | —(CH₂)₂— | phenyl | 1 | 3-carboxy | 4-phenyl | 1 | 3-methylcarbonyl- |
| | | | | | | | | | amino |
| 74 | 4-t-butyl | phenyl | —(CH₂)₂— | phenyl | 1 | 3-carboxy | 4-phenyl | 1 | 3-hydroxy |
| 75 | 4-trifluoromethyl | phenyl | —(CH₂)₂— | phenyl | 0 | absent | 4-phenyl | 1 | 3-methylcarbonyl- |
| | | | | | | | | | amino |
| 76 | 4-trifluoromethyl | phenyl | —(CH₂)₂— | phenyl | 0 | absent | 4-phenyl | 1 | 2-hydroxy |
| 77 | 4-trifluoromethyl | phenyl | —(CH₂)₂— | phenyl | 0 | absent | 4-phenyl | 1 | 3-hydroxy |
| 78 | 4-trifluoromethyl | phenyl | —(CH₂)₂— | phenyl | 0 | absent | 4-phenyl | 1 | 4-hydroxy |
| 79 | 4-trifluoromethyl | phenyl | —(CH₂)₂— | phenyl | 0 | absent | 4-phenyl | 1 | 3-hydroxymethyl |
| 80 | 4-trifluoromethyl | phenyl | —(CH₂)₂— | phenyl | 0 | absent | 4-phenyl | 1 | 4-hydroxymethyl |
| 81 | 4-t-butyl | phenyl | —(CH₂)₂— | naph- | 0 | absent | 4-phenyl | 1 | 3-methylcarbonyl- |
| | | | | thalen-1-yl | | | | | amino |
| 82 | 4-t-butyl | phenyl | —(CH₂)₂— | naph- | 0 | absent | 4-phenyl | 1 | 3-hydroxy |
| | | | | thalen-1-yl | | | | | |
| 83 | 4-trifluoromethyl | phenyl | —(CH₂)₂— | naph- | 0 | absent | 4-phenyl | 1 | 3-aminocarbonyl |
| | | | | thalen-1-yl | | | | | |
| 84 | 4-trifluoromethyl | phenyl | —(CH₂)₂— | naph- | 0 | absent | 4-phenyl | 1 | 3-methylcarbonyl- |
| | | | | thalen-1-yl | | | | | amino |
| 85 | 4-trifluoromethyl | phenyl | —(CH₂)₂— | naph- | 0 | absent | 4-phenyl | 1 | 3-hydroxy |
| | | | | thalen-1-yl | | | | | |
| 86 | 4-t-butyl | phenyl | —(CH₂)₂— | phenyl | 2 | 3,5-dimethyl | 4-phenyl | 1 | 3-hydroxy |
| 87 | 4-t-butyl | phenyl | —(CH₂)₂— | phenyl | 2 | 3,5-dimethyl | 4-phenyl | 1 | 4-hydroxy |
| 88 | 4-t-butyl | phenyl | —(CH₂)₂— | phenyl | 2 | 3,5-dimethyl | 4-phenyl | 1 | 4-hydroxymethyl |
| 89 | 4-t-butyl | phenyl | —(CH₂)₂— | phenyl | 2 | 3,5-difluoro | 4-phenyl | 1 | 3-hydroxy |
| 90 | 4-t-butyl | phenyl | —(CH₂)₂— | phenyl | 2 | 3,5-difluoro | 4-phenyl | 1 | 4-hydroxy |
| 91 | 4-t-butyl | phenyl | —(CH₂)₂— | phenyl | 2 | 3,5-difluoro | 4-phenyl | 1 | 4-hydroxymethyl |
| 92 | 4-t-butyl | phenyl | —(CH₂)₂— | phenyl | 1 | 3-hydroxy- | 4-phenyl | 1 | 2-hydroxy |
| | | | | | | methyl | | | |
| 93 | 4-t-butyl | phenyl | —(CH₂)₂— | phenyl | 1 | 3-hydroxy- | 4-phenyl | 1 | 3-hydroxy |
| | | | | | | methyl | | | |
| 94 | 4-t-butyl | phenyl | —(CH₂)₂— | phenyl | 1 | 3-hydroxy- | 4-phenyl | 1 | 4-hydroxy |
| | | | | | | methyl | | | |
| 95 | 4-t-butyl | phenyl | —(CH₂)₂— | phenyl | 1 | 3-hydroxy- | 4-phenyl | 1 | 3-hydroxymethyl |
| | | | | | | methyl | | | |
| 96 | 4-t-butyl | phenyl | —(CH₂)₂— | phenyl | 1 | 3-hydroxy- | 4-phenyl | 1 | 4-hydroxymethyl |
| | | | | | | methyl | | | |
| 97 | 4-t-butyl | phenyl | —(CH₂)₂— | phenyl | 1 | 3-methyl | 4-phenyl | 1 | 2-hydroxy |
| 98 | 4-t-butyl | phenyl | —(CH₂)₂— | phenyl | 1 | 3-methyl | 4-phenyl | 1 | 4-hydroxy |
| 99 | 4-t-butyl | phenyl | —(CH₂)₂— | phenyl | 1 | 3-methyl | 4-phenyl | 1 | 4-hydroxymethyl |
| 100 | 4-t-butyl | phenyl | —(CH₂)₂— | phenyl | 1 | 3-fluoro | 4-phenyl | 1 | 2-hydroxy |
| 101 | 4-t-butyl | phenyl | —(CH₂)₂— | phenyl | 1 | 3-fluoro | 4-phenyl | 1 | 4-hydroxy |
| 102 | 4-t-butyl | phenyl | —(CH₂)₂— | phenyl | 1 | 3-fluoro | 4-phenyl | 1 | 4-hydroxymethyl |
| 103 | 4-t-butyl | phenyl | —(CH₂)₂— | phenyl | 0 | absent | 2-pyridin-4-yl | 0 | absent |
| 104 | 4-t-butyl | phenyl | —(CH₂)₂— | phenyl | 0 | absent | 3-pyridin-4-yl | 0 | absent |
| 105 | 4-t-butyl | phenyl | —(CH₂)₂— | phenyl | 0 | absent | 2-pyridin-3-yl | 0 | absent |
| 106 | 4-t-butyl | phenyl | —(CH₂)₂— | phenyl | 0 | absent | 3-pyridin-3-yl | 0 | absent |
| 107 | 4-t-butyl | phenyl | —(CH₂)₂— | phenyl | 0 | absent | 4-pyridin-3-yl | 0 | absent |
| 108 | 4-t-butyl | phenyl | —(CH₂)₂— | phenyl | 0 | absent | 2-pyridin-2-yl | 0 | absent |
| 109 | 4-t-butyl | phenyl | —(CH₂)₂— | phenyl | 0 | absent | 3-pyridin-2-yl | 0 | absent |
| 110 | 4-t-butyl | phenyl | —(CH₂)₂— | phenyl | 0 | absent | 4-pyridin-2-yl | 0 | absent |
| 111 | 4-t-butyl | phenyl | —(CH₂)₂— | phenyl | 0 | absent | 4-pyridin-4-yl | 0 | absent |
| 112 | 4-t-butyl | phenyl | —(CH₂)₂— | phenyl | 0 | absent | 4-pyrimidin-2-yl | 0 | absent |
| 113 | 4-t-butyl | phenyl | —(CH₂)₂— | phenyl | 0 | absent | 4-pyrimidin-5-yl | 0 | absent |
| 114 | 4-t-butyl | phenyl | —(CH₂)₂— | phenyl | 0 | absent | 3-pyridin-2-yl | 1 | 6-hydroxy |
| 115 | 4-t-butyl | phenyl | —(CH₂)₂— | phenyl | 0 | absent | 4-pyridin-2-yl | 1 | 3-hydroxy |
| 116 | 4-t-butyl | phenyl | —(CH₂)₂— | phenyl | 0 | absent | 3-pyrimidin-2-yl | 0 | absent |
| 117 | 4-t-butyl | phenyl | —(CH₂)₂— | phenyl | 0 | absent | 3-pyrimidin-5-yl | 0 | absent |
| 118 | 4-t-butyl | phenyl | —(CH₂)₂— | phenyl | 0 | absent | 3-pyridin-2-yl | 1 | 6-methoxy |
| 119 | 4-t-butyl | phenyl | —(CH₂)₂— | phenyl | 0 | absent | 3-pyridin-3-yl | 1 | 6-methoxy |
| 120 | 4-t-butyl | phenyl | —(CH₂)₂— | phenyl | 0 | absent | 4-pyridin-2-yl | 1 | 6-methoxy |
| 121 | 4-t-butyl | phenyl | —(CH₂)₂— | phenyl | 0 | absent | 4-pyridin-3-yl | 1 | 6-methoxy |
| 122 | 4-t-butyl | phenyl | —(CH₂)₂— | phenyl | 0 | absent | 4-1H-indol-4-yl | 0 | absent |
| 123 | 4-t-butyl | phenyl | —(CH₂)₂— | phenyl | 0 | absent | 4-1H-indol-6-yl | 0 | absent |
| 124 | 4-t-butyl | phenyl | —(CH₂)₂— | py- | 0 | absent | 5-phenyl | 1 | 3-aminocarbonyl |
| | | | | ridin-2-yl | | | | | |
| 125 | 4-t-butyl | phenyl | —(CH₂)₂— | py- | 0 | absent | 5-phenyl | 1 | 3-methylcarbonyl- |
| | | | | ridin-2-yl | | | | | amino |
| 126 | 4-t-butyl | phenyl | —(CH₂)₂— | py- | 0 | absent | 6-phenyl | 1 | 3-hydroxy |
| | | | | ridin-2-yl | | | | | |
| 127 | 4-t-butyl | phenyl | —(CH₂)₂— | py- | 0 | absent | 6-phenyl | 1 | 3-aminocarbonyl |
| | | | | ridin-3-yl | | | | | |
| 128 | 4-t-butyl | phenyl | —(CH₂)₂— | py- | 0 | absent | 6-phenyl | 1 | methylcarbonylamino |
| | | | | ridin-3-yl | | | | | |
| 129 | 4-t-butyl | phenyl | —(CH₂)₂— | py- | 0 | absent | 6-phenyl | 1 | 3-hydroxy |
| | | | | ridin-3-yl | | | | | |
| 130 | 4-t-butyl | phenyl | —(CH₂)₂— | pyri- | 0 | absent | 5-phenyl | 1 | 3-aminocarbonyl |
| | | | | midin-2-yl | | | | | |
| 131 | 4-t-butyl | phenyl | —(CH₂)₂— | py- | 0 | absent | 5-phenyl | 1 | 3-aminocarbonyl |
| | | | | razin-2-yl | | | | | |
| 132 | 4-t-butyl | phenyl | —(CH₂)₂— | py- | 0 | absent | 6-phenyl | 1 | 2-hydroxy |
| | | | | ridin-3-yl | | | | | |
| 133 | 4-t-butyl | phenyl | —(CH₂)₂— | py- | 0 | absent | 6-phenyl | 1 | 4-hydroxy |
| | | | | ridin-3-yl | | | | | |
| 134 | 4-t-butyl | phenyl | —(CH₂)₂— | py- | 0 | absent | 6-phenyl | 1 | 3-hydroxymethyl |
| | | | | ridin-3-yl | | | | | |
| 135 | 4-t-butyl | phenyl | —(CH₂)₂— | py- | 0 | absent | 6-phenyl | 1 | 4-hydroxymethyl |
| | | | | ridin-3-yl | | | | | |
| 136 | 4-t-butyl | phenyl | —(CH₂)₂— | py- | 0 | absent | 6-pyridin-3-yl | 0 | absent |
| | | | | ridin-3-yl | | | | | |
| 137 | 4-t-butyl | phenyl | —(CH₂)₂— | py- | 0 | absent | 6-pyridin-4-yl | 0 | absent |
| | | | | ridin-3-yl | | | | | |
| 138 | 4-t-butyl | thio- | —(CH₂)₂— | phenyl | 0 | absent | 4-pyridin-4-yl | 0 | absent |
| | | phen-2-yl | | | | | | | |
| 139 | 4-t-butyl | phenyl | —(CH₂)₂— | phenyl | 1 | 2-carboxy | 4-pyridin-4-yl | 0 | absent |
| 140 | 4-t-butyl | phenyl | —(CH₂)₂— | phenyl | 1 | 2-hydroxy- | 4-pyridin-4-yl | 0 | absent |
| | | | | | | methyl | | | |
| 141 | 4-t-butyl | phenyl | —(CH₂)₂— | phenyl | 1 | 3-carboxy | 4-pyridin-4-yl | 0 | absent |
| 142 | 4-trifluoromethyl | phenyl | —(CH₂)₂— | phenyl | 0 | absent | 4-pyridin-3-yl | 0 | absent |
| 143 | 4-trifluoromethyl | phenyl | —(CH₂)₂— | phenyl | 0 | absent | 4-pyridin-4-yl | 0 | absent |
| 144 | 4-trifluoromethyl | phenyl | —(CH₂)₂— | py- | 0 | absent | 6-phenyl | 1 | 3-aminocarbonyl |
| | | | | ridin-3-yl | | | | | |
| 145 | 4-trifluoromethyl | phenyl | —(CH₂)₂— | py- | 0 | absent | 6-phenyl | 1 | 3-methylcarbonyl- |
| | | | | ridin-3-yl | | | | | amino |
| 146 | 4-trifluoromethyl | phenyl | —(CH₂)₂— | py- | 0 | absent | 6-phenyl | 1 | 2-hydroxy |
| | | | | ridin-3-yl | | | | | |
| 147 | 4-trifluoromethyl | phenyl | —(CH₂)₂— | py- | 0 | absent | 6-phenyl | 1 | 3-hydroxy |
| | | | | ridin-3-yl | | | | | |
| 148 | 4-trifluromethyl | phenyl | —(CH₂)₂— | py- | 0 | absent | 6-phenyl | 1 | 4-hydroxy |
| | | | | ridin-3-yl | | | | | |
| 149 | 4-trifluoromethyl | phenyl | —(CH₂)₂— | py- | 0 | absent | 6-phenyl | 1 | 3-hydroxymethyl |
| | | | | ridin-3-yl | | | | | |
| 150 | 4-trifluoromethyl | phenyl | —(CH₂)₂— | py- | 0 | absent | 6-phenyl | 1 | 4-hydroxymethyl |
| | | | | ridin-3-yl | | | | | |
| 151 | 4-trifluoromethyl | phenyl | —(CH₂)₂— | py- | 0 | absent | 6-pyridin-3-yl | 0 | absent |
| | | | | ridin-3-yl | | | | | |
| 152 | 4-trifluoromethyl | phenyl | —(CH₂)₂— | py- | 0 | absent | 6-pyridin-4-yl | 0 | absent |
| | | | | ridin-3-yl | | | | | |
| 153 | 4-t-butyl | phenyl | —(CH₂)₂— | naph- | 0 | absent | 4-pyridin-4-yl | 0 | absent |
| | | | | thalen-1-yl | | | | | |
| 154 | 4-t-butyl | phenyl | —(CH₂)₂— | isoquino- | 0 | absent | 8-phenyl | 1 | 3-methylcarbonyl- |
| | | | | lin-5-yl | | | | | amino |
| 155 | 4-t-butyl | phenyl | —(CH₂)₂— | isoquino- | 0 | absent | 8-phenyl | 1 | 3-hydroxy |
| | | | | lin-5-yl | | | | | |
| 156 | 4-t-butyl | phenyl | —(CH₂)₂— | isoquino- | 0 | absent | 8-pyridin-4-yl | 0 | absent |
| | | | | lin-5-yl | | | | | |
| 157 | 4-trifluoromethyl | phenyl | —(CH₂)₂— | naph- | 0 | absent | 4-pyridin-4-yl | 0 | absent |
| | | | | thalen-1-yl | | | | | |
| 158 | 4-trifluoromethyl | phenyl | —(CH₂)₂— | isoquino- | 0 | absent | 8-phenyl | 1 | 3-hydroxy |
| | | | | lin-5-yl | | | | | |
| 159 | 4-trifluoromethyl | phenyl | —(CH₂)₂— | isoquino- | 0 | absent | 8-pyridin-4-yl | 0 | absent |
| | | | | lin-5-yl | | | | | |
| 160 | 4-t-butyl | phenyl | —(CH₂)₂— | py- | 1 | 5-methyl | 6-phenyl | 1 | 2-hydroxy |
| | | | | ridin-3-yl | | | | | |
| 161 | 4-t-butyl | phenyl | —(CH₂)₂— | py- | 1 | 5-methyl | 6-phenyl | 1 | 3-hydroxy |
| | | | | ridin-3-yl | | | | | |
| 162 | 4-t-butyl | phenyl | —(CH₂)₂— | py- | 1 | 5-methyl | 6-phenyl | 1 | 4-hydroxy |
| | | | | ridin-3-yl | | | | | |
| 163 | 4-t-butyl | phenyl | —(CH₂)₂— | py- | 1 | 5-methyl | 6-phenyl | 1 | 4-hydroxymethyl |
| | | | | ridin-3-yl | | | | | |
| 164 | 4-t-butyl | phenyl | —(CH₂)₂— | py- | 1 | 5-methyl | 6-pyridin-3-yl | 0 | absent |
| | | | | ridin-3-yl | | | | | |
| 165 | 4-t-butyl | phenyl | —(CH₂)₂— | py- | 1 | 5-methyl | 6-pyridin-4-yl | 0 | absent |
| | | | | ridin-3-yl | | | | | |
| 166 | 4-t-butyl | phenyl | —(CH₂)₂— | phenyl | 2 | 3,5-dimethyl | 4-pyridin-3-yl | 0 | absent |
| 167 | 4-t-butyl | phenyl | —(CH₂)₂— | phenyl | 2 | 3,5-dimethyl | 4-pyridin-4-yl | 0 | absent |
| 168 | 4-t-butyl | phenyl | —(CH₂)₂— | phenyl | 2 | 3,5-difluoro | 4-pyridin-3-yl | 0 | absent |
| 169 | 4-t-butyl | phenyl | —(CH₂)₂— | phenyl | 2 | 3,5-difluoro | 4-pyridin-4-yl | 0 | absent |
| 170 | 4-t-butyl | phenyl | —(CH₂)₂— | phenyl | 1 | 3-hydroxy- | 4-pyridin-3-yl | 0 | absent |
| | | | | | | methyl | | | |
| 171 | 4-t-butyl | phenyl | —(CH₂)₂— | phenyl | 1 | 3-hydroxy- | 4-pyridin-4-yl | 0 | absent |
| | | | | | | methyl | | | |
| 172 | 4-t-butyl | phenyl | —(CH₂)₂— | phenyl | 1 | 3-methyl | 4-pyridin-2-yl | 0 | absent |
| 173 | 4-t-butyl | phenyl | —(CH₂)₂— | phenyl | 1 | 3-fluoro | 4-pyridin-2-yl | 0 | absent |
| 174 | 4-t-butyl | phenyl | —(CH₂)₂— | phenyl | 2 | 3,5-dimethyl | 4-pyridin-2-yl | 0 | absent |
| 175 | 4-t-butyl | phenyl | —(CH₂)₂— | phenyl | 2 | 3,5-difluoro | 4-pyridin-2-yl | 0 | absent |
| 176 | 4-t-butyl | phenyl | —(CH₂)₂— | py- | 0 | absent | 6-pyridin-2-yl | 0 | absent |
| | | | | ridin-3-yl | | | | | |
| 177 | 4-t-butyl | phenyl | —(CH₂)₂— | py- | 1 | 5-methyl | 6-pyridin-2-yl | 0 | absent |
| | | | | ridin-3-yl | | | | | |
| 178 | 4-t-butyl | phenyl | —(CH₂)₂— | py- | 0 | absent | 5-pyridin-2-yl | 0 | absent |
| | | | | razin-2-yl | | | | | |
| 179 | 4-t-butyl | phenyl | —(CH₂)₂— | phenyl | 1 | 3-methyl | 4-pyridin-3-yl | 0 | absent |
| 180 | 4-t-butyl | phenyl | —(CH₂)₂— | phenyl | 1 | 3-methyl | 4-pyridin-4-yl | 0 | absent |
| 181 | 4-t-butyl | phenyl | —(CH₂)₂— | phenyl | 1 | 3-fluoro | 4-pyridin-3-yl | 0 | absent |
| 182 | 4-t-butyl | phenyl | —(CH₂)₂— | phenyl | 1 | 3-fluoro | 4-pyridin-4-yl | 0 | absent |
| 183 | 4-t-butyl | phenyl | —(CH₂)₂— | py- | 0 | absent | 5-phenyl | 1 | 2-hydroxy |
| | | | | razin-2-yl | | | | | |
| 184 | 4-t-butyl | phenyl | —(CH₂)₂— | py- | 0 | absent | 5-phenyl | 1 | 3-hydroxy |
| | | | | razin-2-yl | | | | | |
| 185 | 4-t-butyl | phenyl | —(CH₂)₂— | py- | 0 | absent | 5-phenyl | 1 | 4-hydroxy |
| | | | | razin-2-yl | | | | | |
| 186 | 4-t-butyl | phenyl | —(CH₂)₂— | py- | 0 | absent | 5-phenyl | 1 | 4-hydroxymethyl |
| | | | | razin-2-yl | | | | | |
| 187 | 4-t-butyl | phenyl | —(CH₂)₂— | py- | 0 | absent | 5-pyridin-3-yl | 0 | absent |
| | | | | razin-2-yl | | | | | |
| 188 | 4-t-butyl | phenyl | —(CH₂)₂— | py- | 0 | absent | 5-pyridin-4-yl | 0 | absent |
| | | | | razin-2-yl | | | | | |
| 189 | 4-t-butyl | phenyl | —(CH₂)₂— | phenyl | 0 | absent | 4-imidazol-2-yl | 1 | 1-methyl |
| 190 | 4-t-butyl | phenyl | —(CH₂)₂— | phenyl | 0 | absent | 4-imidazol-4-yl | 1 | 1-methyl |
| 191 | 4-t-butyl | phenyl | —(CH₂)₂— | phenyl | 0 | absent | 4-imidazol-5-yl | 1 | 1-methyl |
| 192 | 4-t-butyl | phenyl | —(CH₂)₂— | phenyl | 0 | absent | 4-imidazol-4-yl | 0 | absent |
| 193 | 4-t-butyl | phenyl | —(CH₂)₂— | phenyl | 0 | absent | 4-oxazol-5-yl | 0 | absent |
| 194 | 4-t-butyl | phenyl | —(CH₂)₂— | phenyl | 0 | absent | 4-(4,5-dihydro- | 0 | absent |
| | | | | | | | oxazol-5-yl) | | |
| 195 | 4-t-butyl | phenyl | —(CH₂)₂— | phenyl | 0 | absent | 4-thiazol-4-yl | 1 | 2-methyl |
| 196 | 4-t-butyl | phenyl | —(CH₂)₂— | phenyl | 0 | absent | 4-pyrazol-1-yl | 2 | 3,5-dimethyl |
| 197 | 4-t-butyl | phenyl | —(CH₂)₂— | phenyl | 0 | absent | 4-thiadiazol-4-yl | 0 | absent |
| 198 | 4-t-butyl | phenyl | —(CH₂)₂— | phenyl | 0 | absent | 4-(1,2,4-triazol-1-yl) | 0 | absent |
| 199 | 4-t-butyl | phenyl | —(CH₂)₂— | phenyl | 0 | absent | 4-1H-tetrazol-5-yl | 0 | absent |
| 200 | 4-t-butyl | phenyl | —(CH₂)₂— | phenyl | 0 | absent | 4-imidazol-3-yl | 2 | 4,5-dichloro |
| 201 | 4-t-butyl | phenyl | —(CH₂)₂— | phenyl | 0 | absent | 4-(2,4-dihydro-py- | 2 | 3-oxo, 5-methyl |
| | | | | | | | razol-2-yl) | | |
| 202 | 4-t-butyl | phenyl | —(CH₂)₂— | py- | 1 | 5-trifluoro- | 6-imidazol-1-yl | 0 | absent |
| | | | | ridin-3-yl | | methyl | | | |
| 203 | 4-trifluoromethyl | phenyl | —(CH₂)₂— | py- | 1 | 5-methyl | 6-imidazol-1-yl | 2 | 4,5-dichloro |
| | | | | ridin-3-yl | | | | | |
| 204 | 4-trifluoromethyl | phenyl | —(CH₂)₂— | phenyl | 1 | 3-trifluoro- | 4-imidazol-1-yl | 1 | 4-methyl |
| | | | | | | methyl | | | |
| 205 | 4-trifluoromethylsulphonyl | phenyl | trans-CH═CH— | phenyl | 1 | 3-trifluoro- | 4-imidazol-1-yl | 1 | 4-methyl |
| | | | | | | methyl | | | |
| 206 | 4-trifluoromethyl | phenyl | —(CH₂)₂— | phenyl | 1 | 3-trifluoro- | 4-phenyl | 1 | 3-hydroxy |
| | | | | | | methyl | | | |
| 207 | 4-trifluoromethyl | phenyl | —(CH₂)₂— | phenyl | 1 | 3-trifluoro- | 4-imidazol-1-yl | 0 | absent |
| | | | | | | methyl | | | |
| 208 | 4-trifluoromethylthio | phenyl | trans-CH═CH— | phenyl | 1 | 3-trifluoro- | 4-imidazol-1-yl | 1 | 4-methyl |
| | | | | | | methyl | | | |
| 209 | 4-trifluoromethylsulphonyl | phenyl | —(CH₂)₂— | phenyl | 1 | 3-trifluoro- | 4-imidazol-1-yl | 1 | 4-methyl |
| | | | | | | methyl | | | |
| 210 | 4-trifluoromethyl | phenyl | —(CH₂)₂— | py- | 1 | 5-trifluoro- | 6-phenyl | 1 | 3-hydroxy |
| | | | | ridin-3-yl | | methyl | | | |
| 211 | 4-t-butyl | phenyl | —(CH₂)₂— | phenyl | 0 | absent | 4-imidazol-1-yl | 2 | 4,5-dichloro |
| 212 | 4-t-butyl | phenyl | —(CH₂)₂— | phenyl | 1 | 3-trifluoro- | 4-imidazol-1-yl | 2 | 4,5-dichloro |
| | | | | | | methyl | | | |
| 213 | 4-trifluoromethyl | phenyl | —(CH₂)₂— | py- | 1 | 5-trifluoro- | 6-phenyl | 1 | 2-hydroxy |
| | | | | ridin-3-yl | | methyl | | | |
| 214 | 4-t-butyl | phenyl | —(CH₂)₂— | py- | 1 | 5-methyl | 6-imidazol-1-yl | 0 | absent |
| | | | | ridin-3-yl | | | | | |
| 215 | 4-t-butyl | phenyl | —(CH₂)₂— | py- | 1 | 5-methyl | 6-imidazol-1-yl | 2 | 4,5-dichloro |
| | | | | ridin-3-yl | | | | | |
| 216 | 4-t-butyl | phenyl | —(CH₂)₂— | py- | 1 | 5-methyl | 6-phenyl | 1 | 3-methylcarbonyl- |
| | | | | ridin-3-yl | | | | | amino |
| 217 | 4-trifluoromethylsulphonyl | phenyl | trans-CH═CH— | phenyl | 1 | 3-trifluoro- | 4-imidazol-1-yl | 2 | 4,5-dichloro |
| | | | | | | methyl | | | |
| 218 | 4-trifluoromethyl | phenyl | —(CH₂)₂— | phenyl | 1 | 3-trifluoro- | 4-imidazol-1-yl | 2 | 4,5-dichloro |
| | | | | | | methyl | | | |
| 219 | 4-trifluoromethylthio | phenyl | —(CH₂)₂— | phenyl | 1 | 3-trifluoro- | 4-imidazol-1-yl | 1 | 4-methyl |
| | | | | | | methyl | | | |
| 220 | 4-trifluoromethyl | phenyl | —(CH₂)₂— | phenyl | 1 | 3-trifluoro- | 4-pyridin-4-yl | 0 | absent |
| | | | | | | methyl | | | |
| 221 | 4-trifluoromethyl | phenyl | —(CH₂)₂— | py- | 1 | 5-trifluoro- | 6-phenyl | 1 | 4-hydroxy |
| | | | | ridin-3-yl | | methyl | | | |
| 222 | 4-trifluoromethylsulphinyl | phenyl | —(CH₂)₂— | phenyl | 1 | 3-trifluoro- | 4-imidazol-1-yl | 1 | 4-methyl |
| | | | | | | methyl | | | |
| 223 | 4-trifluoromethyl | phenyl | —(CH₂)₂— | phenyl | 1 | 3-trifluoro- | 4-phenyl | 1 | 4-hydroxymethyl |
| | | | | | | methyl | | | |
| 224 | t-butyl | phenyl | —(CH₂)₂— | py- | 1 | 5-trifluoro- | 6-imidazol-1-yl | 2 | 4,5-dichloro |
| | | | | ridin-3-yl | | methyl | | | |
| 225 | 4-trifluoromethyl | phenyl | —(CH₂)₂— | py- | 1 | 5-trifluoro- | 6-phenyl | 1 | 4-hydroxymethyl |
| | | | | ridin-3-yl | | methyl | | | |
| 226 | 4-trifluoromethyl | phenyl | —(CH₂)₂— | py- | | 5-methyl | 6-imidazol-1-yl | 1 | 4-methyl |
| | | | | ridin-3-yl | | | | | |
| 227 | 4-trifluoromethyl | phenyl | —(CH₂)₂— | phenyl | 1 | 3-trifluoro- | 4-imidazol-1-yl | 1 | 4-chloro |
| | | | | | | methyl | | | |
| 228 | 4-trifluoromethylthio | phenyl | trans-CH═CH— | phenyl | 1 | 3-trifluoro- | 4-imidazol-1-yl | 2 | 4,5-dichloro |
| | | | | | | methyl | | | |
| 229 | 4-trifluoromethyl | phenyl | —(CH₂)₂— | phenyl | 1 | 3-triflouoro- | 4-phenyl | 1 | 4-hydroxy |
| | | | | | | methyl | | | |
| 230 | 4-chloro | phenyl | —(CH₂)₂— | phenyl | 1 | 3-trifluoro- | 4-pyridin-4-yl | 0 | absent |
| | | | | | | methyl | | | |
| 231 | 4-chloro | phenyl | —(CH₂)₂— | phenyl | 1 | 3-trifluoro- | 4-imidazol-1-yl | 2 | 4,5-dichloro |
| | | | | | | methyl | | | |
| 232 | 4-N-cyclo- | phenyl | —(CH₂)₂— | py- | 1 | 5-methyl | 6-imidazol-1-yl | | 4,5-dichloro |
| | hexyl-N-methylamino | | | ridin-3-yl | | | | | |
| 233 | 5-trifluoromethyl | py- | —(CH₂)₂— | phenyl | 1 | 3-trifluoro- | 4-imidazol-1-yl | 1 | 4-methyl |
| | | ridin-2-yl | | | | methyl | | | |
| 234 | 4-chloro | phenyl | —(CH₂)₂— | py- | 1 | 5-methyl | 6-imidazol-1-yl | 2 | 4,5-dichloro |
| | | | | ridin-3-yl | | | | | |
| 235 | 5-trifluoromethyl | py- | trans-CH═CH— | phenyl | 1 | 3-trifluoro- | 4-imidazol-1-yl | 2 | 4,5-dichloro |
| | | ridin-2-yl | | | | methyl | | | |
| 236 | 5-trifluoromethylsulfonyl | phenyl | —(CH₂)₂— | phenyl | 1 | 3-trifluoro- | 4-imidazol-1-yl | 2 | 4,5-dichloro |
| | | | | | | methyl | | | |
| 237 | 5-trifluoromethylsulfonyl | phenyl | —(CH₂)₂— | py- | 1 | 5-methyl | 6-imidazol-1-yl | 2 | 4,5-dichloro |
| | | | | ridin-3-yl | | | | | |
| 238 | 4-trifluoromethyl | phenyl | —(CH₂)₂— | phenyl | 1 | 3-trifluoro- | 4-imidazol-1-yl | 1 | 4-trifluoromethyl |
| | | | | | | methyl | | | |
| 239 | 4-trifluoromethylthio | phenyl | —(CH₂)₂— | phenyl | 1 | 3-trifluoro- | 4-imidazol-1-yl | 2 | 4,5-dichloro |
| | | | | | | methyl | | | |
| 240 | 4-trifluoromethyl | phenyl | —(CH₂)₂— | py- | 1 | 5-trifluoro- | 6-imidazol-1-yl | 0 | absent |
| | | | | ridin-3-yl | | methyl | | | |
| 241 | 4-N-cyclo- | phenyl | trans-CH═CH— | py- | 1 | 5-methyl | 6-imidazol-1-yl | 2 | 4,5-dichloro |
| | hexyl-N-methylamino | | | ridin-3-yl | | | | | |
| 242 | 4-chloro | phenyl | —(CH₂)₂— | phenyl | 1 | 3-trifluoro- | 4-imidazol-1-yl | 0 | absent |
| | | | | | | methyl | | | |
| 243 | 4-fluoro | phenyl | —(CH₂)₂— | phenyl | 1 | 3-trifluoro- | 4-imidazol-1-yl | 2 | 4,5-dichloro |
| | | | | | | methyl | | | |
| 244 | 4-trifluoromethylsulfinyl | phenyl | —(CH₂)₂— | phenyl | 1 | 3-trifluoro- | 4-imidazol-1-yl | 2 | 4,5-dichloro |
| | | | | | | methyl | | | |
| 245 | 4-trifluoromethyl | phenyl | —(CH₂)₂— | phenyl | 1 | 3-trifluoro- | 4-(2-oxo-2,3-di- | 0 | absent |
| | | | | | | methyl | hydro-2λ4-[1,2,3,5]ox- | | |
| | | | | | | | athiadizol-4-yl) | | |
| 246 | 4-t-butyl | phenyl | —(CH₂)₂— | py- | 1 | 5-methyl | 6-phenyl | 1 | 3-cyano |
| | | | | ridin-3-yl | | | | | |
| 247 | 4-trifluoromethyl | phenyl | —(CH₂)₂— | py- | 1 | 5-methyl | 6-imidazol-1-yl | 0 | absent |
| | | | | ridin-3-yl | | | | | |
| 248 | 5-trifluoromethyl | py- | —(CH₂)₂— | phenyl | 1 | 3-trifluoro- | 4-imidazol-1-yl | 2 | 4,5-dichloro |
| | | ridin-2-yl | | | | methyl | | | |
| 249 | 4-trifluoromethyl | phenyl | —(CH₂)₂— | py- | 1 | 5-trifluoro- | 6-pyridin-3-yl | 0 | absent |
| | | | | ridin-3-yl | | methyl | | | |
| 250 | 4-trifluoromethyl | phenyl | —(CH₂)₂— | phenyl | 1 | 3-trifluoro- | 4-phenyl | 1 | 4-hydroxymethyl |
| | | | | | | methyl | | | |
| 251 | 4-trifluoromethyl | phenyl | —(CH₂)₂— | py- | 1 | 5-trifluoro- | 6-imidazol-1-yl | 2 | 4,5-dichloro |
| | | | | ridin-3-yl | | methyl | | | |
| 252 | absent | phenyl | —(CH₂)₂— | phenyl | 1 | 3-trifluoro- | 4-pyridin-4-yl | 0 | absent |
| | | | | | | methyl | | | |
| 253 | 4-trifluoromethyl | phenyl | —(CH₂)₂— | phenyl | 1 | 3-trifluoro- | 4-benzoimidazol-1-yl | 0 | absent |
| | | | | | | methyl | | | |
| 254 | 4-trifluoromethyl | phenyl | —(CH₂)₂— | phenyl | 1 | 3-trifluoro- | 4-pyridin-4-yl | 1 | 2-fluoro |
| | | | | | | methyl | | | |
| 255 | 4-trifluoromethyl | phenyl | —(CH₂)₂— | py- | 1 | 5-trifluoro- | 6-pyridin-4-yl | 0 | absent |
| | | | | ridin-3-yl | | methyl | | | |
| 256 | 3,4-difluoro | phenyl | —(CH₂)₂— | phenyl | 1 | 3-trifluoro- | 4-imidazol-1-yl | 0 | absent |
| | | | | | | methyl | | | |
| 257 | 3,4-difluoro | phenyl | —(CH₂)₂— | py- | 1 | 5-methyl | 6-imidazol-1-yl | 2 | 4,5-dichloro |
| | | | | ridin-3-yl | | | | | |
| 258 | 4-trifluoromethyl | phenyl | —(CH₂)₂— | phenyl | 1 | 3-trifluoro- | 4-pyridin-3-yl | 1 | 2-fluoro |
| | | | | | | methyl | | | |
| 259 | 4-t-butyl | phenyl | —(CH₂)₂— | py- | 1 | 5-methyl | 6-phenyl | 1 | 3-methoxy |
| | | | | ridin-3-yl | | | | | |
| 260 | 4-t-butyl | phenyl | —(CH₂)₂— | py- | 1 | 5-methyl | 6-phenyl | 1 | 3-nitro |
| | | | | ridin-3-yl | | | | | |
| 261 | 4-t-butyl | phenyl | —(CH₂)₂— | phenyl | 0 | absent | 4-imidazol-1-yl | | absent |
| 262 | 4-trifluoromethyl | phenyl | —(CH₂)₂— | phenyl | 0 | absent | 4-imidazol-1-yl | 0 | absent |
| 263 | 4-trifluoromethyl | phenyl | —(CH₂)₂— | phenyl | 0 | absent | 4-imidazol-1-yl | 2 | 4,5-dichloro |
| 264 | 4-t-butyl | phenyl | —(CH₂)₂— | phenyl | 1 | 3-trifluoro- | 4-imidazol-1-yl | 0 | absent |
| | | | | | | methyl | | | |
| 265 | 4-trifluoromethyl | phenyl | —(CH₂)₂— | phenyl | 1 | 3-trifluoro- | 4-([1,2,4]oxa- | 1 | 5-trifluoromethyl |
| | | | | | | methyl | diazol-3-yl) | | |
| 266 | 4-trifluoromethyl | phenyl | —(CH₂)₂— | phenyl | 1 | 3-trifluoro- | 4-([1,2,4]oxa- | 1 | 5-methyl |
| | | | | | | methyl | diazol-3-yl) | | |
| 267 | 4-trifluoromethyl | phenyl | —(CH₂)₂— | phenyl | 1 | 3-trifluoro- | 4-imidazol-1-yl | 1 | 2-methyl |
| | | | | | | methyl | | | |
| 268 | 4-trifluoromethyl | phenyl | —(CH₂)₂— | phenyl | 1 | 3-trifluoro- | 4-imidazol-1-yl | 1 | 2-isopropyl |
| | | | | | | methyl | | | |
| 269 | 4-trifluoromethyl | phenyl | —(CH₂)₂— | phenyl | 1 | 3-trifluoro- | 4-indol-1-yl | 0 | absent |
| | | | | | | methyl | | | |
| 270 | 4-trifluoromethyl | phenyl | —(CH₂)₂— | phenyl | 1 | 3-trifluoro- | 4-(4,5,6,7-tetrahydro- | 0 | absent |
| | | | | | | methyl | benzoimidazol-1-yl) | | |
| 271 | 4-trifluoromethyl | phenyl | —(CH₂)₂— | phenyl | 1 | 3-trifluoro- | 4-imidazol-1-yl | 1 | 4-methoxycarbonyl |
| | | | | | | methyl | | | |
| 272 | 4-trifluoromethylthio | phenyl | —(CH₂)₂— | py- | 1 | 5-methyl | 6-imidazol-1-yl | 2 | 4,5-dichloro |
| | | | | ridin-3-yl | | | | | |
| 273 | 4-trifluoromethylsulfinyl | phenyl | —(CH₂)₂— | py- | 1 | 5-methyl | 6-imidazol-1-yl | 2 | 4,5-dichloro |
| | | | | ridin-3-yl | | | | | |
| 274 | 4-trifluoromethylsulfinyl | phenyl | trans-CH═CH— | py- | 1 | 5-methyl | 6-imidazol-1-yl | 2 | 4,5-dichloro |
| | | | | ridin-3-yl | | | | | |
| 275 | 4-trifluoromethylthio | phenyl | trans-CH═CH— | py- | 1 | 5-methyl | 6-imidazol-1-yl | 2 | 4,5-dichloro |
| | | | | ridin-3-yl | | | | | |
| 276 | 5-trifluoromethyl | py- | trans-CH═CH— | py- | 1 | 5-methyl | 6-imidazol-1-yl | 2 | 4,5-dichloro |
| | | ridin-2-yl | | ridin-3-yl | | | | | |
| 277 | 5-trifluoromethyl | py- | trans-CH═CH— | phenyl | 1 | 3-trifluoro- | 4-imidazol-1-yl | 1 | 4-methyl |
| | | ridin-2-yl | | | | methyl | | | |
| 278 | 5-trifluoromethyl | py- | —(CH₂)₂— | py- | 1 | 5-methyl | 6-imidazol-1-yl | 2 | 4,5-dichloro |
| | | ridin-2-yl | | ridin-3-yl | | | | | |
| 279 | 4-fluoro | phenyl | —(CH₂)₂— | phenyl | 1 | 3-trifluoro- | 4-imidazol-1-yl | 0 | absent |
| | | | | | | methyl | | | |
| 280 | 4-fluoro | phenyl | —(CH₂)₂— | py- | 1 | 5-methyl | 6-imidazol-1-yl | 2 | 4,5-dichloro |
| | | | | ridin-3-yl | | | | | |

### BIOLOGICAL EXAMPLES

### EXAMPLE 1

### Human VR1 binding Assay

Compounds of the present invention were tested for their ability to inhibit the binding of tritiated resiniferatoxin ([³H] RTX) to human VR1 receptors in a [³H] RTX binding assay, as previously described (Zhang, Sui-Po. Improved ligand binding assays for vanilloid receptors. PCT Int. Appl. (2002), WO 0233411 A1 20020425 AN 2002:315209; Grant, Elfrida R., Dubin, Adrienne E., Zhang, Sui-Po, Zivin, Robert A., Zhong, Zhong Simultaneous intracellular calcium and sodium flux imaging in human VR1-transfected human embryonic kidney cells: a method to resolve ionic dependence of VR1-mediated cell death. J. Pharmacol. Exp. ther., 2002, 300(1), 9-17.) HEK293 cells were transfected with human VR1 and washed with Hank's balanced Salt Solution, dissociated with cell dissociation buffer (Sigma), and then centrifuged at 1000× g for 5 min. Cell pellets were homogenized in cold 20 mM HEPES buffer (pH 7.4), containing 5.8 mM NaCl, 320 mM sucrose, 2 mM MgCl₂, 0.75 CaCl₂ and 5 mM KCl and centrifuged at 1000× g for 15 min. The resultant supernate was then centrifuged at 40000× g for 15 min. The pelleted membranes were stored in a freezer at −80 C.

Approximately 120 μg protein/ml from membranes were incubated with indicated concentrations of [³H]RTX in 0.5 ml of the HEPES buffer (pH 7.4) containing 0.25 mg/mL fatty acid-free bovine serum albumin at 37 C for 60 min. After cooling the reaction mixture to 4 C, 0.1 mg (x-acid glycoprotein was added to each sample, which was incubated at 4 C for 15 min and then centrifuged at 18500× g for 15 min. The tip of the microcentrifuge tube containing the pellet was cut off. Bound radioactivity was quantified by scintillation counting. Non-specific binding was assessed in the presence of 200 nM unlabeled resiniferatoxin.

Alternatively, a binding assay using rat tissue was used. Rat spinal cord was homogenized twice with a Polytron and centrifuged at 3000 rpm for 10 min in 20 mM HEPES buffer (pH =7.4), containing 5.8 mM NaCl, 320 mM sucrose, 2 mM MgCl₂, 0.75 mM CaCl₂ and 5 mM KCl. The-supernatant was then centrifuged at 40000× g for 15 min. The pellet was saved in a tube to which 10 ml assay buffer was added. The pellet and buffer were mixed with a Polytron. The assay contained 120 μg/ml membrane protein and 0.3-0.6 nM [³H]-RTX (PerkinElmer, Boston) in a total volume of 0.5 ml HEPES buffer. Following incubation for 60 min at 37 C, the samples were cooled on ice, and 0.1 mg of α₁-acid glycoprotein were added into the samples. After centrifugation at 18500× g for 15 min, the supernatant was aspirated, and the tips of tubes were cut off and placed into 6 ml vials.

Data were calculated according to the equation:
% inhibtion=(total binding−binding)*100/(total binding−non specific binding).

Kᵢ values were calculated based on an average of duplicate measurements using a GraphPad Prism program.

The resultant binding data, as well as mass spectal data, are shown below.

| | | | MS | |
|---|---|---|---|---|
| | h | | Parent | |
| | Binding | h Ki | Peak | MS |
| Cpd No. | % I (1 μM) | (nM) | Obs'd | Calc'd |
| 1 | 1 | | 374.00 | 373.50 |
| 2 | 11 | | 374.00 | 373.50 |
| 3 | 90 | 51 | 373.90 | 373.50 |
| 4 | 1 | | 373.70 | 373.50 |
| 5 | 4 | | 373.80 | 373.50 |
| 6 | 67 | 86 | 374.30 | 373.50 |
| 8 | | | 374.00 | 373.50 |
| 9 | 1 | | 373.80 | 373.50 |
| 10 | 89 | 50 | 373.60 | 373.50 |
| 11 | 84 | 145 | 403.00 | 402.49 |
| 12 | 36 | | 426.10 | 425.49 |
| 13 | 35 | | 400.10 | 399.53 |
| 14 | 55 | 4620 | 392.00 | 391.94 |
| 15 | 41 | | 376.10 | 375.49 |
| 16 | 68 | 169 | 415.00 | 414.55 |
| 17 | 90 | 48 | 373.20 | 372.51 |
| 19 | 1 | | 388.10 | 387.52 |
| 20 | 1 | | 387.80 | 387.52 |
| 21 | 1 | | 387.90 | 387.52 |
| 22 | 1 | | 388.10 | 387.52 |
| 23 | 40 | | 388.20 | 387.52 |
| 24 | 87 | 84 | 388.00 | 387.52 |
| 25 | 94 | 103 | 383.10 | 382.51 |
| 26 | 10 | | 442.10 | 441.49 |
| 27 | 53 | 846 | 388.10 | 387.52 |
| 28 | 48 | | 372.10 | 371.52 |
| 29 | 43 | | 416.20 | 415.53 |
| 30 | 2 | | 400.0 | 401.51 |
| | | | (negative | |
| | | | mode) | |
| 31 | 4 | | 401.10 | 400.52 |
| 32 | 4 | | 401.00 | 400.52 |
| 33 | 82 | 171 | 398.80 | 398.51 |
| 34 | 17 | | 427.40 | 426.56 |
| 35 | 30 | | 398.80 | 398.51 |
| 36 | 16 | | 427.10 | 426.56 |
| 37 | 21 | | 401.10 | 400.52 |
| 38 | 1 | | 429.40 | 428.58 |
| 39 | 5 | | 429.20 | 428.58 |
| 40 | 41 | | 451.10 | 450.60 |
| 41 | 78.83 | 108.62 | 473.20 | 472.63 |
| 42 | 1.1 | | 431.1(M − t- | |
| | | | Bu + H) | 486.66 |
| 43 | 23 | | 387.30 | 386.54 |
| 44 | 97 | 52 | 416.20 | 415.54 |
| 45 | 16 | | 417.20 | 416.56 |
| 46 | 34 | | 417.00 | 416.56 |
| 47 | 1 | | 411.20 | 410.39 |
| 48 | 2.1 | | 412.80 | 412.41 |
| 50 | 11 | | 414.90 | 414.55 |
| 51 | 19 | | 414.90 | 414.55 |
| 52 | 1 | | 419.00 | 418.51 |
| 53 | 27 | | 419.00 | 418.51 |
| 54 | 5 | | 429.00 | 428.58 |
| 55 | 66 | 184 | 429.00 | 428.58 |
| 56 | 16 | | 432.90 | 432.54 |
| 57 | 66 | 266 | 432.90 | 432.54 |
| 58 | 43 | | 387.90 | 387.52 |
| 59 | 103 | 10.7 | 387.90 | 387.52 |
| 60 | 52 | 541 | 392.00 | 391.49 |
| 61 | 96 | 53.3 | 392.00 | 391.49 |
| 62 | 23 | | n/a | 373.50 |
| 63 | 1 | | 406.80 | 406.55 |
| 64 | 33 | | 420.90 | 420.57 |
| 65 | 77 | 168 | 379.80 | 379.52 |
| 66 | 1 | | 444.80 | 444.53 |
| 67 | 1 | | 458.90 | 458.56 |
| 68 | 1 | | 417.90 | 417.50 |
| 69 | 1 | | 430.80 | 430.55 |
| 70 | 1 | | 444.90 | 444.57 |
| 71 | 1 | | 404.00 | 403.52 |
| 72 | 1 | | 444.90 | 444.53 |
| 73 | 1 | | 458.90 | 458.56 |
| 74 | 1 | | 417.90 | 417.50 |
| 75 | 6 | | 426.70 | 426.44 |
| 76 | 50 | 421 | 385.80 | 385.38 |
| 77 | 72 | 134 | 385.80 | 385.38 |
| 78 | 56 | 289 | 385.80 | 385.38 |
| 79 | 19 | | 399.70 | 399.41 |
| 80 | 62 | 269 | 399.70 | 399.41 |
| 81 | 6 | | 465.30 | 464.61 |
| 82 | 28 | | 424.30 | 423.56 |
| 83 | 1 | | 463.20 | 462.47 |
| 84 | 1 | | 477.20 | 476.50 |
| 85 | 1 | | 436.20 | 435.44 |
| 86 | | | 402.30 | 401.55 |
| 87 | | | 402.30 | 401.55 |
| 88 | | | 416.20 | 415.58 |
| 89 | | | 410.20 | 409.48 |
| 90 | | | 410.20 | 409.48 |
| 91 | | | 424.20 | 423.50 |
| 92 | | 89 | 404.20 | 403.52 |
| 93 | | 34 | 404.20 | 403.52 |
| 94 | | 42 | 404.20 | 403.52 |
| 95 | | | 418.20 | 417.55 |
| 96 | | 77 | 418.20 | 417.55 |
| 97 | | 15 | 388.20 | 387.52 |
| 98 | | 58 | 388.20 | 387.52 |
| 99 | | 29 | 402.20 | 401.55 |
| 100 | | 68 | 392.10 | 391.49 |
| 101 | | 50 | 392.10 | 391.49 |
| 102 | | 39 | 406.10 | 405.51 |
| 103 | 1 | | 358.90 | 358.48 |
| 104 | 73 | 180 | 358.80 | 358.48 |
| 105 | 1 | | 359.10 | 358.48 |
| 106 | 1 | | 359.00 | 358.48 |
| 107 | 82 | 120 | 359.00 | 358.48 |
| 108 | 16 | | 359.20 | 358.48 |
| 109 | 1 | | 359.30 | 358.48 |
| 110 | 97 | 11.3 | 359.10 | 358.48 |
| 111 | 61 | 168 | 359.20 | 358.48 |
| 112 | 68 | 216 | 360.00 | 359.47 |
| 113 | 67 | 324 | 359.80 | 359.47 |
| 114 | | | 375.50 | 374.48 |
| 115 | 34 | | 375.40 | 374.48 |
| 116 | 1.1 | | 360.10 | 359.47 |
| 117 | 1 | | 360.10 | 359.47 |
| 118 | | | 389.20 | 388.51 |
| 119 | 1 | | 389.20 | 388.51 |
| 120 | | | 389.10 | 388.51 |
| 121 | 36 | | 389.20 | 388.51 |
| 122 | 64 | 335 | 397.00 | 396.53 |
| 123 | 39 | | 397.00 | 396.53 |
| 124 | 1 | | 402.30 | 401.51 |
| 125 | 1 | | 416.30 | 415.54 |
| 126 | 9 | | 375.30 | 374.48 |
| 127 | 16 | | 402.30 | 401.51 |
| 128 | 60 | 356 | 416.40 | 415.54 |
| 129 | 88 | 41 | 375.30 | 374.48 |
| 130 | 1 | | 403.20 | 402.50 |
| 131 | 1 | | 403.30 | 402.50 |
| 132 | 45 | | 374.90 | 374.48 |
| 133 | 101 | 10.9 | 374.90 | 374.48 |
| 134 | 25 | | 389.00 | 388.51 |
| 135 | 90 | 31.2 | 389.00 | 388.51 |
| 136 | 84 | 103 | 359.90 | 359.47 |
| 137 | 66 | 93 | 359.90 | 359.47 |
| 138 | 61 | 461 | 364.80 | 364.51 |
| 139 | 1 | | 402.90 | 402.49 |
| 140 | 1 | | 389.00 | 388.51 |
| 141 | 1 | | 402.90 | 402.49 |
| 142 | 45 | | 370.80 | 370.37 |
| 143 | 44 | | 370.80 | 370.37 |
| 144 | 1 | | 413.70 | 413.40 |
| 145 | 16 | | 427.80 | 427.43 |
| 146 | 8 | | 386.90 | 386.37 |
| 147 | 62 | 319 | 386.90 | 386.37 |
| 148 | 73 | 98.6 | 386.90 | 386.37 |
| 149 | 9 | | 400.80 | 400.40 |
| 150 | 75 | 149 | 400.80 | 400.40 |
| 151 | 35 | | 371.90 | 371.36 |
| 152 | 37 | | 371.90 | 371.36 |
| 153 | 1 | | 409.30 | 408.54 |
| 154 | 15 | | 466.30 | 465.60 |
| 155 | 89 | | 425.20 | 424.54 |
| 156 | 33 | | 410.20 | 409.53 |
| 157 | 1 | | 421.20 | 420.43 |
| 158 | 77 | | 437.20 | 436.43 |
| 159 | 1 | | 422.20 | 421.42 |
| 160 | 101 | | 389.20 | 388.51 |
| 161 | | | 389.20 | 388.51 |
| 162 | | | 389.20 | 388.51 |
| 163 | | | 403.20 | 402.54 |
| 164 | | | 374.20 | 373.50 |
| 165 | | | 374.20 | 373.50 |
| 166 | | | 387.30 | 386.54 |
| 167 | | | 387.30 | 386.54 |
| 168 | | | 395.20 | 394.46 |
| 169 | | 48 | 395.20 | 394.46 |
| 170 | | | 389.20 | 388.51 |
| 171 | | 292 | 389.20 | 388.51 |
| 172 | | | 373.20 | 372.51 |
| 173 | | | 377.10 | 376.47 |
| 174 | | | 387.20 | 386.54 |
| 175 | | 142 | 395.10 | 394.46 |
| 176 | | 66 | 360.10 | 359.47 |
| 177 | | | 374.20 | 373.50 |
| 178 | | | 361.10 | 360.46 |
| 179 | | 67 | 373.20 | 372.51 |
| 180 | | 42 | 373.20 | 372.51 |
| 181 | | | 377.10 | 376.47 |
| 182 | | 55 | 377.10 | 376.47 |
| 183 | | | 376.10 | 375.47 |
| 184 | | | 376.10 | 375.47 |
| 185 | | | 376.10 | 375.47 |
| 186 | | | 390.10 | 389.50 |
| 187 | | | 361.10 | 360.46 |
| 188 | | | 361.10 | 360.46 |
| 189 | 4 | | 362.10 | 361.489 |
| 190 | 56 | | 362.10 | 361.489 |
| 191 | 19 | | 362.10 | 361.489 |
| 192 | 50 | | 348.10 | 347.462 |
| 193 | 49 | | 349.10 | 348.446 |
| 194 | 45 | | 351.10 | 350.462 |
| 195 | 75 | | 379.10 | 378.538 |
| 196 | 76 | | 376.10 | 375.516 |
| 197 | 62 | | 366.10 | 365.499 |
| 198 | 37 | | 349.10 | 348.45 |
| 199 | 3 | | 350.10 | 349.438 |
| 200 | 100 | | 416.00 | 416.352 |
| 201 | 1 | | 378.10 | 377.488 |
| 202 | | 212 | 417.2 | 416.45 |
| 203 | | 34.0 | 443.0 | 443.26 |
| 204 | | 31.9 | 442.2 | 441.38 |
| 205 | | 44.4 | 504.0 | 503.43 |
| 206 | | 64.6 | 454.20 | 453.39 |
| 207 | | 47.8 | 428.1 | 427.35 |
| 208 | | 88.7 | 472.0 | 471.43 |
| 209 | | | 505.8 | 505.44 |
| 210 | | 54.5 | 455.1 | 454.38 |
| 211 | | 22.2 | 416.1 | 416.35 |
| 212 | | 5.9 | 484.1 | 484.35 |
| 213 | | | 455.1 | 454.38 |
| 214 | | 536 | 363.3 | 362.48 |
| 215 | | 7.4 | 431.1 | 431.37 |
| 216 | | 70.4 | 430.2 | 429.57 |
| 217 | | 47.7 | 557.9 | 558.29 |
| 218 | | 19.9 | 496.1 | 496.24 |
| 219 | | | 473.7 | 473.44 |
| 220 | | 99.8 | 438.90 | 438.38 |
| 221 | | 57.8 | 455.1 | 454.38 |
| 222 | | | 489.7 | 489.44 |
| 223 | | 82.6 | 467.10 | 467.41 |
| 224 | | 18.4 | 485.1 | 485.34 |
| 225 | | 161.8 | 469.1 | 468.40 |
| 226 | | 761 | 389.2 | 388.40 |
| 227 | | 90.8 | 462.1 | 461.80 |
| 228 | | 41.6 | 526.0 | 526.29 |
| 229 | | 99.2 | 454.20 | 453.39 |
| 230 | | 1020 | 405.10 | 404.82 |
| 231 | | 26.2 | 461.70 | 462.69 |
| 232 | | 80.0 | 486.3 | 486.45 |
| 233 | | 1000 | 443.0 | 442.37 |
| 234 | | 85.5 | 408.80 | 409.71 |
| 235 | | | 494.9 | 495.21 |
| 236 | | | 559.7 | 560.31 |
| 237 | | | 506.7 | 507.32 |
| 238 | | | 496.2 | 495.35 |
| 239 | | | 527.7 | 528.31 |
| 240 | | 3852 | 429.1 | 428.34 |
| 241 | | 79.8 | 484.3 | 484.43 |
| 242 | | 2110 | 393.80 | 393.8 |
| 243 | | | 445.80 | 446.23 |
| 244 | | | 543.6 | 544.31 |
| 245 | | 974 | 466.0 | 465.38 |
| 246 | | 370.6 | 398.2 | 397.52 |
| 247 | | 0 | 375.1 | 374.37 |
| 248 | | | 496.9 | 497.23 |
| 249 | | | 440.1 | 439.36 |
| 250 | | 199 | 468.10 | 467.41 |
| 251 | | 134.5 | 497.1 | 497.23 |
| 252 | | 5350 | 371.10 | 370.38 |
| 253 | | 44.3 | 478.2 | 477.41 |
| 254 | | 293 | 457.20 | 456.37 |
| 255 | | 329.2 | 440.1 | 439.36 |
| 256 | | | 395.90 | 395.33 |
| 257 | | | 410.70 | 411.24 |
| 258 | | 500 | 457.20 | 456.37 |
| 259 | | | 403.2 | 402.54 |
| 260 | | 391 | 418.2 | 417.51 |
| 261 | | 311 | 348.3 | 347.46 |
| 262 | | | 360.1 | 359.35 |
| 263 | | 84.4 | 428.1 | 428.24 |
| 264 | | | 416.2 | 415.46 |
| 265 | | | 497.7 | 497.32 |
| 266 | | | 443.9 | 443.35 |
| 267 | | | 442.2 | 441.38 |
| 268 | | | 470.2 | 469.43 |
| 269 | | | 477.2 | 476.43 |
| 270 | | | 482.2 | 481.44 |
| 271 | | | 486.2 | 485.39 |
| 272 | | | 474.8 | 475.32 |
| 273 | | | 490.6 | 491.32 |
| 274 | | | 489.0 | 489.31 |
| 275 | | | 472.9 | 473.31 |
| 276 | | | 441.9 | 442.23 |
| 277 | | 140 | 441.0 | 440.35 |
| 278 | | | 443.9 | 444.25 |
| 279 | | | 377.90 | 377.34 |
| 280 | | | 392.90 | 393.25 |

### EXAMPLE 2

### Human VR 1 Functional Assay

The functional activity of the test compounds was determined by measuring changes in intracellular calcium concentration using a Ca²⁺-sensitive fluorescent dye and FLIPR™ technology. Increases in Ca²⁺ concentration were readily detected upon challenge with capsaicin.

HEK293 Cells expressing human VR1 were grown on poly-D-lysine coated 384 well black-walled plates (BD 354663) and 1 day later loaded with Calcium 3 Dye for 35 min at 37 C, 5% CO₂ and then for 25 min at room temperature, and subsequently tested for agonist-induced increases in intracellular Ca²⁺ levels using FLIPR™ technology. Cells were challenged with test compounds (at varying concentrations, which are indicated in parentheses in the heading of the table below) and intracellular Ca²⁺ was measured for 5 min prior to the addition of capsaicin to all wells to achieve a final concentration of eliciting an approximate 80% maximal response (0.020-0.030 JM). EC₅₀ or IC₅₀ values were determined from dose-response studies. Curves were generated using the average of quadruplicate wells for each data point.

VR1 in vitro Functional Data

| | | | | | % Response | % Response | |
|---|---|---|---|---|---|---|---|
| | | | | | Relative to | Relative to | |
| | | | | | EC80 of | EC80 of | |
| | h % I | h % I | h % I | h IC50 | Capsaicin | Capsaicin | h EC50 |
| Cpd No. | (10 μM) | (5 μM) | (1 μM) | (nM) | (12 μM) | (6 μM) | (nM) |
| 1 | 45 | | | | | | |
| 2 | 41 | | | | | | |
| 3 | 44 | | | | 65 | | |
| 4 | 35 | | | | | | |
| 5 | 35 | | | | | | |
| 6 | | | | | 24 | | |
| 8 | 45 | | | | | | |
| 9 | 16 | | | | | | |
| 10 | 40 | | | | 67 | | |
| 11 | 36, 20 | | | | | | |
| 12 | | | | | 47 | | |
| 13 | 35 | | | | | | |
| 14 | | | | | 53 | | |
| 15 | | | | | 80 | | |
| 16 | | | | | 101 | 63 | |
| 17 | | | | | 67 | | |
| 19 | 20 | | | | | | |
| 20 | 70 | | | 2081 | | | |
| 21 | | | | | 45 | | |
| 22 | 1 | | | | | | |
| 23 | ND | | | | | | |
| 24 | 32 | | | | 68 | | |
| 25 | 12, 43 | | | | | | |
| 26 | | | | | 65 | | |
| 27 | 28, 1 | | | | | | |
| 28 | | | | | 81 | | |
| 29 | 36 | | | | | | |
| 30 | | | | | 94 | | 1423 |
| 31 | 28, 47 | | | | | | |
| 33 | 27, 54 | | | | | | |
| 34 | 14 | | | | | | |
| 35 | | | | | 53 | | |
| 36 | | | | | 55 | | |
| 37 | 16 | | | | | | |
| 38 | 17 | | | | | | |
| 39 | 24 | | | | | | |
| 40 | | | | | 74 | | |
| 41 | 30 | | | | 43 | | |
| 42 | 9 | | | | | | |
| 43 | | | | | 104 | | 1663 |
| 44 | | | | | 102 | | 289 |
| 45 | | | | | 84 | | 2396 |
| 46 | 53 | | | 4774 | | | |
| 47 | | | | | | | |
| 48 | | | | | 57 | | |
| 50 | | | | | | | |
| 51 | | | | | | | |
| 52 | | | | | | | |
| 53 | | | | | | | |
| 54 | | | | | | | |
| 55 | | | | | 69 | | |
| 56 | | | | | | | |
| 57 | | | | | 84 | | |
| 58 | | | | | | 19 | |
| 59 | 101 | | | 490 | | | |
| 60 | | | | | 84 | | |
| 61 | | | | | 68 | | |
| 62 | | 16 | | | | | |
| 63 | | | | | | | |
| 64 | | | | | | | |
| 65 | | | | | 87 | | |
| 66 | | | | | | | |
| 67 | | | | | | | |
| 68 | | | | | | | |
| 69 | 6 | | | | | | |
| 70 | 7 | | | | | | |
| 71 | 5 | | | | | | |
| 72 | 11 | | | | | | |
| 73 | 20 | | | | | | |
| 74 | 8 | | | | | | |
| 75 | | | | | 82 | | |
| 76 | | | | | 77 | | |
| 77 | | | | | 88 | | |
| 78 | | | | | 70 | | |
| 79 | | | | | 74 | | |
| 80 | | | | | 77 | | |
| 81 | | 11 | | | | | |
| 82 | | 39 | | | | | |
| 83 | | 31 | | | | | |
| 84 | | 43 | | | | | |
| 85 | | 21 | | | | | |
| 86 | | 97 | | 109 | | | |
| 87 | | 92 | | 857 | | | |
| 88 | | 84 | | 1707 | | | |
| 89 | | 65 | | | | | |
| 90 | | | | | | 17 | |
| 91 | | | | | | 22 | |
| 92 | | | 84 | | | 133 | 111 |
| 93 | | | 93 | | | 153 | 65 |
| 94 | | | 92 | | | 115 | 94 |
| 95 | | | 40 | | | 127 | 248 |
| 96 | | | 91 | | | 65 | |
| 97 | | 104 | 95 | 228 | | | |
| 98 | | 102 | 97 | 418 | | | |
| 99 | | 79 | 97 | 209 | | | |
| 100 | | | 87 | | | 96 | |
| 101 | | | 95 | | | 71 | |
| 102 | | | 97 | | | 74 | |
| 103 | | | | 3520 | | | |
| 104 | | | | | 62 | | |
| 105 | | | | 3950 | | | |
| 106 | | | | | | | |
| 107 | | | | | 78 | | |
| 108 | | | | | 98 | | 935 |
| 109 | | | | | 47 | | |
| 110 | | | | | | | |
| 111 | | | | | 119 | | 18 |
| 112 | | | | | | | |
| 113 | | | | | 101 | | 773 |
| 114 | | | | | | | |
| 115 | | | | | 58 | | |
| 116 | | | | 377 | | | |
| 117 | | | | | | | |
| 118 | | | | | | | |
| 119 | | | | | | | |
| 120 | | | | | | | |
| 121 | | | | | | | |
| 122 | | | | | | | |
| 123 | | | | | | | |
| 124 | | | | | | | |
| 125 | | | | | | | |
| 126 | | | | | | | |
| 127 | | | | | | | |
| 128 | | | | | 125 | | 6 |
| 129 | | | | | 126 | | 17 |
| 130 | | | | | | | |
| 131 | | | | | | | |
| 132 | | 30 | | | | | |
| 133 | | | | | 95 | | |
| 134 | | | | | | | |
| 135 | | | | | 79 | | |
| 136 | | | | | 78 | | |
| 137 | | | | | 96 | | |
| 138 | | | | | 113 | | |
| 139 | | | | | | | |
| 140 | | | | | | | |
| 141 | | | | | | | |
| 142 | | | | | 78 | | |
| 143 | | | | | 111 | | |
| 144 | | | | | 73 | | |
| 145 | | | | | 92 | | |
| 146 | | | | | | | |
| 147 | | | | | 92 | | |
| 148 | | | | | 74 | | |
| 149 | | | | | 101 | | |
| 150 | | | | | 79 | | |
| 151 | | | | | 91 | | |
| 152 | | | | | 100 | | |
| 153 | | 25 | | | | | |
| 154 | | | | | | | |
| 155 | | | | | | | |
| 156 | | | | | | | |
| 157 | | 12 | | | | | |
| 158 | | | | | | | |
| 159 | | | | | | | |
| 160 | | 96 | | 27 | | | |
| 161 | | 94 | | 36 | | | |
| 162 | | 99 | | 126 | | | |
| 163 | | 100 | | 97 | | | |
| 164 | | 99 | | 193 | | | |
| 165 | | 99 | | 73 | | | |
| 166 | | 76 | | 2864 | | | |
| 167 | | 93 | | 183 | | | |
| 168 | | 95 | | 1557 | | | |
| 169 | | | 96 | | | 45 | |
| 170 | | | 37 | | | 101 | |
| 171 | | | 60 | | | 119 | |
| 172 | | | | 142 | | | |
| 173 | | | | 388 | | | |
| 174 | | | | 2891 | | | |
| 175 | | | 75 | | | | |
| 176 | | 22 | 99 | | | | |
| 177 | | | | 295.3 | | | |
| 178 | | | | | | | |
| 179 | | 100 | 81 | 266.2 | | | |
| 180 | | | 91 | | | 96 | |
| 181 | | | | 399.5 | | | |
| 182 | | | 80 | | | 97 | |
| 183 | | 9 | 1 | | | | |
| 184 | | 14 | 1 | | | | |
| 185 | | 24 | 1 | | | | |
| 186 | | 39 | 6 | | | | |
| 187 | | 15 | 11 | | | | |
| 188 | | 33 | 4 | | | | |
| 189 | | 63 | | | | | |
| 190 | | 98 | | | | | |
| 191 | | | | 0.972 | | | |
| 192 | | | | | | | |
| 193 | | | | | | | |
| 194 | | 94 | | | | | |
| 195 | | 20 | | | | | |
| 196 | | | | | | | |
| 197 | | | | 0.283 | | | |
| 198 | | | | 3.82 | | | |
| 199 | | 19 | | | | | |
| 200 | | 99 | | | | | |
| 201 | | 57 | | | | | |
| 202 | | 100 | | 30.1 | | | |
| 203 | | 100 | 99 | 35.2 | | | |
| 204 | | | 100 | 37.0 | | | |
| 205 | | | 100 | 38.0 | | | |
| 206 | | 100 | | 39.4 | | | |
| 207 | | 100 | 95 | 40.2 | | | |
| 208 | | | 100 | 41.0 | | | |
| 209 | | | 100 | 48.0 | | | |
| 210 | | 100 | | 57.0 | | | |
| 211 | | 99 | | 60.0 | | | |
| 212 | | 100 | 101 | 62.7 | | | |
| 213 | | 100 | | 64.5 | | | |
| 214 | | 91. | | 73.2 | | | |
| 215 | | 100 | | 74.3 | | | |
| 216 | | 99 | | 76.2 | | | |
| 217 | | | 101 | 87.0 | | | |
| 218 | | 101 | | 97.2 | | | |
| 219 | | | 100 | 100 | | | |
| 220 | | 100 | | 121 | | | |
| 221 | | 100 | | 127 | | | |
| 222 | | | 100 | 137 | | | |
| 223 | | 100 | | 142 | | | |
| 224 | | 101 | | 147 | | | |
| 225 | | 100 | | 152 | | | |
| 226 | | | 83 | 164 | | | |
| 227 | | | 100 | 168 | | | |
| 228 | | | 92 | 171 | | | |
| 229 | | 100 | | 195 | | | |
| 230 | | 101 | | 202 | | | |
| 231 | | | 90 | 240 | | | |
| 232 | | | 99 | 249 | | | |
| 233 | | | 98 | 259 | | | |
| 234 | | | 88 | 290 | | | |
| 235 | | | 99 | 294 | | | |
| 236 | | | 98 | 306 | | | |
| 237 | | | 97 | 325 | | | |
| 238 | | | 100 | 340 | | | |
| 239 | | | 88 | 358 | | | |
| 240 | | 101 | | 379 | | | |
| 241 | | | 92 | 396 | | | |
| 242 | | | 76 | 400 | | | |
| 243 | | | 92 | 440 | | | |
| 244 | | | 85 | 452 | | | |
| 245 | | 9 | 70 | 491 | | | |
| 246 | | 105 | | 543 | | | |
| 247 | | 97 | | 549 | | | |
| 248 | | | 83 | 602 | | | |
| 249 | | 100 | | 613 | | | |
| 250 | | 100 | | 718 | | | |
| 251 | | 101 | | 724 | | | |
| 252 | | 100 | | 896 | | | |
| 253 | | | 52 | 952 | | | |
| 254 | | 98 | | 995 | | | |
| 255 | | 100 | | 1.07 | | | |
| 256 | | | 42 | 1.10 | | | |
| 257 | | | 41 | 1.10 | | | |
| 258 | | 93 | | 1.13 | | | |
| 259 | | 90 | | 1.53 | | | |
| 260 | | 93 | | 2.13 | | | |
| 261 | | | | | | | |
| 262 | | | | | | | |
| 263 | | 67 | | | | | |
| 264 | | 48 | 24 | | | | |
| 265 | | 63 | 18 | | | | |
| 266 | | 64 | 47 | | | | |
| 267 | | | 20 | | | | |
| 268 | | | 15 | | | | |
| 269 | | | 21 | | | | |
| 270 | | | 47 | | | | |
| 271 | | | 26 | | | | |
| 272 | | | 41 | | | | |
| 273 | | | 36 | | | | |
| 274 | | | 18 | | | | |
| 275 | | | 16 | | | | |
| 276 | | | 19 | | | | |
| 277 | | | 38 | | | | |
| 278 | | | 36 | | | | |
| 279 | | | 30 | | | | |
| 280 | | | 16 | | | | |

### EXAMPLE 3

### In Vivo Model for Chronic Inflammation Induced by Complete Freund's Adjuvant (CFA)

Intraplantar injection of Complete Freund's Adjuvant (CFA) in rodents results in a long-lasting inflammatory reaction, characterized by a pronounced hyperalgesia to both thermal and mechanical stimuli. This effect peaks between 24-72 hours following injection and can last for several weeks. To assess the antihyperalgesic potential of test compounds, Sprague-Dawley rats (typically males ranging from 150-350 g) are given a 100 μL intraplantar injection of CFA (suspended in a 1:1 emulsion of saline and heat-killed Mycobacterium. tuberculosis in mineral oil) into a single hind paw.

### EXAMPLE 3a

### CFA-Induced Mechanical Hyperalgesia

Quantification of the nociceptive pressure thresholds in the hind paws is performed using an analgesy-meter (Stoelting, Wood Dale Ill.). The test consists of placing the left hind paw on a Teflon platform and applying a linearly increasing mechanical force on the dorsum of the hind paw, with a dome-tipped plinth. The endpoint is reached upon hind paw withdrawal or vocalization, at which time the terminal force is noted (in grams) and recorded. Following a 24-48 hour CFA incubation period, rats are retested. Only rats that exhibit at least a 25% reduction in response threshold (i.e. hyperalgesia) are included in further analysis. Immediately following the post-CFA threshold assessment, rats are dosed with a test compound or vehicle (usually hydroxypropyl methylcellulose, hydroxypropyl beta-cyclodextrin, or PEG-400). Post-treatment withdrawal thresholds are assessed at fixed time intervals typically 60, 120 and 180 minutes. Paw withdrawal thresholds are expressed as raw data or converted to percentage of baseline according to the following formula:
% Baseline=(Treatment Response)/(pre-CFA Response)×100.
This paradigm may also be conducted with a multiple dosing or prophylactic dosing regime designed to alter the course of hyperalgesia development. The clinically important NSAID diclofenac reversed the hyperalgesic effect of zymosan in this model (Belichard, P. Immunopharmacol. 46:139-147, 2000,). Similarly, the VR1 receptor antagonist BCTC reduced hyperalgesia in this model (Pomonis, J D et al., JPET 306:387-393, 2003), predicting the effectiveness of VR1 antagonists in inflammation induced pain in humans.

### EXAMPLE 3b

### CFA-Induced Paw Radiant Heat Hyperalgesia

Each rat is placed in a test chamber on a warm glass surface and allowed to acclimate for approximately 10 minutes. A radiant thermal stimulus (beam of light) is then focused through the glass onto the sole of each hind paw in turn. The light stimulus is automatically shut off by a photoelectric relay when the foot moves or when the cut-off time is reached (20 seconds for radiant heat at ˜5Amps). An initial (baseline) response time to the thermal stimuli is recorded for each animal prior to the injection of CFA. Twenty-four hours following intraplantar CFA injection, the response latency of the animal to the thermal stimulus is then evaluated and compared to the animal's baseline response time. Only rats that exhibit at least a 25% reduction in response latency (i.e. hyperalgesia) are included in further analysis. Immediately following the post-CFA latency assessment, rats are dosed with test compound or vehicle (usually hydroxypropyl methylcellulose, hydroxypropyl beta-cyclodextrin or PEG-400). Post-treatment withdrawal latencies are assessed at fixed time intervals, typically 60, 120 and 180 minutes. Paw withdrawal latencies are expressed as raw data or converted to percentage of baseline according to the following formula:
% Baseline=(Treatment Response)/(pre-CFA Response)×100.
This paradigm may also be conducted with a multiple dosing or prophylactic dosing regime designed to alter the course of hyperalgesia development.

This test predicts the analgesic effect of numerous effective clinical agents, including acetaminophen, NSAIDS including aspirin and ibuprofen, and opioids such as morphine. Thus the effectiveness of VR1 antagonists in this model (Gomtsyan, A et al., J Med Chem 48:744-752, 2005) is predictive of their human clinical effect.

### EXAMPLE 4

### Radiant Heat Paw Hyperalgesia

In rodents, intraplantar injection of TRPV1 agonists (such as capsaicin) or inflammogens (such as zymosan) results in a robust neuronal sensitization, which can be characterized by a markedly reduced response latency (hyperalgesia) to thermal stimulation. To assess thermal response latencies, rats are placed individually on a warm (˜30° C.) glass surface and allowed to acclimate to the test chamber for approximately 10 minutes. A radiant thermal stimulus (beam of light) is then focused on the sole of each hind paw in turn. The light stimulus is automatically shut off by a photoelectric relay when the foot moves or when the cut-off time is reached (20 seconds for radiant heat at ˜5 Amps). An initial (baseline) response time to the thermal stimuli is recorded for each animal prior to the intraplantar injection. Hyperalgesia is then induced by an intraplantar injection of a sensitizing agent such as 10-20 μL capsaicin (1 mg/mL) or 100-200 μL zymosan (25 mg/mL) into a single hind paw. Fifteen minutes following capsaicin injection or 180 minutes following zymosan following injection, the rats are retested to verify hyperalgesia (>25% reduction in response latency relative to baseline response), after which the test compounds are administered. The rats are then re-tested at regular intervals to elucidate the effect of compound treatment on the course of hyperalgesia. Paw withdrawal latencies are expressed as raw data or converted to percentage of baseline according to the following formula:
% Baseline=(Treatment Response)/(Baseline Response)×100.

The ability of test compounds to mitigate the development of hyperalgesia may also be tested. In such a paradigm animals are treated with test compound prior to intraplantar injection of inflammogen or sensitizing agent followed by additional hourly assessments in order to assess the degree of hyperalgesia. Responses in rats pretreated with test compound prior to intraplantar injection are compared with those treated with vehicle.

Pungent TRPV1 agonists such as capsaicin initially cause neuronal excitation (e.g. thermal hyperalgesia) followed by a long lasting desensitization to thermal stimuli. Test compounds administered by intraplantar injection are assessed using radiant heat response latencies to characterize both initial sensitization (pungency) over minutes and chronic desensitization over days. In the case of capsaicin, intraplantar injection of 20 μL (1 mg/ mL) results in a dramatic thermal hyperalgesia peaking around 10-15 minutes, followed by a marked insensitivity to radiant heat, which lasts for days.

### EXAMPLE 5a

### In Vivo Model for Abdominal Irritant Tests: Graded Abdominal Irritant Test

A chemical irritant (such as acetic acid, kaolin, bradykinin, phenyl-p-(benzo) quinone or zymosan) is injected in mice intraperitoneally at a dose determined from the literature or through routine preliminary testing. Following the administration of the chemical agent, the animals are placed in glass bell jars (approximately 15 cm in diameter). The animals are observed to determine the number of occurrences of a characteristic behavioral response. A contraction of the abdominal musculature and an elongation of the body, which extends through to the hind limbs, characterize this response. The responses are counted during the test period (typically 15-minutes) following injection of the chemical agent. A mechanical counter or a personal computer is used to collect the number of counts per animal. The mean number of counts for a group of animals receiving pretreatment with a known analgesic or test compound is compared to the mean for the group of animals that received only vehicle pretreatment.

These tests not only predict the analgesic effect of numerous effective agents, but the potency of these agents in the abdominal irritant test parallels the magnitude of the dose needed in the relief of clinical pain. Such agents include acetaminophen, NSAIDS including aspirin and ibuprofen, opioids such as morphine and codeine, and other centrally acting analgesics such as tramadol. Thus the effectiveness of VR1 antagonists in this model (Gomtsyan, A et al., J Med Chem 48:744-752, 2005) is predictive of their human clinical effect.

### EXAMPLE 5b

### In Vivo Model for Abdominal Irritant Tests: Pre-Inflamed Graded Abdominal Irritant Test

Agents such as LPS, zymosan, and thioglycolate are known to induce inflammatory responses following intraperitoneal injection. A small intraperitoneal dose of such an inflammogen hours or days before the acute challenge with chemical irritant will increase the number of abdominal contractions observed. This is a form of viscerochemical hyperalgesia. While some test compounds are effective at mitigating acute viscerochemical nociception, others, particularly those dependent upon receptor induction are more effective at preventing or reversing the enhancement of behavioral responses caused by a preconditioning inflammatory stimulus.

These tests not only predict the analgesic effect of numerous effective agents, but the potency of these agents in the abdominal irritant test parallels the magnitude of the dose needed in the relief of clinical pain. Such agents include acetaminophen, NSAIDS including aspirin and ibuprofen, opioids such as morphine and codeine, and other centrally acting analgesics such as tramadol. Thus the effectiveness of VR1 antagonists in this model (Gomtsyan, A et al., J Med Chem 48:744-752, 2005) is predictive of their human clinical effect.

### EXAMPLE 6

### In Vivo Model for Rodent Neuropathic Pain

The sciatic nerve is the major sensorimotor innervation of the (hind) leg and foot. Injury to the sciatic nerve or its constituent spinal nerves often results in pain behaviors. In rats and mice, tight ligation of the L5 spinal nerve with silk suture, partial tight ligation of the sciatic nerve with silk suture, or loose ligation of the sciatic nerve with chromic gut suture all result in behaviors reminiscent of neuropathic pain in humans. These lesions (one per animal) are performed surgically in anesthetized rodents. Both the spinal nerve and sciatic nerve lesions result in allodynia, a painful response to innocuous stimuli, and hyperalgesia, an exaggerated response to normally painful stimuli. It is important to note that both of these pain behaviors are evoked by the testing procedures and that normal use of the paw (walking) is relatively uncompromised apart from occasional “guarding” of the paw. Subsequent to the surgery, the subjects' behaviors, such as grooming, feeding, and weight gain, are normal except for sensitivity of the affected paw.

In addition to induction by nerve damage resulting from accidents or surgical procedures, neuropathic pain can also be induced by diabetes (Fox, A et al., Pain 81:307-316,1999) or by treatment with chemotherapeutic agents such as vincristine (Yaksh, T L et al., Pain 93:69-76, 2001).

Agents that attenuate neuropathic pain in the clinic, also demonstrate effect in rodent neuropathic pain models. These agents include the recently approved Cymbala (Duloxetine, Iyengar, S., et al., JPET 311:576-584, 2004), morphine (Suzuki, R et al., Pain 80:215-228,1999), and gabapentin (Hunter, J C et al., Eur J Pharmacol 324:153-160, 1997). The VR1 receptor antagonist BCTC reduced mechanical hyperalgesia and tactile allodynia in the chronic constriction injury rodent neuropathic pain model (Pomonis, J D et al., JPET 306:387-393, 2003). The effect of VR1 receptor antagonists in this model is predictive of clinical effect for these novel agents.

### EXAMPLE 6a

### Behavioral Testing/Mechanical Allodynia

At various time points, days and weeks following surgery, the subjects are placed in elevated observation chambers having wire mesh floors. Through the mesh floor a series of tactile stimuli are applied to the bottom of the paw using either an electronic force transducing probe or filaments calibrated to bend at specified forces. Mechanical response thresholds are measured by recording the force that provokes an abrupt withdrawal or lifting of the paw. Mechanical response thresholds in unlesioned rats are typically greater than 50 grams of force while lesioned paws exhibiting allodynia may respond to applied mechanical stimuli below 1 gram of force. Test compounds are assessed for their ability to return mechanical thresholds to pre-lesion levels following systemic administration.

### EXAMPLE 6b

### Behavioral Testing/Thermal Hyperalgesia

In order to measure thermal hyperalgesia, a radiant heat device is used. This device consists of Plexiglas chambers with glass bottoms that allow a focused light beam to be shown on the undersurface of each hind paw individually. The intensity of the light beam is adjusted to elicit paw withdrawal latencies in the desired range (typically 10-15 seconds) in normal animals. Paw withdrawal latencies are reduced in the hind paw corresponding to the nerve injury, thus representing thermal hyperalgesia. The radiant heat device automatically shuts the light beam off immediately upon photoelectric detection of a withdrawal response or when a “cut off” time (e.g., 20 seconds at 4.66 amps) is reached in the absence of a withdrawal response. Test compounds are assessed for their ability to return thermal response latencies to pre-lesion levels following systemic administration.

### EXAMPLE 6c

### Other Behavioral Endpoints

Additional observations are made to determine nerve injury-induced changes in other sensory modalities. Cold allodynia is assessed using either a cooled surface or evaporative liquid cooling to determine reduced response latencies or exaggerated duration of response. Measurement of differences in hind paw weight bearing between injured and uninjured hind paws represents a spontaneous pain-like response to nerve injury. Test compounds are assessed for their ability to return cold responses and hind paw weight distribution to pre-lesion levels following systemic administration.

### EXAMPLE 7

### Pyresis/Antipyresis

Fever is a frequent accompaniment of inflammatory disease. Animal models make use of the pyretic properties of yeast and other inflammatory agents, injecting a yeast suspension or other agent subcutaneously. The modification of the subsequent pyretic response by therapeutic agents can be monitored by rectal telemetry or other measurements of body temperature. Several clinically relevant agents such as acetaminophen, aspirin and ibuprofen, reduce fever in these models. The antipyretic effect of VR1 antagonists in these tests would also be predictive of their clinical effect.

### EXAMPLE 8

### In Vivo Model for Colitis

Inflammatory bowel diseases such as ulcerative colitis, Crohn's disease, and celiac disease are characterized by numerous intestinal pathologies in structure and function. Furthermore, VR1 is elevated in colonic tissues from patients suffering from these disorders. Treatment of animals with dextran sulfate or other inflammogen induces in animals conditions similar to human inflammatory bowel diseases. The salicyate 5-ASA protects against dextran sulfate induced colitis in an animal model of the disease (Okayama, M., et al., Digestion, 70:240-249, 2005), and 5-ASA protects against disease progression in clinical trials (Pica, R. et al., Inflamm Bowel Dis. 10:731-736, 2004). VR1 antagonists are effective in alleviating colitis induced in rodents by dextran sulfate (Kimball, 2004).

### EXAMPLE 9

### In Vivo Model for Arthritis-CFA Tail Vein

Chronic arthritis can be induced in an animal by injection of a 1% Mycobacterium butyricum suspension or other pro-arthritic agent into a tail vein. After about two weeks, the development of arthritis can be evidenced by vocalization of the animal in response to gentle flexion of the hind paw or by other provocative action. Analgesic treatment reduces the vocalization or other response to the probe. In this model, the centrally acting analgesics morphine and tramadol fully relieved pain, whereas the NSAIDs indomethacin and diclofenac were partially effective, evidencing the model's clinical predictability. The analgesic effect of VR1 modulators in this test would predict their clinical usefulness in treating arthritis.

### EXAMPLE 10,

### In Vivo Model for Inflammogen-Induced Arthritis in Knee Joint Model

Injection of a 10% kaolin suspension or other pro-arthritic agent. into a knee joint is used to induce arthritis in animals. The gait of the animal or other pain response is scored as a measure of the painful effect of the arthritis on the animal's activity. The effect of test drug on the animal's normal behavior is quantified from zero, meaning non-painful response, to three for incapacitating impairment. Effective analgesic treatment includes the clinically used indomethacin (Motta, A. F. et al., Life Sci, 73:1995-2004, 2003). Thus the benefit of VR1 modulators in this model would predict their clinical relevance.

### EXAMPLE 11

### In Vivo Model for Cough

Vanilloid receptor modulators are tested in an animal model of cough, according to previously documented and validated methods, such as those described by Tanaka M, et al., Nippon Yakurigaku Zasshi 120:237-243, 2002 and Hall E, et al., Journal of Medical Microbiology 48:95-98, 1999. Testing is conducted in restrained or anesthetized mouse, rat, guinea prig, dog, cat, pig or human in response to the inhalation or microinjection into the larynx of irritants or infectious agents such as capsaicin, citric acid, sulfur dioxide gas or Bordetella pertussis. In some cases, animals are sensitized by pre-exposure to certain agents such as ovalbumin. Prior to or following irritant administration, the test subject receives, respectively, the prophylactic or therapeutic administration one or more times of a vanilloid receptor modulator, or vehicle control, by the enteral or parenteral route. The number of coughs per unit time are counted. Significant differences in the cough rate for the compound-treated subjects compared with vehicle-treated subjects are taken as evidence of antitussive activity. Clinically effective antitussive agents have activity in these preclinical models (Braga P C. Drugs under experimental and clinical research 20:199-203,1994). The antitussive action of the ultrapotent TRPV1 antagonist iodo-resiniferatoxin in both capsaicin and citric acid induced cough in a guinea pig model of cough (Trevisani M, et al., Thorax 59:769-772., 2004) is predictive of the clinical utility of TRPV1 antagonists as antitussive agents.

### EXAMPLE 12

### In Vivo Model for Bone Cancer Pain

Bone cancer causes intense pain in humans, mimicked in an animal model of bone cancer pain in rodents. Intramedullary injection of osteolytic sarcoma cells into the femur of mice is followed by bone resorption, neurochemical alterations in the ipsilateral spinal cord, and pain behaviors consequent to normally non-noxious palpitation of the bone. Analgesic treatments that are effective in this model include COX-2 inhibitors (Sabino, M A C et al., Cancer Res. 62:7343-7349, 2002) and high doses of morphine (Luger, N M et al., Pain 99:397-406, 2002), agents used clinically for pain relief in patients experiencing bone cancer pain. Because this model so closely mimics the human disease state, the finding that VR1 antagonists provide pain relief in this model (Ghilardi, J R et al., J Neurosci., 2005, 25:3126-3121) strongly supports their relief of pain associated with human bone cancer.

### EXAMPLE 13

### In Vivo Model for Itch, Contact Dermatitis, Eczema and Other Manifestations of Dermal Allergy, Hypersensitivity and/or Inflammation

Vanilloid receptor modulators are tested in an animal model of contact dermatitis or itch, according to previously documented and validated methods, such as those described by Saint-Mezard P et al., Journal of Investigative Dermatology 120:641-647, 2003; Gonzalez S. et al., American Journal of Contact Dermatitis: official journal of the American Contact Dermatitis Society 12:162-165, 2001; Wille J J, et al., Skin Pharmacology and Applied Skin Physiology 11:279-288,1998; Weisshaar E, Experimental Dermatology 8:254-260, 1999; and Thomsen J S et al., Experimental Dermatology 11:370-375, 2002). In models of contact dermatitis, testing is conducted in mouse, guinea pig or human in response to a single (primary allergic dermatitis) or repeated (sensitized allergic dermatitis) topical or photomechanical exposure of the skin to one or more haptens, such as 12-myristate-13 acetate, picryl chloride, oxazolone, capsaicin, arachidonic acid, lactic acid, trans-retinoic acid or sodium lauryl sulfate. Animals may be sensitized by pre-exposure to certain agents. The test subject receives the prophylactic or therapeutic administration of a vanilloid receptor modulator, or vehicle control by the enteral or parenteral route. Significant differences in skin inflammation (erythema, edema, hyperthermia, etc.) for the test compound-treated subjects compared with vehicle-treated subjects are taken as evidence of anti-allergy activity. Cumulative scratching behavior and/or number of scratches per unit time are measured. Significant differences in scratching behavior for the test compound-treated subjects compared with vehicle-treated subjects are taken as evidence of anti-pruritic activity. The ability of these models to predict the therapeutic effect of compounds in human dermal conditions is supported by the cross-species ability of serotonin to induce itch (Weisshaar et al., 1999). Additionally, the contact sensitizing property of commercially important drugs and the ability of ion channel modulators to prevent and treat skin sensitization in these models (Kydonieus A, et al., Proceedings of the International Symposium on Controlled Release of Bioactive Materials 24th:23-24, 1997) demonstrate the therapeutic utility of TRPV1 antagonists in dermal sensitization.

### EXAMPLE 14

### In Vivo Model for Rhinitis and Other Manifestations of Nasal Hypersensitivity and/or Inflammation

Vanilloid receptor modulators are tested in an animal model of rhinitis, according to previously documented and validated methods, such as those described by Hirayama Y, et al., European Journal of Pharmacology 467:197-203, 2003; Tiniakov R L et al., Journal of applied physiology 94:1821-1828, 2003; and Magyar T et al., Vaccine 20:1797-1802, 2002. Testing is conducted in mouse, guinea pig, dog or human in response to intranasal challenge with one or more irritants such as capsaicin, bradykinin, histamine, pollens, dextran sulfate, 2,4-tolylene diisocyanate, Bordetella bronchiseptica, Pasteurella multodica or acetic acid. In some cases, animals are sensitized by pre-exposure to certain agents including but not limited to ragweed or ovalbumin. Prior to or following irritant administration, the test subject receives, respectively, the prophylactic or therapeutic administration one or more times of a vanilloid receptor modulator, or vehicle control, by the enteral or parenteral route. Significant differences indicative of nasal rhinitis or sensitization for the test compound-treated subjects compared with vehicle-treated subjects are taken as evidence of anti-rhinitis activity. Independent variables include dose, frequency and route of administraon, time interval between prophylactic or therapeutic test compound administration and irritant challenge as well as sex and non-sex genotype of the test subject. The intimate role of neurogenic inflammation in these hypersensitivity states demonstrates that TRPV1 receptor modulators desensitize or block the sensitization underlying these disease states (Szallasi A and Blumberg P M Pain 68:195-208, 1996).

### EXAMPLE 15

### In Vivo-Model for Anxiety, Panic Disorder and Other Non-Adaptive Stressful or Phobic Responses

Vanilloid receptor modulators are tested in an animal model of anxiety, according to previously documented and validated methods, such as those described by Imaizumi M and Onodera K. Japanese Journal of Pharmacology 115:5-12, 2000. Testing is conducted in mouse or rat and consists of methods to measure avoidance of aversive environmental stimuli such as Geller or Vogel anticonflict tests, the light/dark test, the hole-board test, the elevated plus-maze and the elevated T-maze. Prior to environmental exposure, the test subject receives the prophylactic administration one or more times of a vanilloid receptor modulator, or vehicle control, by the enteral or parenteral route. The cumulative time or number of times spent engaged in the aversive behavior is measured. Significant differences in one or more of these measures for the test compound-treated subjects compared with vehicle-treated subjects are taken as evidence of anxiolytic activity. Because these models are pharmacologically validated by the effectiveness of clinically useful anxiolytics (Imaizumi and Onodera, 2000), they will be useful for the detection of anxiolytic vanilloid modulators.

### EXAMPLE 16

### In Vivo Model for Urinary Incontinence

VR1 selective ligands have clinical efficacy for treating conditions associated with urge urinary incontinence and overactive bladder (Anderson K E K., Urology 63:32-41, 2004; Lazzeri, M. et al., Urologia Intemationalis 72:145-9, 2004). To assess novel compounds for potential urinary incontinence activity, Sprague-Dawley rats are surgically implanted with bladder catheters allowing for the delivery of fluid (typically saline) and the monitoring of pressure (using a pressure transducer). Cystometry recordings can be monitored with a polygraph to evaluate voiding interval, threshold pressure, bladder capacity, bladder compliance, and the number of spontaneous bladder contractions. Compounds can also be evaluated under conditions of bladder hypertrophy and instability. Under anesthesia, a silk ligature is tied around the proximal urethra of rodents producing a partial outlet obstruction and subsequent hypertrophied bladder development within 6-9 weeks (Woods M. et al., J. Urology 166:1142-47, 2001). Cystometry recordings can then be evaluated as described above. Such preclinical procedures are sensitive to compounds having clinical utility for the treatment of urinary incontinence (Soulard et al., JPET 260: 1152-58,-1992), and the activity of VR1 ligands in this model would be predictive of clinical utility.

1. A compound of Formula (I): 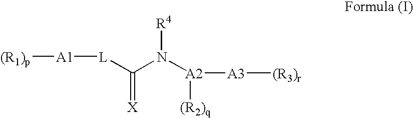
wherein:
A1 is phenyl, naphthalenyl, pyridinyl, or thienyl;
R₁ is independently hydroxy; halogen; C₁₋₈alkanyl optionally substituted with one or more substituents independently selected from the group consisting of halogen, trifluoromethylsulfonyl, trifluoromethylsulfinyl, fluorinated alkanyl, and C₁₋₈alkanyloxy; C₁₋₈alkanyloxy optionally substituted with one or more substituents independently selected from the group consisting of halogen, fluorinated alkanyl, and C₁₋₈alkanyloxy; fluorinated alkanyloxy; fluorinated alkanyl; C₁₋₈alkanylthio optionally substituted with one or more substituents independently selected from the group consisting of halogen, fluorinated alkanyl and C₁₋₈alkanyloxy; C₁₋₈alkanylsulfinyl optionally substituted with one or more substituents independently selected from the group consisting of halogen and C₁₋₈alkanyloxy; C₁₋₈alkanylsulfonyl optionally substituted with one or more substituents independently selected from the group consisting of halogen and C₁₋₈alkanyloxy; C₃₋₈cycloalkanyl; C₃₋₈cycloalkanyloxy; nitro; amino; C₁₋₈alkanylamino; C₁₋₈dialkanylamino; C₃₋₈cycloalkanylamino; N—C₁₋₈alkanyl-N—C₃₋₈cycloalkanylamino, cyano; carboxy; C₁₋₇alkanyloxycarbonyl; C₁₋₇alkanylcarbonyloxy; C₁₋₇alkanylaminocarbonyl; C₁₋₇alkanylcarbonylamino; diC₁₋₇alkanylaminocarbonyl; formyl; aminosulfonyl; C₁₋₈alkanylaminosulfonyl; di(C₁₋₈)alkanylaminosulfonyl; or cyano;
p is 0, 1 or 2;
L is C₂₋₈alkandiyl, C₂₋₈alkendiyl, or C₂₋₈alkyndiyl and L is optionally substituted with a substituent selected from the group consisting of C₁₋₈alkanyl, C₃₋₈cycloalkanyl and phenyl optionally substituted with one to three substituents independently selected from the group consisting of C₁₋₈alkanyl, C₁₋₈alkanyloxy, halogen, hydroxy, fluorinated alkanyl, fluorinated alkanyloxy, amino, di(C₁₋₃)alkanylamino, and C₁₋₃alkanylamino;
X is O or S;
A2 is selected from the group consisting of phenyl, thien-2-yl, naphthalenyl, pyridinyl, pyrimidinyl, pyrazinyl, quinolinyl, and isoquinolinyl;
R₂ is independently selected from the group consisting of C₁₋₈alkanyl, C₁₋₈alkanyloxy, hydroxy(C₁₋₈)alkanyl, fluorinated C₁₋₈alkanyl, hydroxyl, halogen, carboxy, C₁₋₈alkanyloxycarbonyl, and aminocarbonyl;
q is 0, 1, or 2;
A3 is selected from the group consisting of phenyl, thienyl, naphthalenyl, pyridinyl, pyrimidinyl, pyrazinyl, imidazolyl, oxazolyl, 4,5-dihydro-oxazolyl, pyrazolyl, dihydro-pyrazolyl, thiazolyl, thiadiazolyl, triazolyl, tetrazolyl, quinolinyl, isoquinolinyl, oxathiadiazolyl, benzimidazolyl, tetrahydrobenzimidazolyl, tetrahydroindazolyl, oxathiadiazolyl 2-oxide, oxadiazolyl, tetrahydroisoquinolinyl, tetrahydroquinolinyl, and indolyl;
such that when A3 is tetrahydrobenzimidazolyl, tetrahydroindazolyl, oxathiadiazolyl 2-oxide, tetrahydroisoquinolinyl, or tetrahyd roquinolinyl, then r is 0;
R₃ is independently selected from the group consisting of hydroxy; halogen; C₁₋₈alkanyl; hydroxy(C₁₋₈)alkanyl; C₁₋₈alkanyloxy optionally substituted with amino, C₁₋₈alkanylamino, or C₁₋₈dialkanylamino; fluorinated alkanyloxy; fluorinated alkanyl; C₁₋₈alkanylthio; nitro; amino; C₁₋₈alkanylamino; C₁₋₈dialkanylamino; C₃₋₈cycloalkanylamino; cyano; aminosulfonyl; carboxy; C₁₋₈alkanylsulfonylamino; aminocarbonyl; C₁₋₈alkanyloxycarbonyl; C₁₋₄alkanyloxycarbonylamino; C₁₋₈alkanylaminocarbonyl; C₁₋₈alkanylcarbonylamino; diC₁₋₈alkanylaminocarbonyl; oxo when A3 is dihydro-pyrazolyl; and formyl; and wherein the C₁₋₈alkanyl group of any C₁₋₈alkanylamino and C₁₋₈dialkanylamino containing substituent of R₃ is optionally substituted with hydroxy;
r is 0, 1, or 2;
R₄ is hydrogen or C₁₋₈alkyl;
provided that a compound of Formula 1 is other than
a compound wherein p is 1, R₁ is 4-trifluoromethylsulfonyl, A1 is phenyl, L is trans —CH═CH—, X is O, A2 is pyridin-3-yl, q is 1, R₂ is 5-methyl, A3 is imidazol-1-yl, r is 2, R₃ is 4,5-dichloro, and R₄ is hydrogen;
a compound wherein p is 1, R₁ is 4-trifluoromethyl, A1 is phenyl, L is —(CH₂)₂—, X is O, A2 is phenyl, q is 1, R₂ is 3-trifluoromethyl, A3 is pyrazol-1-yl, r is 2, R₃ is 3,5-dimethyl, and R₄ is hydrogen;
a compound wherein p is 1, R₁ is 4-trifluoromethyl, A1 is phenyl, L is —(CH₂)₂—, X is O, A2 is phenyl, q is 1, R₂ is 3-trifluoromethyl, A3 is imidazol-1-yl, r is 1, R₃ is 4-carboxy, and R₄ is hydrogen; and
enantiomers, diastereomers, tautomers, solvates, and
pharmaceutically acceptable salts thereof.

2. A compound of Formula (I) 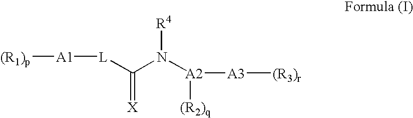
wherein:
A1 is phenyl, naphthalenyl, or thienyl;
R₁ is independently hydroxy; halogen; C₁₋₈alkanyl optionally substituted with one or more substituents independently selected from the group consisting of halogen, fluorinated alkanyl and C₁₋₈alkanyloxy; C₁₋₈alkanyloxy optionally substituted with one or more substituents independently selected from the group consisting of halogen, fluorinated alkanyl, and C₁₋₈alkanyloxy; fluorinated alkanyloxy; fluorinated alkanyl; C₁₋₈alkanylthio optionally substituted with one or more substituents independently selected from the group consisting of halogen, fluorinated alkanyl and C₁₋₈alkanyloxy; C₃₋₈cycloalkanyl; C₃₋₈cycloalkanyloxy; nitro; amino; C₁₋₈alkanylamino; C₁₋₈dialkanylamino; C₃₋₈cycloalkanylamino; cyano; carboxy; C₁₋₇alkanyloxycarbonyl; C₁₋₇alkanylcarbonyloxy; C₁₋₇alkanylaminocarbonyl; C₁₋₇alkanylcarbonylamino; diC₁₋₇alkanylaminocarbonyl; formyl; aminosulfonyl; C₁₋₈alkanylaminosulfonyl; di(C₁₋₈)alkanylaminosulfonyl; or cyano;
p is 0, 1 or 2;
L is C₂₋₈alkandiyl, C₂₋₈alkendiyl, or C₂₋₈alkyndiyl and L is optionally substituted with a substituent selected from the group consisting of C₁₋₈alkanyl, C₃₋₈cycloalkanyl and phenyl optionally substituted with one to three substituents independently selected from the group consisting of C₁₋₈alkanyl, C₁₋₈alkanyloxy, halogen, hydroxy, fluorinated alkanyl, fluorinated alkanyloxy, amino, di(C₁₋₃)alkanylamino, and C₁₋₃alkanylamino;
X is O or S;
A2 is selected from the group consisting of phenyl, thien-2-yl, naphthalenyl, pyridinyl, pyrimidinyl, pyrazinyl, quinolinyl, and isoquinolinyl;
R₂ is independently selected from the group consisting of C₁₋₈alkanyl, C₁₋₈alkanyloxy, hydroxy(C₁₋₈)alkanyl, fluorinated C₁₋₈alkanyl, hydroxyl, halogen, carboxy, C₁₋₈alkanyloxycarbonyl, and aminocarbonyl;
q is 0, 1, or 2;
A3 is selected from the group consisting of phenyl, thienyl, naphthalenyl, pyridinyl, pyrimidinyl, pyrazinyl, imidazolyl, oxazolyl, 4,5-dihydro-oxazolyl, pyrazolyl, dihydro-pyrazolyl, thiazolyl, thiadiazolyl, triazolyl, tetrazolyl, quinolinyl, isoquinolinyl, and indolyl;
R₃ is independently selected from the group consisting of hydroxy; halogen; C₁₋₈alkanyl; hydroxy(C₁₋₈)alkanyl; C₁₋₈alkanyloxy optionally substituted with amino, C₁₋₈alkanylamino, or C₁₋₈dialkanylamino; fluorinated alkanyloxy; fluorinated alkanyl; C₁₋₈alkanylthio; nitro; amino; C₁₋₈alkanylamino; C₁₋₈dialkanylamino; C₃₋₈cycloalkanylamino; cyano; aminosulfonyl; carboxy; C₁₋₈alkanylsulfonylamino; aminocarbonyl; C₁₋₈alkanyloxycarbonyl; C₁₋₄alkanyloxycarbonylamino; C₁₋₈alkanylaminocarbonyl; C₁₋₈alkanylcarbonylamino; diC₁₋₈alkanylaminocarbonyl; oxo when A3 is dihydro-pyrazolyl; and formyl; and wherein the C₁₋₈alkanyl group of any C₁₋₈alkanylamino and C₁₋₈dialkanylamino containing substituent of R₃ is optionally substituted with hydroxy;
r is 0, 1, or 2;
R₄ is hydrogen or C₁₋₈alkyl; and enantiomers, diastereomers, tautomers, solvates, and pharmaceutically acceptable salts thereof.

3. The compound according to claim 1 wherein A1 is phenyl, pyridinyl, or thienyl.

4. The compound according to claim 1 wherein A1 is phenyl, pyridinyl, or thienyl, and p is 1 or 2.

5. The compound according to claim 1 or 2 wherein A1 is phenyl or thienyl.

6. The compound according to claim 1 or 2 wherein A1 is phenyl or thienyl, and p is 1 or 2.

7. The compound according to claim 1 or 2 wherein A1 is phenyl substituted at the 4-position with R₁, or thiophen-2-yl substituted at the 5-position with R₁.

8. The compound according to claim 1 wherein R₁ is independently C₁₋₆alkanyl, fluorinated C₁₋₆alkanyl; C₁₋₈alkanylsulfonyl substituted with one to three fluoro substituents; C₁₋₈alkanylthio substituted with one to three fluoro substituents; C₁₋₈alkanylsulfinyl substituted with one to three fluoro substituents; chloro; fluoro; or N—C₁₋₄alkanyl-N-cyclohexylamino.

9. The compound according to claim 1 wherein R₁ is C₁₋₄alkanyl; fluorinated C₁₋₄alkanyl; trifluoromethylsulfonyl; trifluoromethylthio; trifluoromethylsulfinyl; chloro; or N-methyl-N-cyclohexylamino.

10. The compound according to claim 1 wherein R₁ is independently t-butyl, trifluoromethyl, trifluoromethylsulfonyl, or trifluoromethylthio.

11. The compound according to claim 1 or 2 wherein R₁ is independently C₁₋₆alkanyl or fluorinated C₁₋₆alkanyl.

12. The compound according to claim 1 or 2 wherein R₁ is independently C₁₋₄alkanyl or fluorinated C₁₋₄alkanyl.

13. The compound according to claim 1 or 2 wherein R₁ is independently t-butyl or trifluoromethyl.

14. The compound according to claim 1 or 2 wherein p is 1 or 2.

15. The compound according to claim 1 or 2 wherein p is 1.

16. The compound according to claim 1 or 2 wherein L is C₂₋₈alkandiyl, C₂₋₈alkendiyl, or C₂₋₈alkyndiyl and L is optionally substituted with a substituent selected from the group consisting of C₁₋₄alkanyl, C₃₋₈cycloalkanyl, and phenyl.

17. The compound according to claim 1 or 2 wherein L is C₂₋₄alkandiyl or C₂₋₄alkendiyl, and L is optionally substituted with a substituent selected from the group consisting of C₁₋₄alkanyl, C₃₋₈cycloalkanyl, and phenyl.

18. The compound according to claim 1 or 2 wherein L is —CH₂CH₂— or —CH═CH—.

19. The compound according to claim 1 or 2 wherein X is O or S.

20. The compound according to claim 1 or 2 wherein X is O.

21. The compound according to claim 1 or 2 wherein q is 0, 1, or 2, and A2 is selected from the group consisting of phenyl, pyridinyl, pyrimidinyl, pyrazinyl, quinolinyl, and isoquinolinyl.

22. The compound according to claim 1 or 2 wherein q is 0, 1, or 2, and A2 is selected from the group consisting of phenyl, pyridinyl, and isoquinolinyl.

23. The compound according to claim 1 or 2 wherein q is 0, 1, or 2 and A2 is selected from the group consisting of phenyl, pyridin-3-yl, and isoquinolin-5-yl.

24. The compound according to claim 1 or 2 wherein R₂ is independently selected from the group consisting of C₁₋₄alkanyl, hydroxy(C₁₋₄)alkanyl, fluorinated C₁₋₄alkanyl, and fluoro.

25. The compound according to claim 1 or 2 wherein R₂ is independently selected from the group consisting of methyl, hydroxymethyl, and fluoro.

26. The compound according to claim 1 or 2 wherein q is 1 or 2.

27. The compound according to claim 1 wherein r is 1 or 2 and A3 is selected from the group consisting of phenyl, thienyl, pyridinyl, pyrimidinyl, and imidazolyl.

28. The compound according to claim 1 wherein r is 1 or 2 and when A2 is phenyl, A3 is 4-phenyl, 3-phenyl, 2-pyridin-4-yl, 2-pyridin-2-yl, 3-pyridin-4-yl, 2-pyridin-3-yl, 4-pyridin-4-yl, 4-pyridin-3-yl, 4-pyridin-2-yl, 4-pyrimidin-5-yl, 3-pyrimidin-2-yl or 4-imidazol-1-yl; and when A2 is isoquinolin-5-yl, A3 is 8-pyridin-4-yl or 8-phenyl; and when A2 is pyridin-3-yl, A3 is 6-phenyl, 6-pyridin-3-yl, 6-pyridin-2-yl, 6-pyridin-4-yl, or 6-imidazol-1-yl.

29. The compound according to claim 1 wherein r is 1 or 2 and when A2 is phenyl, A3 is 4-phenyl, 3-phenyl, 2-pyridin-2-yl, 4-pyridin-4-yl, 4-pyridin-3-yl, 4-pyridin-2-yl, 4-pyrimidin-5-yl, 3-pyrimidin-2-yl, or 4-imidazol-1-yl; and when A2 is isoquinolin-5-yl, A3 is 8-pyridin-4-yl or 8-phenyl; and when A2 is pyridin-3-yl, A3 is 6-phenyl, 6-pyridin-3-yl, 6-pyridin-2-yl, 6-pyridin-4-yl, or 6-imidazol-1-yl.

30. The compound according to claim 1 or 2 wherein r is 1 or 2 and A3 is selected from the group consisting of phenyl, pyridinyl, and pyrimidinyl.

31. The compound according to claim 1 or 2 wherein r is 1 or 2 and when A2 is phenyl, A3 is 4-phenyl, 3-phenyl, 2-pyridin-4-yl, 2-pyridin-2-yl, 3-pyridin-4-yl, 2-pyridin-3-yl, 4-pyridin-4-yl, 4-pyridin-3-yl, 4-pyridin-2-yl, 4-pyrimidin-5-yl or 3-pyrimidin-2-yl; and when A2 is isoquinolin-5-yl, A3 is 8-pyridin-4-yl or 8-phenyl; and when A2 is pyridin-3-yl, A3 is 6-phenyl, 6-pyridin-3-yl, 6-pyridin-2-yl, or 6-pyridin-4-yl.

32. The compound according to claim 1 or 2 wherein r is 1 or 2 and when A2 is phenyl, A3 is 4-phenyl, 3-phenyl, 2-pyridin-2-yl, 4-pyridin-4-yl, 4-pyridin-3-yl, 4-pyridin-2-yl, 4-pyrimidin-5-yl or 3-pyrimidin-2-yl; and when A2 is isoquinolin-5-yl, A3 is 8-pyridin-4-yl or 8-phenyl; and when A2 is pyridin-3-yl, A3 is 6-phenyl, 6-pyridin-3-yl, 6-pyridin-2-yl, or 6-pyridin-4-yl.

33. The compound according to claim 1 or 2 wherein R₃ is independently selected from the group consisting of hydroxy, fluoro, chloro, C₁₋₄alkanyl, hydroxy(C₁₋₄)alkanyl, C₁₋₄alkanyloxy optionally substituted with amino, C₁₋₄alkanylamino, or C₁₋₄dialkanylamino, fluorinated alkanyloxy, fluorinated alkanyl, amino, C₁₋₄alkanylamino, C₁₋₄dialkanylamino, C₃₋₈cycloalkanylamino, cyano, aminosulfonyl, C₁₋₄alkanylsuIfonylamino, C₁₋₄alkanyloxycarbonyl, C₁₋₄alkanyloxycarbonylamino, aminocarbonyl, C₁₋₄alkanylaminocarbonyl, C₁₋₄alkanylcarbonylamino, diC₁₋₄alkanylaminocarbonyl, and formyl; and wherein C₁₋₄alkanyl in any of the foregoing C₁₋₄alkanylamino and C₁₋₄dialkanylamino containing substituents of R₃ is optionally substituted with hydroxy.

34. The compound according to claim 1 wherein R₃ is independently selected from the group consisting of hydroxyl, fluoro, chloro, methyl, hydroxymethyl, C₁₋₃alkanyloxy optionally substituted with amino, methylamino, or dimethylamino, trifluoromethoxy, trifluoromethyl, methylsulfonylamino, t-butoxycarbonylamino, aminocarbonyl, and methylcarbonylamino.

35. The compound according to claim 1 wherein R₃ is independently selected from the group consisting of hydroxy, fluoro, chloro, methyl, hydroxymethyl, 2-aminoethoxy, methylsulfonylamino, and methylcarbonylamino.

36. The compound according to claim 1 or 2 wherein R₃ is independently selected from the group consisting of hydroxyl, fluoro, methyl, hydroxymethyl, C₁₋₃alkanyloxy optionally substituted with amino, methylamino, or dimethylamino, trifluoromethoxy, trifluoromethyl, methylsulfonylamino, t-butoxycarbonylamino, aminocarbonyl, and methylcarbonylamino.

37. The compound according to claim 1 or 2 wherein R₃ is independently selected from the group consisting of hydroxy, fluoro, methyl, hydroxymethyl, 2-aminoethoxy, methylsulfonylamino, and methylcarbonylamino.

38. The compound according to claim 1 or 2 wherein r is 1 or 2.

39. The compound according to claim 1 or 2 wherein r is 1.

40. The compound according to claim 1 or 2 wherein R₄ is hydrogen or C₁₋₄alkyl.

41. The compound according to claim 1 wherein R₄ is hydrogen.

42. A compound of Formula (I) 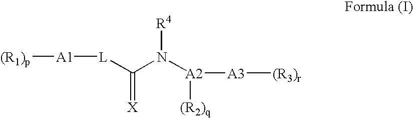
wherein:
A1 is phenyl, pyridinyl, or thienyl;
R₁ independently is C₁₋₆alkanyl; fluorinated C₁₋₆alkanyl; C₁₋₈alkanylsulfonyl substituted with one to three fluoro substituents; C₁₋₈alkanylthio substituted with one to three fluoro substituents; C₁₋₈alkanylsulfinyl substituted with one to three fluoro substituents; chloro; fluoro; or N—C₁₋₄alkanyl-N-cyclohexylamino;
p is 1 or 2;
L is C₂₋₈alkandiyl, C₂₋₈alkendiyl, or C₂₋₈alkyndiyl and L is optionally substituted with a substituent selected from the group consisting of C₁₋₄alkanyl, C₃₋₈cycloalkanyl and phenyl;
X is O or S;
A2 is selected from the group consisting of phenyl, thienyl, pyridinyl, pyrimidinyl, pyrazinyl, quinolinyl, and isoquinolinyl;
R₂ is independently selected from the group consisting of C₁₋₄alkanyl, hydroxy(C₁₋₄)alkanyl, fluorinated C₁₋₄alkanyl, and fluoro;
q is 1 or 2;
A3 is selected from the group consisting of phenyl, thienyl, pyridinyl, pyrimidinyl, and imidazolyl;
R₃ is independently selected from the group consisting of hydroxy, fluoro, chloro, C₁₋₄alkanyl, hydroxy(C₁₋₄)alkanyl, C₁₋₄alkanyloxy optionally substituted with amino, C₁₋₄alkanylamino, or C₁₋₄dialkanylamino, fluorinated alkanyloxy, fluorinated alkanyl, amino, C₁₋₄alkanylamino, C₁₋₄dialkanylamino, C₃₋₈cycloalkanylamino, cyano, aminosulfonyl, C₁₋₄alkanylsulfonylamino, C₁₋₄alkanyloxycarbonyl, C₁₋₄alkanyloxycarbonylamino, aminocarbonyl, C₁₋₄alkanylaminocarbonyl, C₁₋₄alkanylcarbonylamino, diC₁₋₄alkanylaminocarbonyl, and formyl, and wherein C₁₋₄alkanyl in any of the foregoing C₁₋₄alkanylamino and C₁₋₄dialkanylamino containing substituent of R₃ is optionally substituted with hydroxy;
R₄ is hydrogen or C₁₋₄alkyl,
provided that a compound of Formula 1 is other than
a compound wherein p is 1, R₁ is 4-trifluoromethylsulfonyl, A1 is phenyl, L is trans —CH═CH—, X is O, A2 is pyridin-3-yl, q is 1, R₂ is 5-methyl, A3 is imidazol-1-yl, r is 2, R₃ is 4,5-dichloro, and R₄ is hydrogen; and
enantiomers, diastereomers, tautomers, solvates, and pharmaceutically acceptable salts thereof.

43. A compound of Formula 1 wherein:
A1 is phenyl, pyridinyl, or thienyl;
R₁ is independently C₁₋₄alkanyl; fluorinated C₁₋₄alkanyl; trifluoromethylsulfonyl; trifluoromethylthio; trifluoromethylsulfinyl; chloro; or N-methyl-N-cyclohexylamino;
p is 1;
L is C₂₋₄alkandiyl or C₂₋₄alkendiyl, and L is optionally substituted with a substituent selected from the group consisting of C₁₋₄alkanyl, C₃₋₈cycloalkanyl and phenyl;
X is O;
A2 is selected from the group consisting of phenyl, thienyl, pyridinyl, and isoquinolinyl;
R₂ is independently selected from the group consisting of methyl, hydroxymethyl, trifluoromethyl, and fluoro;
q is 0, 1, or 2;
A3 is 4-phenyl, 3-phenyl, 2-pyridin-4-yl, 2-pyridin-2-yl, 3-pyridin-4-yl, 2-pyridin-3-yl, 4-pyridin-4-yl, 4-pyridin-3-yl, 4-pyridin-2-yl, 4-pyrimidin-5-yl, 3-pyrimidin-2-yl or 4-imidazol-1-yl when A2 is phenyl; and when A2 is isoquinolin-5-yl, A3 is 8-pyridin-4-yl or 8-phenyl; and when A2 is pyridin-3-yl, A3 is 6-phenyl, 6-pyridin-3-yl, 6-pyridin-2-yl, 6-pyridin-4-yl, or 6-imidazol-1-yl;
R₃ is independently selected from the group consisting of hydroxy, fluoro, chloro, methyl, hydroxymethyl, C₁₋₃alkanyloxy optionally substituted with amino, methylamino, or dimethylamino, trifluoromethoxy, trifluoromethyl, methylsulfonylamino, t-butoxycarbonylamino, aminocarbonyl, and methylcarbonylamino;
r is 1;
R₄ is hydrogen, and
enantiomers, diastereomers, tautomers, solvates, and pharmaceutically acceptable salts thereof.

44. A compound of Formula (I) 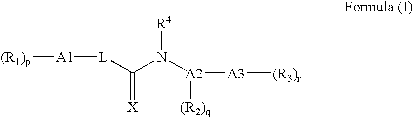
wherein:
A1 is phenyl or thienyl;
R₁ independently is C₁₋₆alkanyl or fluorinated C₁₋₆alkanyl;
p is 1 or 2;
L is C₂₋₈alkandiyl, C₂₋₈alkendiyl, or C₂₋₈alkyndiyl and L is optionally substituted with a substituent selected from the group consisting of C₁₋₄alkanyl, C₃₋₈cycloalkanyl and phenyl;
X is O or S;
A2 is selected from the group consisting of phenyl, pyridinyl, pyrimidinyl, pyrazinyl, quinolinyl, and isoquinolinyl;
R₂ is independently selected from the group consisting of C₁₋₄alkanyl, hydroxy(C₁₋₄)alkanyl, fluorinated C₁₋₄alkanyl, and fluoro;
q is 1 or 2;
A3 is selected from the group consisting of phenyl, pyridinyl, and pyrimidinyl;
R₃ is independently selected from the group consisting of hydroxy, fluoro, chloro, C₁₋₄alkanyl, hydroxy(C₁₋₄)alkanyl, C₁₋₄alkanyloxy optionally substituted with amino, C₁₋₄alkanylamino, or C₁₋₄dialkanylamino, fluorinated alkanyloxy, fluorinated alkanyl, amino, C₁₋₄alkanylamino, C₁₋₄dialkanylamino, C₃₋₈cycloalkanylamino, cyano, aminosulfonyl, C₁₋₄alkanylsuIfonylamino, C₁₋₄alkanyloxycarbonyl, C₁₋₄alkanyloxycarbonylamino, aminocarbonyl, C₁₋₄alkanylaminocarbonyl, C₁₋₄alkanylcarbonylamino, diC₁₋₄alkanylaminocarbonyl, and formyl, and wherein C₁₋₄alkanyl in any of the foregoing C₁₋₄alkanylamino and C₁₋₄dialkanylamino containing substituent of R₃ is optionally substituted with hydroxy;
R₄ is hydrogen or C₁₋₄alkyl, and
enantiomers, diastereomers, tautomers, solvates, and pharmaceutically acceptable salts thereof.

45. A compound of Formula 1 wherein:
A1 is phenyl or thienyl;
R₁ is independently C₁₋₄alkanyl or fluorinated C₁₋₄alkanyl;
p is 1;
L is C₂₋₄alkandiyl or C₂₋₄alkendiyl, and L is optionally substituted with a substituent selected from the group consisting of C₁₋₄alkanyl, C₃₋₈cycloalkanyl and phenyl;
X is O;
A2 is selected from the group consisting of phenyl, pyridinyl, and isoquinolinyl;
R₂ is independently selected from the group consisting of methyl, hydroxymethyl, and fluoro;
q is 0, 1, or 2;
A3 is 4-phenyl, 3-phenyl, 2-pyridin-4-yl, 2-pyridin-2-yl, 3-pyridin-4-yl, 2-pyridin-3-yl, 4-pyridin-4-yl, 4-pyridin-3-yl, 4-pyridin-2-yl, 4-pyrimidin-5-yl or3-pyrimidin-2-yl when A2 is phenyl; and when A2 is isoquinolin-5-yl, A3 is 8-pyridin-4-yl or 8-phenyl; and when A2 is pyridin-3-yl, A3 is 6-phenyl, 6-pyridin-3-yl, 6-pyridin-2-yl, or 6-pyridin-4-yl;
R₃ is independently selected from the group consisting of hydroxy, fluoro, methyl, hydroxymethyl, C₁₋₃alkanyloxy optionally substituted with amino, methylamino, or dimethylamino, trifluoromethoxy, trifluoromethyl, methylsulfonylamino, t-butoxycarbonylamino, aminocarbonyl, and methylcarbonylamino;
r is 1;
R₄ is hydrogen, and
enantiomers, diastereomers, tautomers, solvates, and pharmaceutically acceptable salts thereof.

46. A compound of Formula Ia: 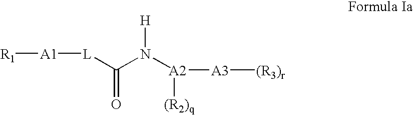
wherein:
A1 is phenyl substituted at the 4-position with R₁, or thiophen-2-yl substituted at the 5-position with R₁;
R₁ is independently t-butyl or trifluoromethyl,
L is —CH₂CH₂— or —CH═CH—;
A2 is selected from the group consisting of phenyl, pyridin-3-yl, and isoquinolin-5-yl optionally substituted with R₂;
R₂ is independently selected from the group consisting of hydrogen, methyl, hydroxymethyl, and fluoro;
q is 0, 1, or 2;
A3 is 4-phenyl, 3-phenyl, 2-pyridin-2-yl, 4-pyridin-4-yl, 4-pyridin-3-yl, 4-pyridin-2-yl, 4-pyrimidin-5-yl or 3-pyrimidin-2-yl when A2 is phenyl; and when A2 is isoquinolin-5-yl, A3 is 8-pyridin-4-yl or 8-phenyl; and when A2 is pyridin-3-yl, A3 is 6-phenyl, 6-pyridin-3-yl, 6-pyridin-2-yl, or 6-pyridin-4-yl;
R₃ is independently selected from the group consisting of hydroxy, fluoro, methyl, hydroxymethyl, 2-aminoethoxy, methylsulfonylamino, and methylcarbonylamino, and
enantiomers, diastereomers, tautomers, solvates, and pharmaceutically acceptable salts thereof.

47. A compound of Formula Ia: 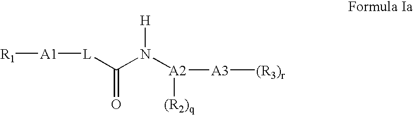
wherein:
A1 is phenyl substituted at the 4-position with R₁, or thiophen-2-yl substituted at the 5-position with R₁;
R₁ is independently t-butyl, trifluoromethyl, trifluoromethylsulfonyl, or trifluoromethylthio;
L is —CH₂CH₂— or —CH═CH—;
A2 is selected from the group consisting of phenyl, pyridin-3-yl, and isoquinolin-5-yl optionally substituted with R₂;
R₂ is independently selected from the group consisting of methyl, hydroxymethyl, trifluoromethyl, and fluoro;
q is 0, 1, or 2;
A3 is 4-phenyl, 3-phenyl, 2-pyridin-2-yl, 4-pyridin-4-yl, 4-pyridin-3-yl, 4-pyridin-2-yl, 4-pyrimidin-5-yl, 3-pyrimidin-2-yl or 4-imidazol-1-yl when A2 is phenyl; and when A2 is isoquinolin-5-yl, A3 is 8-pyridin-4-yl or 8-phenyl; and when A2 is pyridin-3-yl, A3 is 6-phenyl, 6-pyridin-3-yl, 6-pyridin-2-yl, 6-pyridin-4-yl, or 6-imidazol-1-yl;
R₃ is independently selected from the group consisting of hydroxy, fluoro, chloro, methyl, hydroxymethyl, 2-aminoethoxy, methylsulfonylamino, and methylcarbonylamino,
provided that a compound of Formula 1 is other than a compound wherein p is 1, R₁ is 4-trifluoromethylsulfonyl, A1 is phenyl, L is trans —CH═CH—, X is O, A2 is pyridin-3-yl, q is 1, R₂ is 5-methyl, A3 is imidazol-1-yl, r is 2, R₃ is 4,5-dichloro, and R₄ is hydrogens
and
enantiomers, diastereomers, tautomers, solvates, and pharmaceutically acceptable salts thereof.

48. A compound of Formula Ia 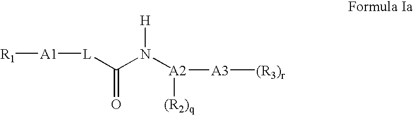
selected from the group consisting of:
a compound of Formula (Ia) wherein R₁ is 4-t-butyl, A1 is phenyl, L is —(CH₂)₂—, A2 is phenyl, q is 0; R₂ is absent; A3 is 2-phenyl, r is 1, and R₃ is 4-hydroxy;
a compound of Formula (Ia) wherein R₁ is4-t-butyl, A1 is phenyl, L is —(CH₂)₂—, A2 is phenyl, q is 0, R₂ is absent, A3 is 3-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 4-hydroxy;
a compound of Formula (Ia) wherein R₁ is4-t-butyl, A1 is phenyl, L is —(CH₂)₂—, A2 is phenyl, q is 0, R₂ is absent, A3 is 2-phenyl, r is 1, and R₃ is 3-hydroxy;
a compound of Formula (Ia) wherein R₁ is4-t-butyl, A1 is phenyl, L is —(CH₂)₂—, A2 is phenyl, q is 0, R₂ is absent, A3 is 3-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 0, R₂ is absent, A3 is 2-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-nitro;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-trifluoromethyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-methylcarbonyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-chloro;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-fluoro;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-amino;
a compound of Formula (Ia) wherein R₁ is 4-t-butyl, A1 is phenyl, L is —(CH₂)₂—, A2 is phenyl, q is 0, R₂ is absent, A3 is 2-phenyl, r is 1, and R₃ is 3-hydroxymethyl;
a compound of Formula (Ia) wherein R₁ is 4-t-butyl, A1 is phenyl, L is —(CH₂)₂—, A2 is phenyl, q is 0, R₂ is absent, A3 is 2-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 3-hydroxymethyl;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-hydroxymethyl;
a compound of Formula (Ia) wherein R₁ is 4-t-butyl, A1 is phenyl, L-is —(CH₂)₂—, A2 is phenyl, q is 0, R₂ is absent, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-cyano;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-trifluoromethoxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-methoxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-methyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-methoxycarbonyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-carboxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-aminocarbonyl;
a compound of Formula (Ia) wherein R₁ is 4-t-butyl, A1 is phenyl, L is trans —CH═CH—, A2 is phenyl, q is 0, R₂ is absent, A3 is 3-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 3-dimethylaminocarbonyl;
a compound of Formula (Ia) wherein R₁ is 4-t-butyl, A1 is phenyl, L is trans —CH═CH—, A2 is phenyl, q is 0, R₂ is absent, A3 is 4-phenyl, r is 1, and R₃ is 3-aminocarbonyl;
a compound of Formula (Ia) wherein R₁ is 4-t-butyl, A1 is phenyl, L is trans —CH═CH—, A2 is phenyl, q is 0, R₂ is absent, A3 is 4-phenyl, r is 1, and R₃ is 3-dimethylaminocarbonyl;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 3-dimethylaminocarbonyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-dimethylaminocarbonyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-methylsulfonylamino;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-t-butoxycarbonylamino;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-t-butoxycarbonylaminomethyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-aminomethyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-ureido;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-(2-amino-ethoxy);
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-(2-hydroxy-ethylamino);
a compound of Formula (Ia) wherein R₁ is 4-trifluoromethyl, A1 is phenyl, L is trans —CH═CH—, A2 is phenyl, q is 0, R₂ is absent, A3 is 4-phenyl, r is 1, and R₃ is 3-aminocarbonyl;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 2-methyl, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 2-fluoro, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 3-fluoro, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 2-methyl, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 2-fluoro, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 3-fluoro, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 2-methyl, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 2-fluoro, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 3-fluoro, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-hydroxy;
a compound of Formula (Ia) wherein R₁ is 5-t-butyl, A1 is thiophen-2-yl, L is —(CH₂)₂—, A2 is phenyl, q is 0, R₂ is absent, A3 is 4-phenyl, r is 1, and R₃ is 3-aminocarbonyl;
a compound of Formula (Ia) wherein R₁ is 5-t-butyl, A1 is thiophen-2-yl, L is —(CH₂)₂—, A2 is phenyl, q is 0, R₂ is absent, A3 is 4-phenyl, r is 1, and R₃ is 3-methylcarbonylamino;
a compound of Formula (Ia) wherein R, is 5-t-butyl, A1 is thiophen-2-yl, L is —(CH₂)₂—, A2 is phenyl, q is 0, R₂ is absent, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 2-carboxy, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 2-carboxy, A3 is 4-phenyl, r is 1, and R₃ is 3-methylaminocarbonyl;
a compound of Formula (Ia) wherein R₁ is 4-t-butyl, A1 is phenyl, L is —(CH₂)₂—, A2 is phenyl, q is 1, R₂ is 2-carboxy, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 2-hydroxymethyl, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 2-hydroxymethyl, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 2-hydroxymethyl, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 3-carboxy, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 3-carboxy, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 3-carboxy, A3 is 4-phenyl, r is 1, and R₃ is 3-hydroxy;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 3-methylcarbonylamino;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 2-hydroxy;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 3-hydroxy;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 4-hydroxy;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 3-hydroxymethyl;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is naphthalen-1-yl, q is 0, R₂ is absent, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is naphthalen-1-yl, q is 0, R₂ is absent, A3 is 4-phenyl, r is 1, and R₃ is 3-hydroxy;
a compound of Formula (Ia) wherein R₁ is 4-trifluoromethyl, A1 is phenyl, L is —(CH₂)₂—, A2 is naphthalen-1-yl, q is 0, R₂ is absent, A3 is 4-phenyl, r is 1, and R₃ is 3-aminocarbonyl;
a compound of Formula (Ia) wherein R₁ is 4-trifluoromethyl, A1 is phenyl, L is —(CH₂)₂—, A2 is naphthalen-1-yl, q is 0, R₂ is absent, A3 is 4-phenyl, r is 1, and R₃ is 3-methylcarbonylamino;
a compound of Formula (Ia) wherein R₁ is 4-trifluoromethyl, A1 is phenyl, L is —(CH₂)₂—, A2 is naphthalen-1-yl, q is 0, R₂ is absent, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 2, R₂ is 3,5-dimethyl, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 2, R₂ is 3,5-dimethyl, A3 is 4-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 2, R₂ is 3,5-dimethyl, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 2, R₂ is 3,5-difluoro, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 2, R₂ is 3,5-difluoro, A3 is 4-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 2, R₂ is 3,5-difluoro, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-phenyl, r is 1, and R₃ is 3-hydroxymethyl;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 1, R₂ is 3-fluoro, A3 is 4-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 1, R₂ is 3-fluoro, A3 is 4-phenyl, r is 1, and R₃ is 4-hydroxy
a compound of Formula (Ia) wherein R₁ is 4-t-butyl, A1 is phenyl, L is —(CH₂)₂—, A2 is phenyl, q is 1, R₂ is 3-fluoro, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 0, R₂ is absent, A3 is 2-pyridin-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 3-pyridin-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 2-pyridin-3-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 3-pyridin-3-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-pyridin-3-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 2-pyridin-2-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 3-pyridin-2-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-pyridin-2-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-pyrimidin-2-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-pyrimidin-5-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 3-pyridin-2-yl, r is 1, and R₃ is 6-hydroxy;
a compound of Formula (Ia) wherein R₁ is 4-t-butyl, A1 is phenyl, L is —(CH₂)₂—, A2 is phenyl, q is 0, R₂ is absent, A3 is 4-pyridin-2-yl, r is 1, and R₃ is 3-hydroxy;
a compound of Formula (Ia) wherein R₁ is 4-t-butyl, A1 is phenyl, L is —(CH₂)₂—, A2 is phenyl, q is 0, R₂ is absent, A3 is 3-pyrimidin-2-yl, r is 0, and R₃ is absent;
a compound of Formula (Ia) wherein R, is 4-t-butyl, A1 is phenyl, L is —(CH₂)₂—, A2 is phenyl, q is 0, R₂ is absent, A3 is 3-pyrimidin-5-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 3-pyridin-2-yl, r is 1, and R₃ is 6-methoxy;
a compound of Formula (Ia) wherein R₁ is 4-t-butyl, A1 is phenyl, L is —(CH₂)₂—, A2 is phenyl, q is 0, R₂ is absent, A3 is 3-pyridin-3-yl, r is 1, and R₃ is 6-methoxy;
a compound of Formula (Ia) wherein R₁ is 4-t-butyl, A1 is phenyl, L is —(CH₂)₂—, A2 is phenyl, q is 0, R₂ is absent, A3 is 4-pyridin-2-yl, r is 1, and R₃ is 6-methoxy;
a compound of Formula (Ia) wherein R₁ is 4-t-butyl, A1 is phenyl, L is —(CH₂)₂—, A2 is phenyl, q is 0, R₂ is absent, A3 is 4-pyridin-3-yl, r is 1, and R₃ is 6-methoxy;
a compound of Formula (Ia) wherein R₁ is 4-t-butyl, A1 is phenyl, L is —(CH₂)₂—, A2 is phenyl, q is 0, R₂ is absent, A3 is 4-1 H-indol-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-1H-indol-6-yl, r is 0, and R₃ is absent;
a compound of Formula (Ia) wherein R₁ is 4-t-butyl, A1 is phenyl, L is —(CH₂)₂—, A2 is pyridin-2-yl, q is 0, R₂ is absent, A3 is 5-phenyl, r is 1, and R₃ is 3-aminocarbonyl;
a compound of Formula (Ia) wherein R₁ is 4-t-butyl, A1 is phenyl, L is —(CH₂)₂—, A2 is pyridin-2-yl, q is 0, R₂ is absent, A3 is 5-phenyl, r is 1, and R₃ is 3-methylcarbonylamino;
a compound of Formula (Ia) wherein R₁ is 4-t-butyl, A1 is phenyl, L is —(CH₂)₂—, A2 is pyridin-2-yl, q is 0, R₂ is absent, A3 is 6-phenyl, r is 1, and R₃ is 3-hydroxy;
a compound of Formula (Ia) wherein R₁ is 4-t-butyl, A1 is phenyl, L is —(CH₂)₂—, A2 is pyridin-3-yl, q is-0, R₂ is absent, A3 is 6-phenyl, r is 1, and R₃ is 3-aminocarbonyl;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 3-methylcarbonylamino;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 3-hydroxy;
a compound of Formula (Ia) wherein R₁ is 4-t-butyl, A1 is phenyl, L is —(CH₂)₂—, A2 is pyrimidin-2-yl, q is 0, R₂ is absent, A3 is 5-phenyl, r is 1, and R₃ is 3-aminocarbonyl;
a compound of Formula (Ia) wherein R₁ is 4-t-butyl, A1 is phenyl, L is —(CH₂)₂—, A2 is pyrazin-2-yl, q is 0, R₂ is absent, A3 is 5-phenyl, r is 1, and R₃ is 3-aminocarbonyl;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 2-hydroxy;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 4-hydroxy;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 3-hydroxymethyl;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 4-hydroxymethyl;
a compound of Formula (Ia) wherein R₁ is 4-t-butyl, A1 is phenyl, L is —(CH₂)₂—, A2 is pyridin-3-yl, q is 0, R₂ is absent, A3 is 6-pyridin-3-yl, r is 0, and R₃ absent;
a compound of Formula (Ia) wherein R₁ is 4-t-butyl, A1 is phenyl, L is —(CH₂)₂—, A2 is pyridin-3-yl, q is 0, R₂ is absent, A3 is 6-pyridin-4-yl, r is 0, and R₃ is absent;
a compound of Formula (Ia) wherein R₁ is 4-t-butyl, A1 is thiophen-2-yl, L is —(CH₂)₂—, A2 is phenyl, q is 0, R₂ is absent, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 2-carboxy, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 2-hydroxymethyl, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-carboxy, A3 is 4-pyridin-4-yl, r is 0, and R₃ is absent;
a compound of Formula (Ia) wherein R₁ is 4-trifluoromethyl A1 is phenyl, L is —(CH₂)₂—, A2 is phenyl, q is 0, R₂ is absent, A3 is 4-pyridin-3-yl, r is 0, and R₃ is absent;
a compound of Formula (Ia) wherein R₁ is 4-trifluoromethyl, A1 is phenyl, L is —(CH₂)₂—, A2 is phenyl, q is 0, R₂ is absent, A3 is 4-pyridin-4-yl, r is 0, and R₃ is absent;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-phenyl, r is 1, and R₃ is 3-aminocarbonyl;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-phenyl, r is 1, and R₃ is 3-methylcarbonylamino;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-phenyl, r is 1, and R₃ is 2-hydroxy;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-phenyl, r is 1, and R₃ is 3-hydroxy;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-phenyl, r is 1, and R₃ is 4-hydroxy;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-phenyl, r is 1, and R₃ is 3-hydroxymethyl;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-phenyl, r is 1, and R₃ is 4-hydroxymethyl;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-pyridin-3-yl, r is 0, and R₃ is absent;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-pyridin-4-yl, r is 0, and R₃ is absent;
a compound of Formula (Ia) wherein R₁ is 4-t-butyl, A1 is phenyl, L is —(CH₂)₂—, A2 is naphthalen-1-yl, q is 0, R₂ is absent, A3 is 4-pyridin-4-yl, r is 0, and R₃ is absent;
a compound of Formula (Ia) wherein R₁ is 4-t-butyl, A1 is phenyl, L is —(CH₂)₂—, A2 is isoquinolin-5-yl, q is 0, R₂ is absent, A3 is 8-phenyl, r is 1, and R₃ is 3-methylcarbonylamino;
a compound of Formula (Ia) wherein R₁ is 4-t-butyl, A1 is phenyl, L is —(CH₂)₂—, A2 is isoquinolin-5-yl, q is 0, R₂ is absent, A3 is 8-phenyl, r is 1, and R₃ is 3-hydroxy;
a compound of Formula (Ia) wherein R₁ is 4-t-butyl, A1 is phenyl, L is —(CH₂)₂—, A2 is isoquinolin-5-yl, q is 0, R₂ is absent, A3 is 8-pyridin-4-yl, r is 0, and R₃ is absent;
a compound of Formula (Ia) wherein R₁ is 4-trifluoromethyl, A1 is phenyl, L is —(CH₂)₂—, A2 is naphthalen- -yl, q is 0, R₂ is absent, A3 is 4-pyridin-4-yl, r is 0, and R₃ is absent;
a compound of Formula (Ia) wherein R₁ is 4-trifluoromethyl, A1 is phenyl, L is —(CH₂)₂—, A2 is isoquinolin-5-yl, q is 0, R₂ is absent, A3 is 8-phenyl, r is 1, and R₃ is 3-hydroxy;
a compound of Formula (Ia) wherein R₁ is 4-trifluoromethyl, A1 is-phenyl, L is —(CH₂)₂—, A2 is isoquinolin-5-yl, q is 0, R₂ is absent, A3 is 8-pyridin-4-yl, r is 0, and R₃ is absent;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-phenyl, r is 1, and R₃ is 2-hydroxy;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-phenyl, r is 1, and R₃ is 3-hydroxy;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-phenyl, r is 1, and R₃ is 4-hydroxy;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-phenyl, r is 1, and R₃ is 4-hydroxymethyl;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-pyridin-3-yl, r is 0, and R₃ is absent;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-pyridin-4-yl, r is 0, and R₃ is absent;
a compound of Formula (Ia) wherein R₁ is 4-t-butyl, A1 is phenyl, L is —(CH₂)₂—, A2 is phenyl, q is 2, R₂ is 3,5-difluoro, A3 is 4-pyridin-3-yl, r is 0, and R₃ is absent;
a compound of Formula (Ia) wherein R₁ is 4-t-butyl, A1 is phenyl, L is —(CH₂)₂—, A2 is phenyl, q is 2, R₂ is 3,5-dimethyl, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 2, R₂ is 3,5-difluoro, A3 is 4-pyridin-3-yl, r is 0, and R₃ is absent;
a compound of Formula (Ia) wherein R₁ is 4-t-butyl, A1 is phenyl, L is —(CH₂)₂—, A2 is phenyl, q is 2, R₂ is 3,5-difluoro, A3 is 4-pyridin-4-yl, r is 0, and R₃ is absent,
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-pyridin-3-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-methyl, A3 is 4-pyridin-2-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-fluoro, A3 is 4-pyridin-2-yl, r is 0, and R₃ is absent;
a compound of Formula (Ia) wherein R₁ is 4-t-butyl, A1 is phenyl, L is —(CH₂)₂—, A2 is phenyl, q is 2, R₂ is 3,5-dimethyl, A3 is 4-pyridin-2-yl, r is 0, and R₃ is absent;
a compound of Formula (Ia) wherein R₁ is 4-t-butyl, A1 is phenyl, L is —(CH₂)₂—, A2 is phenyl, q is 2, R₂ is 3,5-difluoro, A3 is 4-pyridin-2-yl, r is 0, and R₃ is absent;
a compound of Formula (Ia) wherein R₁ is 4-t-butyl, A1 is phenyl, L is —(CH₂)₂—, A2 is pyridin-3-yl, q is 0, R₂ is absent, A3 is 6-pyridin-2-yl, r is 0, and R₃ is absent;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-pyridin-2-yl, r is 0, and R₃ is absent;
a compound of Formula (Ia) wherein R₁ is 4-t-butyl, A1 is phenyl, L is —(CH₂)₂—, A2 is pyrazin-2-yl, q is 0, R₂ is absent, A3 is 5-pyridin-2-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-methyl, A3 is 4-pyridin-3-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-methyl, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-fluoro, A3 is 4-pyridin-3-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-fluoro, A3 is 4-pyridin-4-yl, r is 0, and R₃ is absent;
a compound of Formula (Ia) wherein R₁ is 4-t-butyl, A1 is phenyl, L is —(CH₂)₂—, A2 is pyrazin-2-yl, q is 0, R₂ is absent, A3 is 5-phenyl, r is 1, and R₃ is 2-hydroxy;
a compound of Formula (Ia) wherein R₁ is 4-t-butyl, A1 is phenyl, L is —(CH₂)₂—, A2 is pyrazin-2-yl, q is 0, R₂ is absent, A3 is 5-phenyl, r is 1, and R₃ is 3-hydroxy;
a compound of Formula (Ia) wherein R₁ is 4-t-butyl, A1 is phenyl, L is —(CH₂)₂—, A2 is pyrazin-2-yl, q is 0, R₂ is absent, A3 is 5-phenyl, r is 1, and R₃ is 4-hydroxy;
a compound of Formula (Ia) wherein R₁ is 4-t-butyl, A1 is phenyl, L is —(CH₂)₂—, A2 is pyrazin-2-yl, q is 0, R₂ is absent, A3 is 5-phenyl, r is 1, and R₃ is 4-hydroxymethyl;
a compound of Formula (Ia) wherein R₁ is 4-t-butyl, A1 is phenyl, L is —(CH₂)₂—, A2 is pyrazin-2-yl, q is 0, R₂ is absent, A3 is 5-pyridin-3-yl, r is 0, and R₃ is absent;
a compound of Formula (Ia) wherein R₁ is 4-t-butyl, A1 is phenyl, L is —(CH₂)₂—, A2 is pyrazin-2-yl, q is 0, R₂ is absent, A3 is 5-pyridin-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-imidazol-2-yl, r is 1, and R₃ is 1-methyl;
a compound of Formula (Ia) wherein R₁ is 4-t-butyl, A1 is phenyl, L is —(CH₂)₂—, A2 is phenyl, q is 0, R₂ is absent, A3 is 4-imidazol-4-yl, r is 1, and R₃ is 1-methyl;
a compound of Formula (Ia) wherein R₁ is 4-t-butyl, A1 is phenyl, L is —(CH₂)₂—, A2 is phenyl, q is 0, R₂ is absent, A3 is 4-imidazol-5-yl, r is 1, and R₃ is 1-methyl;
a compound of Formula (Ia) wherein R₁ is 4-t-butyl, A1 is phenyl, L is —(CH₂)₂—, A2 is phenyl, q is 0, R₂ is absent, A3 is 4-imidazol-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-oxazol-5-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-(4,5-dihydro-oxazol-5-yl), r is 0, and R₃ is absent;
a compound of Formula (Ia) wherein R₁ is 4-t-butyl, A1 is phenyl, L is ′—(CH₂)₂—, A2 is phenyl, q is 0, R₂ is absent, A3 is 4-thiazol-4-yl, r is 1, and R₃ is 2-methyl;
a compound of Formula (Ia) wherein R₁ is 4-t-butyl, A1 is phenyl, L is —(CH₂)₂—, A2 is phenyl, q is 0, R₂ is absent, A3 is 4-pyrazol-1-yl, r is 2, and R₃ is 3,5-dimethyl
a compound of Formula (Ia) wherein R₁ is 4-t-butyl, A1 is phenyl, L is —(CH₂)₂—, A2 is phenyl, q is 0, R₂ is absent, A3 is 4-thiadiazol-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-(1,2,4-triazol-1-yl), r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-1 H-tetrazol-5-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-imidazol-3-yl, r is 2, and R₃ is 4,5-dichloro;
a compound of Formula (Ia) wherein R₁ is 4-t-butyl, A1 is phenyl, L is —(CH₂)₂—, A2 is phenyl, q is 0, R₂ is absent, A3 is 4-(2,4-dihydro-pyrazol-2-yl), r is 2, and R₃ is 3-oxo, 5-methyl;
a compound of Formula (Ia) wherein R₁ is 4-t-butyl, A1 is phenyl, L is —(CH₂)₂—, A2 is pyridin-3-yl, q is 1, R₂ is 5-trifluoromethyl, A3 is 6-imidazol-1-yl, r is 0, and R₃ is absent;
a compound of Formula (Ia) wherein R₁ is 4-trifluoromethyl, A1 is phenyl, L is —(CH₂)₂—, A2 is pyridin-3-yl, q is 1, R₂ is 5-methyl, A3 is 6-imidazol-1-yl, r is 2, and R₃ is 4,5-dichloro;
a compound of Formula (Ia) wherein R₁ is 4-trifluoromethyl, A1 is phenyl, L is —(CH₂)₂—, A2 is phenyl, q is 1, R2 is 3-trifluoromethyl, A3 is 4-imidazol-1-yl, r is 1, and R₃ is 4-methyl;
a compound of Formula (Ia) wherein R₁ is 4-trifluoromethylsulphonyl, A1 is phenyl, L is trans —CH═CH—, A2 is phenyl, q is 1, R₂ is 3-trifluoromethyl, A3 is 4-imidazol-1-yl, r is 1, and R₃ is 4-methyl;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-phenyl, and r is 1, R₃ is 3-hydroxy;
a compound of Formula (Ia) wherein a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-imidazol-1-yl, r is 0, and R₃ is absent;
a compound of Formula (Ia) wherein R₁ is 4-trifluoromethylthio, A1 is phenyl, L is trans —CH═CH—, A2 is phenyl, q is 1, R₂ is 3-trifluoromethyl, A3 is 4-imidazol-1-yl, r is 1, and R₃ is 4-methyl;
a compound of Formula (Ia) wherein R₁ is 4-trifluoromethylsulphonyl, A1 is phenyl, L is —(CH₂)₂—, A2 is phenyl, q is 1, R₂ is 3-trifluoromethyl, A3 is 4-imidazol-1-yl, r is 1, and R₃ is 4-methyl;
a compound of Formula (Ia) wherein R₁ is 4-trifluoromethyl, A1 is phenyl, L is —(CH₂)₂—, A2 is pyridin-3-yl, q is 1, R₂ is 5-trifluoromethyl, A3 is 6-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 0, R₂ is absent, A3 is 4-imidazol-1-yl, r is 2, and R₃ is 4,5-dichloro;
a compound of Formula (Ia) wherein R₁ is 4-t-butyl, A1 is phenyl, L is —(CH₂)₂—, A2 is phenyl, q is 1, R₂ is 3-trifluoromethyl, A3 is 4-imidazol-1-yl, r is 2, and R₃ is 4,5-dichloro;
a compound of Formula (Ia) wherein R₁ is 4-trifluoromethyl, A1 is phenyl, L is —(CH₂)₂—, A2 is pyridin-3-yl, q is 1, R₂ is 5-trifluoromethyl, A3 is 6-phenyl, r is 1, and R₃ is 2-hydroxy;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-imidazol-1-yl, and r is 0, R₃ is absent;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-imidazol-1-yl, r is 2, and R₃ is 4,5-dichloro;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-phenyl, r is 1, and R₃ is 3-methylcarbonylamino;
a compound of Formula (Ia) wherein R₁ is 4-trifluoromethylsulphonyl, A1 is phenyl, L is trans —CH═CH—, A2 is phenyl, q is 1, R₂ is 3-trifluoromethyl, A3 is 4-imidazol-1-yl, r is 2, and R₃ is 4,5-dichloro;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-imidazol-1-yl, r is 2, and R₃ is 4,5-dichloro;
a compound of Formula (Ia) wherein R₁ is 4-trifluoromethylthio, A1 is phenyl, L is —(CH₂)₂—, A2 is phenyl, q is 1, R₂ is 3-trifluoromethyl, A3 is 4-imidazol-1-yl, r is 1, and R₃ is 4-methyl;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-pyridin-4-yl, and R₃ is absent;
a compound of Formula (Ia) wherein R₁ is 4-trifluoromethyl, A1 is phenyl, L is —(CH₂)₂—, A2 is pyridin-3-yl, q is 1, R₂ is 5-trifluoromethyl, A3 is 6-phenyl, r is 1, and R₃ is 4-hydroxy;
a compound of Formula (Ia) wherein R₁ is 4-trifluoromethylsulphanyl, A1 is phenyl, L is —(CH₂)₂—, A2 is phenyl, q is 1, R₂ is 3-trifluoromethyl, A3 is 4-imidazol-1-yl, r is 1, and. R₃ is 4-methyl;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-phenyl, r is 1, and R₃ is 4-hydroxymethyl;
a compound of Formula (Ia) wherein R₁ is t-butyl, A1 is phenyl, L is —(CH₂)₂—, A2 is pyridin-3-yl, q is 1, R₂ is 5-trifluoromethyl, A3 is 6-imidazol-1-yl, r is 2, and R₃ is 4,5-dichloro;
a compound of Formula (Ia) wherein R₁ is 4-trifluoromethyl, A1 is phenyl, L is —(CH₂)₂—, A2 is pyridin-3-yl, q is 1, R₂ is 5-trifluoromethyl, A3 is 6-phenyl, r is 1, and R₃ is 4-hydroxymethyl;
a compound of Formula (Ia) wherein R₁ is 4-trifluoromethyl, A1 is phenyl, L is —(CH₂)₂—, A2 is pyridin-3-yl, q is 1, R₂ is 5-methyl, A3 is 6-imidazol-1-yl, r is 1, and R₃ is 4-methyl;
a compound of Formula (Ia) wherein ₁ is 4-trifluoromethyl, A1 is phenyl, L is —(CH₂)₂—, A2 is phenyl, q is 1, R₂ is 3-trifluoromethyl, A3 is 4-imidazol-1-yl, r is 1, and R₃ is 4-chloro;
a compound of Formula (Ia) wherein R₁ is 4-trifluoromethylthio, A1 is phenyl, L is trans —CH═CH—, A2 is phenyl, q is 1, R₂ is 3-trifluoromethyl, A3 is 4-imidazol-1-yl, r is 2, and R₃ is 4,5-dichloro;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-phenyl, r is 1, and R₃ is 4-hydroxy;
a compound of Formula (Ia) wherein R₁ is 4-chloro, A1 is phenyl, L is —(CH₂)₂—, A2 is phenyl, q is 1, R₂ is 3-trifluoromethyl, A3 is 4-pyridin-4-yl, r is 0, and R₃ is absent;
a compound of Formula (Ia) wherein R₁ is 4-chloro, A1 is phenyl, L is —(CH₂)₂—, A2 is phenyl, q is 1, R₂ is 3-trifluoromethyl, A3 is 4-imidazol-1-yl, r is 2, and R₃ is 4,5-dichloro;
a compound of Formula (Ia) wherein R₁ is 4-N-cyclohexyl-N-methylamino, A1 is phenyl, L is —(CH₂)₂—, A2 is pyridin-3-yl, q is 1, R₂ is 5-methyl, A3 is 6-imidazol-1-yl, r is 2, and R₃ is 4,5-dichloro;
a compound of Formula (Ia) wherein R₁ is 5-trifluoromethyl, A1 is pyridin-2-yl, L is —(CH₂)₂—, A2 is phenyl, q is 1, R₂ is 3-trifluoromethyl, A3 is 4-imidazol-1-yl, r is 1, and R₃ is 4-methyl;
a compound of Formula (Ia) wherein R₁ is 4-chloro, A1 is phenyl, L is —(CH₂)₂—, A2 is pyridin-3-yl, q is 1, R₂is 5-methyl, A3 is 6-imidazol-1-yl, r is 2, and R₃ is 4,5-dichloro;
a compound of Formula (Ia) wherein R₁ is 5-trifluoromethyl, A1 is pyridin-2-yl, L is trans —CH═CH—, A2 is phenyl, q is 1, R₂ is 3-trifluoromethyl, A3 is 4-imidazol-1-yl, r is 2, and R₃ is 4,5-dichloro;
a compound of Formula (Ia) wherein R₁ is 5-trifluoromethylsulfonyl, A1 is phenyl, L is —(CH₂)₂—, A2 is phenyl, q is 1, R₂ is 3-trifluoromethyl, A3 is 4-imidazol-1-yl, r is 2, and R₃ is 4,5-dichloro;
a compound of Formula (Ia) wherein R₁ is 5-trifluoromethylsulfonyl, A1 is phenyl, L is —(CH₂)₂—, A2 is pyridin-3-yl, q is 1, R₂ is 5-methyl, A3 is 6-imidazol-1-yl, r is 2, and R₃ is 4,5-dichloro;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-imidazol-1-yl, r is 1, and R₃ is 4-trifluoromethyl;
a compound of Formula (Ia) wherein R₁ is 4-trifluoromethylthio, A1 is phenyl, L is —(CH₂)₂—, A2 is phenyl, q is 1, R₂ is 3-trifluoromethyl, A3 is 4-imidazol-1-yl, r is 2, and R₃ is 4,5-dichloro;
a compound of Formula (Ia) wherein R₁ is 4-trifluoromethyl, A1 is phenyl, L is —(CH₂)₂—, A2 is pyridin-3-yl, q is 1, R₂ is 5-trifluoromethyl, A3 is 6-imidazol-1-yl, r is 0, and R₃ is absent;
a compound of Formula (Ia) wherein R₁ is 4-N-cyclohexyl-N-methylamino, A1 is phenyl, L is trans —CH═CH—, A2 is pyridin-3-yl, q is 1, R₂ is 5-methyl, A3 is 6-imidazol-1-yl, r is 2, and R₃ is 4,5-dichloro;
a compound of Formula (Ia) wherein R₁ is 4-chloro, A1 is phenyl, L is —(CH₂)₂—, A2 is phenyl, q is 1, R₂ is 3-trifluoromethyl, A3 is 4-imidazol-1-yl, r is 0, and R₃ is absent;
a compound of Formula (Ia) wherein R₁ is 4-fluoro, A1 is phenyl, L is —(CH₂)₂—, A2 is phenyl, q is 1, R₂ is 3-trifluoromethyl, A3 is 4-imidazol-1-yl, r is 2, and R₃ is 4,5-dichloro;
a compound of Formula (Ia) wherein R₁ is 4-trifluoromethylsulfinyl, A1 is phenyl, L is —(CH₂)₂—, A2 is phenyl, q is 1, R₂ is 3-trifluoromethyl, A3 is 4-imidazol-1-yl, r is 2, and R₃ is 4,5-dichloro;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-(2-oxo-2,3-dihydro-2λ4-[1,2,3,5]oxathiadiazol-4-yl), r is 0, and R₃ is absent;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-phenyl, r is 1, and R₃ is 3-cyano;
a compound of Formula (Ia) wherein R₁ is 4-trifluoromethyl, A1 is phenyl, L is —(CH₂)₂—, A2 is pyridin-3-yl, q is 1, R₂ is 5-methyl, A3 is 6-imidazol-1-yl, r is 0, and R₃ is absent;
a compound of Formula (Ia) wherein R₁ is 5-trifluoromethyl, A1 is pyridin-2-yl, L is —(CH₂)₂—, A2 is phenyl, q is 1, R₂ is 3-trifluoromethyl, A3 is 4-imidazol-1-yl, and r is 2, R₃ is 4,5-dichloro;
a compound of Formula (Ia) wherein R₁ is 4-trifluoromethyl, A1 is phenyl, L is —(CH₂)₂—, A2 is pyridin-3-yl, q is 1, R₂ is 5-trifluoromethyl, A3 is 6-pyridin-3-yl, r is 0, and R₃ is absent;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-phenyl, r is 1, and R₃ is 4-hydroxymethyl;
a compound of Formula (Ia) wherein R₁ is 4-trifluoromethyl, A1 is phenyl, L is —(CH₂)₂—, A2 is pyridin-3-yl, q is 1, R₂ is 5-trifluoromethyl, A3 is 6-imidazol-1-yl, r is 2, and R₃ is 4,5-dichloro;
a compound of Formula (Ia) wherein R₁ is hydrogen, A1 is phenyl, L is —(CH₂)₂—, A2 is phenyl, q is 1, R₂ is 3-trifluoromethyl, A3 is 4-pyridin-4-yl, r is 0, and R₃ is absent;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-benzoimidazol-1-yl, r is 0, and R₃ is absent;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-pyridin-4-yl, r is 1, and R₃ is 2-fluoro;
a compound of Formula (Ia) wherein R₁ is 4-trifluoromethyl, A1 is phenyl, L is —(CH₂)₂—, A2 is pyridin-3-yl, q is 1, R₂ is 5-trifluoromethyl, A3 is 6-pyridin-4-yl, r is 0, and R₃ is absent;
a compound of Formula (Ia) wherein R₁ is 3,4-difluoro, A1 is phenyl, L is —(CH₂)₂—, A2 is phenyl, q is 1, R₂ is 3-trifluoromethyl, A3 is 4-imidazol-1-yl, r is 0, and R₃ is absent;
a compound of Formula (Ia) wherein R₁ is 3,4-difluoro, A1 is phenyl, L is —(CH₂)₂—, A2 is pyridin-3-yl, q is 1, R₂ is 5-methyl, A3 is 6-imidazol-1-yl, r is 2, and R₃ is 4,5-dichloro;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-pyridin-3-yl, r is 1, and R₃ is 2-fluoro;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-phenyl, r is 1, and R₃ is 3-methoxy;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-phenyl, r is 1, and R₃ is 3-nitro;
a compound of Formula (Ia) wherein R₁ is 4-t-butyl, A1 is phenyl, L is —(CH₂)₂—, A2 is phenyl, q is 0, R₂ is absent, A3 is 4-imidazol-1-yl, r is 0, and R₃ is absent;
a compound of Formula (Ia) wherein R₁ is 4-trifluoromethyl, A1 is phenyl, L is —(CH₂)₂—, A2 is phenyl, q is 0, R₂ is absent, A3 is 4-imidazol-1-yl, r is 0, and R₃ is absent;
a compound of Formula (Ia) wherein R₁ is 4-trifluoromethyl, A1 is phenyl, L is —(CH₂)₂—, A2 is phenyl, q is 0, R₂ is absent, A3 is 4-imidazol-1-yl, r is 2, and R₃ is 4,5-dichloro;
a compound of Formula (Ia) wherein R₁ is 4-t-butyl, A1 is phenyl, L is —(CH₂)₂—, A2 is phenyl, q is 1, R₂ is 3-trifluoromethyl, A3 is 4-imidazol-1-yl, r is 0, and R₃ is absent;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-(1,2,4-oxadiazol-3-yl), r is 1, and R₃ is 5-trifluoromethyl;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-(1,2,4-oxadiazol-3-yl), r is 1, and R₃ is 5-methyl;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-imidazol-1-yl, r is 1, and R₃ is 2-methyl;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-imidazol-1-yl, r is 1, and R₃ is 2-isopropyl;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-indol-1-yl, r is 0, and R₃ is absent;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-(4,5,6,7-tetrahydro-benzoimidazol-1-yl), r is 0, and R₃ is absent;
a compound of Formula (Ia) wherein R₁ is 4-trifluoromethyl, A1 is phenyl, L is —(CH₂)₂—, A2 is phenyl, q is 1, R₂ is 3-trifluoromethyl, A3 is 4-imidazol-1-yl, r is 1, and R₃ is 4-methoxycarbonyl;
a compound of Formula (Ia) wherein R₁ is 4-trifluoromethylthio, A1 is phenyl, L is —(CH₂)₂—, A2 is pyridin-3-yl, q is 1, R₂ is 5-methyl, A3 is 6-imidazol-1-yl, r is 2, and R₃ is 4,5-dichloro;
a compound of Formula (Ia) wherein R₁ is 4-trifluoromethylsulfinyl, A1 is phenyl, L is —(CH₂)₂—, A2 is pyridin-3-yl, q is 1, R₂ is 5-methyl, A3 is 6-imidazol-1-yl, r is 2, and R₃ is 4,5-dichloro;
a compound of Formula (Ia) wherein R₁ is 4-trifluoromethylsulfinyl, A1 is phenyl, L is trans —CH═CH—, A2 is pyridin-3-yl, q is 1, R₂ is 5-methyl, A3 is 6-imidazol-1-yl, r is 2, and R₃ is 4,5-dichloro;
a compound of Formula (Ia) wherein R₁ is 4-trifluoromethylthio, A1 is phenyl, L is trans —CH═CH—, A2 is pyridin-3-yl, q is 1, R₂ is 5-methyl, A3 is 6-imidazol-1-yl, r is 2, and R₃ is 4,5-dichloro;
a compound of Formula (Ia) wherein R₁ is 5-trifluoromethyl, A1 is pyridin-2-yl, L is trans —CH═CH—, A2 is pyridin-3-yl, q is 1, R₂ is 5-methyl, A3 is 6-imidazol-1-yl, r is 2, and R₃ is 4,5-dichloro;
a compound of Formula (Ia) wherein R₁ is 5-trifluoromethyl, A1 is pyridin-2-yl, L is trans —CH═CH—, A2 is phenyl, q is 1, R₂ is 3-trifluoromethyl, A3 is 4-imidazol-1-yl, r is 1, and R₃ is 4-methyl;
a compound of Formula (Ia) wherein R₁ is 5-trifluoromethyl, A1 is pyridin-2-yl, L is —(CH₂)₂—, A2 is pyridin-3-yl, q is 1, R₂ is 5-methyl, A3 is 6-imidazol-1-yl, r is 2, and R₃ is 4,5-dichloro;
a compound of Formula (Ia) wherein R₁ is 4-fluoro, A1 is phenyl, L is —(CH₂)₂—, A2 is phenyl, q is 1, R₂ is 3-trifluoromethyl, A3 is 4-imidazol-1-yl, r is 0, and R₃ is absent;
and
a compound of Formula (Ia) wherein R₁ is 4-fluoro, A1 is phenyl, L is —(CH₂)₂—, A2 is pyridin-3-yl, q is 1, R₂ is 5-methyl, A3 is 6-imidazol-1-yl, r is 2, and R₃ is 4,5-dichloro.

49. A compound of Formula Ia 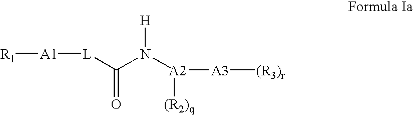
selected from the group consisting of:
a compound of Formula (Ia) wherein R₁ is 4-t-butyl, A1 is phenyl, L is —(CH₂)₂—, A2 is phenyl, q is 0; R₂ is absent; A3 is 2-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 0, R₂ is absent, A3 is 2-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 0, R₂ is absent, A3 is 2-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-nitro;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-trifluoromethyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-methylcarbonyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-chloro;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-fluoro;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-amino;
a compound of Formula (Ia) wherein R₁ is 4-t-butyl, A1 is phenyl, L is —(CH₂)₂—, A2 is phenyl, q is 0, R₂ is absent, A3 is 2-phenyl, r is 1, and R₃ is 3-hydroxymethyl;
a compound of Formula (Ia) wherein R₁ is 4-t-butyl, A1 is phenyl, L is —(CH₂)₂—, A2 is phenyl, q is 0, R₂ is absent, A3 is 2-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 3-hydroxymethyl;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-hydroxymethyl;
a compound of Formula (Ia) wherein R₁ is 4-t-butyl, A1 is phenyl, L is —(CH₂)₂—, A2 is phenyl, q is 0, R₂is absent, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-cyano;
a compound of Formula (Ia) wherein R₁ is 4-t-butyl, A1 is phenyl, L is —(CH₂)₂—, A2 is phenyl, q is 0, R₂is absent, A3 is 4-phenyl, r is 1, and R₃ is 3-trifluoromethoxy,
a compound of Formula (Ia) wherein R₁ is 4-t-butyl, A1 is phenyl, L is —(CH₂)₂—, A2 is phenyl, q is 0, R₂is absent, A3 is 4-phenyl, r is 1, and R₃ is 3-methoxy;
a compound of Formula (Ia) wherein R₁ is 4-t-butyl, A1 is phenyl, L is —(CH₂)₂—, A2 is phenyl, q is 0, R₂is absent, A3 is 4-phenyl, r is 1, and R₃ is 3-methyl;
a compound of Formula (Ia) wherein R₁ is 4-t-butyl, A1 is phenyl, L is —(CH₂)₂—, A2 is phenyl, q is 0, R₂is absent, A3 is 4-phenyl, r is 1, and R₃ is 3-methoxycarbonyl;
a compound of Formula (Ia) wherein R₁ is 4-t-butyl, A1 is phenyl, L is —(CH₂)₂—, A2 is phenyl, q is 0, R₂is absent, A3 is 4-phenyl, r is 1, and R₃ is 3-carboxy;
a compound of Formula (Ia) wherein R₁ is 4-t-butyl, A1 is phenyl, L is —(CH₂)₂—, A2 is phenyl, q is 0, R₂is absent, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-aminocarbonyl;
a compound of Formula (Ia) wherein R₁ is 4-t-butyl, A1 is phenyl, L is trans —CH═CH—, A2 is phenyl, q is 0, R₂is absent, A3 is 3-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 3-dimethylaminocarbonyl;
a compound of Formula (Ia) wherein R₁ is 4-t-butyl, A1 is phenyl, L is trans —CH═CH—, A2 is phenyl, q is 0, R₂ is absent, A3 is 4-phenyl, r is 1, and R₃ is 3-aminocarbonyl;
a compound of Formula (Ia) wherein R₁ is 4-t-butyl, A1 is phenyl, L is trans —CH═CH—, A2 is phenyl, q is 0, R₂ is absent, A3 is 4-phenyl, r is 1, and R₃ is 3-dimethylaminocarbonyl;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 0, R₂ is absent, A3 is 3-phenyl, r is 1, and R₃ is 3-dimethylaminocarbonyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-dimethylaminocarbonyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-methylsulfonylamino;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-t-butoxycarbonylamino;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-t-butoxycarbonylaminomethyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-aminomethyl;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-ureido;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-(2-amino-ethoxy);
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-(2-hydroxy-ethylamino);
a compound of Formula (Ia) wherein R₁ is 4-trifluoromethyl, A1 is phenyl, L is trans —CH═CH—, A2 is phenyl, q is 0, R₂ is absent, A3 is 4-phenyl, r is 1, and R₃ is 3-aminocarbonyl;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 2-methyl, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 2-fluoro, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 3-fluoro, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 2-methyl, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 2-fluoro, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 3-fluoro, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 2-methyl, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 2-fluoro, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 3-fluoro, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 0, R₂ is absent, A3 is 4-phenyl, r is 1, and R₃ is 3-hydroxy;
a compound of Formula (Ia) wherein R₁ is 5-t-butyl, A1 is thiophen-2-yl, L is —(CH₂)₂—, A2 is phenyl, q is 0, R₂ is absent, A3 is 4-phenyl, r is 1, and R₃ is 3-aminocarbonyl;
a compound of Formula (Ia) wherein R₁ is 5-t-butyl, A1 is thiophen-2-yl, L is —(CH₂)₂—, A2 is phenyl, q is 0, R₂ is absent, A3 is 4-phenyl, r is 1, and R₃ is 3-methylcarbonylamino;
a compound of Formula (Ia) wherein R₁ is 5-t-butyl, A1 is thiophen-2-yl, L is —(CH₂)₂—, A2 is phenyl, q is 0, R₂ is absent, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 2-carboxy, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 2-carboxy, A3 is 4-phenyl, r is 1, and R₃ is 3-methylaminocarbonyl;
a compound of Formula (Ia) wherein R₁ is 4-t-butyl, A1 is phenyl, L is —(CH₂)₂—, A2 is phenyl, q is 1, R₂ is 2-carboxy, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 2-hydroxymethyl, A3 is 4-phenyl, r is 1, and R₃ is 3-aminocarbonyl;
a compound of Formula (Ia) wherein R₁ is 4-t-butyl, A1 is phenyl, L is —(CH₂)₂—, A2 is phenyl, q is 1, R₂ is 2-hydroxymethyl, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 2-hydroxymethyl, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 3-carboxy, A3 is 4-phenyl, r is 1, and R₃ is 3-aminocarbonyl;
a compourid of Formula (Ia) wherein R₁ is 4-t-butyl, A1 is phenyl, L is —(CH₂)₂—, A2 is phenyl, q is 1, R₂ is 3-carboxy, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is phenyl, q is 1, R₂ is 3-carboxy, A3 is 4-phenyl, r is 1, and R₃ is 3-hydroxy;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 3-methylcarbonylamino;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 2-hydroxy;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 3-hydroxy;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 4-hydroxy;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 3-hydroxymethyl;
a compound of Formula (Ia) wherein R₁ is 4-trifluoromethyl, A1 is phenyl, L is —(CH₂)₂—, A2 is phenyl, q is 0, R₂ is absent, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is naphthalen-1-yl, q is 0, R₂ is absent, A3 is 4-phenyl, r is 1, and R₃ is 3-methylcarbonylamino;
a compound of Formula (Ia) wherein R₁ is 4-t-butyl, A1 is phenyl, L is —(CH₂)₂—, A2 is naphthalen-1-yl, q is 0, R₂ is absent, A3 is 4-phenyl, r is 1, and R₃ is 3-hydroxy;
a compound of Formula (Ia) wherein R₁ is 4-trifluoromethyl, A1 is phenyl, L is —(CH₂)₂—, A2 is naphthalen-1-yl, q is 0, R₂ is absent, A3 is 4-phenyl, r is 1, and R₃ is 3-aminocarbonyl;
a compound of Formula (Ia) wherein R₁ is 4-trifluoromethyl, A1 is phenyl, L is —(CH₂)₂—, A2 is naphthalen-1-yl, q is 0, R₂ is absent, A3 is 4-phenyl, r is 1, and R₃ is 3-methylcarbonylamino;
a compound of Formula (Ia) wherein R₁ is 4-trifluoromethyl, A1 is phenyl, L is —(CH₂)₂—, A2 is naphthalen-1-yl, q is 0, R₂ is absent, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 2, R₂ is 3,5-dimethyl, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 2, R₂ is 3,5-dimethyl, A3 is 4-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 2, R₂ is 3,5-dimethyl, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 2, R₂ is 3,5-difluoro, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 2, R₂ is 3,5-difluoro, A3 is 4-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 2, R₂ is 3,5-difluoro, A3 is. 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-phenyl, r is 1, and R₃ is 3-hydroxy;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-phenyl, r is 1, and R₃ is 3-hydroxymethyl;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 4-hydroxy;
a compound of Formula (Ia) wherein R₁ is 4-t-butyl, A1 is phenyl, L is —(CH₂)₂—, A2 is phenyl, q is 1, R₂ is 3-methyl, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 1, R₂ is 3-fluoro, A3 is 4-phenyl, r is 1, and R₃ is 2-hydroxy;
a compound of Formula (Ia) wherein R₁ is 4-t-butyl, A1 is phenyl, L is —(CH₂)₂—, A2 is phenyl, q is 1, R₂ is 3-fluoro, A3 is 4-phenyi, r is 1, and R₃ is 4-hydroxy
a compound of Formula (Ia) wherein R₁ is 4-t-butyl, A1 is phenyl, L is —(CH₂)₂—, A2 is phenyl, q is 1, R₂ is 3-fluoro, A3 is 4-phenyl, r is 1, and R₃ is 4-hydroxymethyl;
a compound of Formula (Ia) wherein R₁ is 4-t-butyl, A1 is phenyl, L is —(CH₂)₂—, A2 is phenyl, q is 0, R₂ is absent, A3 is 2-pyridin-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 3-pyridin-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 2-pyridin-3-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 3-pyridin-3-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-pyridin-3-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 2-pyridin-2-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 3-pyridin-2-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-pyridin-2-yl, r is 0, and R₃ is absent;
a compound of Formula (Ia) wherein R₁ is 4-t-butyl, A1. is phenyl, L is —(CH₂)₂—, A2 is phenyl, q is 0, R₂ is absent, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-pyrimidin-2-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-pyrimidin-5-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 3-pyridin-2-yl, r is 1, and R₃ is 6-hydroxy;
a compound of Formula (Ia) wherein R₁ is 4-t-butyl, A1 is phenyl, L is —(CH₂)₂—, A2 is phenyl, q is 0, R₂ is absent, A3 is 4-pyridin-2-yl, r is 1, and R₃ is 3-hydroxy;
a compound of Formula (Ia) wherein R₁ is 4-t-butyl, A1 is phenyl, L is —(CH₂)₂—, A2 is phenyl, q is 0, R₂ is absent, A3 is 3-pyrimidin-2-yl, r is 0, and R₃ is absent;
a compound-of Formula (Ia) wherein R₁ is 4-t-butyl, A1 is phenyl, L is —(CH₂)₂—, A2 is phenyl, q is 0, R₂ is absent, A3 is 3-pyrimidin-5-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 3-pyridin-2-yl, r is 1, and R₃ is 6-methoxy;
a compound of Formula (Ia) wherein R₁ is 4-t-butyl, A1 is phenyl, L is —(CH₂)₂—, A2 is phenyl, q is 0, R₂ is absent, A3 is 3-pyridin-3-yl, r is 1, and R₃ is 6-methoxy;
a compound of Formula (Ia) wherein R₁ is 4-t-butyl, A1 is phenyl, L is —(CH₂)₂—, A2 is phenyl, q is 0, R₂ is absent, A3 is 4-pyridin-2-yl, r is 1, and R₃ is 6-methoxy;
a compound of Formula (Ia) wherein R₁ is 4-t-butyl, A1 is phenyl, L is —(CH₂)₂—, A2 is phenyl, q is 0, R₂ is absent, A3 is 4-pyridin-3-yl, r is 1, and R₃ is 6-methoxy;
a compound of Formula (Ia) wherein R₁ is 4-t-butyl, A1 is phenyl, L is —(CH₂)₂—, A2 is phenyl, q is 0, R₂ is absent, A3 is 4-1 H-indol-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-1 H-indol-6-yl, r is 0, and R₃ is absent;
a compound of Formula (Ia) wherein R₁ is 4-t-butyl, A1 is phenyl, L is —(CH₂)₂—, A2 is pyridin-2-yl, q is 0, R₂ is absent, A3 is 5-phenyl, r is 1, and R₃ is 3-aminocarbonyl;
a compound of Formula (Ia) wherein R₁ is 4-t-butyl, A1 is phenyl, L is —(CH₂)₂—, A2 is pyridin-2-yl, q is 0, R₂ is absent, A3 is 5-phenyl, r is 1, and R₃ is 3-methylcarbonylamino;
a compound of Formula (Ia) wherein R₁ is 4-t-butyl, A1 is phenyl, L is —(CH₂)₂—, A2 is pyridin-2-yl, q is 0, R₂ is absent, A3 is 6-phenyl, r is 1, and R₃ is 3-hydroxy;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 3-aminocarbonyl;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 3-methylcarbonylamino;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 3-hydroxy;
a compound of Formula (Ia) wherein R₁ is 4-t-butyl, A1 is phenyl, L is-(CH₂)₂—, A2 is pyrimidin-2-yl, q is 0, R₂ is absent, A3 is 5-phenyl, r is 1, and R₃ is 3-aminocarbonyl;
a compound of Formula (Ia) wherein R₁ is 4-t-butyl, A1 is phenyl, L is —(CH₂)₂—, A2 is pyrazin-2-yl, q is 0, R₂ is absent, A3 is 5-phenyl, r is 1, and R₃ is 3-aminocarbonyl;
a compound of Formula (Ia) wherein R₁ is 4-t-butyl, A1 isfphenyl, L is —(CH₂)₂—, A2 is pyridin-3-yl, q is 0, R₂ is absent, A3 is 6-phenyl, r is 1, and R₃ is 2-hydroxy;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 4-hydroxy;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 3-hydroxymethyl;
a compound of Formula (Ia) wherein R₁ is 4-t-butyl, A1 is phenyl, L is —(CH₂)₂—, A2 is pyridin-3-yl, q is 0, R₂ is absent, A3 is 6-phenyl, r is 1, and R₃ is 4-hydroxymethyl;
a compound of Formula (Ia) wherein R₁ is 4-t-butyl, A1 is phenyl, L is —(CH₂)₂—, A2 is pyridin-3-yl, q is 0, R₂ is absent, A3 is 6-pyridin-3-yl, r is 0, and R₃ is absent;
a compound of Formula (Ia) wherein R₁ is 4-t-butyl, A1 is phenyl, L is —(CH₂)₂—, A2 is pyridin-3-yl, q is 0, R₂ is absent, A3 is 6-pyridin-4-yl, r is 0, and R₃ is absent;
a compound of Formula (Ia) wherein R₁ is 4-t-butyl, A1 is thiophen-2-yl, L is —(CH₂)₂—, A2 is phenyl, q is 0, R₂ is absent, A3 is 4-pyridin-4-yl, r is 0, and absent;
a compound of Formula (Ia) wherein R₁ is 4-t-butyl, A1 is phenyl, L is —(CH₂)₂—, A2 is phenyl, q is 1, R₂ is 2-carboxy, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 2-hydroxymethyl, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-carboxy, A3 is 4-pyridin-4-yl, r is 0, and R₃ is absent;
a compound of Formula (Ia) wherein R₁ is 4-trifluoromethyl, A1 is phenyl, L is —(CH₂)₂—, A2 is phenyl, q is 0, R₂ is absent, A3 is 4-pyridin-3-yl, r is 0, and R₃ is absent;
a compound of Formula (Ia) wherein R₁ is 4-trifluoromethyl, A1 is phenyl, L is —(CH₂)₂—, A2 is phenyl, q is 0, R₂ is absent, A3 is 4-pyridin-4-yl, r is 0, and R₃ is absent;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-phenyl, r is 1, and R₃ is 3-aminocarbonyl;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-phenyl, r is 1, and R₃ is 3-methylcarbonylamino;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-phenyl, r is 1, and R₃ is 2-hydroxy;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-phenyl, r is 1, and R₃ is 3-hydroxy;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-phenyl, r is 1, and R₃ is 4-hydroxy;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-phenyl, r is 1, and R₃ is 3-hydroxymethyl;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-phenyl, r is 1, and R₃ is 4-hydroxymethyl;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-pyridin-3-yl, r is 0, and R₃ is absent;
a compound of Formula (Ia) wherein R₁ is 4-trifluoromethyl, A1 is phenyl, L is —(CH₂)₂—, A2 is pyridin-3-yl, q is 0, R₂ is absent, A3 is 6-pyridin-4-yl, r is 0, and R₃ is absent;
a compound of Formula (Ia) wherein R₁ is 4-t-butyl, A1 is phenyl, L is —(CH₂)₂—, A2 is naphthalen-1-yl, q is 0, R₂ is absent, A3 is 4-pyridin-4-yl, r is 0, and R₃ is absent;
a compound of Formula (Ia) wherein R₁ is 4-t-butyl, A1 is phenyl, L is —(CH₂)₂—, A2 is isoquinolin-5-yl, q is 0, R₂ is absent, A3 is 8-phenyl, r is 1, and R₃ is 3-methylcarbonylamino;
a compound of Formula (Ia) wherein R₁ is 4-t-butyl, A1 is phenyl, L is —(CH₂)₂—, A2 is isoquinolin-5-yl, q is 0, R₂ is absent, A3 is 8-phenyl, r is 1, and R₃ is 3-hydroxy;
a compound of Formula (Ia) wherein R₁ is 4-t-butyl, A1 is phenyl, L is —(CH₂)₂—, A2 is isoquinolin-5-yl, q is 0, R₂ is absent, A3 is 8-pyridin-4-yl, r is 0, and R₃ is absent;
a compound of Formula (Ia) wherein R₁ is 4-trifluoromethyl, A1 is phenyl, L is —(CH₂)₂—, A2 is naphthalen- -yl, q is 0, R₂ is absent, A3 is 4-pyridin-4-yl, r is 0, and R₃ is absent;
a compound of Formula (Ia) wherein R₁ is 4-trifluoromethyl, A1 is phenyl, L is —(CH₂)₂—, A2 is isoquinolin-5-yl, q is 0, R₂ is absent, A3 is 8-phenyl, r is 1, and R₃ is 3-hydroxy;
a compound of Formula (Ia) wherein R₁ is 4-trifluoromethyl, A1 is phenyl, L is —(CH₂)₂—, A2 is isoquinolin-5-yl, q is 0, R₂ is absent, A3 is 8-pyridin-4-yl, r is 0, and R₃ is absent;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-phenyl, r is 1, and R₃ is 2-hydroxy;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-phenyl, r is 1, and R₃ is 3-hydroxy;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-phenyl, r is 1, and R₃ is 4-hydroxy;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-phenyl, r is 1, and R₃ is 4-hydroxymethyl;
a compound of Formula (Ia) wherein R₁ is 4-t-butyl, A1 is phenyl,. L is —(CH₂)₂—, A2 is pyridin-3-yl, q is 1, R₂ is 5-methyl, A3 is 6-pyridin-3-yl, r is 0, and R₃ is absent;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-pyridin-4-yl, r is 0, and R₃ is absent;
a compound of Formula (Ia) wherein R₁ is 4-t-butyl, A1 is phenyl, L is —(CH₂)₂—, A2 is phenyl, q is 2, R₂ is 3,5-difluoro, A3 is 4-pyridin-3-yl, r is 0, and R₃ is absent;
a compound of Formula (Ia) wherein R₁ is 4-t-butyl, A1 is phenyl, L is —(CH₂)₂—, A2 is phenyl, q is 2, R₂ is 3,5-dimethyl, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 2, R₂ is 3,5-difluoro, A3 is 4-pyridin-3-yl, r is 0, and R₃ is absent;
a compound of Formula (Ia) wherein R₁ is 4-t-butyl, A1 is phenyl, L is —(CH₂)₂—, A2 is phenyl, q is 2, R₂ is 3,5-difluoro, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-pyridin-3-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-hydroxymethyl, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-methyl, A3 is 4-pyridin-2-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-fluoro, A3 is 4-pyridin-2-yl, r is 0, and R₃ is absent;
a compound of Formula (Ia) wherein R₁ is 4-t-butyl, A1 is phenyl, L is —(CH₂)₂—, A2 is phenyl, q is 2, R₂ is 3,5-dimethyl, A3 is 4-pyridin-2-yl, r is 0, and R₃ is absent;
a compound of Formula (Ia) wherein R₁ is 4-t-butyl, A1 is phenyl, L is —(CH₂)₂—, A2 is phenyl, q is 2, R₂ is 3,5-difluoro, A3 is 4-pyridin-2-yl, r is 0, and R₃ is absent;
a compound of Formula (Ia) wherein R₁ is 4-t-butyl, A1 is phenyl, L is —(CH₂)₂—, A2 is pyridin-3-yl, q is 0, R₂ is absent, A3 is 6-pyridin-2-yl, r is 0, and R₃ is absent;
a compound of Formula (Ia) wherein R₁ is 4-t-butyl, A1 is phenyl, L is —(CH₂)₂—, A2 is pyridin-3-yl, q is 1, R₂ is 5-methyl, A3 is 6-pyridin-2-yl, r is 0, and R₃ absent;
a compound of Formula (Ia) wherein R₁ is 4-t-butyl, A1 is phenyl, L is —(CH₂)₂—, A2 is pyrazin-2-yl, q is 0, R₂ is absent, A3 is 5-pyridin-2-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-methyl, A3 is 4-pyridin-3-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-methyl, A3 is 4-pyridin-4-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-fluoro, A3 is 4-pyridin-3-yl, r is 0, and R₃ is absent;
a compound of Formula (Ia) wherein R₁ is 4-t-butyl, A1 is phenyl, L is —(CH₂)₂—, A2 is phenyl, q is 1, R₂ is 3-fluoro, A3 is 4-pyridin-4-yl, r is 0, and R₃ is
a compound of Formula (Ia) wherein R₁ is 4-t-butyl, A1 is phenyl, L is —(CH₂)₂—, A2 is pyrazin-2-yl, q is 0, R₂ is absent, A3 is 5-phenyl, r is 1, and R₃ is 2-hydroxy;
a compound of Formula (Ia) wherein R₁ is 4-t-butyl, A1 is phenyl, L is —(CH₂)₂—, A2 is pyrazin-2-yl, q is 0, R₂ is absent, A3 is 5-phenyl, r is 1, and R₃ is 3-hydroxy;
a compound of Formula (Ia) wherein R₁ is 4-t-butyl, A1 is phenyl, L is —(CH₂)₂—, A2 is pyrazin-2-yl, q is 0, R₂ is absent, A3 is 5-phenyl, r is 1, and R₃ is 4-hydroxy;
a compound of Formula (Ia) wherein R₁ is 4-t-butyl, A1 is phenyl, L is —(CH₂)₂—, A2 is pyrazin-2-yl, q is 0, R₂ is absent, A3 is 5-phenyl, r is 1, and R₃ is 4-hydroxymethyl;
a compound of Formula (Ia) wherein R₁ is 4-t-butyl, A1 is phenyl, L is —(CH₂)₂—, A2 is pyrazin-2-yl, q is 0, R₂ is absent, A3 is 5-pyridin-3-yl, r is 0, and R₃ is absent;
a compound of Formula (Ia) wherein R₁ is 4-t-butyl, A1 is phenyl, L is —(CH₂)₂—, A2 is pyrazin-2-yl, q is 0, R₂ is absent, A3 is 5-pyridin-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-imidazol-2-yl, r is 1, and R₃ is 1-methyl;
a compound of Formula (Ia) wherein R₁ is 4-t-butyl, A1 is phenyl, L is —(CH₂)₂—, A2 is phenyl, q is 0, R₂ is absent, A3 is 4-imidazol-4-yl, r is 1, and R₃ is 1-methyl;
a compound of Formula (Ia) wherein R₁ is 4-t-butyl, A1 is phenyl, L is —(CH₂)₂—, A2 is phenyl, q is 0, R₂ is absent, A3 is 4-imidazol-5-yl, r is 1, and R₃ is 1-methyl;
a compound of Formula (Ia) wherein R₁ is 4-t-butyl, A1 is phenyl, L is —(CH₂)₂—, A2 is phenyl, q is 0, R₂ is absent, A3 is 4-imidazol-4-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-oxazol-5-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-(4,5-dihydro-oxazol-5-yl), r is 0, and R₃ is absent;
a compound of Formula (Ia) wherein R₁ is 4-t-butyl, A1 is phenyl, L is ′—-(CH₂)₂—, A2 is phenyl, q is 0, R₂ is absent, A3 is 4-thiazol-4-yl, r is 1, and R₃ is 2-methyl;
a compound of Formula (Ia) wherein R₁ is 4-t-butyl, A1 is phenyl, L is —(CH₂)₂—, A2 is phenyl, q is 0, R₂ is absent, A3 is 4-pyrazol-1-yl, r is 2, and R₃ is 3,5-dimethyl
a compound of Formula (Ia) wherein R₁ is 4-t-butyl, A1 is phenyl, L is —(CH₂)₂—, A2 is phenyl, q is 0, R₂ is absent, A3 is 4-thiadiazol-4-yl, r is 0, and R₃ is absent;
a compound of Formula (Ia) wherein R₁ is 4-t-butyl, A1 is phenyl, L is —(CH2)₂—, A2 is phenyl, q is 0, R₂ is absent, A3 is 4-(1 ,2,4-triazol-1-yl), r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-1H-tetrazol-5-yl, r is 0, and R₃ is absent;
a compound of Formula (Ia) wherein R₁ is 4-t-butyl, A1 is phenyl, L is —(CH₂)₂—, A2 is phenyl, q is 0, R₂ is absent, A3 is 4-imidazol-3-yl, r is 2, and R₃ is 4,5-dichloro; and
a compound of Formula (Ia) wherein R₁ is 4-t-butyl, A1 is phenyl, L is —(CH₂)₂—, A2 is phenyl, q is 0, R₂ is absent, A3 is 4-(2,4-dihydro-pyrazol-2-yl), r is 2, and R₃ is 3-oxo, 5-methyl.

50. A pharmaceutical composition comprising a compound, salt or solvate according to claim 1 or 2 admixed with a pharmaceutically acceptable carrier, excipient or diluent.

51. A veterinary composition comprising a compound, salt or solvate according to claim 1 or 2 admixed with a veterinarily acceptable carrier, excipient or diluent.

52. A method of treating or preventing a disease or condition in a mammal in which the disease or condition is affected by the antagonism of vanilloid type 1 receptors, which method comprises administering to a mammal in need thereof a therapeutically effective amount of a compound of claim 1 or claim 2.

53. A method of treating or preventing a disease or condition in a mammal in which the disease or condition is affected by the agonism of vanilloid type 1 receptors, which method comprises administering to a mammal in need thereof a therapeutically effective amount of a compound of claim 1 or 2.

54. The method of claim 52 or 53 wherein the disease or condition is selected from the group consisting of osteoarthritis, rheumatoid arthritis, fibromyalgia, migraine, headache, toothache, regulation of fever, burn, sunburn, snake bite (in particular, venomous snake bite), spider bite, insect sting, neurogenic bladder, urinary incontinence, benign prostatic hypertrophy, interstitial cystitis, urinary tract infection, cough, asthma, chronic obstructive pulmonary disease, rhinitis, contact dermatitis/hypersensitivity, itch, eczema, anxiety, panic disorders, pharyngitis, mucositis, enteritis, cellulites, peripheral neuropathy, bilateral - peripheral neuropathy, diabetic neuropathy, central pain, neuropathies associated with spinal cord injury, stroke, ALS, Parkinson's disease, or multiple sclerosis, postherpetic neuralgia, trigeminal neuralgia, causalgia, sciatic neuritis, mandibular joint neuralgia, peripheral neuritis, polyneuritis, stump pain, phantom limb pain, bony fractures, post-operative ileus, irritable bowel syndrome, inflammatory bowel diseases such as Crohn's Disease and ulcerative colitis, cholecystitis, pancreatitis, postmastectomy pain syndrome, menstrual pain, endometriosis, dysmenorrhea, oral neuropathic pain, Charcot's pain, radiculopathy, complex regional pain syndrome I and II (CRPS I/II), Guillain-barre syndrome, meralgia paresthetica, burning-mouth syndrome, optic neuritis, postfebrile neuritis, migrating neuritis, segmental neuritis, Gombault's neuritis, neuronitis, cervicobrachial neuralgia, cranial neuralgia, geniculate neuralgia, glossopharyngial neuralgia, migrainous neuralgia, idiopathic neuralgia, intercostals neuralgia, mammary neuralgia, Morton's neuralgia, nasociliary neuralgia, occipital neuralgia, red neuralgia, Sluder's neuralgia, splenopalatine neuralgia, supraorbital neuralgia, vidian neuralgia, sinus headache, tension headache, labor, childbirth, intestinal gas, menstruation, hot flash, cancer, pain associated with bone cancer, and trauma.

55. The method of claim 52 or 53 wherein the condition is caused by a disease selected from the group consisting of inflammatory pain, neuropathic pain, visceral pain, pain associated with inflammatory bowel disease, colitis, mechanical hyperalgesia, LPS-induced fever, pyresis, inflammatory bronchial conditions, cough, anxiety, and panic disorders.

56. The method of claim 53 wherein said therapeutically effective amount comprises a dose range of from about 0.1 mg to about 1,000 mg.

57. The method of claim 53 wherein said therapeutically effective amount comprises a dose range of from about 50 mg to about 1000 mg.

58. The method of claim 53 wherein said therapeutically effective amount comprises a dose range of from about 100 mg to about 1000 mg.
